(19) United States
(12) Patent Application Publication
(10) Pub. No.: US 2016/0324856 A1
(43) Pub. Date: Nov. 10, 2016

(54) USE OF SGC STIMULATORS FOR THE TREATMENT OF NEUROMUSCULAR DISORDERS

(71) Applicant: IRONWOOD PHARMACEUTICALS, INC., Cambridge, MA (US)

(72) Inventors: Kimberly Kafadar LONG, Boston, MA (US); George Todd MILNE, Brookline, MA (US); Mark G. CURRIE, Boston, MA (US)

(21) Appl. No.: 15/110,883

(22) PCT Filed: Jan. 13, 2015
(86) PCT No.: PCT/US2015/011200

§ 371 Date: Jul. 11, 2016

Related U.S. Application Data
(60) Provisional application No. 61/926,897, filed on Jan. 13, 2014.

(51) Int. Cl. A61K 31/506; A61B 5/026; A61B 5/00; A61K 45/06; A61K 31/519

(52) U.S. Cl. CPC A61K 31/506; A61K 45/06; A61K 31/519; A61B 5/4848; A61B 5/026

(57) ABSTRACT

The present disclosure relates to methods, uses, pharmaceutical compositions and kits comprising a sGC stimulator or a pharmaceutically acceptable salt thereof, alone or in combination with one or more additional therapeutic agents, for the treatment of a neuromuscular disorder associated with loss or alteration of function of nitric oxide synthase (NOS).

### CROSS-REFERENCE TO RELATED APPLICATIONS

This application claims priority to U.S. Provisional Application No. 61/926,897, filed Jan. 13, 2014, which is herein incorporated by reference in its entirety.

### TECHNICAL FIELD

The present disclosure relates to methods of using soluble guanylate cyclase (sGC) stimulators and pharmaceutically acceptable salts thereof, alone or in combination with one or more additional therapeutic agents, for the treatment of neuromuscular disorders associated with loss or alteration of function of nitric oxide synthase (NOS).

### BACKGROUND

### Neuromuscular Disorders

Neuromuscular disorders are those that affect the muscles and/or their direct nervous system control. They can be acquired or of genetic origin.

Among the neuromuscular disorders of genetic origin are, for instance, those associated with mutations in genes associated with the dystrophin glycoprotein complex (DGC) or with mutations in the dystrophin gene.

It has previously been shown that neuronal Nitric Oxide Synthase (nNOS) mislocalization from the sarcolemmal membrane to the sarcoplasm is observed in a broad range of neuromuscular conditions associated with impaired mobility status and catabolic stress. Thus, one tool for the evaluating muscle biopsies of patients with a variety of either acquired or inherited forms of neuromuscular disorders is the assessment of sarcolemmal localization of nNOS. It has been found that the level of nNOS at the sarcolemma correlates well with mobility and functional status (“Loss of sarcolemmal nNOS is common in acquired and inherited neuromuscular disorders”; E. L. Finanger Hedderick et al., Neurology, 2011, 76(11), 960-967).

nNOS Mislocalization in Mouse Models of Acquired Muscle Atrophy

Two mouse models have been described that demonstrate muscle atrophy without compromised mobility: high-dose corticosteroids therapy and short-term starvation. Mice treated with steroids or starved for 48 hours showed significant decreases in overall body mass and in normalized wet skeletal muscle mass. Morphometric analysis of skeletal muscle specimens of both models demonstrated muscle atrophy, as defined by a significant decrease in mean minimal Feret fiber diameter as compared to age-matched controls (n=5 for each group). Immunofluorescence staining for dystrophin, α-sarcoglycan, and α-1-syntrophin showed normal dystrophin localization suggestive of an intact DGC complex. However, both steroid-treated and starved mice showed absent or severely reduced sarcolemmal nNOS staining. Real-time PCR for NOS family proteins (nNOS, eNOS, iNOS) revealed no significant differences in expression levels of any of the 3 transcripts in steroid-treated mice (n=8 for each group). Moreover, Western blot analysis for nNOS, iNOS, and eNOS showed no differences in protein levels.

Starved mice exhibited a decrease of nNOS and iNOS transcript expression as compared to wild type mice (n=9 for controls, n=7 for starved). However, the protein level of nNOS, iNOS, and eNOS revealed no differences between control and starved mice (n=4 for each group). These data demonstrate that abnormal localization of nNOS occurs in mice with severe muscle atrophy even if overall mobility is preserved, supporting the notion that, in addition to impaired mobility, other triggers such as catabolic stress may be associated with sarcolemmal loss of nNOS.

Skeletal Muscle nNOS Localization is Maintained During Hibernation, Studies with Squirrels

Skeletal muscle specimens from hibernating 13-lined ground squirrels have been used to evaluate the impact of immobility and catabolic stress on nNOS localization in the context of maintained muscle homeostasis and integrity. These animals are obligate hibernating mammals that are protected against skeletal muscle atrophy during hibernation. Despite hibernating for 5 months with almost complete immobility and no caloric intake, sarcolemmal expression of nNOS is preserved. These data together with patient and mouse data indicate that biochemical control of nNOS localization is complex and, importantly, that preserved sarcolemmal nNOS may be significant in maintaining muscle homeostasis.

### Muscular Dystrophy

Muscular Dystrophy (MD) is a group of muscle diseases that weaken the musculoskeletal system and hamper locomotion. Muscular dystrophies are characterized by progressive skeletal muscle weakness, defects in muscle proteins, and the death of muscle cells and tissue.

In the 1860s, descriptions of boys who grew progressively weaker, lost the ability to walk, and died at an early age became more prominent in medical journals. In the following decade, French neurologist Guillaume Duchenne gave a comprehensive account of thirteen boys with the most common and severe form of the disease, which now carries his name—Duchenne Muscular Dystrophy (DMD).

Other major forms include Becker, limb-girdle, congenital, facioscapulohumeral, myotonic, oculopharyngeal, distal, and Emery-Dreifuss muscular dystrophies. Duchenne and Becker muscular dystrophies, being caused by a mutation of a gene located on the X chromosome, predominantly affect males, although females can sometimes have severe symptoms as well. Most types of MD are multi-system disorders with manifestations in body systems including the heart, gastrointestinal system, nervous system, endocrine glands, respiratory system, eyes and brain.

These conditions are generally inherited, and the different muscular dystrophies follow various inheritance patterns. However, de novo mutations of the dystrophin gene and nutritional defects (with no genetics history) at the prenatal stage are also possible in about 33% of people affected by DMD.

The main cause of the Duchenne and Becker types of muscular dystrophy is mutations in the dystrophin gene that lead to lower levels, altered, or absence of full-length dystrophin protein, resulting in disruption or decreased function of the dystrophin-associated protein complex.

Dystrophin protein is found at muscle fiber membrane (also called the sarcolemma); its helical nature allows it to act like a spring or shock absorber. Dystrophin stabilizes the plasma membrane by linking the actin (cytoskeleton) to the extracellular matrix through its interactions with dystroglycans present in the sarcolemma.

The absence of dystrophin results in contraction induced disruptions to the sarcolemma, resulting in repeated rounds of muscle degeneration. Sarcolemmal disruptions allow excess calcium to penetrate the cell, leading to mitochondrial dysfunction. In muscular dystrophy, mitochondrial dysfunction gives rise to an amplification of stress-induced cytosolic calcium signals and an amplification of stress-induced reactive-oxygen species (ROS) production. In a complex cascading process that involves several pathways and is not clearly understood, increased oxidative stress within the cell damages the sarcolemma and eventually results in the death of the cell. Muscle fibers undergo necrosis and are ultimately replaced with adipose and connective tissue.

The diagnosis of muscular dystrophy is based on the results of muscle biopsy, increased creatine phosphokinase (CpK3), electromyography, electrocardiography and DNA analysis. In addition, a physical examination and the patient's medical history will help the doctor determine the type of muscular dystrophy as specific muscle groups are generally affected by different types of muscular dystrophy. Often, the loss of muscle mass (wasting), may be hard to see because some types of muscular dystrophy cause a buildup of fat and connective tissue that makes the muscle appear larger. This is called pseudohypertrophy.

There is no known cure for muscular dystrophy. Physical therapy, occupational therapy, orthotic intervention (e.g., ankle-foot orthosis), speech therapy and orthopedic instruments (e.g., wheelchairs, standing frames and powered mobile arm supports) may be helpful. Inactivity (such as bed rest, sitting for long periods) and bodybuilding efforts to increase myofibrillar hypertrophy can worsen the disease.

The prognosis for people with muscular dystrophy varies according to the type and progression of the disorder. Some cases may be mild and progress very slowly over a normal lifespan, while others produce severe muscle weakness, functional disability, and loss of the ability to walk. Some children with muscular dystrophy die in infancy while others live into adulthood with only moderate disability. The muscles affected vary, but can be around the pelvis, shoulder, face or elsewhere. Muscular dystrophy can affect adults, but the more severe forms tend to occur in early childhood.

Duchenne muscular dystrophy (DMD) is the most common childhood form of muscular dystrophy; it generally affects only boys (with extremely rare exceptions), becoming clinically evident when a child begins walking. By age 10, the child may need braces for walking and by age 12, most patients are unable to walk. The life span of a typical DMD patient ranges from 15 to 51. In the early 1990s, researchers identified the gene for the protein dystrophin which, when absent, causes DMD. The amount of dystrophin correlates with the severity of the disease (i.e., the less dystrophin present, the more severe the phenotype). Sporadic mutations in this gene occur frequently, accounting for a third of cases. The remaining two-thirds of cases are inherited in a recessive pattern.

Becker muscular dystrophy (BMD) is a less severe variant of Duchene Muscular Dystrophy and is caused by the production of a truncated, but partially functional form of dystrophin. Survival is usually into old age and it almost always affects only boys. It is characterized by progressive skeletal muscle wasting.

### Muscular Dystrophy and the NO-Pathway

When healthy skeletal muscle is exercised, sarcolemmal nNOS derived NO attenuates local α-adrenergic vasoconstriction, thereby optimizing perfusion to meet the metabolic demands of the active muscle. This protective mechanism (termed functional sympatholysis) is lost in mdx mice (a model of BMD and DMD), nNOS null mice, and boys with DMD causing functional muscle ischemia. Repeated bouts of functional ischemia are believed to accelerate use-dependent injury of muscle fibers already weakened by dystrophin deficiency.

In a human study, Martin et al. (see “Tadalafil Alleviates Muscle Ischemia in Patients with Becker Muscular Dystrophy”; Elizabeth A. Martin et al., Sci. Transl. Med. 4, 162ra155 (2012); “Vascular-targeted therapies for Duchenne muscular dystrophy”; Ennen et al., Skeletal Muscle, 2013, 3:9) assessed exercise-induced attenuation of reflex sympathetic vasoconstriction in the muscles of 10 patients with BMD and 7-age matched healthy male controls. Reflex vasoconstriction was induced by simulated orthostatic stress and was measured as the forearm muscles were rested or lightly exercised in the form of rhythmic handgrip. First, the investigators showed that exercise-induced attenuation of reflex vasoconstriction was defective in 9 out of 10 patients with BMD in whom the common dystrophin mutations disrupt targeting of neuronal NO synthase (nNOS) to the muscle sarcolemma. Then, in a double-blind randomized placebo-controlled crossover trial, the authors showed that normal blood flow regulation was restored in eight of nine patients by a single oral dose of 20 mg of tadalafil, a specific PDE5 inhibitor.

In the mdx mouse, many features of the dystrophic phenotype can be improved by multiple strategies that boost NO signaling, including transgenic expression of nNOS, transgenic expression of dystrophin minigenes that restore sarcolemmal nNOS (and thereby restore functional sympatholysis), administration of the NOS substrate L-arginine, treatment with NO-donating drugs, and phosphodiesterase 5A (PDE5A) inhibition with the drug tadalafil or sildenafil. These PDE5A inhibitors, which prolong the halflife of guanosine 3′,5′-monophosphate (cGMP)—the downstream target of NO in vascular smooth muscle—were shown in the mdx mouse to alleviate muscle ischemia, as well as injury and fatigue, after a brief bout of exercise. Also, these drugs were shown to improve cardiac dynamics in mdx mice and to rescue dystrophic skeletal muscle and prolong survival in dystrophin-deficient zebrafish.

There remains a need for novel treatments for muscular dystrophies, including DMD and BMD.

The above findings support an essential role for sarcolemmal nNOS in modulating sympathetic vasoconstriction in exercising human skeletal muscles and suggests that targeting the aberrant NO pathway (for instance by using an sGC stimulator of the disclosure) is a novel useful therapeutic approach for treating neuromuscular disorders, such as for instance BMD and DMD, in humans.

### SUMMARY

In one aspect, the invention provides a method of treating a neuromuscular disorder associated with loss or alteration of function of nitric oxide synthase (NOS) in a patient in need thereof, comprising administering a therapeutically or prophylactically effective amount of an sGC stimulator, or pharmaceutically acceptable salt thereof, alone or in combination with a therapeutically or prophylactically effective amount of one or more additional therapeutic agents to said patient.

In a further aspect, the invention provides a use of a sGC stimulator or a pharmaceutically acceptable salt thereof, in the manufacture of a medicament for the treatment of a neuromuscular disorder associated with loss or alteration of function of nitric oxide synthase (NOS) in a patient in need thereof.

In another aspect, the invention provides pharmaceutical compositions comprising a sGC stimulator or a pharmaceutically acceptable salt thereof, for use in the treatment of a neuromuscular disorder associated with loss or alteration of function of nitric oxide synthase (NOS) in a patient in need thereof. In another aspect, the invention provides pharmaceutical compositions comprising a sGC stimulator, or a pharmaceutically acceptable salt thereof, in combination with one or more additional therapeutic agents, for use in the treatment of a neuromuscular disorder associated with loss or alteration of function of nitric oxide synthase (NOS) in a patient in need thereof.

In still a further aspect, the invention provides a kit comprising at least two separate unit dosage forms (A) and (B), wherein (A) is a therapeutic agent, a combination of more than one therapeutic agent, a pharmaceutically acceptable salt thereof, or a pharmaceutical composition thereof, and (B) is a sGC stimulator, a pharmaceutically acceptable salt thereof, or a pharmaceutical composition comprising an sGC stimulator or a pharmaceutically acceptable salt thereof for use in the treatment of a neuromuscular disorder associated with loss or alteration of function of nitric oxide synthase (NOS) in a patient in need thereof.

In some embodiments of the above methods, uses, pharmaceutical compositions and kits, said neuromuscular disorder is associated with mutations in a gene associated with the dystrophin glycoprotein complex (DGC) or with mutations in the dystrophin gene. In other embodiments, said neuromuscular disorder is Muscular Dystrophy. In some embodiments, said muscular dystrophy is Duchenne Muscular Dystrophy. In other embodiments, said muscular dystrophy is Becker Muscular Dystrophy.

### BRIEF DESCRIPTION OF THE DRAWINGS

FIG. 1 shows the results of a Laser Doppler Blood Flow evaluation after muscle stimulation in mdx mice with and without treatment with an sGC stimulator.

### DETAILED DESCRIPTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls. The compounds described herein may be defined by their chemical structures and/or chemical names. Where a compound is referred to by both a chemical structure and a chemical name, and the chemical structure and chemical name conflict, the chemical structure is determinative of the compound's identity.

Neuronal Nitric Oxide Synthase (nNOS) misslocalization from the sarcolemmal membrane to the sarcoplasm is observed in a broad range of non-dystrophic neuromuscular conditions associated with impaired motility status and catabolic stress. There is also a general reduction in the amount of nNOS present in dystrophic muscle.

One tool for the evaluation of muscle biopsies of patients with a variety of inherited and acquired forms of neuromuscular disorders is the assessment of sarcolemmal localization of nNOS. It has been found that the level of nNOS at the sarcolemma correlates with mobility and functional status.

During exercise of healthy skeletal muscle, sarcolemmal neuronal Nitric Oxide Synthase (nNOS) derived NO attenuates local α-adrenergic vasoconstriction, thereby optimizing perfusion to meet the metabolic demands of the active muscle. This protective mechanism (termed functional sympatholysis) is lost in mdx mice (a murine model of BMD and DMD), nNOS null mice (mice characterized by muscle ischemia, but that are not dystrophic), and boys with DMD, leading to functional muscle ischemia. Repeated bouts of functional ischemia accelerate use-dependent injury of muscle fibers which are already weakened by dystrophin deficiency.

Loss of the sympatholysis mechanism is associated with abnormalities in the enzyme nNOS and leads to decreases in the levels of “cyclic GMP,” which is necessary for proper function of those muscles. It has also been shown that nNOS misslocalization from the sarcolemmal membrane to the sarcoplasm is observed in a broad range of non-dystrophic neuromuscular conditions associated with impaired motility status and catabolic stress, even in the presence of dystrophin.

In the mdx mouse model, many features of the dystrophic phenotype have been shown to improve by multiple strategies that boost NO signaling, including transgenic expression of nNOS, transgenic expression of dystrophin minigenes that restore sarcolemmal nNOS (and thereby restore functional sympatholysis), administration of the NOS substrate α-arginine, treatment with NO-donating drugs, and phosphodiesterase 5A (PDE5A) inhibition with the drugs tadalafil or sildenafil. These PDE5A inhibitors, which prolong the halflife of guanosine 3′,5′-monophosphate (cGMP)—the downstream target of NO in vascular smooth muscle—were shown in the mdx mouse to alleviate muscle ischemia, as well as injury and fatigue, after a brief bout of exercise. Also, these drugs were shown to improve cardiac dynamics in mdx mice and to rescue dystrophic skeletal muscle and prolong survival in dystrophin-deficient zebrafish.

In a related human study, Martin et al. (see “Tadalafil Alleviates Muscle Ischemia in Patients with Becker Muscular Dystrophy”; Elizabeth A. Martin et al., Sci. Transl. Med. 4, 162ra155 (2012); “Vascular-targeted therapies for Duchenne Muscular Dystrophy”; Ennen et al., Skeletal Muscle, 2013, 3:9) assessed exercise-induced attenuation of reflex sympathetic vasoconstriction in the muscles of 10 patients with BMD and 7-age matched healthy male controls. Reflex vasoconstriction was induced by simulated orthostatic stress and was measured as the forearm muscles were rested or lightly exercised in the form of rhythmic handgrip. First, the investigators showed that exercise-induced attenuation of reflex vasoconstriction was defective in 9 out of 10 patients with BMD in whom the common dystrophin mutations disrupt targeting of nNOS to the muscle sarcolemma. Then, in a double-blind randomized placebo-controlled crossover trial, the authors showed that normal blood flow regulation was restored in eight of nine patients by a single oral dose of 20 mg of tadalafil, a specific PDE5 inhibitor.

These findings support an essential role for sarcolemmal nNOS in modulating sympathetic vasoconstriction in exercising human skeletal muscles and suggests that targeting the aberrant NO pathway (for instance by using an sGC stimulator of the disclosure) is a novel and useful therapeutic approach for treating BMD and DMD in humans.

### Therapeutic Methods

As used herein, the terms “subject” and “patient” are used interchangeably to refer to an animal (e.g., a bird such as a chicken, quail or turkey, or a mammal), preferably a “mammal” including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject or patient is a human.

As used herein, the term a “patient in need thereof” is used to refer to a patient suffering from a neuromuscular disorder associated with loss or alteration of the function of nitric oxide synthase (NOS) or a neuromuscular disease associated with a mutation in any of the genes associated with dystrophin glycoprotein complex (DGC) or a neuromuscular disease associated with a mutation in the dystrophin gene. In some embodiments, the “patient in need thereof” is a patient with Muscular Dystrophy (MD) or who has been diagnosed with a Muscular Dystrophy or who is genetically predisposed to the development of a Muscular Dystrophy. In some embodiments, a patient in need thereof is a person that has been diagnosed with Duchenne Muscular Dystrophy (DMD). In other embodiments, a patient in need thereof is a person that has been diagnosed with Becker Muscular Dystrophy (BMD). In still other embodiments a patient in need thereof is a person (usually a child, sometimes an infant; most frequently a male child or infant) that has been genetically tested and found to have a mutation in the dystrophin gene that predisposes him or her to the development of a Muscular Dystrophy, even though he may not show any physical symptoms of MD yet.

Typical symptoms of most forms of muscular dystrophy include progressive muscular wasting, poor balance, drooping eyelids, atrophy, scoliosis (curvature of the spine and the back), inability to walk, frequent falls, waddling gait, calf deformation, limited range of movement, respiratory difficulty, joint contractures, cardiomyopathy, arrhythmias and muscle spasms.

The main symptom of Duchenne Muscular Dystrophy is muscle weakness associated with muscle wasting with the voluntary muscles being first affected, especially the muscles of the hips, pelvic area, thighs, shoulders, and calf muscles. Muscle weakness also occurs in the arms, neck, and other areas, but not as early as in the lower half of the body. Calves are often enlarged. Symptoms usually appear before age 6 and may appear as early as infancy. Problems with muscles in the upper part of the body (e.g., intercostals and diaphragm) are generally manifested as respiratory difficulties.

Other physical symptoms of DMD include but are not limited to: awkward manner of walking, stepping, or running (patients tend to walk on their forefeet, because of an increased calf tonus; toe walking is a compensatory adaptation to knee extensor weakness); frequent falls; fatigue; difficulty with motor skills (e.g., running, hopping and jumping); increased lumbar lordosis, leading to shortening of the hip-flexor muscles which has an effect on overall posture and the manner of walking, stepping, or running; muscle contractures of Achilles tendon and hamstrings; impaired functionality because the muscle fibers shorten and fibrosis occurs in connective tissue; progressive difficulty walking; muscle fiber deformities; pseudohypertrophy or enlarging of tongue and calf muscles (calf enlargement often happens during the ages of 5-15, and the muscle tissue is eventually replaced by fat and connective tissue as the legs become less used, hence the term pseudohypertrophy); use of Gower's maneuver to raise from the floor; higher risk of neurobehavioral disorders (e.g., ADHD), learning disorders (dyslexia), and non-progressive weaknesses in specific cognitive skills (in particular short-term verbal memory), which are believed to be the result of absent or dysfunctional dystrophin in the brain; eventual loss of ability to walk (usually by the age of 12); skeletal deformities (including scoliosis); and cardiomyopathy.

People with Becker Muscular Dystrophy typically experience progressive muscle weakness of the leg and pelvis muscles, which is associated with a loss of muscle mass (wasting). Muscle weakness also occurs in the arms, neck, and other areas, but is not as noticeably severe as in the lower half of the body. Calf muscles initially enlarge during the ages of 5-15 (an attempt by the body to compensate for loss of muscle strength), but the enlarged muscle tissue is eventually replaced by fat and connective tissue (pseudohypertrophy) as the legs become less used. BMD is typically less severe than DMD and patients frequently survive into adulthood. In addition, the phenotype of BMD is usually much more heterogeneous than that of DMD.

Additional symptoms of BMD may also include but are not limited to: muscle weakness; slowly progressive difficulty running, hopping, jumping; difficulty walking (however, ability to walk may or may not continue well into adulthood); severe upper extremity and trunk muscle weakness; toe-walking or walking on toes (also known as equinus); use of Gower's Maneuver or a modified form of Gower's Maneuver to get up from floor; frequent falls; difficulty breathing; skeletal deformities, for instance of chest and back (scoliosis); muscle deformities (e.g., contractions of heels, legs, pseudohypertrophy of calf muscles); fatigue; heart disease, particularly dilated cardiomyopathy; and elevated creatine phosphokinase (CPK) levels in blood.

Furthermore, muscle contractions, which may be painful, occur in the legs and heels of DMD and BMD patients, causing inability to use the muscles because of shortening of muscle fibers and fibrosis of connective tissue. Bones may also develop abnormally, causing skeletal deformities of the chest and other areas in both DMD and BMD.

As used herein, the term “treat”, “treating” or “treatment” with regard to a disorder or disease refers to alleviating or abrogating the cause and/or effects or symptoms of the disorder or disease. As used herein, the terms “treat”, “treatment” and “treating” refer to the reduction or amelioration or slowing down of the progression, severity and/or duration of a neuromuscular disorder (e.g., a Muscular Dystrophy), or the reduction, amelioration or slowing down of the progression, the severity and/or the duration of one or more symptoms (preferably, one or more measurable symptoms) of the condition, as a result of the administration of one or more therapies (e.g., an sGC stimulator or a pharmaceutically acceptable salt thereof, either alone or in combination therapy). In some embodiments, the terms “treat,” “treatment” and “treating” refer to delaying the onset of a symptom or set of symptoms or to delaying the onset of a loss in certain physical function (e.g., muscular function, walking). In some embodiments, the terms “treat,” “treatment” and “treating” refer to the amelioration of at least one measurable physical parameter of a neuromuscular disorder (e.g., a Muscular Dystrophy). In other embodiments the terms “treat”, “treatment” and “treating” refer to the reduction, inhibition or slowing down of the progression of said condition, either physically by, e.g., stabilization of a measurable symptom (e.g., fatigue), or physiologically by, e.g., stabilization of a measurable parameter (e.g., skeletal Troponin I levels), or both. As used herein, the term “treating”, “treat” or “treatment” also refer to averting the cause and/or effects of a disease or disorder or one of the symptoms developed as a result of the disease or disorder prior to the disease or disorder fully manifesting itself.

In one aspect, the invention provides a method of treating a neuromuscular disorder associated with loss or alteration of function of nitric oxide synthase (NOS) in a patient in need thereof, comprising administering a therapeutically or prophylactically effective amount of an sGC stimulator, or pharmaceutically acceptable salt thereof, alone or in combination with a therapeutically or prophylactically effective amount of one or more additional therapeutic agents to said patient.

In a further aspect, the invention provides a use of an sGC stimulator or a pharmaceutically acceptable salt thereof, in the manufacture of a medicament for the treatment of a neuromuscular disorder associated with loss or alteration of function of nitric oxide synthase (NOS) in a patient in need thereof.

In another aspect, the invention provides pharmaceutical compositions comprising a sGC stimulator or a pharmaceutically acceptable salt thereof, for use in the treatment of a neuromuscular disorder associated with loss or alteration of function of nitric oxide synthase (NOS) in a patient in need thereof. In another aspect, the invention provides pharmaceutical compositions comprising an sGC stimulator, or a pharmaceutically acceptable salt thereof, in combination with one or more additional therapeutic agents, for use in the treatment of a neuromuscular disorder associated with loss or alteration of function of nitric oxide synthase (NOS) in a patient in need thereof.

In still a further aspect, the invention provides a kit comprising at least two separate unit dosage forms (A) and (B), wherein (A) is a therapeutic agent, a combination of more than one therapeutic agent, a pharmaceutically acceptable salt thereof, or a pharmaceutical composition thereof, and (B) is an sGC stimulator, a pharmaceutically acceptable salt thereof, or a pharmaceutical composition comprising an sGC stimulator or a pharmaceutically acceptable salt thereof, for use in the treatment of a neuromuscular disorder associated with loss or alteration of function of nitric oxide synthase (nNOS) in a patient in need thereof.

In some embodiments of the above methods, uses, pharmaceutical compositions and kits, said neuromuscular disorder is associated with mutations in a gene associated with the dystrophin glycoprotein complex (DGC) or with mutations in the dystrophin gene. In other embodiments, said neuromuscular disorder is Muscular Dystrophy. In some embodiments, said muscular dystrophy is Duchenne Muscular Dystrophy. In other embodiments, said muscular dystrophy is Becker Muscular Dystrophy.

In some embodiments of the above methods, uses, compositions and kits, the patient in need thereof is an adult. In other embodiments the patient is a child or an infant. In some embodiments the patient is a male. In other embodiments the patient is a female.

In some embodiments of the above methods, uses, compositions and kits, the administration of an sGC stimulator or a pharmaceutically acceptable salt thereof, alone or in combination with another therapeutic agent, results in an observable or measurable decrease in the progression of muscle wasting. In other embodiments, it results in an observable or measurable increase in the degree of muscle function. In other embodiments, it results in an observable or measurable decrease in the degree of muscle necrosis. In still other embodiments, it results in an observable or measurable increase in muscle strength. In further embodiments, it results in an observable or measurable decrease in fatigue. In yet other embodiments, it results in an observable or measurable reduction in the risk of muscular injury. In yet other embodiments, it results in an observable or measurable reduction in the level of muscle fibrosis. In yet other embodiments, it results in an observable or measurable reduction in the rate of appearance of new muscle fibrosis. In yet other embodiments, it results in an observable or measurable reduction in the level of muscle or bone deformity. In still other embodiments, it results in an observable or measurable reduction in the rate of appearance of new muscle or bone deformity.

In some embodiments of the above methods, uses, compositions and kits, the observable or measurable decrease in the progression of muscle wasting, increase in the degree of muscle function, increase in muscle strength and/or reduction in the risk of muscular injury are measured by using a 6-minute walking distance test.

In some embodiments of the above methods, uses, compositions and kits, the observable or measurable decrease in the progression of muscle wasting, increase in the degree of muscle function, increase in muscle strength and/or reduction in the risk of muscular injury are measured by using a stair climbing test, in which the time necessary to climb a certain number of steps is measured (e.g., time to climb 4 stairs).

In some embodiments of the above methods, uses, compositions and kits, the observable or measurable decrease in the progression of muscle wasting, increase in the degree of muscle function, increase in muscle strength and/or reduction in the risk of muscular injury are measured by measuring the time required for going from seating to a standing position.

In some embodiments of the above methods, uses, compositions and kits, the observable or measurable decrease in the progression of muscle wasting, increase in the degree of muscle function, increase in muscle strength and/or reduction in the risk of muscular injury are measured by measuring the improvement in tissue blood flow after exercise or muscle stimulation.

In some embodiments of the above methods, uses, compositions and kits, the observable or measurable decrease in the level of fatigue is determined by using a 6-minute walking distance test.

In some embodiments of the above methods, uses, compositions and kits, the observable or measurable decrease in the level of fatigue is determined by using a stair climbing test, in which the time necessary to climb a certain number of steps is measured (e.g., time to climb 4 stairs).

In some embodiments of the above methods, uses, compositions and kits, the observable or measurable decrease in the level of fatigue is determined by measuring the time required for going from seating to a standing position.

In some embodiments of the above methods, uses, compositions and kits, the observable or measurable decrease in the level of fatigue is determined by the improvement in tissue blood flow after exercise or muscle stimulation.

In some embodiments of the above methods, uses, compositions and kits, the administration of an sGC stimulator, or a pharmaceutically acceptable salt thereof, or a pharmaceutical composition comprising an sGC stimulator or a pharmaceutically acceptable salt thereof, alone or in combination with another therapeutic agent, results in the improvement or reduction, or slowing down in the development of one or more symptoms selected from: progressive muscular wasting; progressive muscle weakness; poor balance; drooping eyelids; atrophy; skeletal deformities; scoliosis (curvature of the spine and the back); awkward manner of walking, stepping or running; difficulty with motor skills; lumbar lordosis, worsening posture, inability to walk or difficulty walking, running, jumping or hopping; frequent falls, waddling gait, calf deformation, use of Gower's maneuver to raise from the floor, pseudohypertrophy, limited range of movement, respiratory difficulty, joint or muscle contractures, muscle fiber shortening, fibrosis, cardiomyopathy, arrhythmias, muscle spasms or elevated levels of CPK in blood. In other embodiments the symptom is progressive muscular wasting. In still other embodiments, the symptom is progressive muscular wasting associated with muscular ischemia. In still other embodiments the symptom is muscular injury. In still other embodiments the symptom is fatigue.

In some embodiments of the above methods, uses, compositions and kits, the administration of an sGC stimulator or a pharmaceutically acceptable salt thereof, or a pharmaceutical composition comprising an sGC stimulator or a pharmaceutically acceptable salt thereof, alone or in combination with another therapeutic agent, to a patient in need thereof, is aimed at treating one or more symptoms selected from: progressive muscular wasting, progressive muscle weakness, poor balance, drooping eyelids, atrophy, skeletal deformities, scoliosis (curvature of the spine and the back), awkward manner of walking, stepping or running; difficulty with motor skills, lumbar lordosis, worsening posture, inability to walk or difficulty walking, running, jumping or hopping; frequent falls, waddling gait, calf deformation, pseudohypertrophy, limited range of movement, respiratory difficulty, joint or muscle contractures, muscle fiber shortening, fibrosis, cardiomyopathy, arrhythmias, muscle spasms or elevated levels of CPK in blood. In other embodiments the symptom is progressive muscular wasting. In still other embodiments, the symptom is progressive muscular wasting associated with muscular ischemia. In still other embodiments the symptom is muscular injury. In still other embodiments the symptom is fatigue.

### sGC Stimulators

In some embodiments of the above methods, uses, pharmaceutical compositions and kits, the sGC stimulator is selected from those described in patent application publications WO2013101830 (e.g., any one of compounds 1 to 122), WO2012064559 (e.g., any one of compounds I-1 to I-68), WO2012003405 (e.g., any one of compounds I-1 to I-312), WO2011115804 (e.g., any one of compounds I-1 to I-63), WO2014047111 (e.g., any one of compounds I-1 to I-5), WO2014/047325 (e.g., any one of compounds I-1 to I-10) or is a pharmaceutically acceptable salt thereof.

In other embodiments of the above methods, uses, pharmaceutical compositions and kits, the sGC stimulator is a compound described in one or more of the following publications: US20140088080 (WO2012165399), WO2014084312, U.S. Pat. No. 6,414,009, U.S. Pat. No. 6,462,068, U.S. Pat. No. 6,387,940, U.S. Pat. No. 6,410,740 (WO 98 16507), U.S. Pat. No. 6,451,805 (WO 98 23619), U.S. Pat. No. 6,180,656 (WO 98 16223), US20040235863 (WO2003004503), US 20060052397, U.S. Pat. No. 7,173,037 (WO2003095451), US 20060167016, U.S. Pat. No. 7,091,198 (WO2004009589), US 20060014951, U.S. Pat. No. 7,410,973 (WO2004009590), US 20100004235 (WO2007124854, e.g., Examples 1, 2, 3, 6, 7, 18 or 19), US20100029653 (WO 2008031513, e.g., Examples 1, 2, 3, 4 or 7), US20100113507 (WO2007128454, e.g, Example 1, 4 or 7), US 20110038857, U.S. Pat. No. 8,114,400 (WO2008061657), US20110218202 (WO 2010065275, e.g., Examples 1, 3, 59, 60 or 111), US20110245273 (WO 2010078900, e.g., Examples 1 or 5), US2012029002 (WO 2010079120), US20120022084, US 20130237551, U.S. Pat. No. 8,420,656 (WO 2011147809, WO 2011147810), US20130210824 (WO2013104598), US20130172372 (WO2012004259, e.g., Examples 2, 3 or 4), US20130267548 (WO2012059549, e.g., Examples 1, 2, 7, 8 or 13), WO 2012143510 (e.g., Examples 1, 2, 3, 4, 5, 6, 7, 8, 9 or 10), WO2012004258 (e.g., Examples 1, 18, 19 or 27), WO2012152629 (e.g., Examples 11 or 12), WO2012152630 (e.g., Examples 1, 5, 8, 11, 15 or 19), WO2012010577 (e.g., Examples 3-1, 4, 5 or 6), WO2012028647 (e.g., Examples 1, 2 or 3), WO2013104597 (e.g., Examples 16, 18, 22 or 23), WO2013131923 (e.g., Examples 1, 2, 7, 8 or 9), WO2013104703, WO2013004785 (e.g., Examples 1, 3 or 6), WO2013030288, US20090209556, U.S. Pat. No. 8,455,638, US20110118282 (WO2009032249), US20100292192, US20110201621, U.S. Pat. No. 7,947,664, U.S. Pat. No. 8,053,455 (WO2009094242), US20100216764, U.S. Pat. No. 8,507,512, (WO2010099054), US20110218202 (WO2010065275), US20130012511 (WO2011119518), US20130072492 (WO2011149921, e.g., Example #160, Example #164 and Example #181), US20130210798 (WO2012058132), U.S. Pat. No. 8,796,305 (WO2014068095), US20140128372 and US20140179672 (WO2014068099), U.S. Pat. No. 8,778,964 (US20140128386, US20140128424, WO2014068104), WO2014131741 and US20140249168 (WO2014131760).

In still other embodiments of the above methods, uses, pharmaceutical compositions and kits, the sGC stimulator is a compound according to Formula I′, or a pharmaceutically acceptable salt thereof,

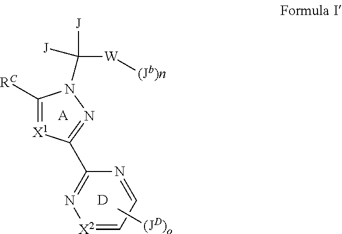

wherein X¹ is selected from N, CH, C(C₁₋₄ alkyl), C(C₁₋₄ haloalkyl), CCl and CF;
X² is independently selected from N or C;
W is either
i) absent, with J^{B} connected directly to the carbon atom bearing two J groups, each J is independently selected from hydrogen or methyl, n is 1 and J^{B} is a C₁₋₇ alkyl chain optionally substituted by up to 9 instances of fluorine; wherein, optionally, one —CH₂— unit of said C₁₋₇ alkyl chain can be replaced by —O— or —S—.
ii) a ring B that is a phenyl or a 5 or 6-membered heteroaryl ring, containing 1 or 2 ring heteroatoms selected from N, O or S; wherein with ring B being the phenyl or 5 or 6-membered heteroaryl ring; each J is hydrogen; n is an integer selected from 0 to 3; and each J^{B} is independently selected from halogen, —CN, a C₁₋₆ aliphatic, —OR^{B} or a C₃₋₈ cycloaliphatic group; wherein each said C₁₋₆ aliphatic and each said C₃₋₈ cycloaliphatic group is optionally and independently substituted with up to 3 instances of R³; each R^{B} is independently selected from hydrogen, a C₁₋₆ aliphatic or a C₃₋₈ cycloaliphatic; wherein each said R^{B} that is a C₁₋₆ aliphatic and each said R^{B} that is a C₃₋₈ cycloaliphatic ring is optionally and independently substituted with up to 3 instances of R^{3a};
each R³ is independently selected from halogen, —CN, C₁₋₄ alkyl, C₁₋₄ haloalkyl, —O(C₁₋₄ alkyl) or —O(C₁₋₄ haloalkyl);
each R^{3a} is independently selected from halogen, —CN, C₁₋₄ alkyl, C₁₋₄ haloalkyl, —O(C₁₋₄ alkyl) or —O(C₁₋₄ haloalkyl);
o is an integer selected from 1, 2 and 3;
each J^{D} is independently selected from J^{A}, halogen, —CN, —NO₂, —OR^{D}, —SR^{D}, —C(O)R^{D}, —C(O)OR^{D}, —OC(O)R^{D}, —C(O)N(R^{D})₂, —N(R^{D})₂, —N(R^{d})C(O)R^{D}, —N(R^{d})C(O)OR^{D}, —N(R^{d})C(O)N(R^{D})₂, —OC(O)N(R^{D})₂, —SO₂R^{D}, —SO₂N(R^{D})₂, —N(R^{d})SO₂R^{D}, a C₁₋₆ aliphatic, —(C₁₋₆ aliphatic)-R^{D}, a C₃₋₈ cycloaliphatic ring, a 6 to 10-membered aryl ring, a 4 to 8-membered heterocyclic ring or a 5 to 10-membered heteroaryl ring; wherein each said 4 to 8-membered heterocylic ring and each said 5 to 10-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{D} moiety, each said C₃₋₈ cycloaliphatic ring, each said 6 to 10-membered aryl ring, each said 4 to 8-membered heterocyclic ring and each said 5 to 10-membered heteroaryl ring is optionally and independently substituted with up to 5 instances of R^{5d};
J^{A} is selected from hydrogen, halogen, methyl, hydroxyl, methoxy, trifluoromethyl, trifluoromethoxy or —NR^{a}R^{b}; wherein R^{a} and R^{b} are each independently selected from hydrogen, C₁₋₆ alkyl or a 3-6 cycloalkyl ring; or wherein R^{a} and R^{b}, together with the nitrogen atom to which they are both attached, form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 6 instances of fluorine;
each R^{D} is independently selected from hydrogen, a C₁₋₆ aliphatic, —(C₁₋₆ aliphatic)-R^{f}, a C₃₋₈ cycloaliphatic ring, a 4 to 10-membered heterocyclic ring, phenyl or a 5 to 6-membered heteroaryl ring; wherein each said 4 to 10-membered heterocylic ring and each said 5 to 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 10-membered heterocyclic ring, each said phenyl and each said 5 to 6-membered heteroaryl ring is optionally and independently substituted with up to 5 instances of R^{5a}; wherein when any R^{D} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{d})—, —CO— or —O—; provided that when X¹ is one of CH, C(C₁₋₄ alkyl), C(C₁₋₄ haloalkyl), CCl or CF; X² is C; and at least one J^{D} is —N(R^{D})₂ and is attached to one of the pyrimidine ring D carbons ortho to the two nitrogen atoms of said ring D, one instance of R^{D} is not a pyridine or a pyrimidine;
each R^{d} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocylic ring and each said 5 or 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5b}; wherein when any R^{D} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{d})—, —CO— or —O—;
each R^{f} is independently selected from a C₁₋₃ alkyl, a C₃₋₈ cycloaliphatic ring, a 4 to 10-membered heterocyclic ring, phenyl or a 5 to 6-membered heteroaryl ring; wherein each said 4 to 10-membered heterocylic ring and each said 5 to 6-membered heteroaryl ring contains between 1 and 4 heteroatoms independently selected from O, N or S; and wherein each said C₃₋₈ cycloaliphatic ring, each said 4 to 10-membered heterocyclic ring, each said phenyl and each said 5 to 6-membered heteroaryl ring is optionally and independently substituted by up to 5 instances of R^{5c};
when J^{D} is —C(O)N(R^{D})₂, —N(R^{D})₂, —N(R^{d})C(O)N(R^{D})₂, —OC(O)N(R^{D})₂ or —SO₂N(R^{D})₂, the two R^{D} groups together with the nitrogen atom attached to the two R^{D} groups may form a 4 to 8-membered heterocyclic ring or a 5-membered heteroaryl ring; wherein each said 4 to 8-membered heterocyclic ring and each said 5-membered heteroaryl ring optionally contains up to 3 additional heteroatoms independently selected from N, O or S, in addition to the nitrogen atom to which the two R^{D} groups are attached; and wherein each said 4 to 8-membered heterocyclic ring and each said 5-membered heteroaryl ring is optionally and independently substituted by up to 5 instances of R⁵;
when J^{D} is —N(R^{d})C(O)R^{D}, the R^{D} group together with the carbon atom attached to the R^{D} group, with the nitrogen atom attached to the R^{d} group, and with the R^{d} group may form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S, in addition to the nitrogen atom to which the R^{d} group is attached; and wherein each said 4 to 8-membered heterocyclic ring and each said 5-membered heteroaryl ring is optionally and independently substituted by up to 5 instances of R⁵;
when J^{D} is —N(R^{d})C(O)OR^{D}, the R^{D} group together with the oxygen atom attached to the R^{D} group, with the carbon atom of the —C(O)— portion of the —N(R^{d})C(O)OR^{D} group, with the nitrogen atom attached to the R^{d} group, and with said R^{d} group, may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})C(O)N(R^{D})₂, one of the R^{D} groups attached to the nitrogen atom, together with said nitrogen atom, and with the N atom attached to the R^{d} group and said R^{d} group may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})SO₂R^{D}, the R^{D} group together with the sulfur atom attached to the R^{D} group, with the nitrogen atom attached to the R^{d} group, and with said R^{d} group may combine to form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
each R⁵ is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of the —(C₁₋₆ alkyl)-R⁶ moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R⁵, attached to the same or different atoms of J^{D}, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —CONH₂, —OH or halogen; wherein R is hydrogen or a C₁₋₂ alkyl;
each R^{5a} and each R^{5b} is independently selected from halogen, —CN, C₁₋₆ alkyl, —(C₁₋₆ alkyl)R^{6a}, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₁₋₆ alkyl portion of the —(C₁₋₆ alkyl)R^{6a} moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5a} or two instances of R^{5b} attached to the same or different atoms of R^{D} or R^{d}, respectively, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
each R^{5c} is independently selected from halogen, —CN, C₁₋₆ alkyl, —(C₁₋₆ alkyl)-R^{6b}, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂OH, —SO₂NHOH, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR^{6b}, a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R^{6b} moiety, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5c} attached to the same or different atoms of R^{f}, together with said atom or atoms to which it is attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —CONH₂, —NR(CO)O(C₁₋₄ alkyl), —OH or halogen; wherein R is hydrogen or a C₁₋₂ alkyl;
each R^{5d} is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl, C₁₋₆ alkyl portion of the —(C₁₋₆ alkyl)-R⁶moiety, C₇₋₁₂ aralkyl, C₃₋₈ cycloalkyl ring, 4 to 7-membered heterocyclic ring, 5 or 6-membered heteroaryl ring or phenyl group is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5d} attached to the same or different atoms of J^{D}, together with said atom or atoms of J^{D} to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —C(O)NH₂, —OH or halogen; wherein R is hydrogen or a C₁₋₂ alkyl;
each R⁶ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆ alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S; wherein
two instances of R⁶ linked to the same nitrogen atom of R⁵ or R^{5d}, together with said nitrogen atom of R⁵ or R^{5d}, respectively, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6a} linked to a nitrogen atom of R^{5a} or R^{5b}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6b} linked to a nitrogen atom of R^{5c}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two J^{D} groups attached to two vicinal ring D atoms, taken together with said two vicinal ring D atoms, may form a 5 to 7-membered heterocycle or a 5-membered heteroaryl ring that is fused to ring D; wherein said 5 to 7-membered heterocycle or said 5-membered ring heteroaryl contains from 1 to 3 heteroatoms independently selected from N, O or S; and wherein said 5 to 7-membered heterocycle or said 5-membered heteroaryl ring is optionally and independently substituted by up to 3 instances of oxo or —(Y)—R⁹;
wherein Y is either absent or is a linkage in the form of a C₁₋₆ alkyl chain, optionally substituted by up to 6 instances of fluoro; and wherein when Y is said C₁₋₆ alkyl chain, up to 3 methylene units of this alkyl chain, can be replaced by a group selected from —O—, —C(O)— or —N((Y)—R⁹)—;
each R⁹ is independently selected from hydrogen, halogen, —CN, —OR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, C₃₋₆ cycloalkyl ring, a 4-8-membered heterocyclic ring, a phenyl ring or a 5-6 membered heteroaroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹;
each R¹⁰ is independently selected from hydrogen, a C₁₋₆ alkyl, —(C₁₋₆ alkyl)-R¹³, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R¹³ moiety, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of R^{11a};
each R¹³ is independently selected from a phenyl, a benzyl, a C₃₋₆ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each said phenyl, each of said benzyl, each said C₃₋₈ cycloalkyl group, each said 4 to 7-membered heterocyclic ring and each 5 or 6-membered heteroaryl ring is optionally and independently substituted with up to 3 instances of R^{11b};
each R¹¹ is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²;
each R^{11a} is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²; and
each R^{11b} is independently selected from halogen, C₁₋₆ alkyl, oxo, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²;
each R¹² is selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ fluoroalkyl) or oxo;
R^{C} is either
i) a ring C; or
ii) is selected from halogen, —CN, C₁₋₆ alkyl, —(C₁₋₆ alkyl)-R^{N}, —COR⁷, —C(O)OR⁷, —C(O)N(R⁷)₂, —N(R⁷)C(O)R⁷, —N(R⁷)C(O)OR⁷, —N(R⁷)C(O)N(R⁷)₂, —N(R⁷)₂, —SO₂R⁷, —SO₂N(R⁷)₂, —C(O)N(R⁷)SO₂R⁷, —SO₂N(R⁷)COOR⁷, —SO₂N(R⁷)C(O)R⁷ or —N(R⁷)SO₂R⁷; wherein each said C₁₋₆ alkyl, each C₁₋₆ alkyl portion of said —(C₁₋₆ alkyl)-R^{N}, is optionally and independently substituted with up to 6 instances of fluoro and up to 2 instances of —CN, —OR⁸, oxo, —N(R⁸)₂, —N(R⁸)C(O)R⁸, —N(R⁸)C(O)R⁸, —C(O)N(R⁸)₂, —N(R⁸)C(O)N(R⁸)₂, —SO₂R⁸, —SO₂N(R⁸)₂, —NHOR⁸, —SO₂N(R⁸)COOR⁸, —SO₂N(R⁸)C(O)R⁸, —N(R⁷)SO₂R⁸;
wherein each R⁷ is independently selected from hydrogen, C₁₋₆ alkyl, C₁₋₆ fluoroalkyl, a C₃₋₈ cycloalkyl ring, phenyl, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R⁸ is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{N} is independently selected from a phenyl ring, a monocyclic 5 or 6-membered heteroaryl ring, a monocyclic C₃₋₆ cycloaliphatic ring, or a monocyclic 4 to 6-membered heterocycle; wherein said monocyclic 5 or 6-membered heteroaryl ring or said monocyclic 4 to 6-membered heterocycle contain between 1 and 4 heteroatoms selected from N, O or S; wherein said monocyclic 5 or 6-membered heteroaryl ring is not a 1,3,5-triazinyl ring; and wherein said phenyl, said monocyclic 5 to 6-membered heteroaryl ring, said monocyclic C₃₋₆ cycloaliphatic ring, or said monocyclic 4 to 6-membered heterocycle is optionally and independently substituted with up to 6 instances of fluoro and/or up to 3 instances of J^{M};
each J^{M} is independently selected from —CN, a C₁₋₆ aliphatic, —OR^{M}, —SR^{M}, —N(R^{M})₂, a C₃₋₈ cycloaliphatic ring or a 4 to 8-membered heterocyclic ring; wherein said 4 to 8-membered heterocyclic ring contains 1 or 2 heteroatoms independently selected from N, O or S; wherein each said C₁₋₆ aliphatic, each said C₃₋₈ cycloaliphatic ring and each said 4 to 8-membered heterocyclic ring, is optionally and independently substituted with up to 3 instances of R^{7c};
each R^{M} is independently selected from hydrogen, a C₁₋₆ aliphatic, a C₃₋₈ cycloaliphatic ring or a 4 to 8-membered heterocyclic ring; wherein each said 4 to 8-membered heterocylic ring contains between 1 and 3 heteroatoms independently selected from O, N or S; and wherein
ring C is a phenyl ring, a monocyclic 5 or 6-membered heteroaryl ring, a bicyclic 8 to 10-membered heteroaryl ring, a monocyclic 3 to 10-membered cycloaliphatic ring, or a monocyclic 4 to 10-membered heterocycle; wherein said monocyclic 5 or 6-membered heteroaryl ring, said bicyclic 8 to 10-membered heteroaryl ring, or said monocyclic 4 to 10-membered heterocycle contain between 1 and 4 heteroatoms selected from N, O or S; wherein said monocyclic 5 or 6-membered heteroaryl ring is not a 1,3,5-triazinyl ring; and wherein said phenyl, monocyclic 5 to 6-membered heteroaryl ring, bicyclic 8 to 10-membered heteroaryl ring, monocyclic 3 to 10-membered cycloaliphatic ring, or monocyclic 4 to 10-membered heterocycle is optionally and independently substituted with up to p instances of J^{C}′; wherein p is 0 or an integer selected from 1 to 3;
each J^{C} is independently selected from halogen, —CN, —NO₂, a C₁₋₆ aliphatic, —OR^{H}, —SR^{H}, —N(R^{H})₂, a C₃₋₈ cycloaliphatic ring or a 4 to 8-membered heterocyclic ring; wherein said 4 to 8-membered heterocyclic ring contains 1 or 2 heteroatoms independently selected from N, O or S; wherein each said C₁₋₆ aliphatic, each said C₃₋₈ cycloaliphatic ring and each said 4 to 8-membered heterocyclic ring, is optionally and independently substituted with up to 3 instances of R^{7d}; or
alternatively, two J^{C} groups attached to two vicinal ring C atoms, taken together with said two vicinal ring C atoms, form a 5 to 7-membered heterocycle that is a new ring fused to ring C; wherein said 5 to 7-membered heterocycle contains from 1 to 2 heteroatoms independently selected from N, O or S;
each R^{H} is independently selected from hydrogen, a C₁₋₆ aliphatic, a C₃₋₈ cycloaliphatic ring or a 4 to 8-membered heterocyclic ring; wherein each said 4 to 8-membered heterocylic ring contains between 1 and 3 heteroatoms independently selected from O, N or S; alternatively, two instances of R^{H} linked to the same nitrogen atom of —N(R^{H})₂, together with said nitrogen atom of —N(R^{H})₂, form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S;
each R^{7c} is independently selected from hydrogen, halogen, —CN, —NO₂, C₁₋₄ alkyl, C₁₋₄ haloalkyl, C₃₋₈ cycloalkyl ring, —OR^{8b}, —SR^{8b}, —N(R^{8b})₂, —C(O)O(C₁₋₄ alkyl), —C(O)OH, —NR(CO)CO(C₁₋₄ alkyl) or an oxo group; wherein each said cycloalkyl group is optionally and independently substituted with up to 3 instances of halogen; wherein each R^{8b} is independently selected from hydrogen, a C₁₋₄ alkyl, C₁₋₄ haloalkyl, a C₃₋₈ cycloalkyl ring or a C₃₋₈ (halocycloalkyl) ring; and
each R^{7d} is independently selected from hydrogen, halogen, —CN, —NO₂, C₁₋₄ alkyl, C₁₋₄ haloalkyl, C₃₋₈ cycloalkyl ring, —OR^{8c}, —SR^{8c}, —N(R^{8c})₂, or an oxo group; wherein each said cycloalkyl group is optionally and independently substituted with up to 3 instances of halogen; wherein each R^{8c} is independently selected from hydrogen, a C₁₋₄ alkyl, C₁₋₄ haloalkyl, a C₃₋₈ cycloalkyl ring or a C₃₋₈ (halocycloalkyl) ring;
each R^{8b} is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{8c} is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; and provided that the compound is not a compound depicted below:

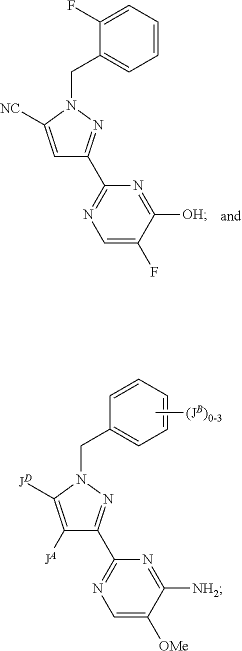

wherein J^{D} is either an ethylene or —N(Me)₂; J^{A} is either hydrogen or methyl and J^{B} is either fluoro or C₁₋₂ alkoxy.

In other embodiments of the above methods, uses, compositions and kits the sGC stimulator is a compound of Formula I′ or a pharmaceutically acceptable salt thereof, wherein W is absent. In other embodiments, it is a compound of Formula I′ represented by Formula II′a:

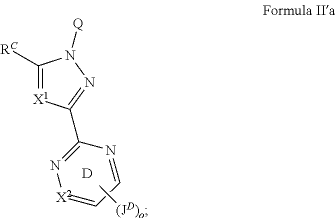

wherein Q represents a C₁₋₇ alkyl group, optionally substituted with up to 9 instances of fluorine.

In some embodiments of the above methods, uses, compositions and kits, the sGC stimulator is a compound of Formula II′a, or a pharmaceutically acceptable salt thereof, wherein Q is substituted with up to 5 instances of fluorine.

In some embodiments of the above methods, uses, compositions and kits, the sGC stimulator is a compound represented by Formula III′a:

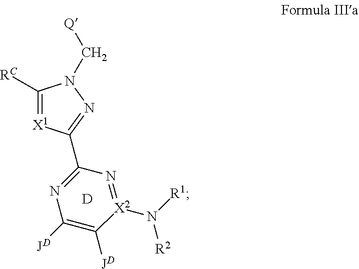

wherein,
Q′ is a C₁₋₆ alkyl chain, optionally substituted by up to 6 instances of fluorine;
when X² is N, the moiety —N(R¹)(R²) is absent;
when X² is C, the moiety —N(R¹)(R²) is present;
R¹ and R², together with the nitrogen atom to which they are attached, form a 4 to 8-membered heterocyclic ring or 5-membered heteroaryl ring; wherein said 4 to 8-membered heterocyclic ring or 5-membered heteroaryl ring optionally contains, in addition to the nitrogen atom to which R¹ and R² are attached, up to 3 ring heteroatoms independently selected from N, O or S, and is optionally substituted by up to 5 instances of R^{5e};
each R^{5e} is independently selected from halogen, —CN, C₁₋₆ alkyl, —(C₁₋₄ alkyl)-R⁶, a C₃₋₈ cycloalkyl ring, C₁₋₄ cyanoalkyl, —OR⁶, —SR⁶, —OCOR⁶, —COR⁶, —C(O)OR⁶, —C(O)N(R⁶)₂, —N(R⁶)C(O)R⁶, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, benzyl, phenyl or an oxo group; wherein each said phenyl ring and each said benzyl group, is optionally and independently substituted with up to 3 instances of halogen, —OH, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl); and wherein each said C₁₋₆ alkyl, each C₁₋₄ alkyl portion of said —(C₁₋₄ alkyl)-R⁶ moiety, and each said C₃₋₈ cycloalkyl ring is optionally and independently substituted with up to 3 instances of halogen; wherein
each R⁶ is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
two of the instances of R^{5e} attached to the same or different atoms of said ring formed by R¹, R² and the nitrogen to which R¹ and R² are attached, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
alternatively, R¹ and R² are each independently selected from hydrogen, C₁₋₆ alkyl, a C₃₋₈ cycloalkyl ring, a 4 to 8-membered heterocyclic ring, a 5 or 6-membered heteroaryl, phenyl or a C₁₋₆ alkyl-R^{Y}; wherein each of said 4 to 8-membered heterocyclic ring and each of said 5 or 6-membered heteroaryl ring contains up to 3 ring heteroatoms independently selected from N, O and S; and wherein each of said C₁₋₆ alkyl, C₁₋₆ alkyl portion of each said C₁₋₆ alkyl-R^{Y} moiety, C₃₋₈ cycloalkyl ring, 4 to 8-membered heterocyclic ring group, 5 or 6-membered heteroaryl, phenyl and C₁₋₆ alkyl-R^{Y} is optionally and independently substituted with up to 5 instances of R^{5f};
R^{Y} is selected from a C₃₋₈ cycloalkyl ring, a 4 to 8-membered heterocyclic ring, phenyl, or a 5 to 6-membered heteroaryl ring; wherein each of said 4 to 8-membered heterocyclic ring or 5 to 6-membered heteroaromatic ring contains between 1 and 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaryl ring is optionally substituted with up to 5 instances of R^{5g};
each R^{5f} is independently selected from halogen, —CN, C₁₋₆ alkyl, —(C₁₋₄ alkyl)-R^{6a}, a C₇₋₁₂ aralkyl, C₃₋₈ cycloalkyl ring, C₁₋₄ cyanoalkyl, —OR^{6a}, —SR^{6a}, —OCOR^{6a}, —COR^{6a}, —C(O)OR^{6a}, —C(O)N(R^{6a})₂, —N(R^{6a})C(O)R^{6a}, —N(R^{6a})₂, —SO₂R^{6a}, —SO₂N(R^{6a})₂, —N(R^{6a})SO₂R^{6a}, —SO₂OH, —SO₂NHOH, —SO₂N(R^{6a})COR^{6a}, phenyl or an oxo group; wherein each said phenyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₇₋₁₂ aralkyl, C₁₋₆ alkyl, C₁₋₄ alkyl portion of each said —(C₁₋₄ alkyl)-R^{6a} and each said C₃₋₈ cycloalkyl group is optionally and independently substituted with up to three instances of halogen;
   each R^{6a} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
   when one of R¹ or R² is the C₃₋₈ cycloalkyl ring, 4 to 8-membered heterocyclic ring or 5 or 6-membered heteroaryl substituted with up to 5 instances of R^{5f}, two of the instances of R^{5f} attached to the same or different ring atoms of said R¹ or R², together with said atom or atoms, form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)O(C₁₋₄ alkyl), —NR′(CO)O(C₁₋₄ alkyl) or halogen; wherein R′ is hydrogen or a C₁₋₂ alkyl;
   each R^{5g} is independently selected from halogen, —CN, C₁₋₆ alkyl, —(C₁₋₄ alkyl)-R^{6b}, a benzyl, C₃₋₈ cycloalkyl ring, C₁₋₄ cyanoalkyl, —OR^{6b}, —SR^{6b}, —OCOR^{6b}, —COR^{6b}, —C(O)OR^{6b}, —C(O)N(R^{6b})₂, —N(R^{6b})C(O)R^{6b}, —N(R^{6b})₂, —SO₂R^{6b}, —SO₂N(R^{6b})₂, —N(R^{6b})SO₂R^{6b}, —SO₂OH, —SO₂NHOH, —SO₂N(R^{6b})COR^{6b}, phenyl or an oxo group; wherein each said phenyl and each said benzyl group is optionally and independently substituted with up to 3 instances of halogen, —OH, —NH₂, —NH(C₁₋₄alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₁₋₆ alkyl, C₁₋₄ alkyl portion of each said (C₁₋₄ alkyl)-R^{6b} moiety and each said C₃₋₈ cycloalkyl group is optionally and independently substituted with up to 3 instances of halogen;
   each R^{6b} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
   alternatively, two instances of R^{5g} attached to the same or different ring atoms of R^{Y}, together with said ring atom or atoms, form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR″(CO)O(C₁₋₄ alkyl), —OH or halogen;
   R″ is hydrogen or a C₁₋₂ alkyl;
   the two J^{D} groups attached to two vicinal ring D atoms, taken together with said two vicinal ring D atoms, may optionally form a 5 to 6-membered heterocycle or a 5-membered heteroaryl ring that is fused to ring D; wherein said 5 to 6-membered heterocycle or said 5-membered ring heteroaryl contains from 1 to 3 heteroatoms independently selected from N, O or S; and wherein said 5 to 6-membered heterocycle or said 5-membered heteroaryl ring is optionally and independently substituted by up to 3 instances of oxo or —(Y)—R⁹.

In other embodiments of the above methods, uses, compositions and kits, the sGC stimulator is a compound of Formula III′a, or a pharmaceutically acceptable salt thereof, wherein at least one of the two instances of X¹ and X² is N. In other embodiments, only one instance of X¹ and X² is N and the other one is C with a substituent. In still other embodiments, X² is C on ring D and is optionally substituted with J^{D}.

In other embodiments of the above methods, uses, compositions and kits, the sGC stimulator is a compound represented by Formula IV′a:

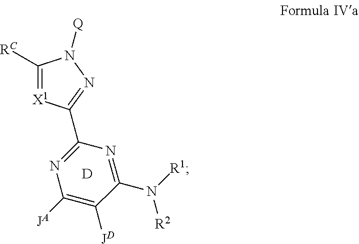

wherein, J^{A} is selected from hydrogen, halogen, methyl, hydroxyl, methoxy, trifluoromethyl, trifluoromethoxy or —NR^{a}R^{b}; wherein R^{a} and R^{b} are each independently selected from hydrogen, C₁₋₆ alkyl or a 3-6 cycloalkyl ring; or wherein R^{a} and R^{b}, together with the nitrogen atom to which they are both attached, form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 6 instances of fluorine; and
J^{D} is selected from hydrogen or fluorine;
R¹ and R², together with the nitrogen atom to which they are attached, form a 4 to 8-membered heterocyclic ring or 5-membered heteroaryl ring; wherein said 4 to 8-membered heterocyclic ring or 5-membered heteroaryl ring optionally contains, in addition to the nitrogen atom to which R¹ and R² are attached, up to 3 ring heteroatoms independently selected from N, O or S, and is optionally substituted by up to 5 instances of R^{5e};
each R^{5e} is independently selected from halogen, —CN, C₁₋₆ alkyl, —(C₁₋₄ alkyl)-R⁶, a C₃₋₈ cycloalkyl ring, C₁₋₄ cyanoalkyl, —OR⁶, —SR⁶, —OCOR⁶, —COR⁶, —C(O)OR⁶, —C(O)N(R⁶)₂, —N(R⁶)C(O)R⁶, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, benzyl, phenyl or an oxo group; wherein each said phenyl ring and each said benzyl group, is optionally and independently substituted with up to 3 instances of halogen, —OH, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl); and wherein each said C₁₋₆ alkyl, each C₁₋₄ alkyl portion of said —(C₁₋₄ alkyl)-R⁶ moiety, and each said C₃₋₈ cycloalkyl ring is optionally and independently substituted with up to 3 instances of halogen; wherein
each R⁶ is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
two of the instances of R^{5e} attached to the same or different atoms of said ring formed by R¹, R² and the nitrogen to which R¹ and R² are attached, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
alternatively, R¹ and R² are each independently selected from hydrogen, C₁₋₆ alkyl, a C₃₋₈ cycloalkyl ring, a 4 to 8-membered heterocyclic ring, a 5 or 6-membered heteroaryl, phenyl or a C₁₋₆ alkyl-R^{Y}; wherein each of said 4 to 8-membered heterocyclic ring and each of said 5 or 6-membered heteroaryl ring contains up to 3 ring heteroatoms independently selected from N, O and S; and wherein each of said C₁₋₆ alkyl, C₁₋₆ alkyl portion of each said C₁₋₆ alkyl-R^{Y} moiety, C₃₋₈ cycloalkyl ring, 4 to 8-membered heterocyclic ring group, 5 or 6-membered heteroaryl, phenyl and C₁₋₆ alkyl-R^{Y} is optionally and independently substituted with up to 5 instances of R^{5f};
R^{Y} is selected from a C₃₋₈ cycloalkyl ring, a 4 to 8-membered heterocyclic ring, phenyl, or a 5 to 6-membered heteroaryl ring; wherein each of said 4 to 8-membered heterocyclic ring or 5 to 6-membered heteroaromatic ring contains between 1 and 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaryl ring is optionally substituted with up to 5 instances of R^{5g};
each R^{5f} is independently selected from halogen, —CN, C₁₋₆ alkyl, —(C₁₋₄ alkyl)-R^{6a}, a C₇₋₁₂ aralkyl, C₃₋₈ cycloalkyl ring, C₁₋₄ cyanoalkyl, —OR^{a}, —SR^{6a}, —OCOR^{6a}, —COR^{6a}, —C(O)OR^{6a}, —C(O)N(R^{6a})₂, —N(R^{6a})C(O)R^{6a}, —N(R^{6a})₂, —SO₂R^{6a}, —SO₂N(R^{6a})₂, —N(R^{6a})SO₂R^{6a}, —SO₂OH, —SO₂NHOH, —SO₂N(R^{6a})COR^{6a}, phenyl or an oxo group; wherein each said phenyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₇₋₁₂ aralkyl, C₁₋₆ alkyl, C₁₋₄ alkyl portion of each said —(C₁₋₄ alkyl)-R^{6a} and each said C₃₋₈ cycloalkyl group is optionally and independently substituted with up to three instances of halogen;
each R^{6a} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
when one of R¹ or R² is the C₃₋₈ cycloalkyl ring, 4 to 8-membered heterocyclic ring or 5 or 6-membered heteroaryl substituted with up to 5 instances of R^{5f}, two of the instances of R^{5f} attached to the same or different ring atoms of said R¹ or R², together with said atom or atoms, form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)O(C₁₋₄ alkyl), —NR′(CO)O(C₁₋₄ alkyl) or halogen; wherein R′ is hydrogen or a C₁₋₂ alkyl;
each R^{5g} is independently selected from halogen, —CN, C₁₋₆ alkyl, —(C₁₋₄ alkyl)-R^{6b}, a benzyl, C₃₋₈ cycloalkyl ring, C₁₋₄ cyanoalkyl, —OR^{6b}, —SR^{6b}, —OCOR^{6b}, —COR^{6b}, —C(O)OR^{6b}, —C(O)N(R^{6b})₂, —N(R^{6b})C(O)R^{6b}, —N(R^{6b})₂, —SO₂R^{6b}, —SO₂N(R^{6b})₂, —N(R^{6b})SO₂R^{6b}, —SO₂OH, —SO₂NHOH, —SO₂N(R^{6b})COR^{6b}, phenyl or an oxo group; wherein each said phenyl and each said benzyl group is optionally and independently substituted with up to 3 instances of halogen, —OH, —NH₂, —NH(C₁₋₄alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₁₋₆ alkyl, C₁₋₄ alkyl portion of each said (C₁₋₄ alkyl)-R^{6b} moiety and each said C₃₋₈ cycloalkyl group is optionally and independently substituted with up to 3 instances of halogen;
each R^{6b} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
alternatively, two instances of R^{5g} attached to the same or different ring atoms of R^{Y}, together with said ring atom or atoms, form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or a 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR″(CO)O(C₁₋₄ alkyl), —OH or halogen;
R″ is hydrogen or a C₁₋₂ alkyl.

In other embodiments of the above methods, uses, compositions and kits, the sGC stimulator is a compound represented by Formula II′b, or a pharmaceutically acceptable salt thereof:

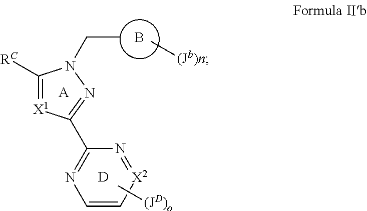

wherein, ring B is a phenyl or a 5 or 6-membered heteroaryl ring, containing 1 or 2 ring heteroatoms selected from N, O or S. In some embodiments of Formula II′b, X² on ring D is carbon, optionally substituted by J^{D}. In other embodiments, X² on ring D is nitrogen. In some embodiments of Formula II′b, each J^{D} is independently selected from J^{A}, halogen, a C₁₋₆ aliphatic, —N(R^{D})₂, —N(R^{d})COR^{D}, —N(R^{d})COOR^{D}, —OR^{D}, —N(R^{d})SO₂R^{D}, or an optionally substituted C₃₋₈ cycloaliphatic ring. In other embodiments, o is 2 and each J^{D} is independently selected from a halogen atom or —N(R^{D})₂, —N(R^{d})COR^{D}, —OH, —N(R^{d})COOR^{D} or —N(R^{d})SO₂R^{D}. In still other embodiments, o is 2 and one instance of J^{D} is fluoro or chloro and the other instance of J^{D} is —OH. In further embodiments, o is 2 and one instance of J^{D} is —NH₂ and the other one is independently selected from —N(R^{D})₂, —NHCOR^{D}, —N(R^{d})COOR^{D} or —N(R^{d})SO₂R^{D}, wherein at least one instance of R^{D} in —N(R^{D})₂ is not hydrogen. In even further embodiments, o is 2 and one instance of J^{D} is independently selected from —N(R^{D})₂ or —NHCOR^{D} and the other instance of J^{D} is selected from fluoro or chloro. In yet further embodiments, o is 1 and J^{D} is amino.

In other embodiments of the above methods, uses, compositions and kits, the sGC stimulator is a compound represented by one of Formula III′b or III′c:

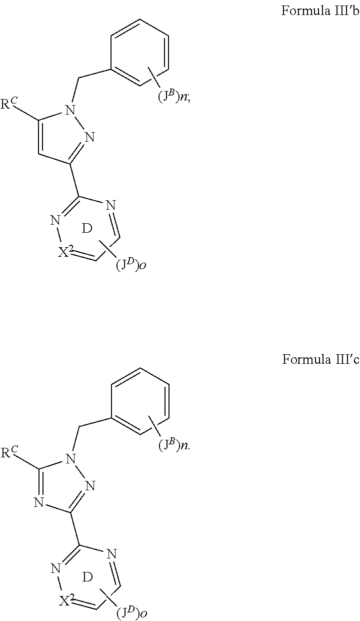

In other embodiments of the above methods, uses, compositions and kits, the sGC stimulator is a compound represented by Formula IV′b or Formula IV′c:

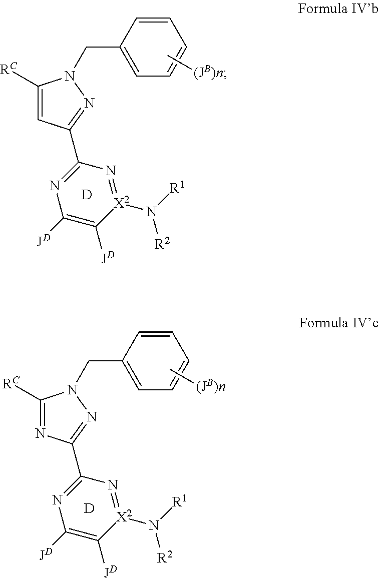

wherein, when X² is N, the moiety —N(R¹)(R²) is absent;
when X² is C, the moiety —N(R¹)(R²) is present;
R¹ and R², together with the nitrogen atom to which they are attached, form a 4 to 8-membered heterocyclic ring or 5-membered heteroaryl ring; wherein said 4 to 8-membered heterocyclic ring or 5-membered heteroaryl ring optionally contains, in addition to the nitrogen atom to which R¹ and R² are attached, up to 3 ring heteroatoms independently selected from N, O or S, and is optionally substituted by up to 5 instances of R^{5e};
each R^{5e} is independently selected from halogen, —CN, C₁₋₆ alkyl, —(C₁₋₄ alkyl)-R⁶, a C₃₋₈ cycloalkyl ring, C₁₋₄ cyanoalkyl, —OR⁶, —SR⁶, —OCOR⁶, —COR⁶, —C(O)OR⁶, —C(O)N(R⁶)₂, —N(R⁶)C(O)R⁶, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, benzyl, phenyl or an oxo group; wherein each said phenyl ring and each said benzyl group, is optionally and independently substituted with up to 3 instances of halogen, —OH, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl); and wherein each said C₁₋₆ alkyl, each C₁₋₄ alkyl portion of said —(C₁₋₄ alkyl)-R⁶ moiety, and each said C₃₋₈ cycloalkyl ring is optionally and independently substituted with up to 3 instances of halogen; wherein
each R⁶ is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
two of the instances of R^{5e} attached to the same or different atoms of said ring formed by R¹, R² and the nitrogen to which R¹ and R² are attached, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
alternatively, R¹ and R² are each independently selected from hydrogen, C₁₋₆ alkyl, a C₃₋₈ cycloalkyl ring, a 4 to 8-membered heterocyclic ring, a 5 or 6-membered heteroaryl, phenyl or a C₁₋₆ alkyl-R^{Y}; wherein each of said 4 to 8-membered heterocyclic ring and each of said 5 or 6-membered heteroaryl ring contains up to 3 ring heteroatoms independently selected from N, O and S; and wherein each of said C₁₋₆ alkyl, C₁₋₆ alkyl portion of each said C₁₋₆ alkyl-R^{Y} moiety, C₃₋₈ cycloalkyl ring, 4 to 8-membered heterocyclic ring group, 5 or 6-membered heteroaryl, phenyl and C₁₋₆ alkyl-R^{Y} is optionally and independently substituted with up to 5 instances of R^{5f}; provided that when the compound is of Formula IV′b; wherein X² is C; one instance of R¹ or R² is not a pyridine or a pyrimidine;
R^{Y} is selected from a C₃₋₈ cycloalkyl ring, a 4 to 8-membered heterocyclic ring, phenyl, or a 5 to 6-membered heteroaryl ring; wherein each of said 4 to 8-membered heterocyclic ring or 5 to 6-membered heteroaromatic ring contains between 1 and 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaryl ring is optionally substituted with up to 5 instances of R^{5g};
each R^{5f} is independently selected from halogen, —CN, C₁₋₆ alkyl, —(C₁₋₄ alkyl)-R^{6a}, a C₇₋₁₂ aralkyl, C₃₋₈ cycloalkyl ring, C₁₋₄ cyanoalkyl, —OR^{6a}, —SR^{6a}, —OCOR^{6a}, —COR^{6a}, —C(O)OR^{6a}, —C(O)N(R^{6a})₂, —N(R^{6a})C(O)R^{6a}, —N(R^{6a})₂, —SO₂R^{6a}, —SO₂N(R^{6a})₂, —N(R^{6a})SO₂R^{6a}, —SO₂OH, —SO₂NHOH, —SO₂N(R^{6a})COR^{6a}, phenyl or an oxo group; wherein each said phenyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₇₋₁₂ aralkyl, C₁₋₆ alkyl, C₁₋₄ alkyl portion of each said —(C₁₋₄ alkyl)-R^{6a} and each said C₃₋₈ cycloalkyl group is optionally and independently substituted with up to three instances of halogen;
each R^{6a} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
when one of R¹ or R² is the C₃₋₈ cycloalkyl ring, 4 to 8-membered heterocyclic ring or 5 or 6-membered heteroaryl substituted with up to 5 instances of R^{5f}, two of the instances of R^{5f} attached to the same or different ring atoms of said R¹ or R², together with said atom or atoms, form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)O(C₁₋₄ alkyl), —NR′(CO)O(C₁₋₄ alkyl) or halogen; wherein R′ is hydrogen or a C₁₋₂ alkyl;
each R^{5g} is independently selected from halogen, —CN, C₁₋₆ alkyl, —(C₁₋₄ alkyl)-R^{6b}, a benzyl, C₃₋₈ cycloalkyl ring, C₁₋₄ cyanoalkyl, —OR^{6b}, —SR^{6b}, —OCOR^{6b}, —COR^{6b}, —C(O)OR^{6b}, —C(O)N(R^{6b})₂, —N(R^{6b})C(O)R^{6b}, —N(R^{6b})₂, —SO₂R^{6b}, —SO₂N(R^{6b})₂, —N(R^{6b})SO₂R^{6b}, —SO₂OH, —SO₂NHOH, —SO₂N(R^{6b})COR^{6b}, phenyl or an oxo group; wherein each said phenyl and each said benzyl group is optionally and independently substituted with up to 3 instances of halogen, —OH, —NH₂, —NH(C₁₋₄alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₁₋₆ alkyl, C₁₋₄ alkyl portion of each said (C₁₋₄ alkyl)-R^{6b} moiety and each said C₃₋₈ cycloalkyl group is optionally and independently substituted with up to 3 instances of halogen;
each R^{6b} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
alternatively, two instances of R^{5g} attached to the same or different ring atoms of R^{Y}, together with said ring atom or atoms, form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR″(CO)O(C₁₋₄ alkyl), —OH or halogen;
R″ is hydrogen or a C₁₋₂ alkyl;
the two J^{D} groups attached to two vicinal ring D atoms, taken together with said two vicinal ring D atoms, may optionally form a 5 to 6-membered heterocycle or a 5-membered heteroaryl ring that is fused to ring D; wherein said 5 to 6-membered heterocycle or said 5-membered ring heteroaryl contains from 1 to 3 heteroatoms independently selected from N, O or S; and wherein said 5 to 6-membered heterocycle or said 5-membered heteroaryl ring is optionally and independently substituted by up to 3 instances of oxo or —(Y)—R⁹.

In other embodiments of the above methods, uses, compositions and kits, the sGC stimulator is a compound of Formula IV′b or IV′c, wherein X² is nitrogen and the moiety —NR₁R₂ is absent. In other embodiments, X² is carbon and the moiety —NR₁R₂ is present.

In other embodiments of the above methods, uses, compositions and kits, the sGC stimulator is a compound represented by Formula V′b:

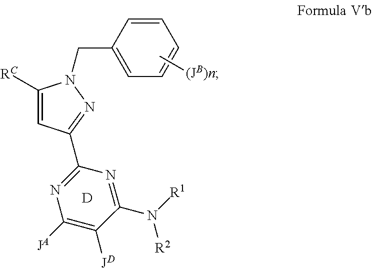

wherein, J^{A} is selected from hydrogen, halogen, methyl, hydroxyl, methoxy, trifluoromethyl, trifluoromethoxy or —NR^{a}R^{b}; wherein R^{a} and R^{b} are each independently selected from hydrogen, C₁₋₆ alkyl or a 3-6 cycloalkyl ring; or wherein R^{a} and R^{b}, together with the nitrogen atom to which they are both attached, form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 6 instances of fluorine;
and J^{D} is either absent or is fluorine.

In other embodiments of the above methods, uses, compositions and kits, the sGC stimulator is a compound of Formula I″ wherein ring B is phenyl or a 6-membered heteroaryl ring. In some of these embodiments, n is an integer selected from 1 to 3 and wherein each J^{B} is independently selected from halogen, a C₁₋₆ aliphatic or —OR^{B}. In some of these embodiments, each J^{B} is independently selected from halogen. In other embodiments, each J^{B} is independently selected from fluoro or chloro. In still other embodiments, each J^{B} is fluoro. In further embodiments, each J^{B} is methyl or ethyl. In further embodiments, n is 1. In some embodiments wherein n is 1, J^{B} is selected from halogen atoms. In other embodiments, J^{B} is fluoro or chloro. In still other embodiments, J^{B} is fluoro. In some embodiments of the compounds of Formula I′, at least one J^{B} is ortho to the attachment of the methylene linker between ring B and ring A. In some of these embodiments, each J^{B} is independently selected from halogen. In other embodiments, each J^{B} is independently selected from fluoro or chloro. In still other embodiments, each J^{B} is fluoro. In further embodiments, n is 1 and the J^{B} ortho to the attachment of the methylene linker between ring B and the pyrazolyl ring is fluoro. In some embodiments of the compounds of Formula I′, ring B is a 6-membered heteroaryl ring. In other embodiments, ring B is a pyridyl ring. In still other embodiments, ring B is a pyrimidinyl ring.

In other embodiments of the above methods, uses, compositions and kits, the sGC stimulator is a compound of Formula I′ wherein o is an integer selected from 1, 2 and 3. In some of these embodiments, each J^{D} is independently selected from halogen, a C₁₋₆ aliphatic, —N(R^{D})₂, —N(R^{d})C(O)R^{D}, —N(R^{d})C(O)OR^{D}, —N(R^{d})C(O)N(R^{D})₂, —SO₂R^{D}, —SO₂N(R^{D})₂, —N(R^{d})SO₂R^{D}, —OR^{D} or an optionally substituted C₃₋₈ cycloaliphatic ring. In other embodiments, o is 1 or 2 and each J^{D} is independently selected from a halogen atom or —N(R^{D})₂, —N(R^{d})COR^{D}, —OH, —N(R^{d})COOR^{D} or —N(R^{d})SO₂R^{D}. In some embodiments, each R^{d} is independently selected from hydrogen or C₁₋₄ alkyl. In other embodiments, o is 1 or 2 and at least one instance of J^{D} is independently selected from fluoro, chloro, hydroxyl or amino.

In other embodiments of the above methods, uses, compositions and kits, the sGC stimulator is a compound represented by one of Formulae Va or VI′a:

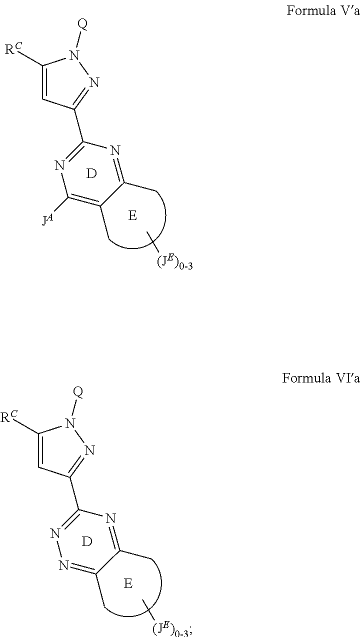

wherein ring E is a 5 or 6-membered heterocyclic ring, containing up to 3 heteroatoms selected from N, O and S; and wherein each J^{E} is independently selected from oxo or —(Y)—R⁹; and
J^{A} is selected from hydrogen, halogen, methyl, hydroxyl, methoxy, trifluoromethyl, trifluoromethoxy or —NR^{a}R^{b}; wherein R^{a} and R^{b} are each independently selected from hydrogen, C₁₋₆ alkyl or a 3-6 cycloalkyl ring; or wherein R^{a} and R^{b}, together with the nitrogen atom to which they are both attached, form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 6 instances of fluorine;

In other embodiments of the above methods, uses, compositions and kits, the sGC stimulator is a compound represented by one of Formulae VI′b or VII′b:

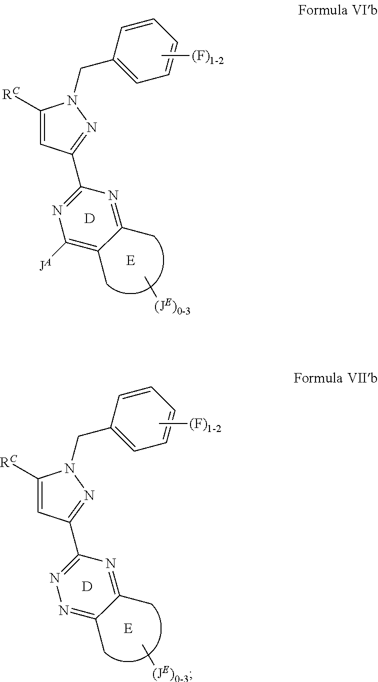

wherein ring E is a 5 or 6-membered heterocyclic ring, containing up to 3 heteroatoms selected from N, O and S; and wherein each J^{E} is independently selected from oxo or —(Y)—R⁹. In some embodiments of the compounds of Formula VI′b or Formula VII′b, J^{A} is selected from halogen, —NH₂, —OH, or hydrogen. In some embodiments of the compounds of Formula VI′b or Formula VII′b, ring E is a heterocyclic ring containing one nitrogen ring atom and wherein at least one instance of J^{E} is oxo. In some of these embodiments, one J^{E} is oxo and two other instances of J^{E} are independently selected from —(Y)—R⁹. In other embodiments of the compounds of Formula VI′b and Formula VII′b, each —(Y)—R⁹ is independently selected from a C₁₋₆ alkyl; a 5 or 6-membered heteroaryl ring containing between 1 and 3 heteroatoms independently selected from N, O or S and optionally substituted by one or more instances of C₁₋₆ alkyl or halogen; or —(CO)NH—R¹⁰. In some of these embodiments, R¹⁰ is a C₃₋₆ cycloalkyl ring.

In other embodiments of the above methods, uses, compositions and kits, the sGC stimulator is a compound represented by Formula VII′a:

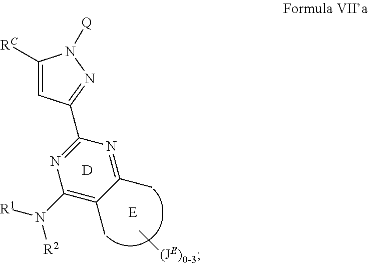

wherein each J^{E} is independently selected from oxo or —(Y)—R⁹.

In other embodiments of the above methods, uses, compositions and kits, the sGC stimulator is a compound represented by Formula VIII′b:

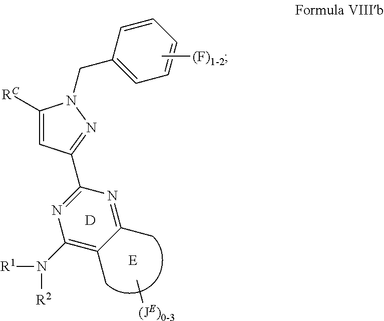

wherein ring E is a 5 or 6-membered heterocyclic ring, containing up to 3 heteroatoms selected from N, O and S; and wherein each J^{E} is independently selected from oxo or —(Y)—R⁹.

In some of the embodiments of the compounds of Formula VII′a and VIII′b, one instance of J^{E} is oxo and two other instances of J^{E} are independently selected from C₁₋₆ alkyl; a 5 or 6-membered heteroaryl ring, containing between 1 and 3 heteroatoms independently selected from N, O or S and optionally substituted by one or more instances of C₁₋₆ alkyl or halogen; and —(CO)NH—R¹⁰. In some embodiments, R¹⁰ is a C₃₋₆ cycloalkyl ring.

In other embodiments of the above methods, uses, compositions and kits, the sGC stimulator is a compound represented by Formula VIII′a or Formula VIII′d:

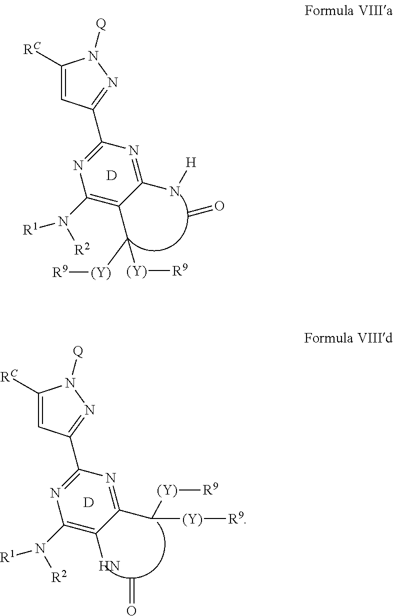

In other embodiments of the above methods, uses, compositions and kits, the sGC stimulator is a compound represented by Formula XIX′b or Formula XIX′d:

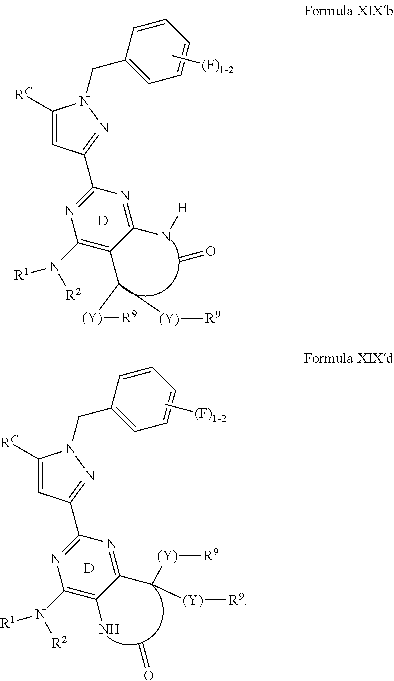

In other embodiments of the above methods, uses, compositions and kits, the sGC stimulator is a compound represented by one of Formulae XIX′a or X′a,

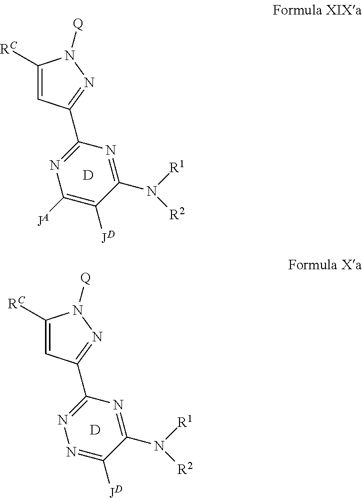

wherein each J^{A} is independently selected from —NH₂ or hydrogen;
wherein each J^{D} is alternatively:
i) when R¹ and R² are not simultaneously hydrogen, each J^{D} is either absent or independently selected from a halogen; or
ii) when R¹ and R² are both simultaneously hydrogen, each J^{D} is independently selected from —C(O)R^{D}, —C(O)OR^{D}, —OC(O)R^{D}, —C(O)N(R^{D})₂, —N(R^{D})₂, —N(R^{d})C(O)R^{D}, —N(R^{d})C(O)OR^{D}, —N(R^{d})C(O)N(R^{D})₂, —OC(O)N(R^{D})₂, —SO₂R^{D}, —SO₂N(R^{D})₂ or —N(R^{d})SO₂R^{D}.

In other embodiments of the above methods, uses, compositions and kits, the sGC stimulator is a compound represented by one of Formulae X′b or XI′b:

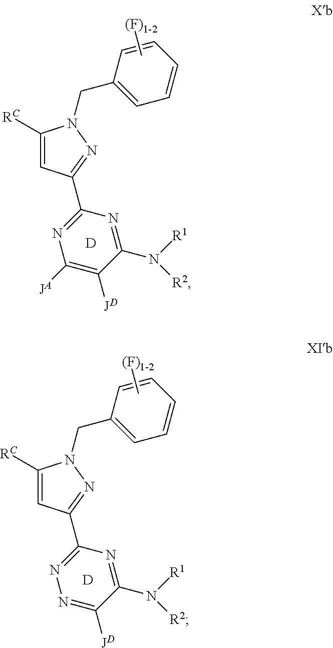

wherein each J^{A} is independently selected from —NH₂ or hydrogen;
wherein each J^{D} is alternatively:
i) when R¹ and R² are not simultaneously hydrogen, each J^{D} is either absent or independently selected from a halogen; or
ii) when R¹ and R² are both simultaneously hydrogen, each J^{D} is independently selected from —C(O)R^{D}, —C(O)OR^{D}, —OC(O)R^{D}, —C(O)N(R^{D})₂, —N(R^{D})₂, —N(R^{d})C(O)R^{D}, —N(R^{d})C(O)OR^{D}, —N(R^{d})C(O)N(R^{D})₂, —OC(O)N(R^{D})₂, —SO₂R^{D}, —SO₂N(R^{D})₂ or —N(R^{d})SO₂R^{D}.

In some embodiments of the compounds of Formula I′, J^{D} is —NH₂, —OH, or is absent. In other embodiments of the compound according to Formula I′, R^{C} is not a ring. In some of these embodiments, R^{C} is selected from halogen, —CN, C₁₋₆ alkyl, —(C₁₋₆ alkyl)-R^{N}, —COOR⁷, —COR⁷, —C(O)OR⁷, —C(O)N(R⁷)₂, —N(R⁷)C(O)R⁷, —N(R⁷)C(O)OR⁷, —N(R⁷)C(O)N(R⁷)₂, —N(R⁷)₂, —SO₂R⁷, —SO₂N(R⁷)₂, or —N(R⁷)SO₂R⁷; wherein when said R^{C} is a C₁₋₆ alkyl or —(C₁₋₆ alkyl)-R^{N}, the C₁₋₆ alkyl or the (C₁₋₆ alkyl) portion of the —(C₁₋₆ alkyl)-R^{N} moiety is optionally and independently substituted with up to 6 instances of fluoro and/or up to 2 instances of R^{7c}. In other embodiments, R^{C} is —CN, C₁₋₆ alkyl, —COR⁷, —C(O)OR⁷, —C(O)N(R⁷)₂, —N(R⁷)₂, —SO₂R⁷, or —SO₂N(R⁷)₂; wherein when said R^{C} is a C₁₋₆ alkyl or —(C₁₋₆ alkyl)-R^{N}, the C₁₋₆ alkyl or the (C₁₋₆ alkyl) portion of the —(C₁₋₆ alkyl)-R^{N} moiety is optionally and independently substituted with up to 6 instances of fluoro and/or up to 2 instances of R^{7c}. In still other embodiments, R^{C} is C₁₋₆ alkyl, —COR⁷, —C(O)OR⁷, —C(O)N(R⁷)₂, —N(R⁷)₂, —SO₂R⁷ or —SO₂N(R⁷)₂. In other embodiments of the compounds of Formula I′, R^{C} is a ring. In some of these embodiments, the compound is represented by Formula I:

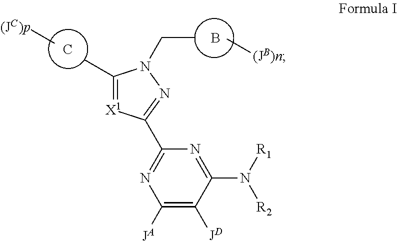

wherein:
X¹ is selected from N, CH, C(C₁₋₄ alkyl), C(C₁₋₄ haloalkyl), CCl and CF;
ring B is a phenyl or a 6-membered heteroaryl ring containing 1 or 2 ring nitrogen atoms, or ring B is a thiophene;
n is 0 or an integer selected from 1 to 3;
each J^{B} is independently selected from halogen, —CN, a C₁₋₆ aliphatic, —OR^{B} or a C₃₋₈ cycloaliphatic ring; wherein each of said C₁₋₆ aliphatic and each of said C₃₋₈ cycloaliphatic group is optionally substituted with up to 3 instances of halogen;
each R^{B} is independently selected from hydrogen, a C₁₋₆ aliphatic or a C₃₋₈ cycloaliphatic ring; wherein each of said C₁₋₆ aliphatic and each said C₃₋₈ cycloaliphatic ring is optionally substituted with up to 3 instances of halogen;
J^{A} is selected from hydrogen, halogen, methyl, methoxy, trifluoromethyl, trifluoromethoxy or —NR^{a}R^{b}, wherein R^{a} and R^{b} are each independently selected from hydrogen, C₁₋₆ alkyl or a 3-6 cycloalkyl ring;
J^{D} is absent or selected from halogen, —CN, —CF₃, methoxy, trifluoromethoxy, nitro, amino or methyl;
R¹ and R², together with the nitrogen atom to which they are attached, form a 4 to 8-membered heterocyclic ring or 5 or 6-membered heteroaryl ring; wherein said 4 to 8-membered heterocyclic ring or 5 or 6-membered heteroaryl ring optionally contains in addition to the nitrogen atom up to 3 ring heteroatoms independently selected from N, O or S, and is optionally substituted by up to 5 instances of R⁵; or
alternatively, R¹ and R² are each independently selected from hydrogen, C₁₋₆ alkyl, a C₃₋₈ cycloalkyl ring, a 4 to 8-membered heterocyclic ring, a 5 or 6-membered heteroaryl or a C₁₋₆ alkyl-R^{Y}; wherein each of said 4 to 8-membered heterocyclic ring and each of said 5 or 6-membered heteroaryl ring contains up to 3 ring heteroatoms independently selected from N, O and S; and wherein each of said C₁₋₆ alkyl, C₃₋₈ cycloalkyl ring, 4 to 8-membered heterocyclic ring group, 5 or 6-membered heteroaryl and the C₁₋₆ alkyl portion of said C₁₋₆ alkyl-R^{Y} is optionally and independently substituted with up to 5 instances of R^{5a}; provided that R¹ and R² are never simultaneously hydrogen;
alternatively, J^{D} and one of R¹ or R² can form a 5-6 membered heterocyclic ring containing up to two heteroatoms selected from O, N and S and optionally substituted with up to 3 instances of oxo or —(Y)—R⁹;
wherein Y is either absent or is a linkage in the form of a C₁₋₆ alkyl chain, optionally substituted by up to 6 instances of fluoro;
each R⁹ is independently selected from hydrogen, fluoro, —CN, —OR¹⁰, —SR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, a C₃₋₆ cycloalkyl ring, a 4-8-membered heterocyclic ring or a 5-6 membered heteroaroaryl ring; wherein each said 4 to 8-membered heterocyclic ring or 5 to 6-membered heteroaromatic ring contains up to 4 ring heteroatoms independently selected from N, O or S; and wherein each of said C₃₋₆ cycloalkyl rings, each of said 4 to 8-membered heterocyclic rings and each of said 5 to 6-membered heteroaromatic rings is optionally substituted with up to 3 instances of R¹¹;
each R¹¹ is independently selected from halogen, C₁₋₆ alkyl, —CN, —OR¹², —SR¹², —COR¹², —OC(O)R¹², —C(O)OR¹², —C(O)N(R¹²)₂, —C(O)N(R¹²)SO₂R¹², —N(R¹²)C(O)R¹², —N(R¹²)C(O)OR¹², —N(R¹²)C(O)N(R¹²)₂, —N(R¹²)₂, —SO₂R¹², —SO₂N(R¹²)₂, —SO₂N(R¹²)COOR¹², —SO₂N(R¹²)C(O)R¹², —N(R¹²)SO₂R¹² and —N═OR¹²; wherein each of said C₁₋₆ alkyl is optionally and independently substituted by up to 3 instances of fluoro, —OH, —O(C₁₋₄ alkyl), phenyl and —O(C₁₋₄ fluoroalkyl);
wherein each R¹⁰ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo; and
wherein each R¹² is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo;
R^{Y} is selected from a C₃₋₈ cycloalkyl ring, a 4 to 8-membered heterocyclic ring, phenyl, or a 5 to 6-membered heteroaromatic ring; wherein each of said 4 to 8-membered heterocyclic ring or 5 to 6-membered heteroaromatic ring contains up to 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaromatic ring is optionally substituted with up to 5 instances of R^{5c};
each R^{5c} is independently selected from halogen, —CN, C₁₋₆ alkyl, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR^{6b}, a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
two instances of R^{5c} attached to the same or different ring atoms of R^{Y}, together with said ring atom or atoms, may form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or a 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR″(CO)CO(C₁₋₄ alkyl), —OH or halogen; wherein R″ is hydrogen or a C₁₋₂ alkyl;
each R^{5a} is independently selected from halogen, —CN, C₁₋₆ alkyl, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆ alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S; or
when one of R¹ or R² is the C₃₋₈ cycloalkyl ring, 4 to 8-membered heterocyclic ring or 5 or 6-membered heteroaryl substituted with up to 5 instances of R^{5a}, two of the instances of R^{5a} attached to the same or different ring atoms of said R¹ or R², together with said atom or atoms, may optionally form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)CO(C₁₋₄ alkyl), —NR′(CO)CO(C₁₋₄ alkyl) or halogen; wherein R′ is hydrogen or a C₁₋₂ alkyl;
each R⁵ is independently selected from halogen, —CN, C₁₋₆ alkyl, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R⁶ is independently selected from hydrogen, a C₁₋₆ alkyl, phenyl, benzyl, a C₃₋₈ cycloalkyl ring or a 4 to 7-membered heterocyclic ring, a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
when R¹ and R² attached to the nitrogen atom form the 4 to 8-membered heterocyclic ring or 5 or 6-membered heteroaryl ring substituted with up to 5 instances of R⁵, two of the instances of R⁵ attached to the same or different atoms of said ring, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen; wherein R is hydrogen or a C₁₋₂ alkyl;
p is an integer selected from 0, 1 or 2;
ring C is a monocyclic 5-membered heteroaryl ring containing up to 4 ring heteroatoms selected from N, O or S; wherein said monocyclic 5-membered heteroaryl ring is not a 1,3,5-triazinyl ring;
each J^{C} is independently selected from halogen or a C₁₋₄ aliphatic optionally and independently substituted by up to 3 instances of C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen.

In other embodiments of the above methods, uses, compositions and kits, the sGC stimulator is a compound of Formula I, wherein each J^{B} is independently selected from halogen, a C₁₋₄ alkyl or —OR^{B}. In other embodiments, each J^{B} is independently selected from halogen atoms. In other embodiments, each J^{B} is independently selected from fluoro or chloro. In still other embodiments, each J^{B} is fluoro. In further embodiments, each J^{B} is a C₁₋₄ alkyl. In even further embodiments, each J^{B} is methyl or ethyl. In some of the embodiments of the compounds of Formula I, n is 1. In some of these embodiments, J^{B} is selected from halogen atoms. In other of these embodiments, J^{B} is fluoro or chloro. In still other embodiments, J^{B} is fluoro. In some of the embodiments of the compounds of Formula I, at least one J^{B} is ortho to the attachment of the methylene linker between ring B and the ring bearing X¹. In some of these embodiments, each J^{B} is independently selected from halogen atoms. In other embodiments, each J^{B} is independently selected from fluoro or chloro. In still other embodiments, each J^{B} is fluoro. In further embodiments, n is 1 and the J^{B} ortho to the attachment of the methylene linker between ring B and the ring bearing X¹ is fluoro. In other embodiments of the compounds of Formula I, n is 2 and each J^{B} is a halogen atom. In some of these embodiments, each J^{B} is fluoro. In other embodiments, one J^{B} is fluoro and the other J^{B} is chloro. In some of the embodiments of the compounds of Formula I, ring B is phenyl. In other embodiments, ring B is a 6-membered heteroaryl ring or a thiophene ring. In still other embodiments, ring B is a pyridyl ring. In further embodiments, ring B is a pyrimidinyl ring. In even further embodiments ring B is a thiophene ring.

In other embodiments of the above methods, uses, compositions and kits, the sGC stimulator is a compound of Formula I, wherein J^{D} is fluoro, chloro or is absent. In some of these embodiments, J^{D} is fluoro. In some of these embodiments, J^{A} is hydrogen.

In other embodiments of the above methods, uses, compositions and kits, the sGC stimulator is a compound of Formula I, wherein ring C is a monocyclic 5-membered heteroaryl ring containing 1 or 2 ring heteroatoms selected from N, O or S. In some of these embodiments, ring C is an oxazole or isoxazole ring. In some embodiments, ring C is unsubstituted. In other embodiments, ring C is an oxazolyl or isoxazolyl group. In some of these embodiments, p is 0.

In other embodiments of the above methods, uses, compositions and kits, the sGC stimulator is a compound of Formula I, wherein X¹ is N.

In other embodiments of the above methods, uses, compositions and kits, the sGC stimulator is a compound of Formula I wherein ring B is phenyl. In some of these embodiments, J^{B} is halogen. In other embodiments, J^{B} is fluoro. In other embodiments, n is 1. In some embodiments, ring B is substituted with J^{B} ortho to the methylene bridge between the ring bearing X¹ and ring B.

In other embodiments of the above methods, uses, compositions and kits, the sGC stimulator is a compound of Formula I, wherein J^{D} is halogen. In some of these embodiments, J^{D} is fluoro.

In other embodiments of the above methods, uses, compositions and kits, the sGC stimulator is a compound of Formula I, wherein ring C is an isoxazolyl group.

In other embodiments of the above methods, uses, compositions and kits, the sGC stimulator is a compound of Formula I wherein ring B is phenyl and J^{B} is halogen. In some of these embodiments, J^{B} is fluoro and n is 1.

In other embodiments of the above methods, uses, compositions and kits, the sGC stimulator is a compound of Formula I, wherein ring B is substituted with J^{B} ortho to the methylene bridge between the ring bearing X¹ and ring B.

In other embodiments of the above methods, uses, compositions and kits, the sGC stimulator is a compound of Formula I, wherein J^{D} is halogen. In some of these embodiments, J^{D} is fluoro.

In other embodiments of the above methods, uses, compositions and kits, the sGC stimulator is a compound of Formula I, wherein X¹ is CH, C(C₁₋₄ alkyl), or CF.

In other embodiments of the above methods, uses, compositions and kits, the sGC stimulator is a compound of Formula I, wherein ring C is an oxazolyl or isoxazolyl group. In some of these embodiments, p is 0.

In other embodiments of the above methods, uses, compositions and kits, the sGC stimulator is a compound of Formula I, represented by one of Formulae IIa or IIb

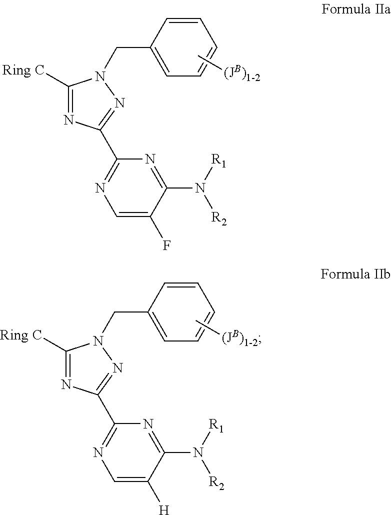

wherein J^{B} is halogen and Ring C is an unsubstituted oxazole or isoxazole ring.

In other embodiments of the above methods, uses, compositions and kits, the sGC stimulator is a compound of Formula I, having one of Formulae IIIa to IIId:

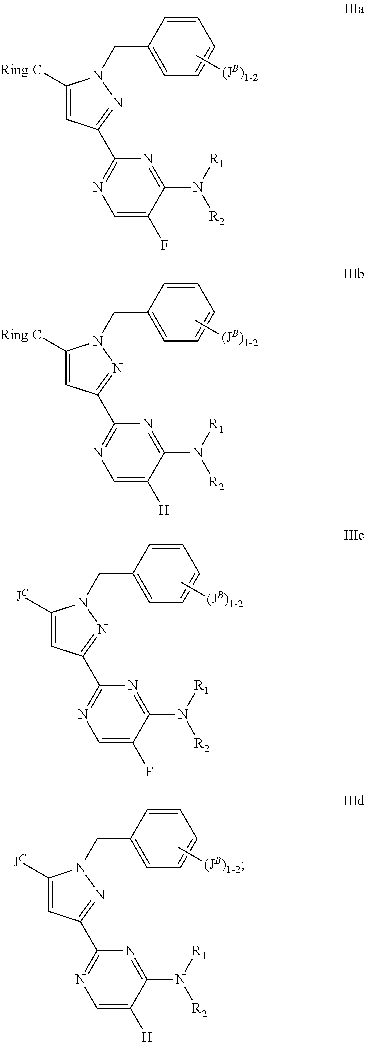

wherein J^{B} is halogen and Ring C is an unsubstituted oxazole or isoxazole ring;
J^{C} is selected from halogen, —CN, C₁₋₆ alkyl, —(C₁₋₆ alkyl)-R^{N}, —OR⁷, —SR⁷, —COR⁷, —OC(O)R⁷, —C(O)OR⁷, —C(O)N(R⁷)₂, —N(R⁷)C(O)R⁷, —N(R⁷)C(O)OR, —N(R⁷)C(O)N(R⁷)₂, —N(R⁷)₂, —SO₂R⁷, —SO₂N(R⁷)₂, —C(O)N(R⁷)SO₂R⁷, —SO₂N(R⁷)COOR⁷, —SO₂N(R⁷)C(O)R⁷, —N(R⁷)SO₂R⁷ or —(C═O)NHOR⁷; wherein each said C₁₋₆ alkyl, each C₁₋₆ alkyl portion of said —(C₁₋₆ alkyl)-R^{N}, is optionally and independently substituted with up to 6 instances of fluoro and up to 2 instances of —CN, —OR⁸, oxo, —N(R⁸)₂, —N(R⁸)C(O)R⁸, —N(R⁸)C(O)OR⁸, —N(R⁸)C(O)N(R⁸)₂, —SO₂R⁸, —SO₂N(R⁸)₂, —NHOR⁸, —SO₂N(R⁸)COOR⁸, —SO₂N(R⁸)C(O)R⁸, —N(R⁷)SO₂R⁸;
R¹ and R², together with the nitrogen atom to which they are attached, form a 4 to 8-membered heterocyclic ring or 5-membered heteroaryl ring; wherein said 4 to 8-membered heterocyclic ring or 5-membered heteroaryl ring optionally contains, in addition to the nitrogen atom to which R¹ and R² are attached, up to 3 ring heteroatoms independently selected from N, O or S, and is optionally substituted by up to 5 instances of R^{5e};
each R^{5e} is independently selected from halogen, —CN, C₁₋₆ alkyl, —(C₁₋₄ alkyl)-R⁶, a C₃₋₈ cycloalkyl ring, C₁₋₄ cyanoalkyl, —OR⁶, —SR⁶, —OCOR⁶, —COR⁶, —C(O)OR⁶, —C(O)N(R⁶)₂, —N(R⁶)C(O)R⁶, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, benzyl, phenyl or an oxo group; wherein each said phenyl ring and each said benzyl group, is optionally and independently substituted with up to 3 instances of halogen, —OH, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl); and wherein each said C₁₋₆ alkyl, each C₁₋₄ alkyl portion of said —(C₁₋₄ alkyl)-R⁶ moiety, and each said C₃₋₈ cycloalkyl ring is optionally and independently substituted with up to 3 instances of halogen; wherein
each R⁶ is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
two of the instances of R^{5e} attached to the same or different atoms of said ring formed by R¹, R² and the nitrogen to which R¹ and R² are attached, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
alternatively, R¹ and R² are each independently selected from hydrogen, C₁₋₆ alkyl, a C₃₋₈ cycloalkyl ring, a 4 to 10-membered heterocyclic ring, a 5 or 6-membered heteroaryl, phenyl or a C₁₋₆ alkyl-R^{Y}; wherein each of said 4 to 10-membered heterocyclic ring and each of said 5 or 6-membered heteroaryl ring contains up to 3 ring heteroatoms independently selected from N, O and S; and wherein each of said C₁₋₆ alkyl, C₁₋₆ alkyl portion of each said C₁₋₆ alkyl-R^{Y} moiety, C₃₋₈ cycloalkyl ring, 4 to 10-membered heterocyclic ring group, 5 or 6-membered heteroaryl, phenyl and C₁₋₆ alkyl-R^{Y} is optionally and independently substituted with up to 5 instances of R^{5f}; provided that one of R¹ or R² may not be pyridine or pyrimidine;
R^{Y} is selected from a C₃₋₈ cycloalkyl ring, a 4 to 8-membered heterocyclic ring, phenyl, or a 5 to 6-membered heteroaryl ring; wherein each of said 4 to 8-membered heterocyclic ring or 5 to 6-membered heteroaromatic ring contains between 1 and 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaryl ring is optionally substituted with up to 5 instances of R^{5g};
each R^{5f} is independently selected from halogen, —CN, C₁₋₆ alkyl, —(C₁₋₄ alkyl)-R^{6a}, a C₇₋₁₂ aralkyl, C₃₋₈ cycloalkyl ring, C₁₋₄ cyanoalkyl, —OR^{6a}, —SR^{6a}, —OCOR^{6a}, —COR^{6a}, —C(O)OR^{6a}, —C(O)N(R^{6a})₂, —N(R^{6a})C(O)R^{6a}, —N(R^{6a})₂, —SO₂R^{6a}, —SO₂N(R^{6a})₂, —N(R^{6a})SO₂R^{6a}, —SO₂OH, —SO₂NHOH, —SO₂N(R^{6a})COR^{6a}, phenyl or an oxo group; wherein each said phenyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₇₋₁₂ aralkyl, C₁₋₆ alkyl, C₁₋₄ alkyl portion of each said —(C₁₋₄ alkyl)-R^{6a} and each said C₃₋₈ cycloalkyl group is optionally and independently substituted with up to three instances of halogen;
each R^{6a} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
when one of R¹ or R² is the C₃₋₈ cycloalkyl ring, 4 to 8-membered heterocyclic ring or 5 or 6-membered heteroaryl substituted with up to 5 instances of R^{5f}, two of the instances of R^{5f} attached to the same or different ring atoms of said R¹ or R², together with said atom or atoms, form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)O(C₁₋₄ alkyl), —NR′(CO)O(C₁₋₄ alkyl) or halogen; wherein R′ is hydrogen or a C₁₋₂ alkyl;
each R^{5g} is independently selected from halogen, —CN, C₁₋₆ alkyl, —(C₁₋₄ alkyl)-R^{6b}, a benzyl, C₃₋₈ cycloalkyl ring, C₁₋₄ cyanoalkyl, —OR^{6b}, —SR^{6b}, —OCOR^{6b}, —COR^{6b}, —C(O)OR^{6b}, —C(O)N(R^{6b})₂, —N(R^{6b})C(O)R^{6b}, —N(R^{6b})₂, —SO₂R^{6b}, —SO₂N(R^{6b})₂, —N(R^{6b})SO₂R^{6b}, —SO₂OH, —SO₂NHOH, —SO₂N(R^{6b})COR^{6b}, phenyl or an oxo group; wherein each said phenyl and each said benzyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₁₋₆ alkyl, C₁₋₄ alkyl portion of each said (C₁₋₄ alkyl)-R^{6b} moiety and each said C₃₋₈ cycloalkyl group is optionally and independently substituted with up to 3 instances of halogen;
each R^{6b} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
alternatively, two instances of R^{5g} attached to the same or different ring atoms of R^{Y}, together with said ring atom or atoms, form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR″(CO)O(C₁₋₄ alkyl), —OH or halogen; and
R″ is hydrogen or a C₁₋₂ alkyl.

In other embodiments of the above methods, uses, compositions and kits, the sGC stimulator is a compound of Formula I, having Formula IV:

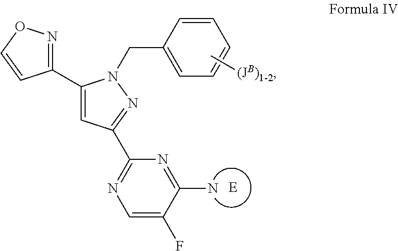

wherein:
J^{B} is halogen; and
ring E is a monocyclic or bicyclic 4 to 10-membered heterocyclic ring or a monocyclic or bicyclic 5 to 10-membered heteroaryl ring; wherein said 4 to 10-membered heterocyclic ring or 5 to 10-membered heteroaryl ring optionally contains up to 3 ring heteroatoms independently selected from N, O or S, and is optionally and independently substituted by up to 3 instances of R⁵.

In some embodiments of the compounds of Formula IV, ring E is substituted by:

(i) 3 instances of R⁵; wherein at least two of said instances are the same, or

(ii) 0, 1 or 2 instances of R⁵; wherein, when ring F is substituted by 2 instances of R⁵, then each of the instances of R⁵ is independently selected;

wherein each R⁵ is selected from fluoro, methyl, ethyl, methoxy, trifluoromethyl, trifluoromethoxy, hydroxyl, C₁₋₆ (hydroxy)alkyl, oxo, —CN, —O(C₁₋₆ alkyl)-COOR^{Z}, —NH(C₁₋₆ alkyl)-COOR^{Z}, —(C₁₋₆ alkyl)-COOR^{Z}, —COOR^{Z}, —COR^{Z}, —CON(R^{Z})₂, —NHCOOR^{Z}, —NHCON(R^{Z})₂, —CONHSO₂R^{Z}, —NHCOR^{Z}, —NH(C₁₋₆ alkyl)-CON(R^{Z})₂, —N(R^{Z})₂, —SO₂R^{Z}, —SO₂N(R^{Z})₂, —SO₂NHCOR^{Z}, —SO₂NHCOOR^{Z}, phenyl, benzyl, or a 5 or 6 membered heterocyclic or heteroaryl ring; wherein each of said phenyl, benzyl or 5-6 membered heteroaryl or heterocyclic ring is optionally substituted by 1 or 2 instances of R^{Za};

wherein each R^{Z} is independently selected from hydrogen, a C₃₋₆ cycloalkyl, a C₁₋₆ alkyl, a C₁₋₆ fluoroalkyl; and

wherein each R^{Za} is independently selected from hydrogen, halogen, a C₃₋₆ cycloalkyl, a C₁₋₆ alkyl, a C₁₋₆ fluoroalkyl, oxo and —COOH.

In other embodiments of the compounds of Formula IV, R⁵ is a —COOH moiety or at least one instance of R⁵ contains a —COOH moiety. In other embodiments of the compounds of Formula IV, the compound is of Formula V:

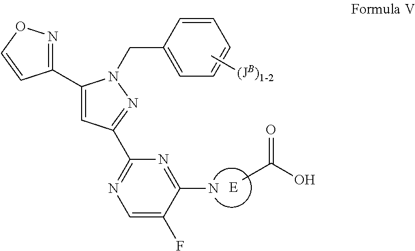

wherein ring E is optionally and independently further substituted by 1 or 2 instances of R⁵.

In other embodiments of the compounds of Formula IV, the compound is of Formula VI:

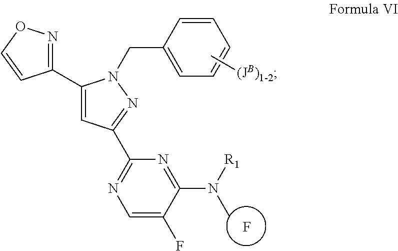

wherein J^{B} is halogen;

R¹ is hydrogen or C₁₋₆ alkyl;

and ring F is a monocyclic or bicyclic 4 to 10-membered heterocyclic ring or a monocyclic or bicyclic 5 to 10-membered heteroaryl ring; wherein said 4 to 10-membered heterocyclic ring or 5 to 10-membered heteroaryl ring optionally contains up to 3 ring heteroatoms independently selected from N, O or S, and is optionally and independently substituted by up to 3 instances of R^{5a}.

In some embodiments of the compounds of Formula VI, at least one instance of R^{5a} is a —COOH moiety or at least one instance of R^{5a} comprises a —COOH moiety.

In some embodiments of the compounds of Formula VI, the compound is of Formula VII:

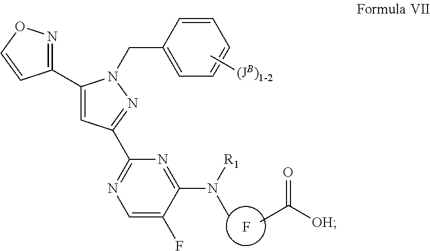

wherein ring F is optionally and independently further substituted by 1 or 2 instances of R^{5a}.

In some embodiments of the compounds of Formula I, the compound is one of Formula VIII:

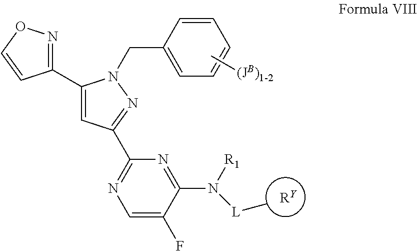

wherein J^{B} is halogen;
R¹ is hydrogen or C₁₋₆ alkyl;
L is a C₁₋₆ alkyl group optionally and independently substituted by up to three instances of R^{5a};
and ring R^{Y} is a monocyclic or bicyclic 4 to 10-membered heterocyclic ring or a monocyclic or bicyclic 5 to 10-membered heteroaryl ring; wherein said 4 to 10-membered heterocyclic ring or 5 to 10-membered heteroaryl ring optionally contains up to 3 additional heteroatoms independently selected from N, O or S, and is optionally and independently substituted by up to 3 instances of R^{5b}.

In some embodiments of the compounds of Formula I, the compound is one of Formula IX or Formula X:

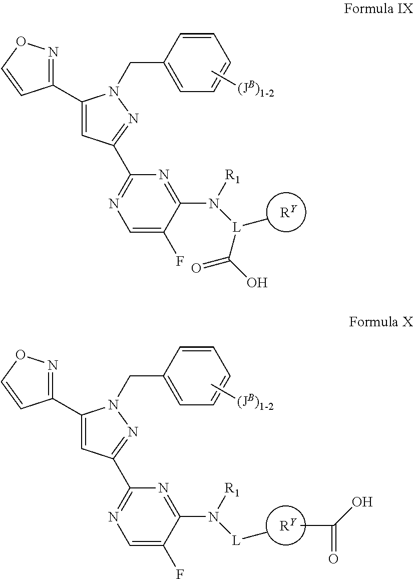

wherein in Formula IX, the linker L is further optionally and independently substituted by up to two instances of R^{5a}; and in Formula X, ring R^{Y} is further optionally and independently substituted by up to two instances of R^{5b}.

In some embodiments of the compounds of Formula I, the compound is one of Formula XI:

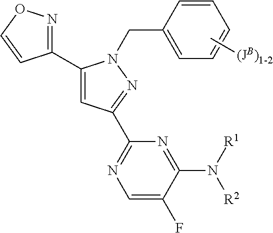

wherein J^{B} is halogen;
R¹ is hydrogen or C₁₋₆ alkyl;
R² is a C₁₋₆ alkyl group optionally and independently substituted by up to three instances of R^{5a}.

In some embodiments of the above methods, uses, compositions and kits, the sGC stimulator is a compound of Formula I′ or Formula I selected from those depicted in Table XA below:

TABLE XA

| | |
|---|---|
| 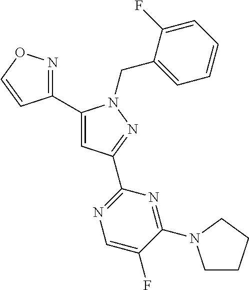 [SMILES: Fc1ccccc1Cn1nc(-c2ncc(F)c(N3CCCC3)n2)cc1-c1ccon1] | I-1 |
| 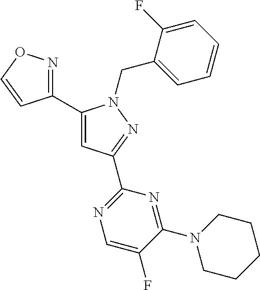 [SMILES: Fc1ccccc1Cn1nc(-c2ncc(F)c(N3CCCCC3)n2)cc1-c1ccon1] | I-2 |
| 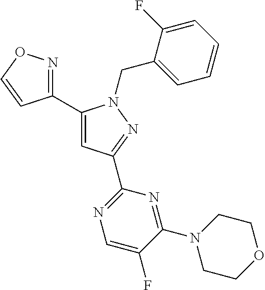 [SMILES: Fc1ccccc1Cn1nc(-c2ncc(F)c(N3CCOCC3)n2)cc1-c1ccon1] | I-3 |
| 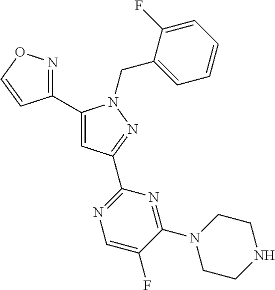 [SMILES: Fc1ccccc1Cn1nc(-c2ncc(F)c(N3CCNCC3)n2)cc1-c1ccon1] | I-4 |
| 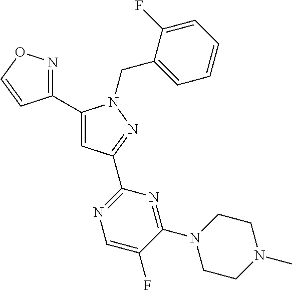 [SMILES: CN1CCN(c2nc(-c3cc(-c4ccon4)n(Cc4ccccc4F)n3)ncc2F)CC1] | I-5 |
| 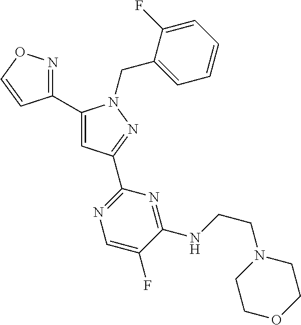 [SMILES: Fc1ccccc1Cn1nc(-c2ncc(F)c(NCCN3CCOCC3)n2)cc1-c1ccon1] | I-6 |
| 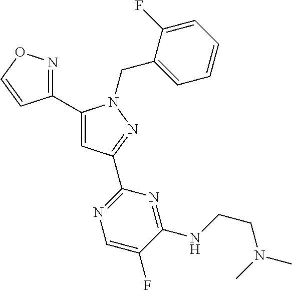 [SMILES: CN(C)CCNc1nc(-c2cc(-c3ccon3)n(Cc3ccccc3F)n2)ncc1F] | I-7 |
| 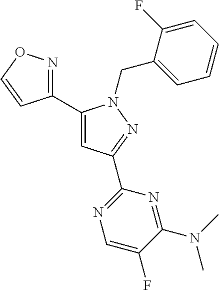 [SMILES: CN(C)c1nc(-c2cc(-c3ccon3)n(Cc3ccccc3F)n2)ncc1F] | I-8 |
| 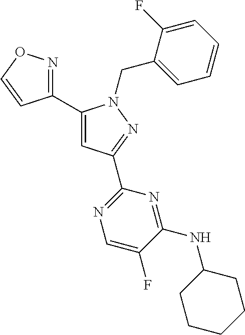 [SMILES: Fc1ccccc1Cn1nc(-c2ncc(F)c(NC3CCCCC3)n2)cc1-c1ccon1] | I-9 |
| 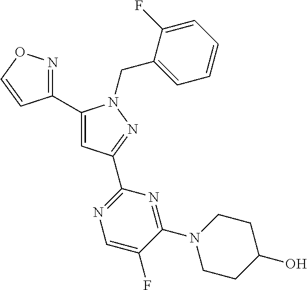 [SMILES: OC1CCN(c2nc(-c3cc(-c4ccon4)n(Cc4ccccc4F)n3)ncc2F)CC1] | I-10 |
| 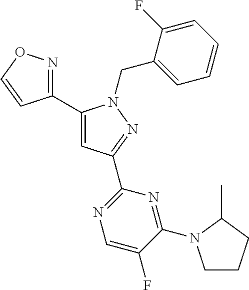 [SMILES: CC1CCCN1c1nc(-c2cc(-c3ccon3)n(Cc3ccccc3F)n2)ncc1F] | I-11 |
| 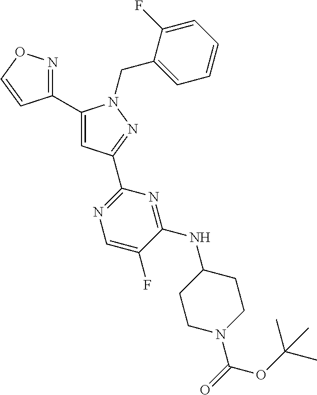 [SMILES: CC(C)(C)OC(=O)N1CCC(Nc2nc(-c3cc(-c4ccon4)n(Cc4ccccc4F)n3)ncc2F)CC1] | I-12 |
| 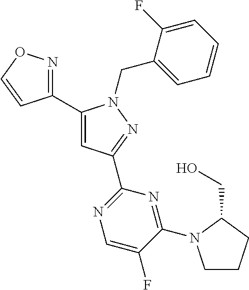 [SMILES: OC[C@@H]1CCCN1c1nc(-c2cc(-c3ccon3)n(Cc3ccccc3F)n2)ncc1F] | I-13 |
| 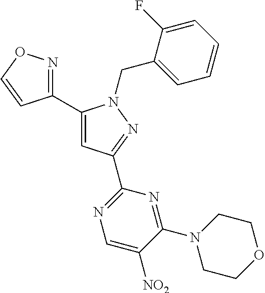 [SMILES: O=[N+]([O-])c1cnc(-c2cc(-c3ccon3)n(Cc3ccccc3F)n2)nc1N1CCOCC1] | I-14 |
| 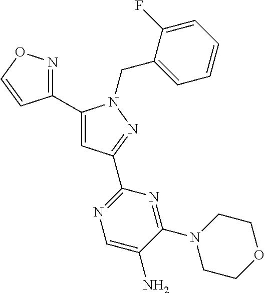 [SMILES: Nc1cnc(-c2cc(-c3ccon3)n(Cc3ccccc3F)n2)nc1N1CCOCC1] | I-15 |
| 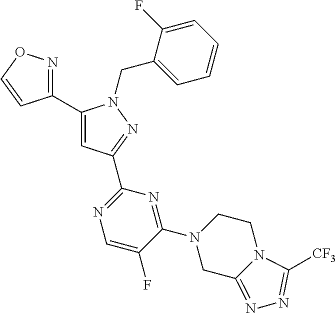 [SMILES: Fc1ccccc1Cn1nc(-c2ncc(F)c(N3CCn4c(nnc4C(F)(F)F)C3)n2)cc1-c1ccon1] | I-16 |
| 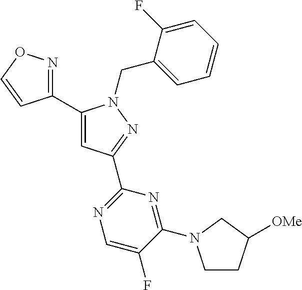 [SMILES: COC1CCN(c2nc(-c3cc(-c4ccon4)n(Cc4ccccc4F)n3)ncc2F)C1] | I-17 |
| 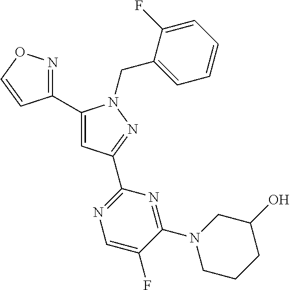 [SMILES: OC1CCCN(c2nc(-c3cc(-c4ccon4)n(Cc4ccccc4F)n3)ncc2F)C1] | I-18 |
| 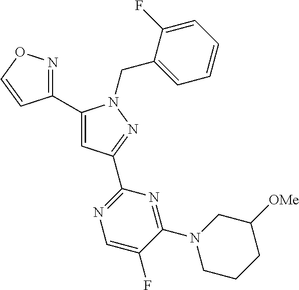 [SMILES: COC1CCCN(c2nc(-c3cc(-c4ccon4)n(Cc4ccccc4F)n3)ncc2F)C1] | I-19 |
| 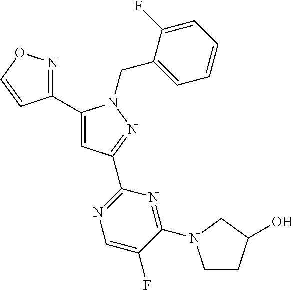 [SMILES: OC1CCN(c2nc(-c3cc(-c4ccon4)n(Cc4ccccc4F)n3)ncc2F)C1] | I-20 |
| 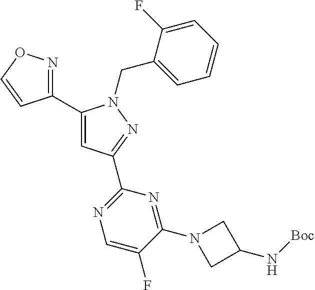 [SMILES: CC(C)(C)OC(=O)NC1CN(c2nc(-c3cc(-c4ccon4)n(Cc4ccccc4F)n3)ncc2F)C1] | I-21 |
| 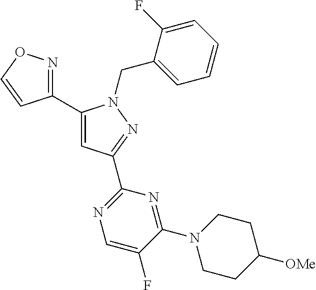 [SMILES: COC1CCN(c2nc(-c3cc(-c4ccon4)n(Cc4ccccc4F)n3)ncc2F)CC1] | I-22 |
| 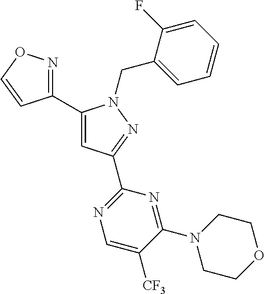 [SMILES: Fc1ccccc1Cn1nc(-c2ncc(C(F)(F)F)c(N3CCOCC3)n2)cc1-c1ccon1] | I-23 |
| 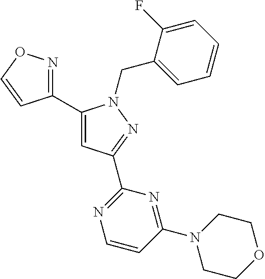 [SMILES: Fc1ccccc1Cn1nc(-c2nccc(N3CCOCC3)n2)cc1-c1ccon1] | I-24 |
| 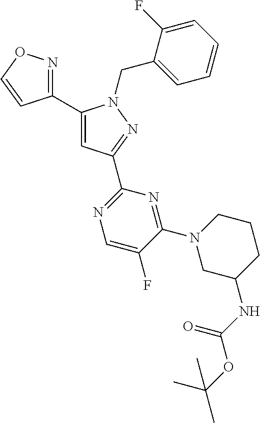 [SMILES: CC(C)(C)OC(=O)NC1CCCN(c2nc(-c3cc(-c4ccon4)n(Cc4ccccc4F)n3)ncc2F)C1] | I-25 |
| 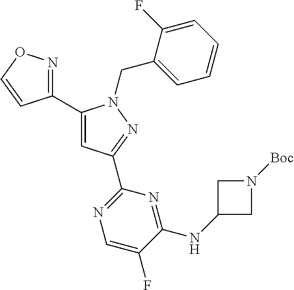 [SMILES: CC(C)(C)OC(=O)N1CC(Nc2nc(-c3cc(-c4ccon4)n(Cc4ccccc4F)n3)ncc2F)C1] | I-26 |
| 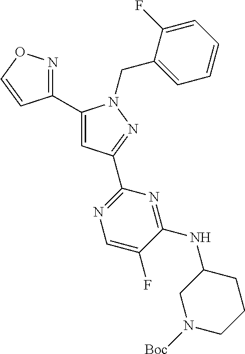 [SMILES: CC(C)(C)OC(=O)N1CCCC(Nc2nc(-c3cc(-c4ccon4)n(Cc4ccccc4F)n3)ncc2F)C1] | I-27 |
| 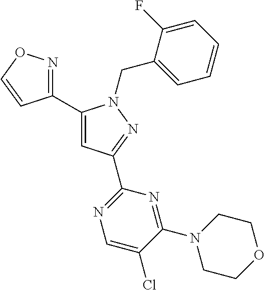 [SMILES: Fc1ccccc1Cn1nc(-c2ncc(Cl)c(N3CCOCC3)n2)cc1-c1ccon1] | I-28 |
| 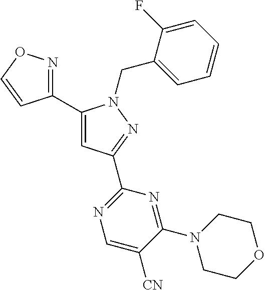 [SMILES: N#Cc1cnc(-c2cc(-c3ccon3)n(Cc3ccccc3F)n2)nc1N1CCOCC1] | I-29 |
| 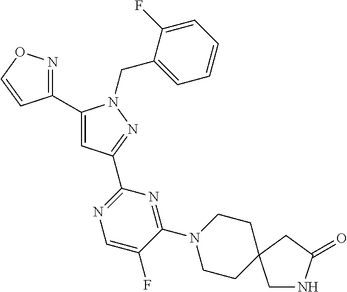 [SMILES: O=C1CC2(CCN(c3nc(-c4cc(-c5ccon5)n(Cc5ccccc5F)n4)ncc3F)CC2)CN1] | I-30 |
| 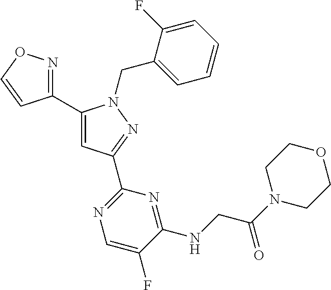 [SMILES: O=C(CNc1nc(-c2cc(-c3ccon3)n(Cc3ccccc3F)n2)ncc1F)N1CCOCC1] | I-31 |
| 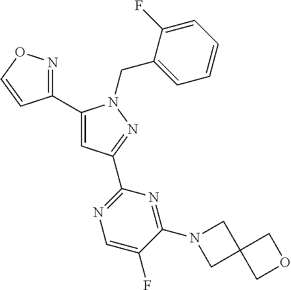 [SMILES: Fc1ccccc1Cn1nc(-c2ncc(F)c(N3CC4(COC4)C3)n2)cc1-c1ccon1] | I-32 |
| 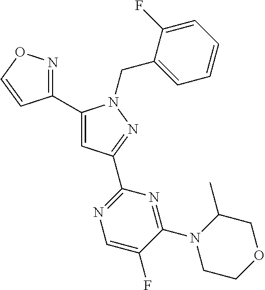 [SMILES: CC1COCCN1c1nc(-c2cc(-c3ccon3)n(Cc3ccccc3F)n2)ncc1F] | I-33 |
| 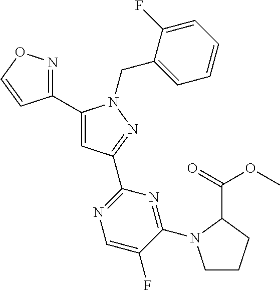 [SMILES: COC(=O)C1CCCN1c1nc(-c2cc(-c3ccon3)n(Cc3ccccc3F)n2)ncc1F] | I-34 |
| 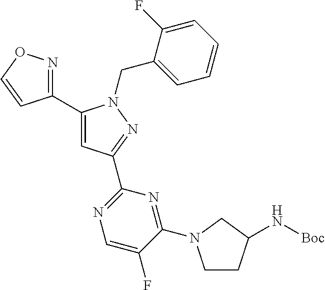 [SMILES: CC(C)(C)OC(=O)NC1CCN(c2nc(-c3cc(-c4ccon4)n(Cc4ccccc4F)n3)ncc2F)C1] | I-35 |
| 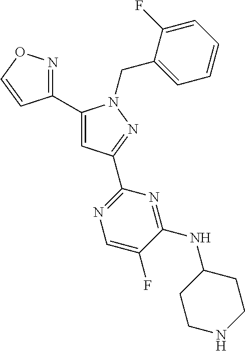 [SMILES: Fc1ccccc1Cn1nc(-c2ncc(F)c(NC3CCNCC3)n2)cc1-c1ccon1] | I-36 |
| 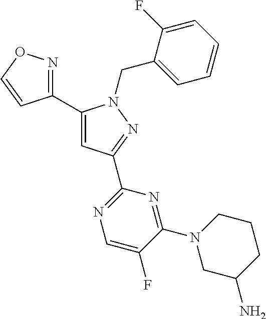 [SMILES: NC1CCCN(c2nc(-c3cc(-c4ccon4)n(Cc4ccccc4F)n3)ncc2F)C1] | I-37 |
| 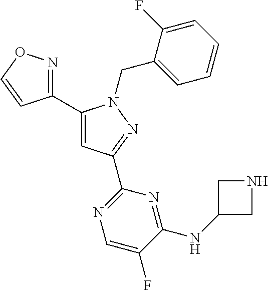 [SMILES: Fc1ccccc1Cn1nc(-c2ncc(F)c(NC3CNC3)n2)cc1-c1ccon1] | I-38 |
| 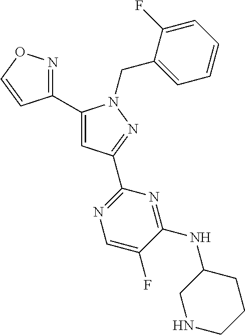 [SMILES: Fc1ccccc1Cn1nc(-c2ncc(F)c(NC3CCCNC3)n2)cc1-c1ccon1] | I-39 |
| 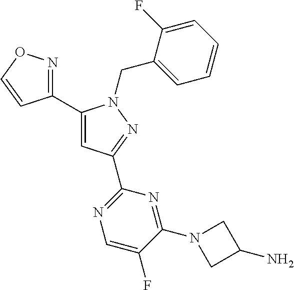 [SMILES: NC1CN(c2nc(-c3cc(-c4ccon4)n(Cc4ccccc4F)n3)ncc2F)C1] | I-40 |
| 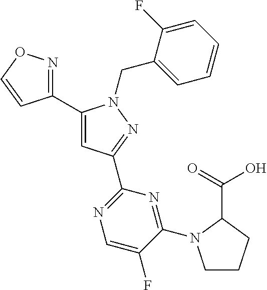 [SMILES: O=C(O)C1CCCN1c1nc(-c2cc(-c3ccon3)n(Cc3ccccc3F)n2)ncc1F] | I-41 |
| 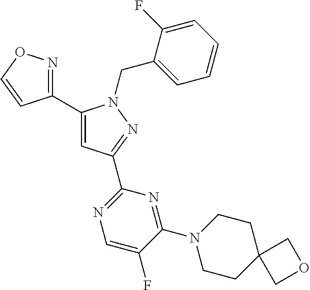 [SMILES: Fc1ccccc1Cn1nc(-c2ncc(F)c(N3CCC4(CC3)COC4)n2)cc1-c1ccon1] | I-42 |
| 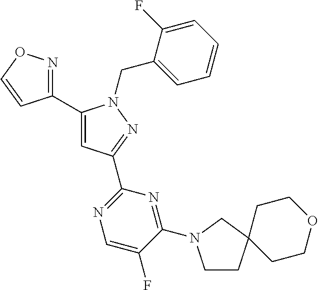 [SMILES: Fc1ccccc1Cn1nc(-c2ncc(F)c(N3CCC4(CCOCC4)C3)n2)cc1-c1ccon1] | I-43 |
| 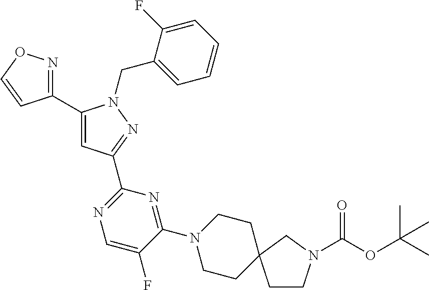 [SMILES: CC(C)(C)OC(=O)N1CCC2(CCN(c3nc(-c4cc(-c5ccon5)n(Cc5ccccc5F)n4)ncc3F)CC2)C1] | I-44 |
| 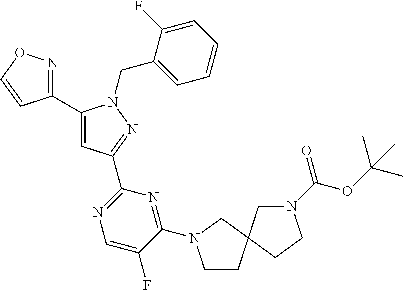 [SMILES: CC(C)(C)OC(=O)N1CCC2(CCN(c3nc(-c4cc(-c5ccon5)n(Cc5ccccc5F)n4)ncc3F)C2)C1] | I-45 |
| 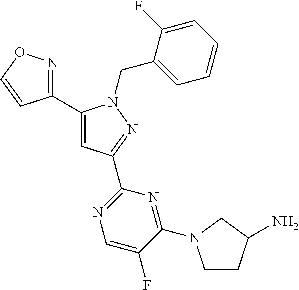 [SMILES: NC1CCN(c2nc(-c3cc(-c4ccon4)n(Cc4ccccc4F)n3)ncc2F)C1] | I-46 |
| 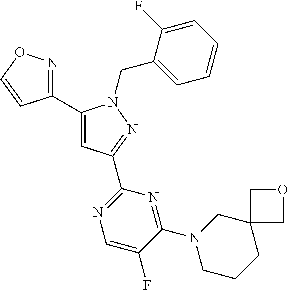 [SMILES: Fc1ccccc1Cn1nc(-c2ncc(F)c(N3CCCC4(COC4)C3)n2)cc1-c1ccon1] | I-47 |
| 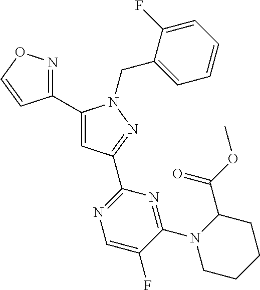 [SMILES: COC(=O)C1CCCCN1c1nc(-c2cc(-c3ccon3)n(Cc3ccccc3F)n2)ncc1F] | I-48 |
| 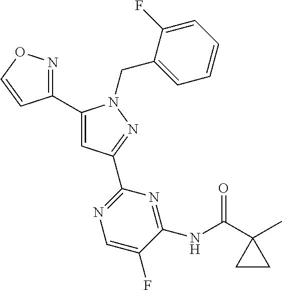 [SMILES: CC1(C(=O)Nc2nc(-c3cc(-c4ccon4)n(Cc4ccccc4F)n3)ncc2F)CC1] | I-49 |
| 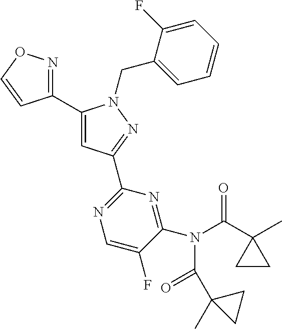 [SMILES: CC1(C(=O)N(C(=O)C2(C)CC2)c2nc(-c3cc(-c4ccon4)n(Cc4ccccc4F)n3)ncc2F)CC1] | I-50 |
| 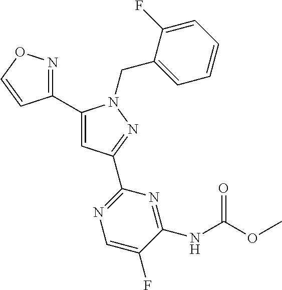 [SMILES: COC(=O)Nc1nc(-c2cc(-c3ccon3)n(Cc3ccccc3F)n2)ncc1F] | I-51 |
| 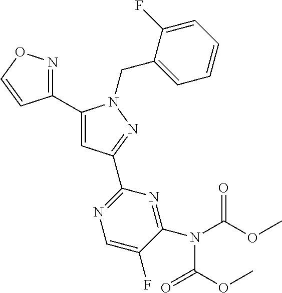 [SMILES: COC(=O)N(C(=O)OC)c1nc(-c2cc(-c3ccon3)n(Cc3ccccc3F)n2)ncc1F] | I-52 |
| 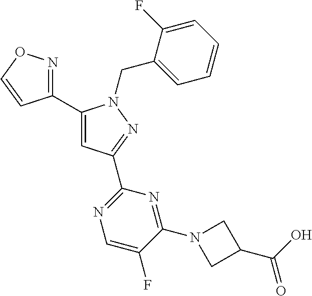 [SMILES: O=C(O)C1CN(c2nc(-c3cc(-c4ccon4)n(Cc4ccccc4F)n3)ncc2F)C1] | I-53 |
| 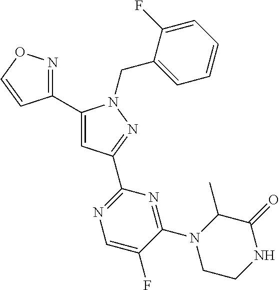 [SMILES: CC1C(=O)NCCN1c1nc(-c2cc(-c3ccon3)n(Cc3ccccc3F)n2)ncc1F] | I-54 |
| 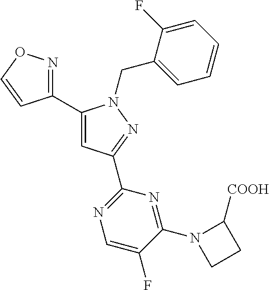 [SMILES: O=C(O)C1CCN1c1nc(-c2cc(-c3ccon3)n(Cc3ccccc3F)n2)ncc1F] | I-55 |
| 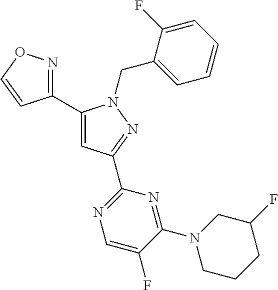 [SMILES: Fc1ccccc1Cn1nc(-c2ncc(F)c(N3CCCC(F)C3)n2)cc1-c1ccon1] | I-56 |
| 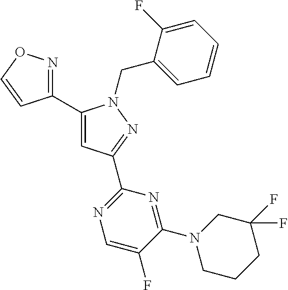 [SMILES: Fc1ccccc1Cn1nc(-c2ncc(F)c(N3CCCC(F)(F)C3)n2)cc1-c1ccon1] | I-57 |
| 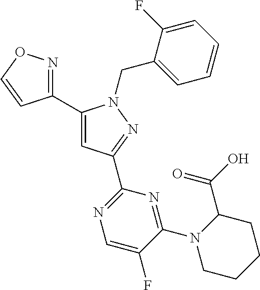 [SMILES: O=C(O)C1CCCCN1c1nc(-c2cc(-c3ccon3)n(Cc3ccccc3F)n2)ncc1F] | I-58 |
| 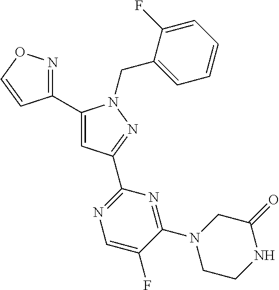 [SMILES: O=C1CN(c2nc(-c3cc(-c4ccon4)n(Cc4ccccc4F)n3)ncc2F)CCN1] | I-59 |
| 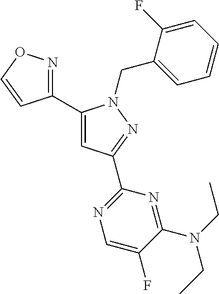 [SMILES: CCN(CC)c1nc(-c2cc(-c3ccon3)n(Cc3ccccc3F)n2)ncc1F] | I-60 |
| 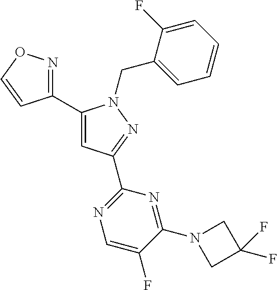 [SMILES: Fc1ccccc1Cn1nc(-c2ncc(F)c(N3CC(F)(F)C3)n2)cc1-c1ccon1] | I-61 |
| 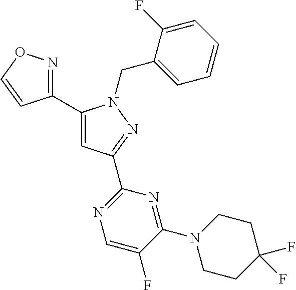 [SMILES: Fc1ccccc1Cn1nc(-c2ncc(F)c(N3CCC(F)(F)CC3)n2)cc1-c1ccon1] | I-62 |
| 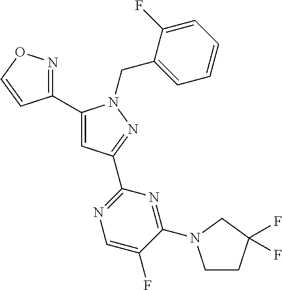 [SMILES: Fc1ccccc1Cn1nc(-c2ncc(F)c(N3CCC(F)(F)C3)n2)cc1-c1ccon1] | I-63 |
| 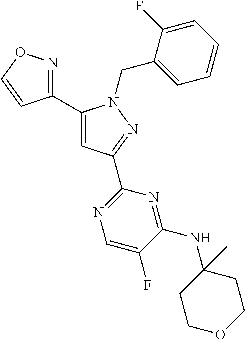 [SMILES: CC1(Nc2nc(-c3cc(-c4ccon4)n(Cc4ccccc4F)n3)ncc2F)CCOCC1] | I-64 |
| 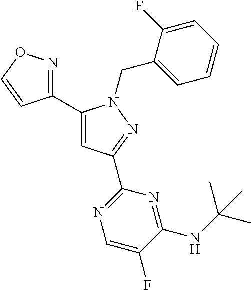 [SMILES: CC(C)(C)Nc1nc(-c2cc(-c3ccon3)n(Cc3ccccc3F)n2)ncc1F] | I-65 |
| 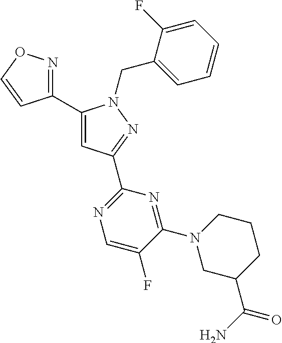 [SMILES: NC(=O)C1CCCN(c2nc(-c3cc(-c4ccon4)n(Cc4ccccc4F)n3)ncc2F)C1] | I-66 |
| 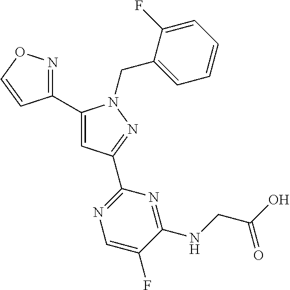 [SMILES: O=C(O)CNc1nc(-c2cc(-c3ccon3)n(Cc3ccccc3F)n2)ncc1F] | I-67 |
| 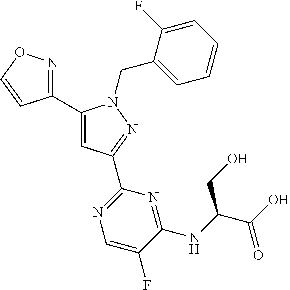 [SMILES: O=C(O)[C@H](CO)Nc1nc(-c2cc(-c3ccon3)n(Cc3ccccc3F)n2)ncc1F] | I-68 |
| 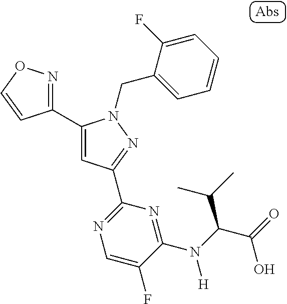 [SMILES: CC(C)[C@H](Nc1nc(-c2cc(-c3ccon3)n(Cc3ccccc3F)n2)ncc1F)C(=O)O] | I-69 |
| 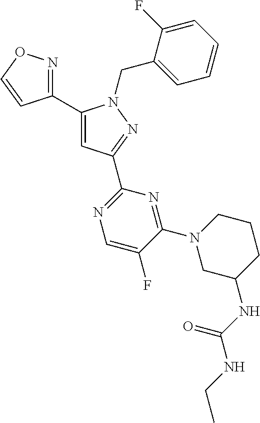 [SMILES: CCNC(=O)NC1CCCN(c2nc(-c3cc(-c4ccon4)n(Cc4ccccc4F)n3)ncc2F)C1] | I-70 |
| 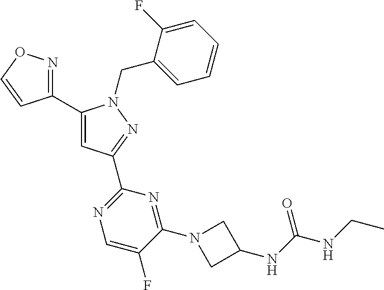 [SMILES: CCNC(=O)NC1CN(c2nc(-c3cc(-c4ccon4)n(Cc4ccccc4F)n3)ncc2F)C1] | I-71 |
| 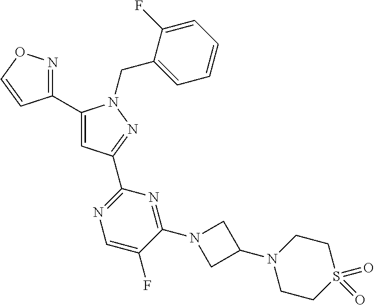 [SMILES: O=S1(=O)CCN(C2CN(c3nc(-c4cc(-c5ccon5)n(Cc5ccccc5F)n4)ncc3F)C2)CC1] | I-72 |
| 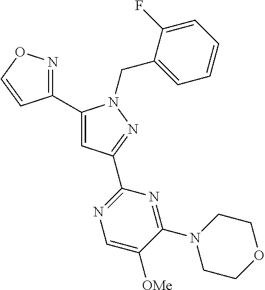 [SMILES: COc1cnc(-c2cc(-c3ccon3)n(Cc3ccccc3F)n2)nc1N1CCOCC1] | I-73 |
| 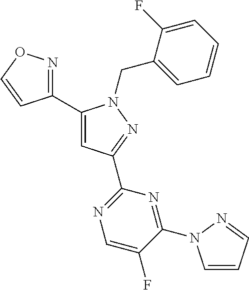 [SMILES: Fc1ccccc1Cn1nc(-c2ncc(F)c(-n3cccn3)n2)cc1-c1ccon1] | I-74 |
| 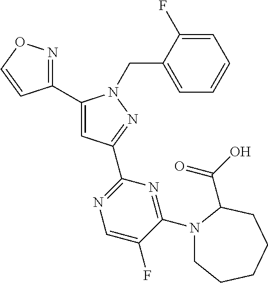 [SMILES: O=C(O)C1CCCCCN1c1nc(-c2cc(-c3ccon3)n(Cc3ccccc3F)n2)ncc1F] | I-75 |
| 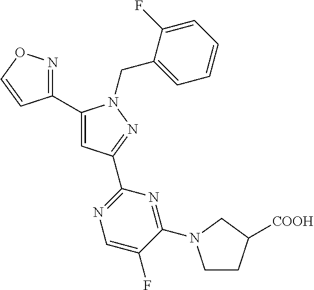 [SMILES: O=C(O)C1CCN(c2nc(-c3cc(-c4ccon4)n(Cc4ccccc4F)n3)ncc2F)C1] | I-76 |
| 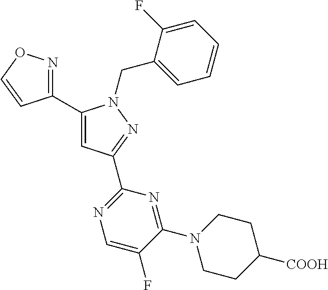 [SMILES: O=C(O)C1CCN(c2nc(-c3cc(-c4ccon4)n(Cc4ccccc4F)n3)ncc2F)CC1] | I-77 |
| 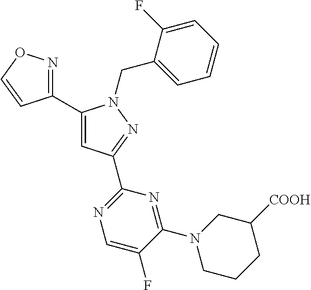 [SMILES: O=C(O)C1CCCN(c2nc(-c3cc(-c4ccon4)n(Cc4ccccc4F)n3)ncc2F)C1] | I-78 |
| 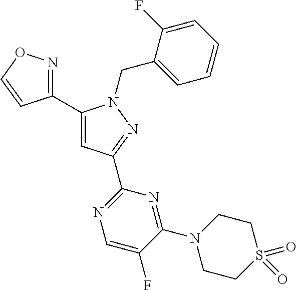 [SMILES: O=S1(=O)CCN(c2nc(-c3cc(-c4ccon4)n(Cc4ccccc4F)n3)ncc2F)CC1] | I-79 |
| 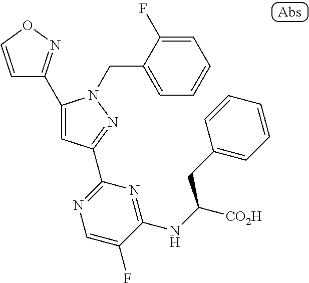 [SMILES: O=C(O)[C@H](Cc1ccccc1)Nc1nc(-c2cc(-c3ccon3)n(Cc3ccccc3F)n2)ncc1F] | I-80 |
| 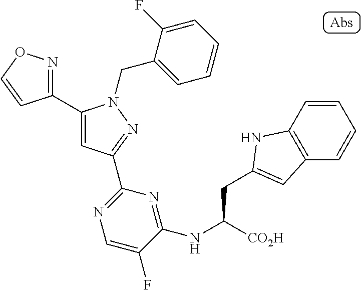 [SMILES: O=C(O)[C@H](Cc1cc2ccccc2[nH]1)Nc1nc(-c2cc(-c3ccon3)n(Cc3ccccc3F)n2)ncc1F] | I-81 |
| 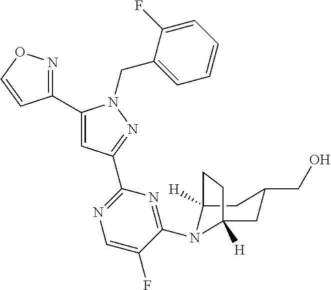 [SMILES: OCC1C[C@H]2CC[C@@H](C1)N2c1nc(-c2cc(-c3ccon3)n(Cc3ccccc3F)n2)ncc1F] | I-82 |
| 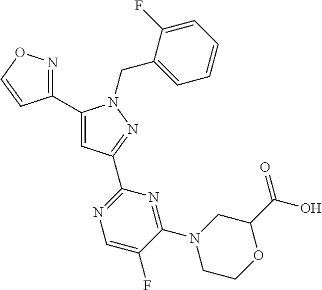 [SMILES: O=C(O)C1CN(c2nc(-c3cc(-c4ccon4)n(Cc4ccccc4F)n3)ncc2F)CCO1] | I-83 |
| 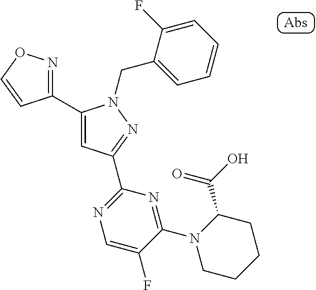 [SMILES: O=C(O)[C@@H]1CCCCN1c1nc(-c2cc(-c3ccon3)n(Cc3ccccc3F)n2)ncc1F] | I-84 |
| 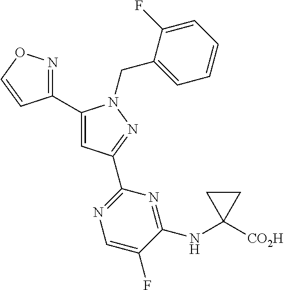 [SMILES: O=C(O)C1(Nc2nc(-c3cc(-c4ccon4)n(Cc4ccccc4F)n3)ncc2F)CC1] | I-85 |
| 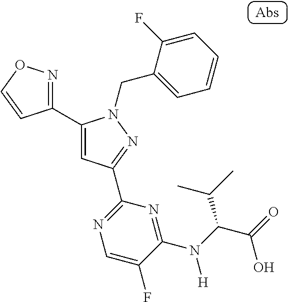 [SMILES: CC(C)[C@@H](Nc1nc(-c2cc(-c3ccon3)n(Cc3ccccc3F)n2)ncc1F)C(=O)O] | I-86 |
| 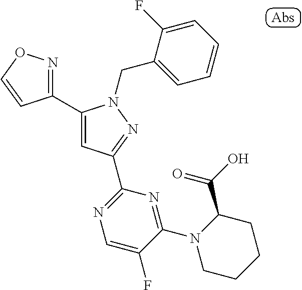 [SMILES: O=C(O)[C@H]1CCCCN1c1nc(-c2cc(-c3ccon3)n(Cc3ccccc3F)n2)ncc1F] | I-87 |
| 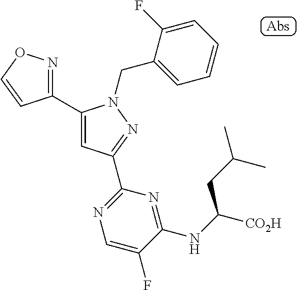 [SMILES: CC(C)C[C@H](Nc1nc(-c2cc(-c3ccon3)n(Cc3ccccc3F)n2)ncc1F)C(=O)O] | I-88 |
| 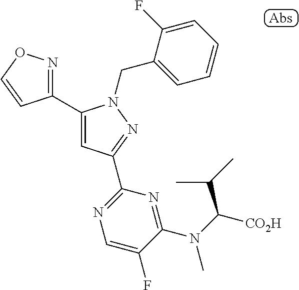 [SMILES: CC(C)[C@@H](C(=O)O)N(C)c1nc(-c2cc(-c3ccon3)n(Cc3ccccc3F)n2)ncc1F] | I-89 |
| 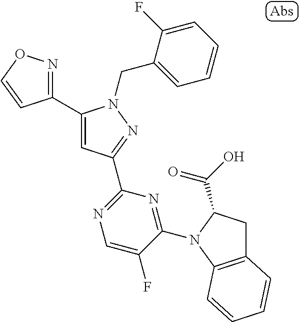 [SMILES: O=C(O)[C@@H]1Cc2ccccc2N1c1nc(-c2cc(-c3ccon3)n(Cc3ccccc3F)n2)ncc1F] | I-90 |
| 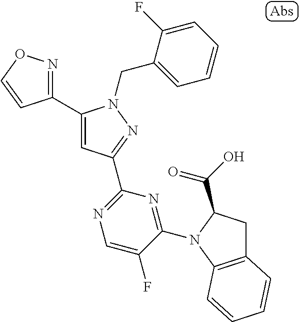 [SMILES: O=C(O)[C@H]1Cc2ccccc2N1c1nc(-c2cc(-c3ccon3)n(Cc3ccccc3F)n2)ncc1F] | I-91 |
| 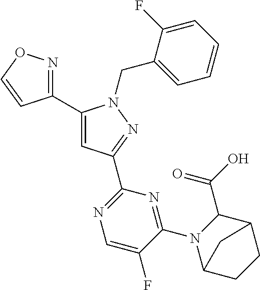 [SMILES: O=C(O)C1C2CCC(C2)N1c1nc(-c2cc(-c3ccon3)n(Cc3ccccc3F)n2)ncc1F] | I-92 |
| 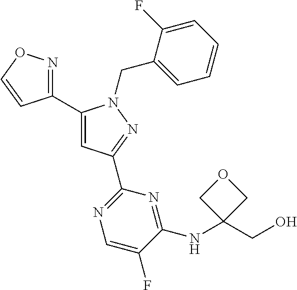 [SMILES: OCC1(Nc2nc(-c3cc(-c4ccon4)n(Cc4ccccc4F)n3)ncc2F)COC1] | I-93 |
| 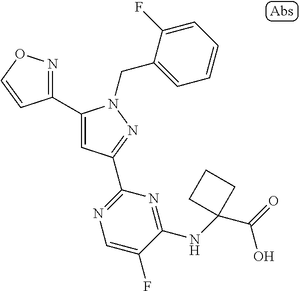 [SMILES: O=C(O)C1(Nc2nc(-c3cc(-c4ccon4)n(Cc4ccccc4F)n3)ncc2F)CCC1] | I-94 |
| 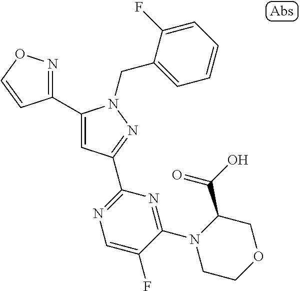 [SMILES: O=C(O)[C@H]1COCCN1c1nc(-c2cc(-c3ccon3)n(Cc3ccccc3F)n2)ncc1F] | I-95 |
| 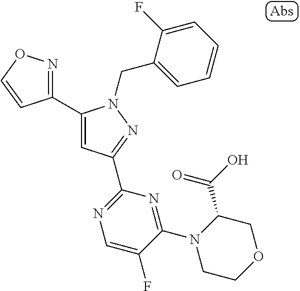 [SMILES: O=C(O)[C@@H]1COCCN1c1nc(-c2cc(-c3ccon3)n(Cc3ccccc3F)n2)ncc1F] | I-96 |
| 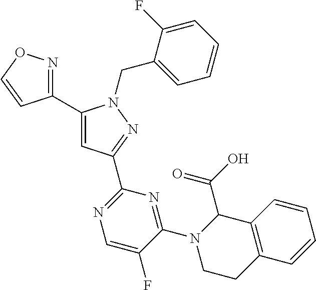 [SMILES: O=C(O)C1c2ccccc2CCN1c1nc(-c2cc(-c3ccon3)n(Cc3ccccc3F)n2)ncc1F] | I-97 |
| 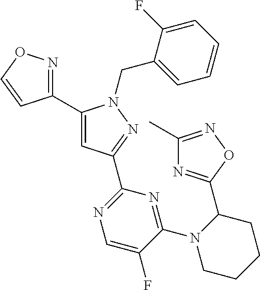 [SMILES: Cc1noc(C2CCCCN2c2nc(-c3cc(-c4ccon4)n(Cc4ccccc4F)n3)ncc2F)n1] | I-98 |
| 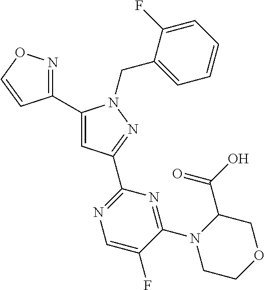 [SMILES: O=C(O)C1COCCN1c1nc(-c2cc(-c3ccon3)n(Cc3ccccc3F)n2)ncc1F] | I-99 |
| 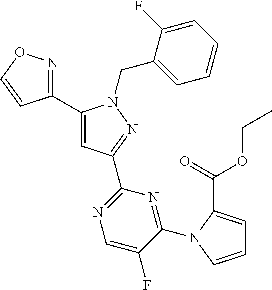 [SMILES: CCOC(=O)c1cccn1-c1nc(-c2cc(-c3ccon3)n(Cc3ccccc3F)n2)ncc1F] | I-100 |
| 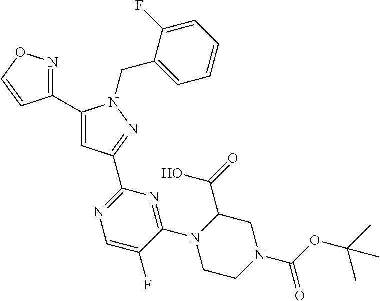 [SMILES: CC(C)(C)OC(=O)N1CCN(c2nc(-c3cc(-c4ccon4)n(Cc4ccccc4F)n3)ncc2F)C(C(=O)O)C1] | I-101 |
| 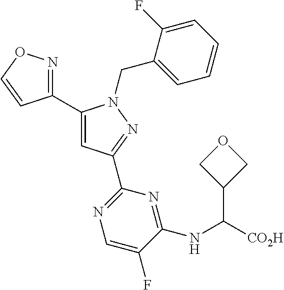 [SMILES: O=C(O)C(Nc1nc(-c2cc(-c3ccon3)n(Cc3ccccc3F)n2)ncc1F)C1COC1] | I-102 |
| 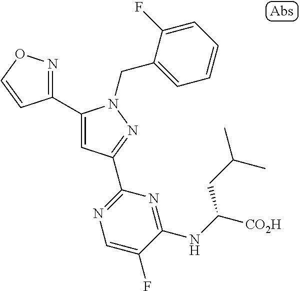 [SMILES: CC(C)C[C@@H](Nc1nc(-c2cc(-c3ccon3)n(Cc3ccccc3F)n2)ncc1F)C(=O)O] | I-103 |
| 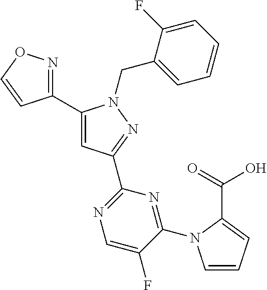 [SMILES: O=C(O)c1cccn1-c1nc(-c2cc(-c3ccon3)n(Cc3ccccc3F)n2)ncc1F] | I-104 |
| 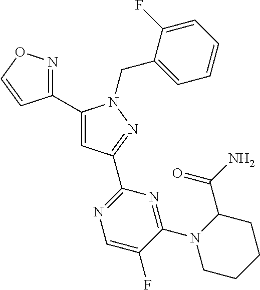 [SMILES: NC(=O)C1CCCCN1c1nc(-c2cc(-c3ccon3)n(Cc3ccccc3F)n2)ncc1F] | I-105 |
| 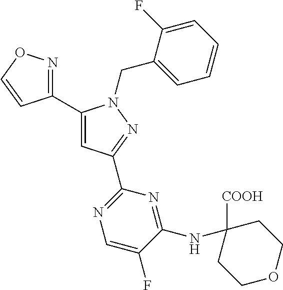 [SMILES: O=C(O)C1(Nc2nc(-c3cc(-c4ccon4)n(Cc4ccccc4F)n3)ncc2F)CCOCC1] | I-106 |
| 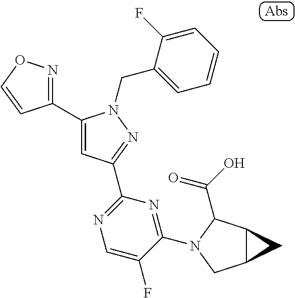 [SMILES: O=C(O)C1[C@H]2C[C@H]2CN1c1nc(-c2cc(-c3ccon3)n(Cc3ccccc3F)n2)ncc1F] | I-107 |
| 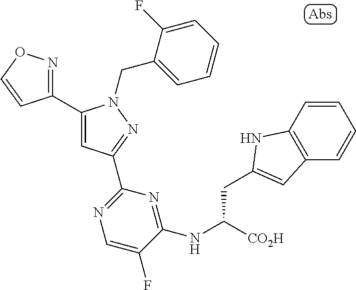 [SMILES: O=C(O)[C@@H](Cc1cc2ccccc2[nH]1)Nc1nc(-c2cc(-c3ccon3)n(Cc3ccccc3F)n2)ncc1F] | I-108 |
| 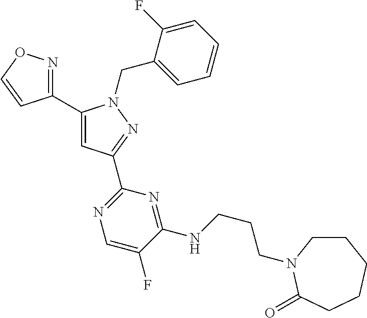 [SMILES: O=C1CCCCCN1CCCNc1nc(-c2cc(-c3ccon3)n(Cc3ccccc3F)n2)ncc1F] | I-109 |
| 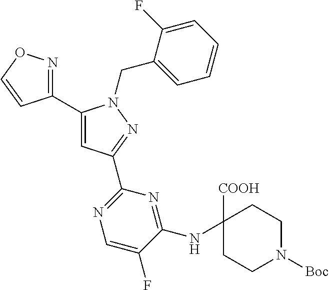 [SMILES: CC(C)(C)OC(=O)N1CCC(Nc2nc(-c3cc(-c4ccon4)n(Cc4ccccc4F)n3)ncc2F)(C(=O)O)CC1] | I-110 |
| 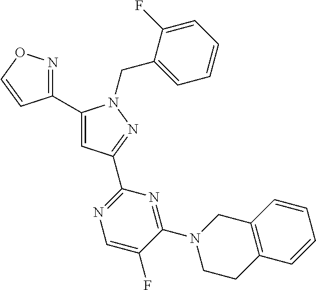 [SMILES: Fc1ccccc1Cn1nc(-c2ncc(F)c(N3CCc4ccccc4C3)n2)cc1-c1ccon1] | I-111 |
| 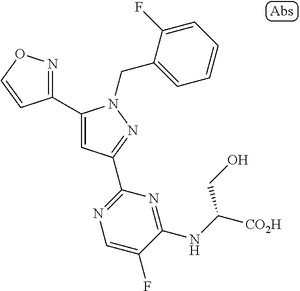 [SMILES: O=C(O)[C@@H](CO)Nc1nc(-c2cc(-c3ccon3)n(Cc3ccccc3F)n2)ncc1F] | I-112 |
| 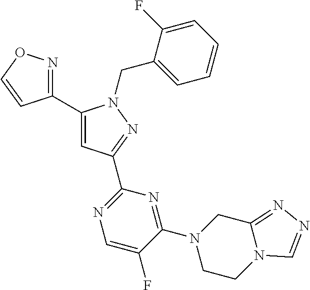 [SMILES: Fc1ccccc1Cn1nc(-c2ncc(F)c(N3CCn4cnnc4C3)n2)cc1-c1ccon1] | I-113 |
| 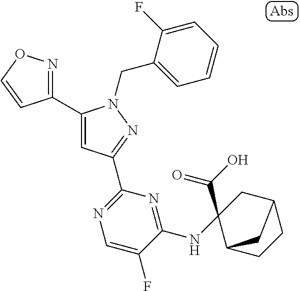 [SMILES: O=C(O)[C@@]1(Nc2nc(-c3cc(-c4ccon4)n(Cc4ccccc4F)n3)ncc2F)CC2CC[C@H]1C2] | I-114 |
| 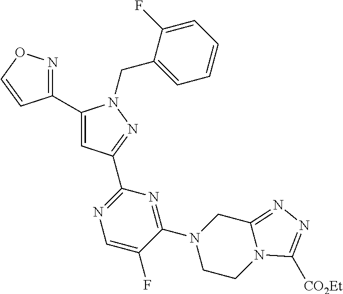 [SMILES: CCOC(=O)c1nnc2n1CCN(c1nc(-c3cc(-c4ccon4)n(Cc4ccccc4F)n3)ncc1F)C2] | I-115 |
| 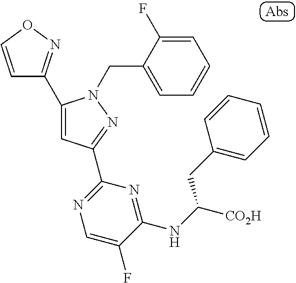 [SMILES: O=C(O)[C@@H](Cc1ccccc1)Nc1nc(-c2cc(-c3ccon3)n(Cc3ccccc3F)n2)ncc1F] | I-116 |
| 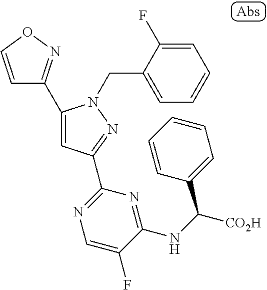 [SMILES: O=C(O)[C@@H](Nc1nc(-c2cc(-c3ccon3)n(Cc3ccccc3F)n2)ncc1F)c1ccccc1] | I-117 |
| 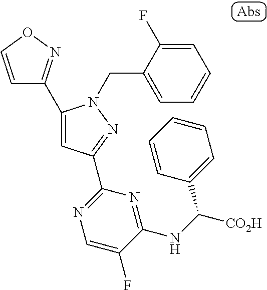 [SMILES: O=C(O)[C@H](Nc1nc(-c2cc(-c3ccon3)n(Cc3ccccc3F)n2)ncc1F)c1ccccc1] | I-118 |
| 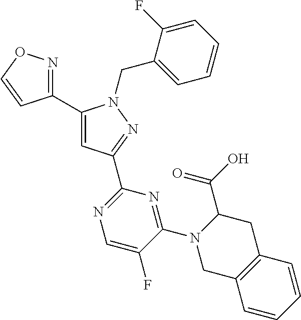 [SMILES: O=C(O)C1Cc2ccccc2CN1c1nc(-c2cc(-c3ccon3)n(Cc3ccccc3F)n2)ncc1F] | I-119 |
| 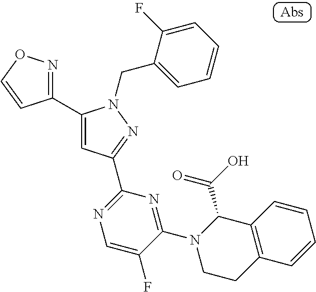 [SMILES: O=C(O)[C@@H]1c2ccccc2CCN1c1nc(-c2cc(-c3ccon3)n(Cc3ccccc3F)n2)ncc1F] | I-120 |
| 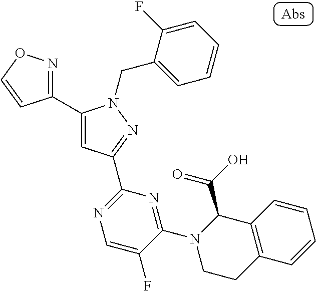 [SMILES: O=C(O)[C@H]1c2ccccc2CCN1c1nc(-c2cc(-c3ccon3)n(Cc3ccccc3F)n2)ncc1F] | I-121 |
| 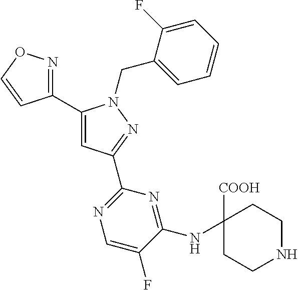 [SMILES: O=C(O)C1(Nc2nc(-c3cc(-c4ccon4)n(Cc4ccccc4F)n3)ncc2F)CCNCC1] | I-122 |
| 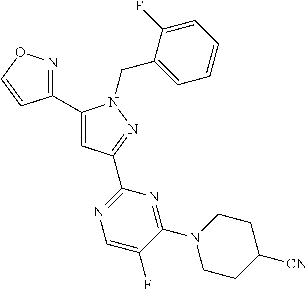 [SMILES: N#CC1CCN(c2nc(-c3cc(-c4ccon4)n(Cc4ccccc4F)n3)ncc2F)CC1] | I-123 |
| 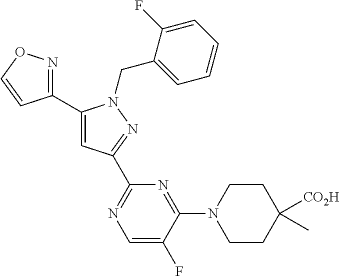 [SMILES: CC1(C(=O)O)CCN(c2nc(-c3cc(-c4ccon4)n(Cc4ccccc4F)n3)ncc2F)CC1] | I-124 |
| 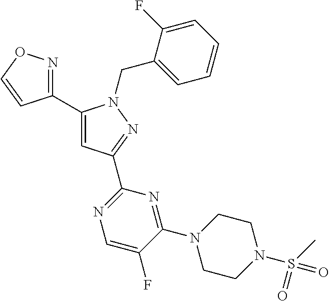 [SMILES: CS(=O)(=O)N1CCN(c2nc(-c3cc(-c4ccon4)n(Cc4ccccc4F)n3)ncc2F)CC1] | I-125 |
| 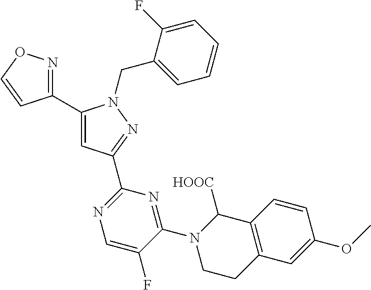 [SMILES: COc1ccc2c(c1)CCN(c1nc(-c3cc(-c4ccon4)n(Cc4ccccc4F)n3)ncc1F)C2C(=O)O] | I-126 |
| 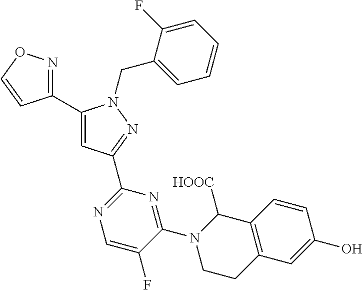 [SMILES: O=C(O)C1c2ccc(O)cc2CCN1c1nc(-c2cc(-c3ccon3)n(Cc3ccccc3F)n2)ncc1F] | I-127 |
| 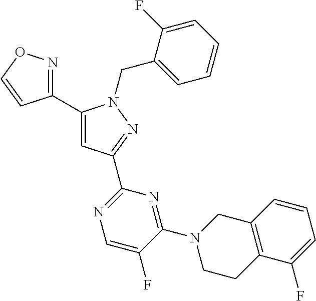 [SMILES: Fc1ccccc1Cn1nc(-c2ncc(F)c(N3CCc4c(F)cccc4C3)n2)cc1-c1ccon1] | I-128 |
| 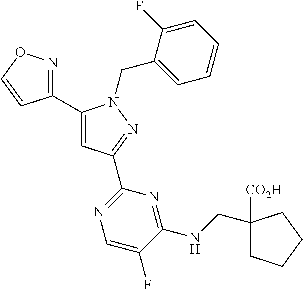 [SMILES: O=C(O)C1(CNc2nc(-c3cc(-c4ccon4)n(Cc4ccccc4F)n3)ncc2F)CCCC1] | I-129 |
| 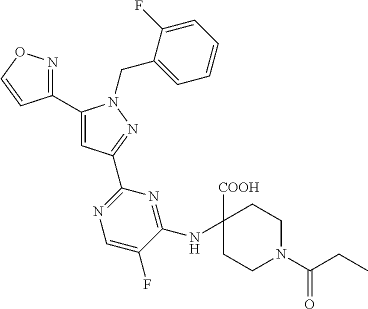 [SMILES: CCC(=O)N1CCC(Nc2nc(-c3cc(-c4ccon4)n(Cc4ccccc4F)n3)ncc2F)(C(=O)O)CC1] | I-130 |
| 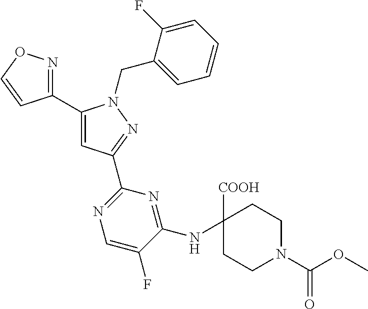 [SMILES: COC(=O)N1CCC(Nc2nc(-c3cc(-c4ccon4)n(Cc4ccccc4F)n3)ncc2F)(C(=O)O)CC1] | I-131 |
| 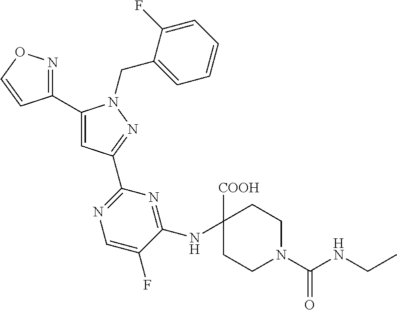 [SMILES: CCNC(=O)N1CCC(Nc2nc(-c3cc(-c4ccon4)n(Cc4ccccc4F)n3)ncc2F)(C(=O)O)CC1] | I-132 |
| 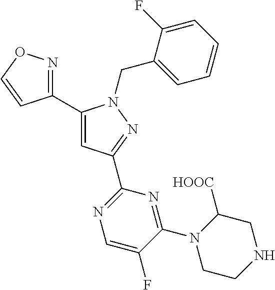 [SMILES: O=C(O)C1CNCCN1c1nc(-c2cc(-c3ccon3)n(Cc3ccccc3F)n2)ncc1F] | I-133 |
| 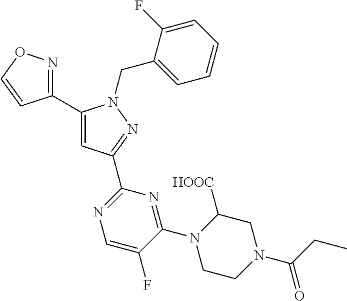 [SMILES: CCC(=O)N1CCN(c2nc(-c3cc(-c4ccon4)n(Cc4ccccc4F)n3)ncc2F)C(C(=O)O)C1] | I-134 |
| 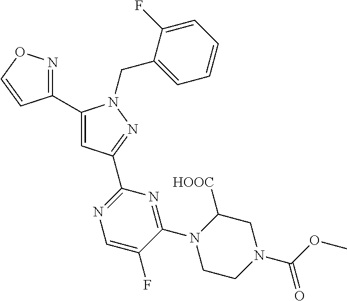 [SMILES: COC(=O)N1CCN(c2nc(-c3cc(-c4ccon4)n(Cc4ccccc4F)n3)ncc2F)C(C(=O)O)C1] | I-135 |
| 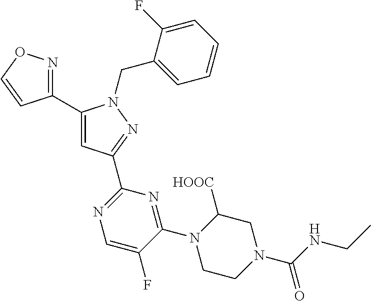 [SMILES: CCNC(=O)N1CCN(c2nc(-c3cc(-c4ccon4)n(Cc4ccccc4F)n3)ncc2F)C(C(=O)O)C1] | I-136 |
| 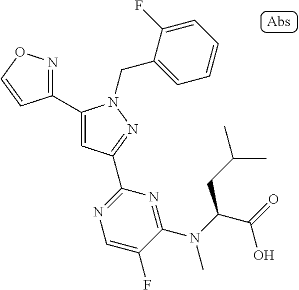 [SMILES: CC(C)C[C@@H](C(=O)O)N(C)c1nc(-c2cc(-c3ccon3)n(Cc3ccccc3F)n2)ncc1F] | I-137 |
| 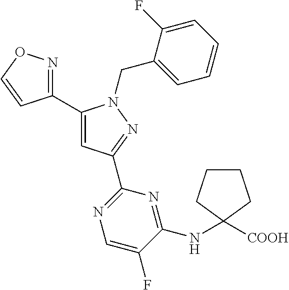 [SMILES: O=C(O)C1(Nc2nc(-c3cc(-c4ccon4)n(Cc4ccccc4F)n3)ncc2F)CCCC1] | I-138 |
| 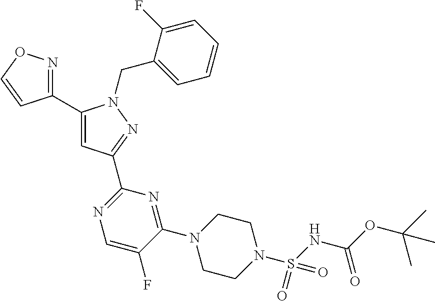 [SMILES: CC(C)(C)OC(=O)NS(=O)(=O)N1CCN(c2nc(-c3cc(-c4ccon4)n(Cc4ccccc4F)n3)ncc2F)CC1] | I-139 |
| 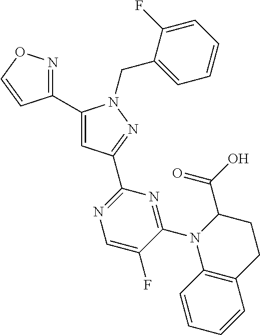 [SMILES: O=C(O)C1CCc2ccccc2N1c1nc(-c2cc(-c3ccon3)n(Cc3ccccc3F)n2)ncc1F] | I-140 |
| 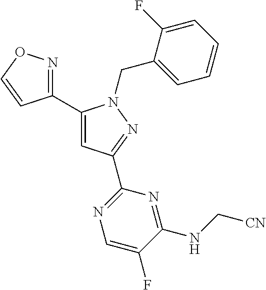 [SMILES: N#CCNc1nc(-c2cc(-c3ccon3)n(Cc3ccccc3F)n2)ncc1F] | I-141 |
| 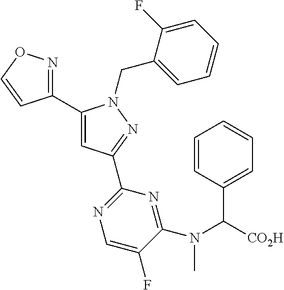 [SMILES: CN(c1nc(-c2cc(-c3ccon3)n(Cc3ccccc3F)n2)ncc1F)C(C(=O)O)c1ccccc1] | I-142 |
| 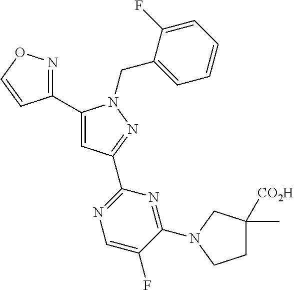 [SMILES: CC1(C(=O)O)CCN(c2nc(-c3cc(-c4ccon4)n(Cc4ccccc4F)n3)ncc2F)C1] | I-143 |
| 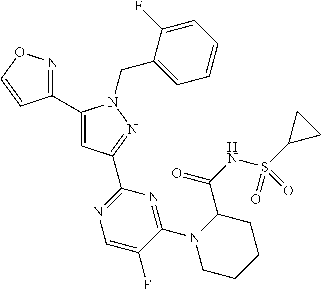 [SMILES: O=C(NS(=O)(=O)C1CC1)C1CCCCN1c1nc(-c2cc(-c3ccon3)n(Cc3ccccc3F)n2)ncc1F] | I-144 |
| 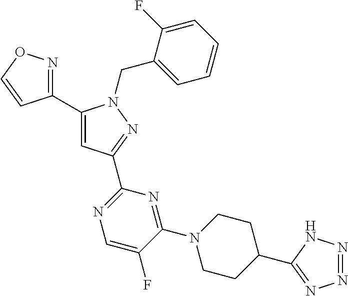 [SMILES: Fc1ccccc1Cn1nc(-c2ncc(F)c(N3CCC(c4nnn[nH]4)CC3)n2)cc1-c1ccon1] | I-145 |
| 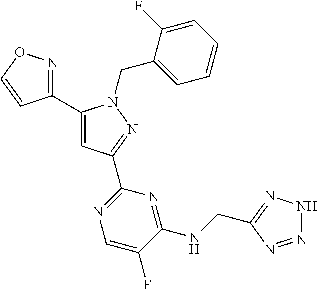 [SMILES: Fc1ccccc1Cn1nc(-c2ncc(F)c(NCc3nn[nH]n3)n2)cc1-c1ccon1] | I-146 |
| 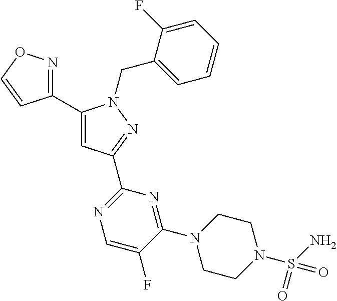 [SMILES: NS(=O)(=O)N1CCN(c2nc(-c3cc(-c4ccon4)n(Cc4ccccc4F)n3)ncc2F)CC1] | I-147 |
| 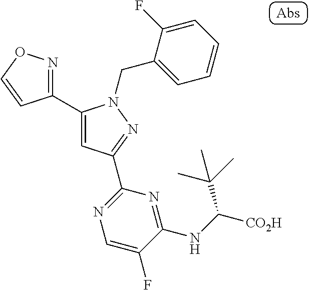 [SMILES: CC(C)(C)[C@@H](Nc1nc(-c2cc(-c3ccon3)n(Cc3ccccc3F)n2)ncc1F)C(=O)O] | I-148 |
| 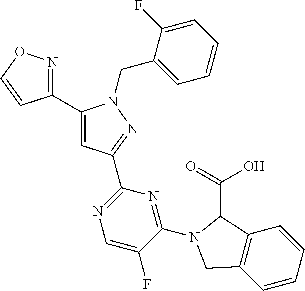 [SMILES: O=C(O)C1c2ccccc2CN1c1nc(-c2cc(-c3ccon3)n(Cc3ccccc3F)n2)ncc1F] | I-149 |
| 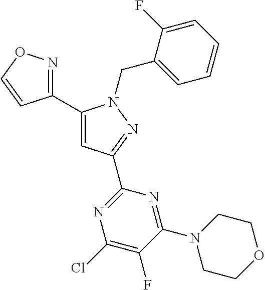 [SMILES: Fc1ccccc1Cn1nc(-c2nc(Cl)c(F)c(N3CCOCC3)n2)cc1-c1ccon1] | I-150 |
| 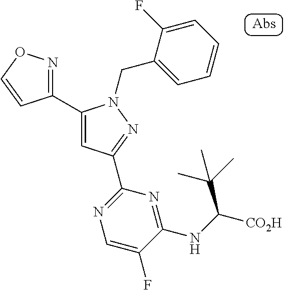 [SMILES: CC(C)(C)[C@H](Nc1nc(-c2cc(-c3ccon3)n(Cc3ccccc3F)n2)ncc1F)C(=O)O] | I-151 |
| 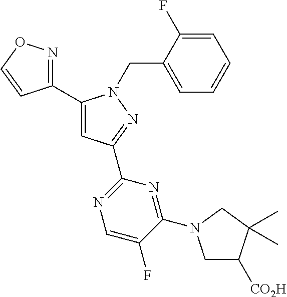 [SMILES: CC1(C)CN(c2nc(-c3cc(-c4ccon4)n(Cc4ccccc4F)n3)ncc2F)CC1C(=O)O] | I-152 |
| 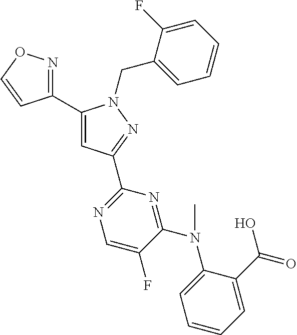 [SMILES: CN(c1ccccc1C(=O)O)c1nc(-c2cc(-c3ccon3)n(Cc3ccccc3F)n2)ncc1F] | I-153 |
| 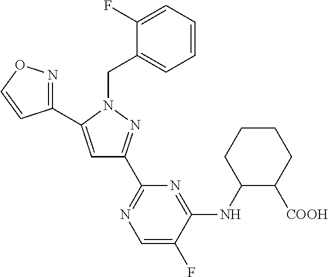 [SMILES: O=C(O)C1CCCCC1Nc1nc(-c2cc(-c3ccon3)n(Cc3ccccc3F)n2)ncc1F] | I-154 |
| 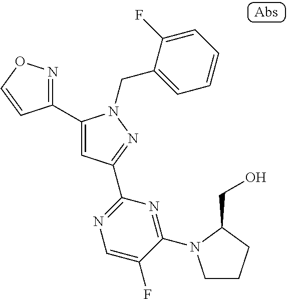 [SMILES: OC[C@H]1CCCN1c1nc(-c2cc(-c3ccon3)n(Cc3ccccc3F)n2)ncc1F] | I-155 |
| 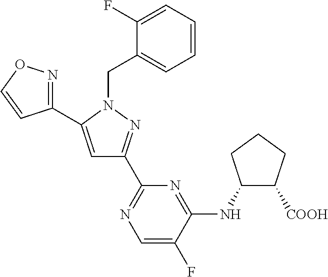 [SMILES: O=C(O)[C@H]1CCC[C@H]1Nc1nc(-c2cc(-c3ccon3)n(Cc3ccccc3F)n2)ncc1F] | I-156 |
| 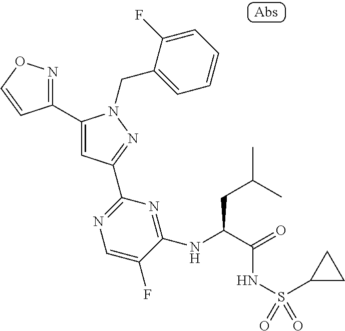 [SMILES: CC(C)C[C@H](Nc1nc(-c2cc(-c3ccon3)n(Cc3ccccc3F)n2)ncc1F)C(=O)NS(=O)(=O)C1CC1] | I-157 |
| 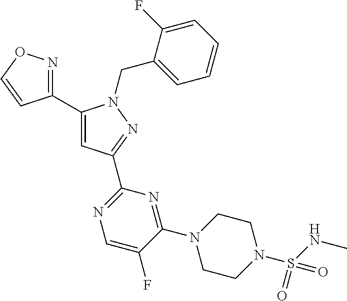 [SMILES: CNS(=O)(=O)N1CCN(c2nc(-c3cc(-c4ccon4)n(Cc4ccccc4F)n3)ncc2F)CC1] | I-158 |
| 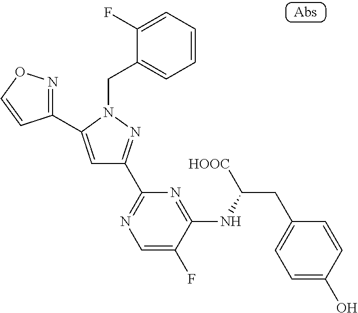 [SMILES: O=C(O)[C@H](Cc1ccc(O)cc1)Nc1nc(-c2cc(-c3ccon3)n(Cc3ccccc3F)n2)ncc1F] | I-159 |
| 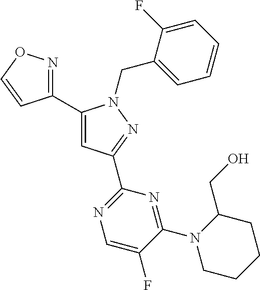 [SMILES: OCC1CCCCN1c1nc(-c2cc(-c3ccon3)n(Cc3ccccc3F)n2)ncc1F] | I-160 |
| 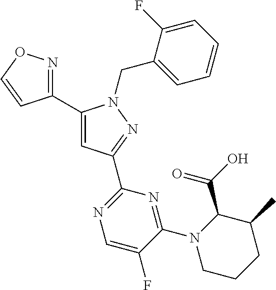 [SMILES: C[C@H]1CCCN(c2nc(-c3cc(-c4ccon4)n(Cc4ccccc4F)n3)ncc2F)[C@H]1C(=O)O] | I-161 |
| 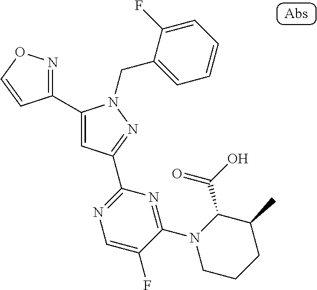 [SMILES: C[C@H]1CCCN(c2nc(-c3cc(-c4ccon4)n(Cc4ccccc4F)n3)ncc2F)[C@@H]1C(=O)O] | I-162 |
| 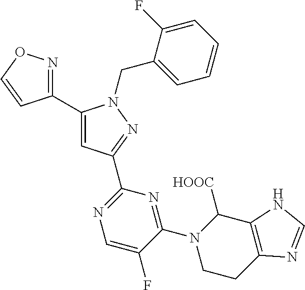 [SMILES: O=C(O)C1c2[nH]cnc2CCN1c1nc(-c2cc(-c3ccon3)n(Cc3ccccc3F)n2)ncc1F] | I-163 |
| 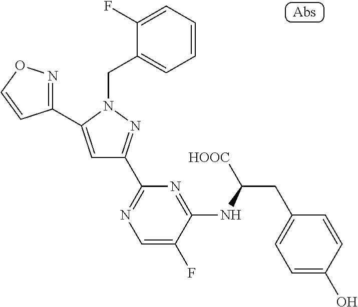 [SMILES: O=C(O)[C@@H](Cc1ccc(O)cc1)Nc1nc(-c2cc(-c3ccon3)n(Cc3ccccc3F)n2)ncc1F] | I-165 |
| 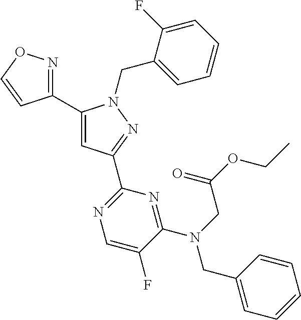 [SMILES: CCOC(=O)CN(Cc1ccccc1)c1nc(-c2cc(-c3ccon3)n(Cc3ccccc3F)n2)ncc1F] | I-166 |
| 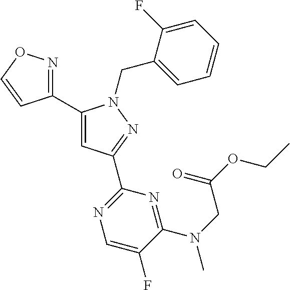 [SMILES: CCOC(=O)CN(C)c1nc(-c2cc(-c3ccon3)n(Cc3ccccc3F)n2)ncc1F] | I-167 |
| 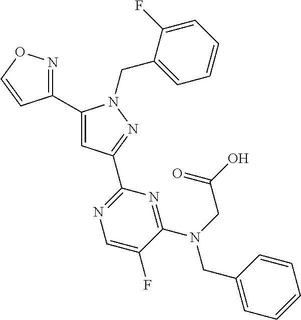 [SMILES: O=C(O)CN(Cc1ccccc1)c1nc(-c2cc(-c3ccon3)n(Cc3ccccc3F)n2)ncc1F] | I-168 |
| 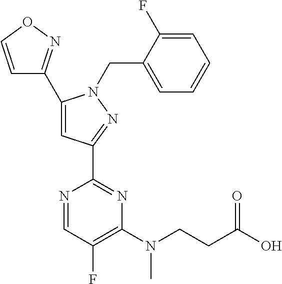 [SMILES: CN(CCC(=O)O)c1nc(-c2cc(-c3ccon3)n(Cc3ccccc3F)n2)ncc1F] | I-169 |
| 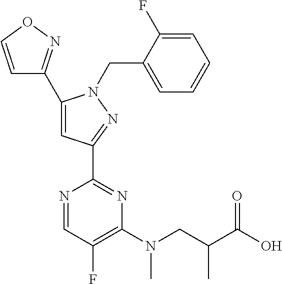 [SMILES: CC(CN(C)c1nc(-c2cc(-c3ccon3)n(Cc3ccccc3F)n2)ncc1F)C(=O)O] | I-170 |
| 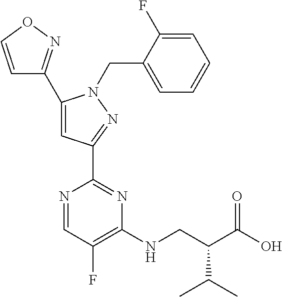 [SMILES: CC(C)[C@H](CNc1nc(-c2cc(-c3ccon3)n(Cc3ccccc3F)n2)ncc1F)C(=O)O] | I-171 |
| 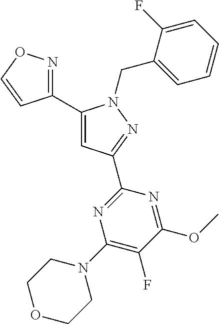 [SMILES: COc1nc(-c2cc(-c3ccon3)n(Cc3ccccc3F)n2)nc(N2CCOCC2)c1F] | I-172 |
| 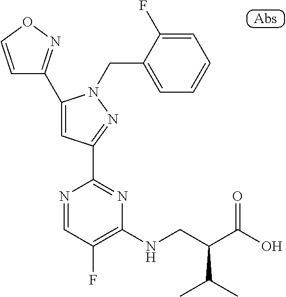 [SMILES: CC(C)[C@@H](CNc1nc(-c2cc(-c3ccon3)n(Cc3ccccc3F)n2)ncc1F)C(=O)O] | I-173 |
| 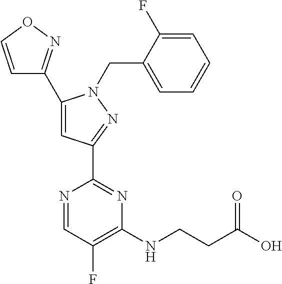 [SMILES: O=C(O)CCNc1nc(-c2cc(-c3ccon3)n(Cc3ccccc3F)n2)ncc1F] | I-174 |
| 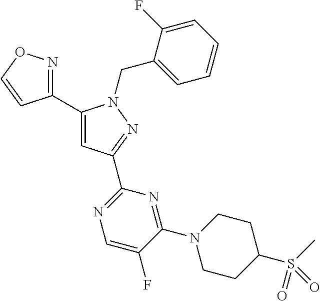 [SMILES: CS(=O)(=O)C1CCN(c2nc(-c3cc(-c4ccon4)n(Cc4ccccc4F)n3)ncc2F)CC1] | I-175 |
| 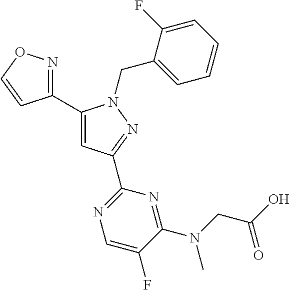 [SMILES: CN(CC(=O)O)c1nc(-c2cc(-c3ccon3)n(Cc3ccccc3F)n2)ncc1F] | I-176 |
| 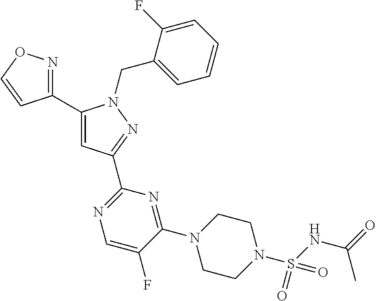 [SMILES: CC(=O)NS(=O)(=O)N1CCN(c2nc(-c3cc(-c4ccon4)n(Cc4ccccc4F)n3)ncc2F)CC1] | I-177 |
| 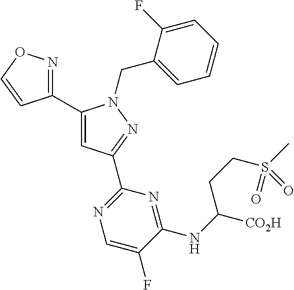 [SMILES: CS(=O)(=O)CCC(Nc1nc(-c2cc(-c3ccon3)n(Cc3ccccc3F)n2)ncc1F)C(=O)O] | I-178 |
| 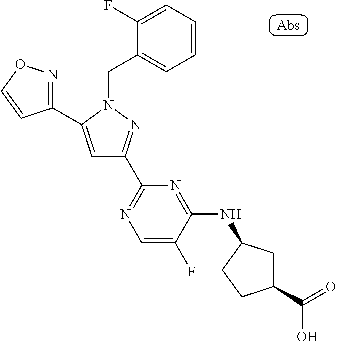 [SMILES: O=C(O)[C@H]1CC[C@@H](Nc2nc(-c3cc(-c4ccon4)n(Cc4ccccc4F)n3)ncc2F)C1] | I-179 |
| 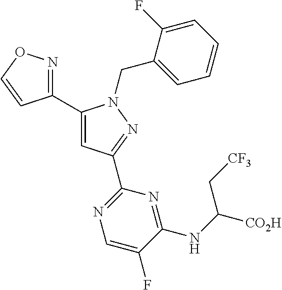 [SMILES: O=C(O)C(CC(F)(F)F)Nc1nc(-c2cc(-c3ccon3)n(Cc3ccccc3F)n2)ncc1F] | I-180 |
| 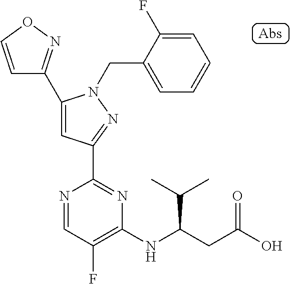 [SMILES: CC(C)[C@@H](CC(=O)O)Nc1nc(-c2cc(-c3ccon3)n(Cc3ccccc3F)n2)ncc1F] | I-181 |
| 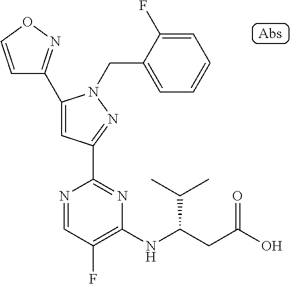 [SMILES: CC(C)[C@H](CC(=O)O)Nc1nc(-c2cc(-c3ccon3)n(Cc3ccccc3F)n2)ncc1F] | I-182 |
| 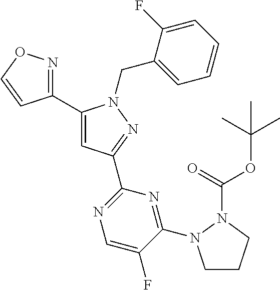 [SMILES: CC(C)(C)OC(=O)N1CCCN1c1nc(-c2cc(-c3ccon3)n(Cc3ccccc3F)n2)ncc1F] | I-183 |
| 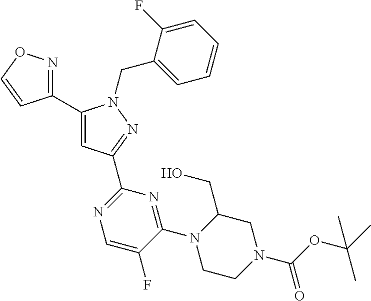 [SMILES: CC(C)(C)OC(=O)N1CCN(c2nc(-c3cc(-c4ccon4)n(Cc4ccccc4F)n3)ncc2F)C(CO)C1] | I-184 |
| 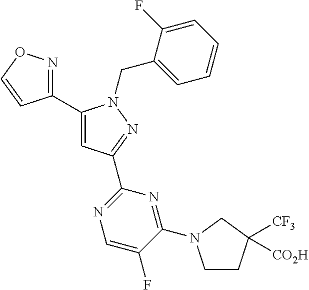 [SMILES: O=C(O)C1(C(F)(F)F)CCN(c2nc(-c3cc(-c4ccon4)n(Cc4ccccc4F)n3)ncc2F)C1] | I-185 |
| 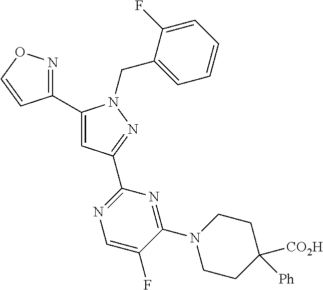 [SMILES: O=C(O)C1(c2ccccc2)CCN(c2nc(-c3cc(-c4ccon4)n(Cc4ccccc4F)n3)ncc2F)CC1] | I-186 |
| 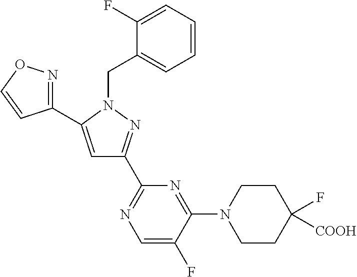 [SMILES: O=C(O)C1(F)CCN(c2nc(-c3cc(-c4ccon4)n(Cc4ccccc4F)n3)ncc2F)CC1] | I-188 |
| 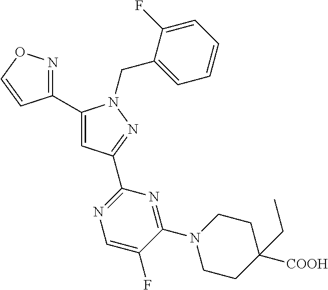 [SMILES: CCC1(C(=O)O)CCN(c2nc(-c3cc(-c4ccon4)n(Cc4ccccc4F)n3)ncc2F)CC1] | I-189 |
| 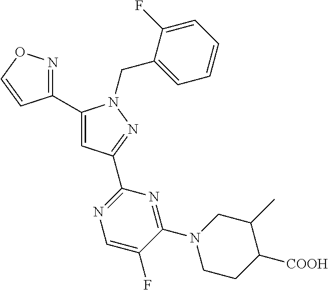 [SMILES: CC1CN(c2nc(-c3cc(-c4ccon4)n(Cc4ccccc4F)n3)ncc2F)CCC1C(=O)O] | I-190 |
| 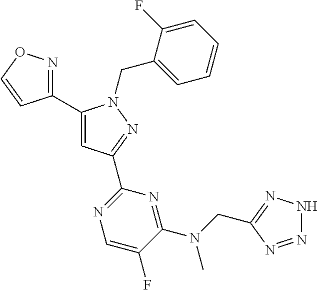 [SMILES: CN(Cc1nn[nH]n1)c1nc(-c2cc(-c3ccon3)n(Cc3ccccc3F)n2)ncc1F] | I-191 |
| 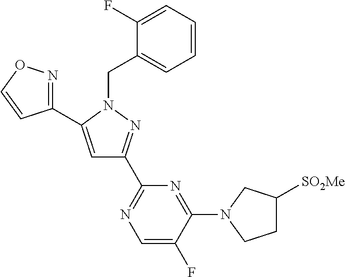 [SMILES: CS(=O)(=O)C1CCN(c2nc(-c3cc(-c4ccon4)n(Cc4ccccc4F)n3)ncc2F)C1] | I-192 |
| 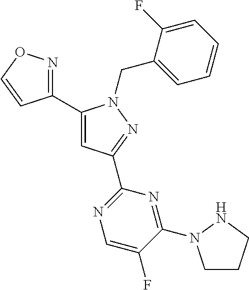 [SMILES: Fc1ccccc1Cn1nc(-c2ncc(F)c(N3CCCN3)n2)cc1-c1ccon1] | I-193 |
| 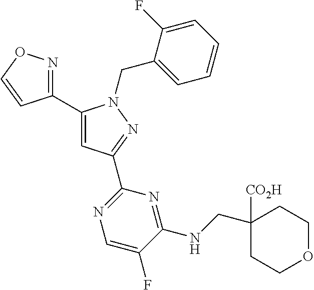 [SMILES: O=C(O)C1(CNc2nc(-c3cc(-c4ccon4)n(Cc4ccccc4F)n3)ncc2F)CCOCC1] | I-194 |
| 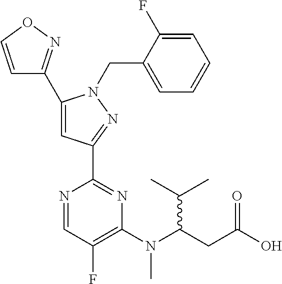 [SMILES: CC(C)C(CC(=O)O)N(C)c1nc(-c2cc(-c3ccon3)n(Cc3ccccc3F)n2)ncc1F] | I-195 |
| 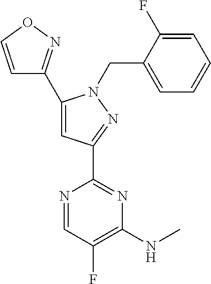 [SMILES: CNc1nc(-c2cc(-c3ccon3)n(Cc3ccccc3F)n2)ncc1F] | I-196 |
| 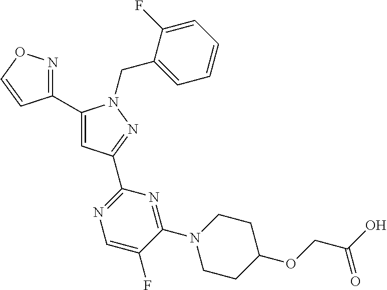 [SMILES: O=C(O)COC1CCN(c2nc(-c3cc(-c4ccon4)n(Cc4ccccc4F)n3)ncc2F)CC1] | I-197 |
| 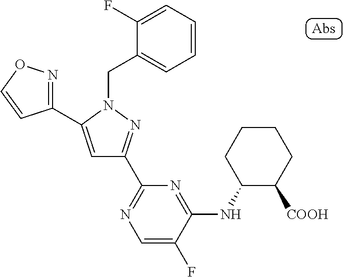 [SMILES: O=C(O)[C@@H]1CCCC[C@H]1Nc1nc(-c2cc(-c3ccon3)n(Cc3ccccc3F)n2)ncc1F] | I-198 |
| 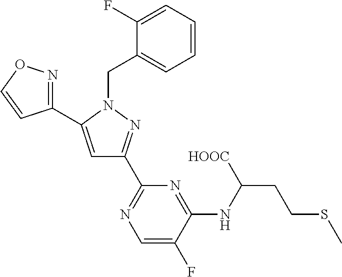 [SMILES: CSCCC(Nc1nc(-c2cc(-c3ccon3)n(Cc3ccccc3F)n2)ncc1F)C(=O)O] | I-199 |
| 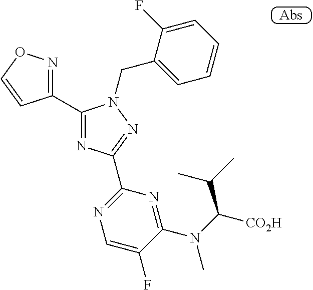 [SMILES: CC(C)[C@@H](C(=O)O)N(C)c1nc(-c2nc(-c3ccon3)n(Cc3ccccc3F)n2)ncc1F] | I-200 |
| 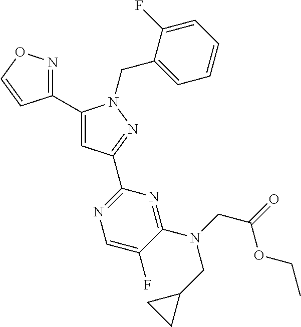 [SMILES: CCOC(=O)CN(CC1CC1)c1nc(-c2cc(-c3ccon3)n(Cc3ccccc3F)n2)ncc1F] | I-201 |
| 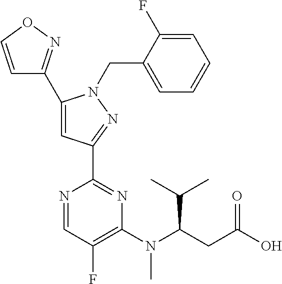 [SMILES: CC(C)[C@@H](CC(=O)O)N(C)c1nc(-c2cc(-c3ccon3)n(Cc3ccccc3F)n2)ncc1F] | I-202 |
| 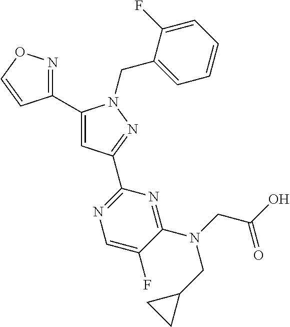 [SMILES: O=C(O)CN(CC1CC1)c1nc(-c2cc(-c3ccon3)n(Cc3ccccc3F)n2)ncc1F] | I-203 |
| 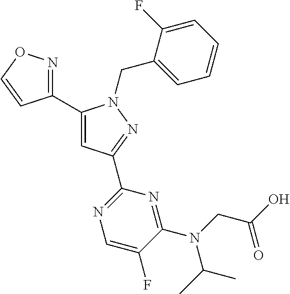 [SMILES: CC(C)N(CC(=O)O)c1nc(-c2cc(-c3ccon3)n(Cc3ccccc3F)n2)ncc1F] | I-204 |
| 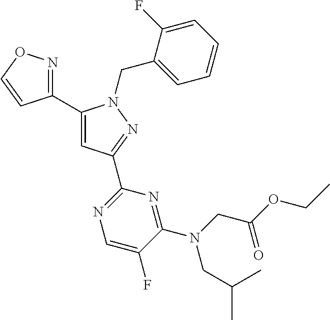 [SMILES: CCOC(=O)CN(CC(C)C)c1nc(-c2cc(-c3ccon3)n(Cc3ccccc3F)n2)ncc1F] | I-205 |
| 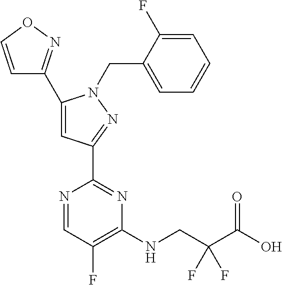 [SMILES: O=C(O)C(F)(F)CNc1nc(-c2cc(-c3ccon3)n(Cc3ccccc3F)n2)ncc1F] | I-206 |
| 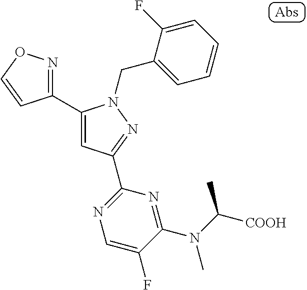 [SMILES: C[C@@H](C(=O)O)N(C)c1nc(-c2cc(-c3ccon3)n(Cc3ccccc3F)n2)ncc1F] | I-207 |
| 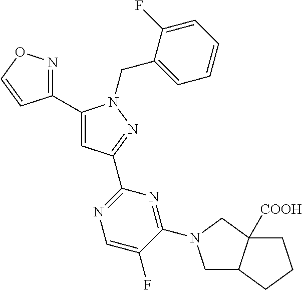 [SMILES: O=C(O)C12CCCC1CN(c1nc(-c3cc(-c4ccon4)n(Cc4ccccc4F)n3)ncc1F)C2] | I-208 |
| 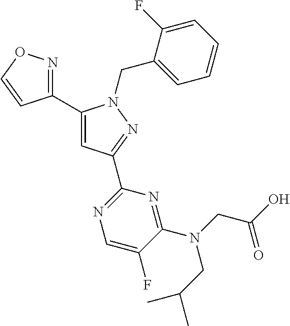 [SMILES: CC(C)CN(CC(=O)O)c1nc(-c2cc(-c3ccon3)n(Cc3ccccc3F)n2)ncc1F] | I-209 |
| 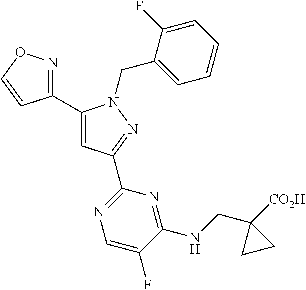 [SMILES: O=C(O)C1(CNc2nc(-c3cc(-c4ccon4)n(Cc4ccccc4F)n3)ncc2F)CC1] | I-210 |
| 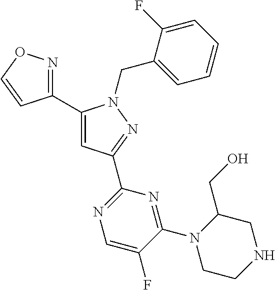 [SMILES: OCC1CNCCN1c1nc(-c2cc(-c3ccon3)n(Cc3ccccc3F)n2)ncc1F] | I-211 |
| 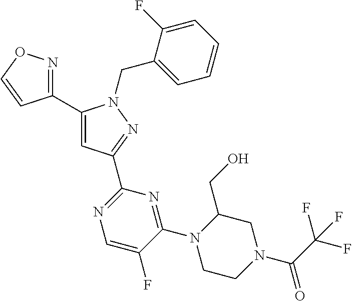 [SMILES: O=C(N1CCN(c2nc(-c3cc(-c4ccon4)n(Cc4ccccc4F)n3)ncc2F)C(CO)C1)C(F)(F)F] | I-212 |
| 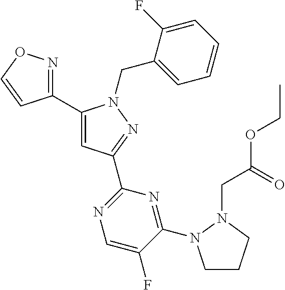 [SMILES: CCOC(=O)CN1CCCN1c1nc(-c2cc(-c3ccon3)n(Cc3ccccc3F)n2)ncc1F] | I-213 |
| 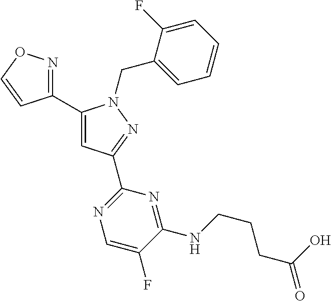 [SMILES: O=C(O)CCCNc1nc(-c2cc(-c3ccon3)n(Cc3ccccc3F)n2)ncc1F] | I-214 |
| 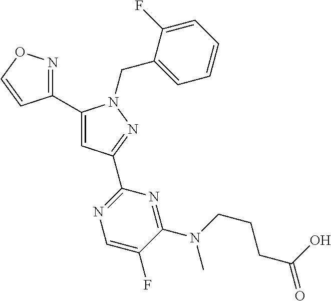 [SMILES: CN(CCCC(=O)O)c1nc(-c2cc(-c3ccon3)n(Cc3ccccc3F)n2)ncc1F] | I-215 |
| 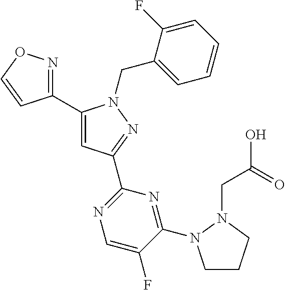 [SMILES: O=C(O)CN1CCCN1c1nc(-c2cc(-c3ccon3)n(Cc3ccccc3F)n2)ncc1F] | I-216 |
| 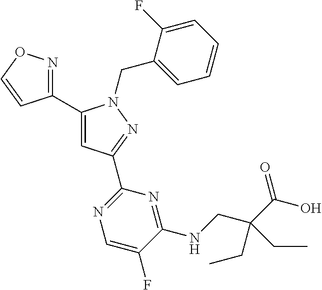 [SMILES: CCC(CC)(CNc1nc(-c2cc(-c3ccon3)n(Cc3ccccc3F)n2)ncc1F)C(=O)O] | I-217 |
| 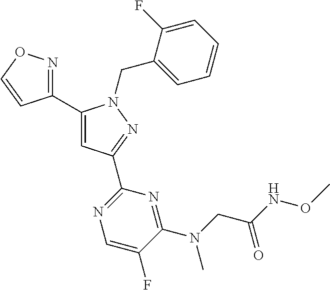 [SMILES: CONC(=O)CN(C)c1nc(-c2cc(-c3ccon3)n(Cc3ccccc3F)n2)ncc1F] | I-218 |
| 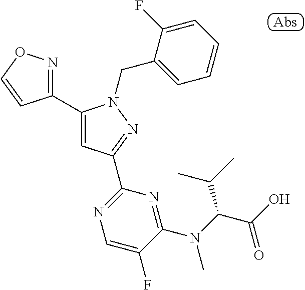 [SMILES: CC(C)[C@H](C(=O)O)N(C)c1nc(-c2cc(-c3ccon3)n(Cc3ccccc3F)n2)ncc1F] | I-219 |
| 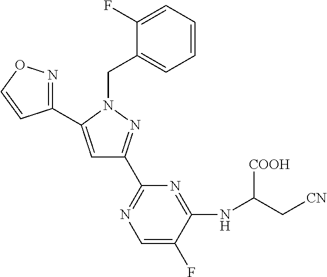 [SMILES: N#CCC(Nc1nc(-c2cc(-c3ccon3)n(Cc3ccccc3F)n2)ncc1F)C(=O)O] | I-220 |
| 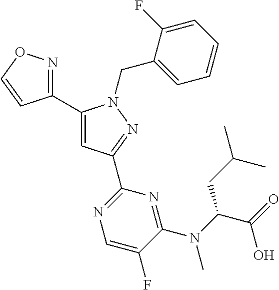 [SMILES: CC(C)C[C@H](C(=O)O)N(C)c1nc(-c2cc(-c3ccon3)n(Cc3ccccc3F)n2)ncc1F] | I-221 |
| 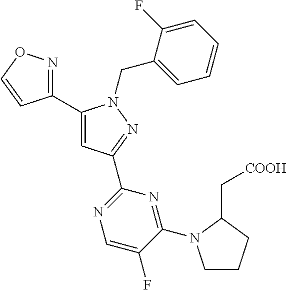 [SMILES: O=C(O)CC1CCCN1c1nc(-c2cc(-c3ccon3)n(Cc3ccccc3F)n2)ncc1F] | I-222 |
| 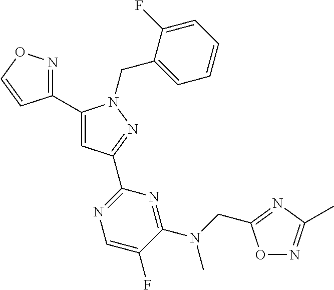 [SMILES: Cc1noc(CN(C)c2nc(-c3cc(-c4ccon4)n(Cc4ccccc4F)n3)ncc2F)n1] | I-223 |
| 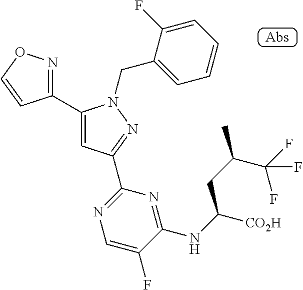 [SMILES: C[C@H](C[C@H](Nc1nc(-c2cc(-c3ccon3)n(Cc3ccccc3F)n2)ncc1F)C(=O)O)C(F)(F)F] | I-224 |
| 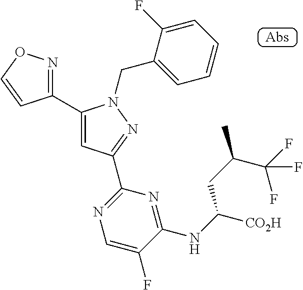 [SMILES: C[C@H](C[C@@H](Nc1nc(-c2cc(-c3ccon3)n(Cc3ccccc3F)n2)ncc1F)C(=O)O)C(F)(F)F] | I-225 |
| 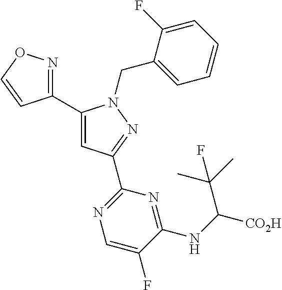 [SMILES: CC(C)(F)C(Nc1nc(-c2cc(-c3ccon3)n(Cc3ccccc3F)n2)ncc1F)C(=O)O] | I-226 |
| 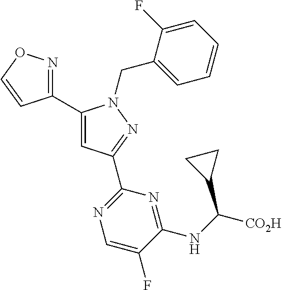 [SMILES: O=C(O)[C@@H](Nc1nc(-c2cc(-c3ccon3)n(Cc3ccccc3F)n2)ncc1F)C1CC1] | I-227 |
| 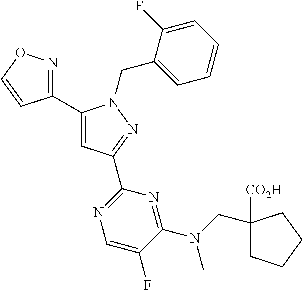 [SMILES: CN(CC1(C(=O)O)CCCC1)c1nc(-c2cc(-c3ccon3)n(Cc3ccccc3F)n2)ncc1F] | I-228 |
| 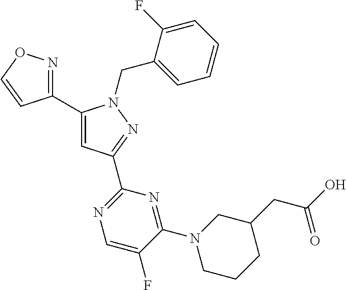 [SMILES: O=C(O)CC1CCCN(c2nc(-c3cc(-c4ccon4)n(Cc4ccccc4F)n3)ncc2F)C1] | I-229 |
| 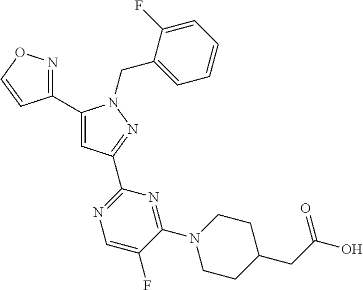 [SMILES: O=C(O)CC1CCN(c2nc(-c3cc(-c4ccon4)n(Cc4ccccc4F)n3)ncc2F)CC1] | I-230 |
| 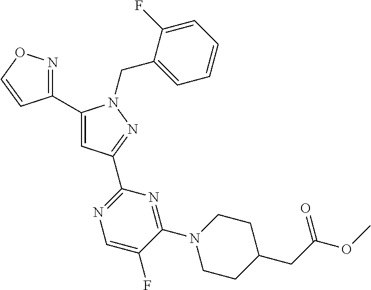 [SMILES: COC(=O)CC1CCN(c2nc(-c3cc(-c4ccon4)n(Cc4ccccc4F)n3)ncc2F)CC1] | I-231 |
| 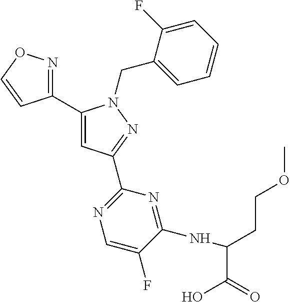 [SMILES: COCCC(Nc1nc(-c2cc(-c3ccon3)n(Cc3ccccc3F)n2)ncc1F)C(=O)O] | I-232 |
| 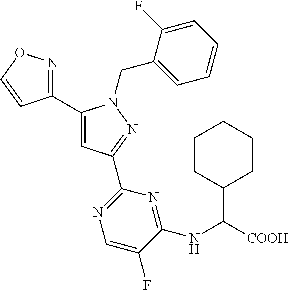 [SMILES: O=C(O)C(Nc1nc(-c2cc(-c3ccon3)n(Cc3ccccc3F)n2)ncc1F)C1CCCCC1] | I-233 |
| 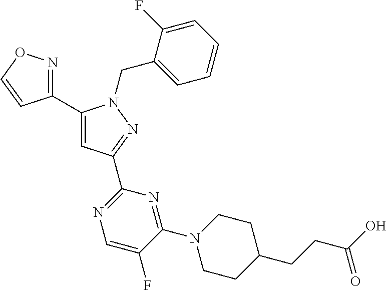 [SMILES: O=C(O)CCC1CCN(c2nc(-c3cc(-c4ccon4)n(Cc4ccccc4F)n3)ncc2F)CC1] | I-234 |
| 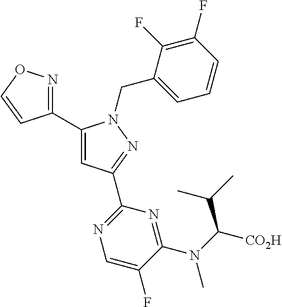 [SMILES: CC(C)[C@@H](C(=O)O)N(C)c1nc(-c2cc(-c3ccon3)n(Cc3cccc(F)c3F)n2)ncc1F] | I-235 |
| 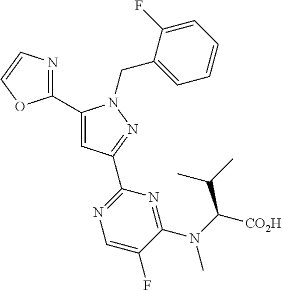 [SMILES: CC(C)[C@@H](C(=O)O)N(C)c1nc(-c2cc(-c3ncco3)n(Cc3ccccc3F)n2)ncc1F] | I-236 |
| 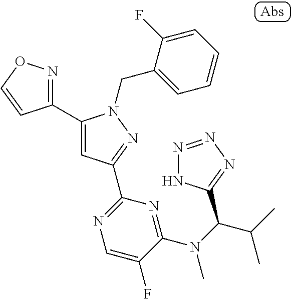 [SMILES: CC(C)[C@H](c1nnn[nH]1)N(C)c1nc(-c2cc(-c3ccon3)n(Cc3ccccc3F)n2)ncc1F] | I-237 |
| 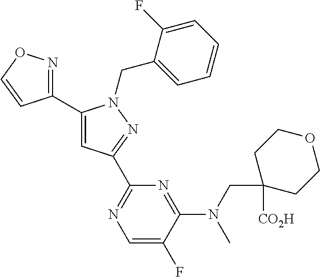 [SMILES: CN(CC1(C(=O)O)CCOCC1)c1nc(-c2cc(-c3ccon3)n(Cc3ccccc3F)n2)ncc1F] | I-238 |
| 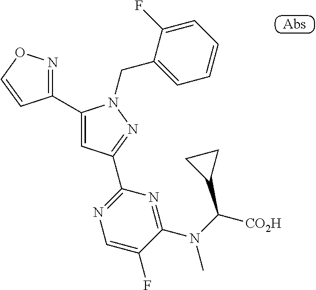 [SMILES: CN(c1nc(-c2cc(-c3ccon3)n(Cc3ccccc3F)n2)ncc1F)[C@H](C(=O)O)C1CC1] | I-239 |
| 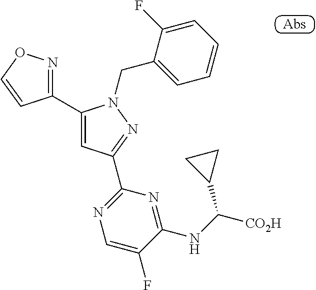 [SMILES: O=C(O)[C@H](Nc1nc(-c2cc(-c3ccon3)n(Cc3ccccc3F)n2)ncc1F)C1CC1] | I-240 |
| 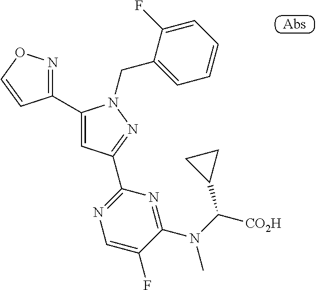 [SMILES: CN(c1nc(-c2cc(-c3ccon3)n(Cc3ccccc3F)n2)ncc1F)[C@@H](C(=O)O)C1CC1] | I-241 |
| 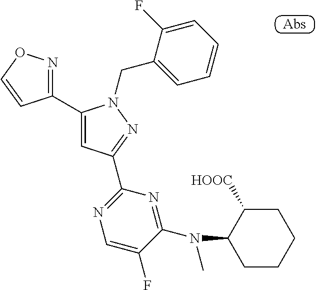 [SMILES: CN(c1nc(-c2cc(-c3ccon3)n(Cc3ccccc3F)n2)ncc1F)[C@@H]1CCCC[C@H]1C(=O)O] | I-242 |
| 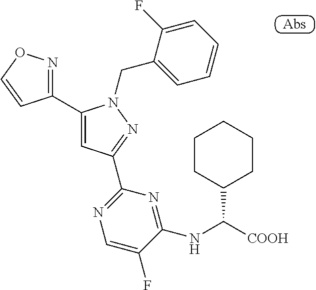 [SMILES: O=C(O)[C@H](Nc1nc(-c2cc(-c3ccon3)n(Cc3ccccc3F)n2)ncc1F)C1CCCCC1] | I-243 |
| 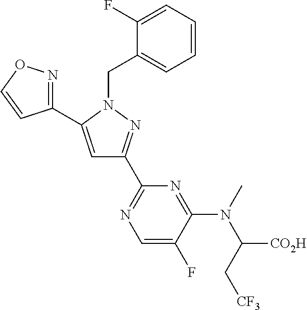 [SMILES: CN(c1nc(-c2cc(-c3ccon3)n(Cc3ccccc3F)n2)ncc1F)C(CC(F)(F)F)C(=O)O] | I-244 |
| 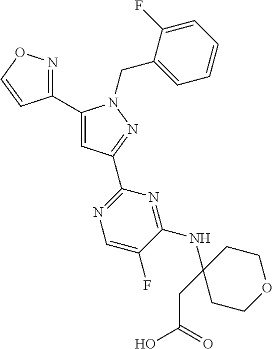 [SMILES: O=C(O)CC1(Nc2nc(-c3cc(-c4ccon4)n(Cc4ccccc4F)n3)ncc2F)CCOCC1] | I-245 |
| 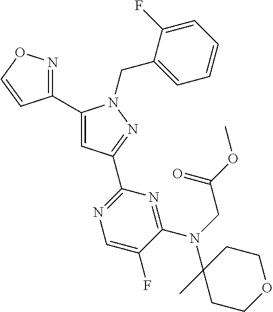 [SMILES: COC(=O)CN(c1nc(-c2cc(-c3ccon3)n(Cc3ccccc3F)n2)ncc1F)C1(C)CCOCC1] | I-246 |
| 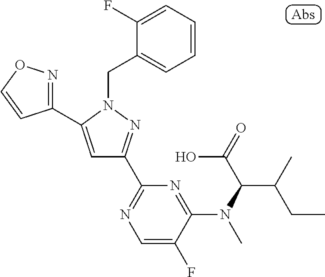 [SMILES: CCC(C)[C@H](C(=O)O)N(C)c1nc(-c2cc(-c3ccon3)n(Cc3ccccc3F)n2)ncc1F] | I-247 |
| 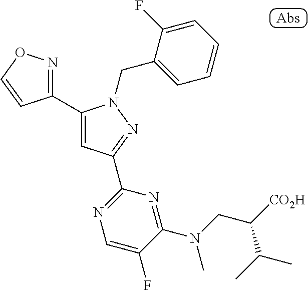 [SMILES: CC(C)[C@H](CN(C)c1nc(-c2cc(-c3ccon3)n(Cc3ccccc3F)n2)ncc1F)C(=O)O] | I-248 |
| 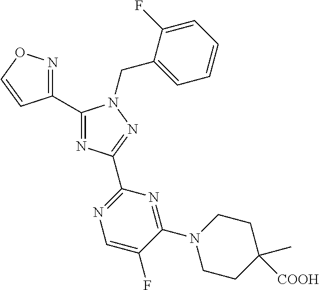 [SMILES: CC1(C(=O)O)CCN(c2nc(-c3nc(-c4ccon4)n(Cc4ccccc4F)n3)ncc2F)CC1] | I-249 |
| 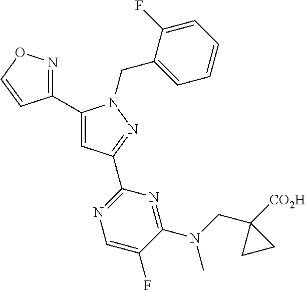 [SMILES: CN(CC1(C(=O)O)CC1)c1nc(-c2cc(-c3ccon3)n(Cc3ccccc3F)n2)ncc1F] | I-250 |
| 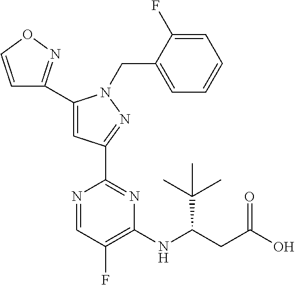 [SMILES: CC(C)(C)[C@H](CC(=O)O)Nc1nc(-c2cc(-c3ccon3)n(Cc3ccccc3F)n2)ncc1F] | I-251 |
| 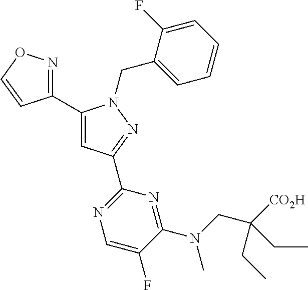 [SMILES: CCC(CC)(CN(C)c1nc(-c2cc(-c3ccon3)n(Cc3ccccc3F)n2)ncc1F)C(=O)O] | I-252 |
| 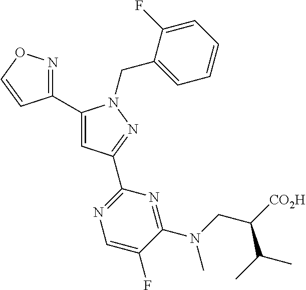 [SMILES: CC(C)[C@@H](CN(C)c1nc(-c2cc(-c3ccon3)n(Cc3ccccc3F)n2)ncc1F)C(=O)O] | I-253 |
| 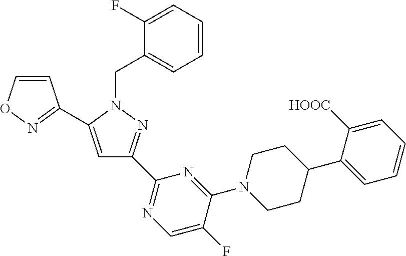 [SMILES: O=C(O)c1ccccc1C1CCN(c2nc(-c3cc(-c4ccon4)n(Cc4ccccc4F)n3)ncc2F)CC1] | I-254 |
| 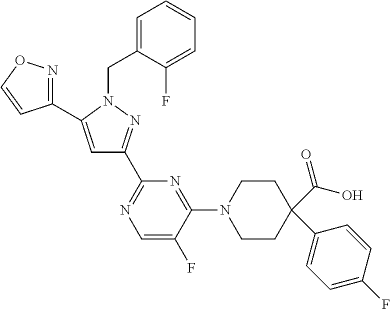 [SMILES: O=C(O)C1(c2ccc(F)cc2)CCN(c2nc(-c3cc(-c4ccon4)n(Cc4ccccc4F)n3)ncc2F)CC1] | I-255 |
| 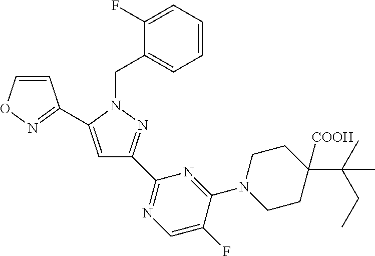 [SMILES: CCC(C)(C)C1(C(=O)O)CCN(c2nc(-c3cc(-c4ccon4)n(Cc4ccccc4F)n3)ncc2F)CC1] | I-256 |
| 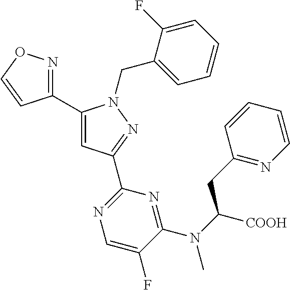 [SMILES: CN(c1nc(-c2cc(-c3ccon3)n(Cc3ccccc3F)n2)ncc1F)[C@@H](Cc1ccccn1)C(=O)O] | I-257 |
| 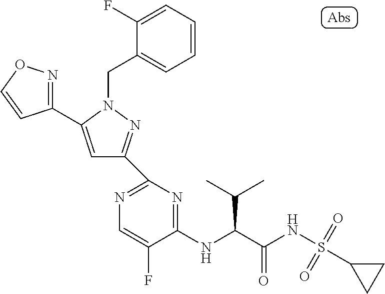 [SMILES: CC(C)[C@H](Nc1nc(-c2cc(-c3ccon3)n(Cc3ccccc3F)n2)ncc1F)C(=O)NS(=O)(=O)C1CC1] | I-258 |
| 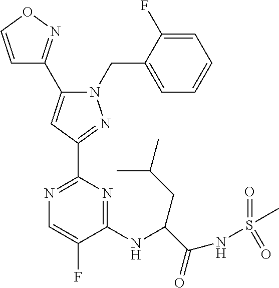 [SMILES: CC(C)CC(Nc1nc(-c2cc(-c3ccon3)n(Cc3ccccc3F)n2)ncc1F)C(=O)NS(C)(=O)=O] | I-259 |
| 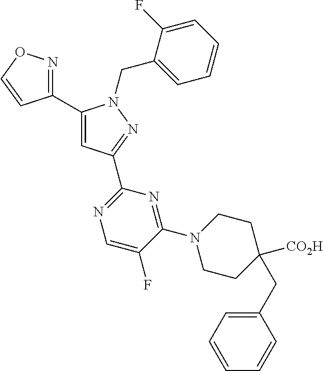 [SMILES: O=C(O)C1(Cc2ccccc2)CCN(c2nc(-c3cc(-c4ccon4)n(Cc4ccccc4F)n3)ncc2F)CC1] | I-260 |
| 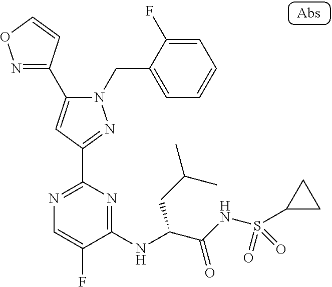 [SMILES: CC(C)C[C@@H](Nc1nc(-c2cc(-c3ccon3)n(Cc3ccccc3F)n2)ncc1F)C(=O)NS(=O)(=O)C1CC1] | I-261 |
| 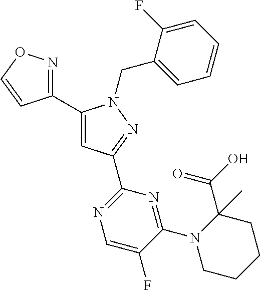 [SMILES: CC1(C(=O)O)CCCCN1c1nc(-c2cc(-c3ccon3)n(Cc3ccccc3F)n2)ncc1F] | I-262 |
| 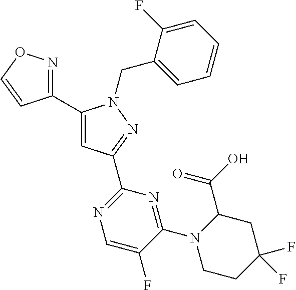 [SMILES: O=C(O)C1CC(F)(F)CCN1c1nc(-c2cc(-c3ccon3)n(Cc3ccccc3F)n2)ncc1F] | I-263 |
| 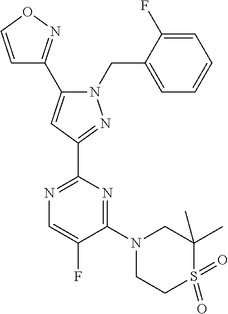 [SMILES: CC1(C)CN(c2nc(-c3cc(-c4ccon4)n(Cc4ccccc4F)n3)ncc2F)CCS1(=O)=O] | I-264 |
| 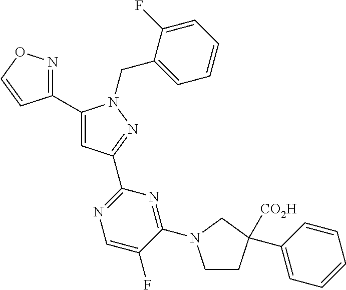 [SMILES: O=C(O)C1(c2ccccc2)CCN(c2nc(-c3cc(-c4ccon4)n(Cc4ccccc4F)n3)ncc2F)C1] | I-265 |
| 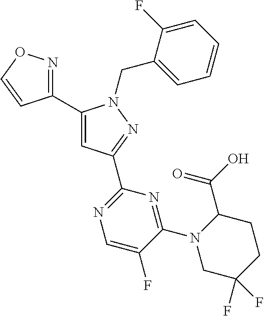 [SMILES: O=C(O)C1CCC(F)(F)CN1c1nc(-c2cc(-c3ccon3)n(Cc3ccccc3F)n2)ncc1F] | I-266 |
| 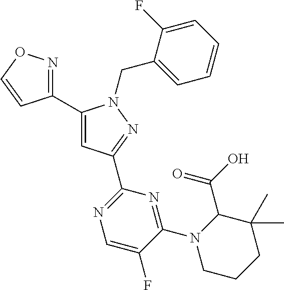 [SMILES: CC1(C)CCCN(c2nc(-c3cc(-c4ccon4)n(Cc4ccccc4F)n3)ncc2F)C1C(=O)O] | I-267 |
| 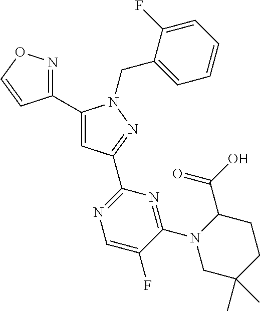 [SMILES: CC1(C)CCC(C(=O)O)N(c2nc(-c3cc(-c4ccon4)n(Cc4ccccc4F)n3)ncc2F)C1] | I-268 |
| 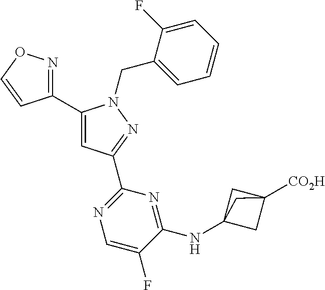 [SMILES: O=C(O)C12CC(Nc3nc(-c4cc(-c5ccon5)n(Cc5ccccc5F)n4)ncc3F)(C1)C2] | I-269 |
| 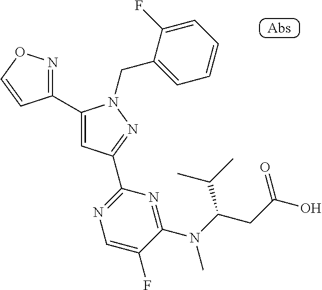 [SMILES: CC(C)[C@H](CC(=O)O)N(C)c1nc(-c2cc(-c3ccon3)n(Cc3ccccc3F)n2)ncc1F] | I-270 |
| 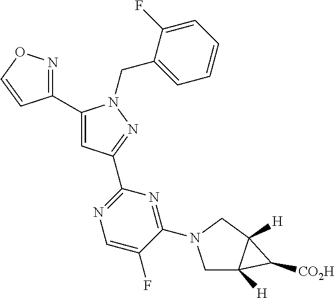 [SMILES: O=C(O)[C@H]1[C@@H]2CN(c3nc(-c4cc(-c5ccon5)n(Cc5ccccc5F)n4)ncc3F)C[C@@H]21] | I-271 |
| 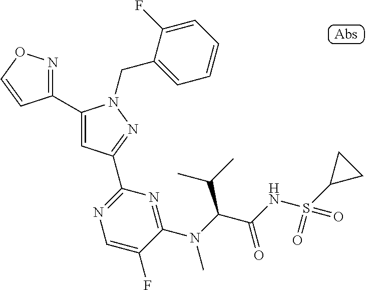 [SMILES: CC(C)[C@@H](C(=O)NS(=O)(=O)C1CC1)N(C)c1nc(-c2cc(-c3ccon3)n(Cc3ccccc3F)n2)ncc1F] | I-187 |
| 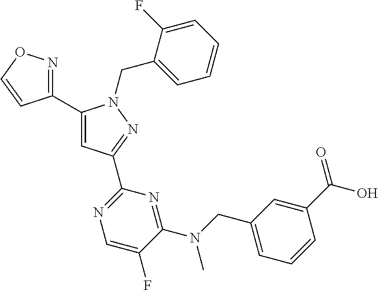 [SMILES: CN(Cc1cccc(C(=O)O)c1)c1nc(-c2cc(-c3ccon3)n(Cc3ccccc3F)n2)ncc1F] | I-274 |
| 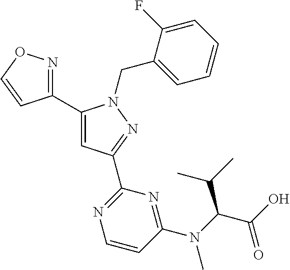 [SMILES: CC(C)[C@@H](C(=O)O)N(C)c1ccnc(-c2cc(-c3ccon3)n(Cc3ccccc3F)n2)n1] | I-272 |
| 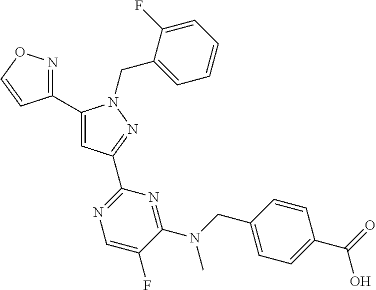 [SMILES: CN(Cc1ccc(C(=O)O)cc1)c1nc(-c2cc(-c3ccon3)n(Cc3ccccc3F)n2)ncc1F] | I-275 |
| 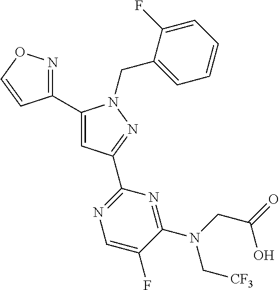 [SMILES: O=C(O)CN(CC(F)(F)F)c1nc(-c2cc(-c3ccon3)n(Cc3ccccc3F)n2)ncc1F] | I-273 |
| 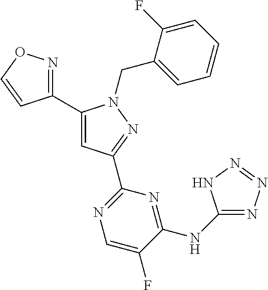 [SMILES: Fc1ccccc1Cn1nc(-c2ncc(F)c(Nc3nnn[nH]3)n2)cc1-c1ccon1] | I-276 |
| 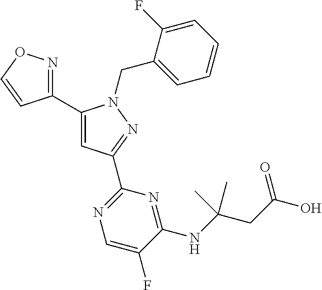 [SMILES: CC(C)(CC(=O)O)Nc1nc(-c2cc(-c3ccon3)n(Cc3ccccc3F)n2)ncc1F] | I-277 |
| 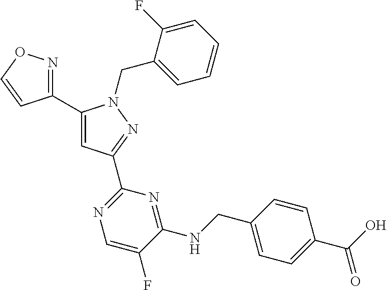 [SMILES: O=C(O)c1ccc(CNc2nc(-c3cc(-c4ccon4)n(Cc4ccccc4F)n3)ncc2F)cc1] | I-280 |
| 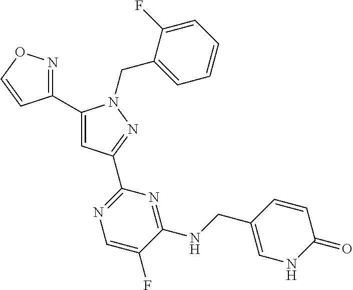 [SMILES: O=c1ccc(CNc2nc(-c3cc(-c4ccon4)n(Cc4ccccc4F)n3)ncc2F)c[nH]1] | I-278 |
| 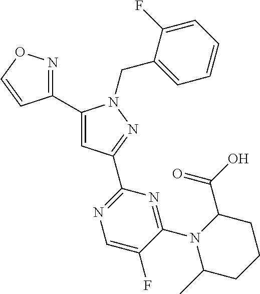 [SMILES: CC1CCCC(C(=O)O)N1c1nc(-c2cc(-c3ccon3)n(Cc3ccccc3F)n2)ncc1F] | I-281 |
| 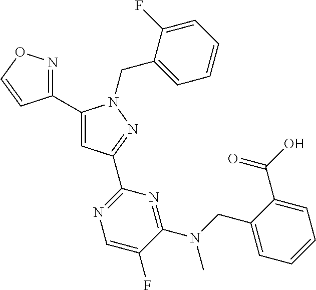 [SMILES: CN(Cc1ccccc1C(=O)O)c1nc(-c2cc(-c3ccon3)n(Cc3ccccc3F)n2)ncc1F] | I-279 |
| 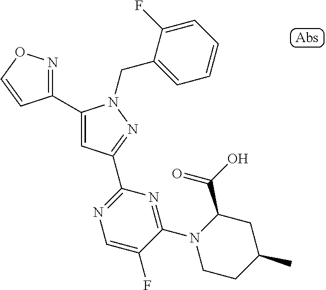 [SMILES: C[C@H]1CCN(c2nc(-c3cc(-c4ccon4)n(Cc4ccccc4F)n3)ncc2F)[C@@H](C(=O)O)C1] | I-282 |
| 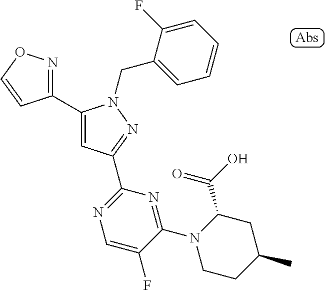 [SMILES: C[C@H]1CCN(c2nc(-c3cc(-c4ccon4)n(Cc4ccccc4F)n3)ncc2F)[C@H](C(=O)O)C1] | I-283 |
| 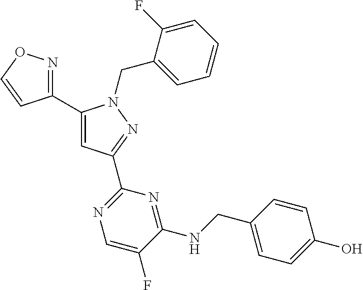 [SMILES: Oc1ccc(CNc2nc(-c3cc(-c4ccon4)n(Cc4ccccc4F)n3)ncc2F)cc1] | I-286 |
| 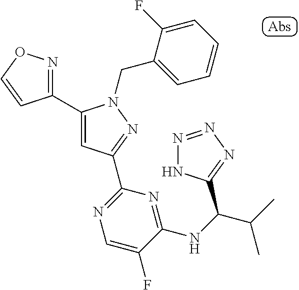 [SMILES: CC(C)[C@@H](Nc1nc(-c2cc(-c3ccon3)n(Cc3ccccc3F)n2)ncc1F)c1nnn[nH]1] | I-284 |
| 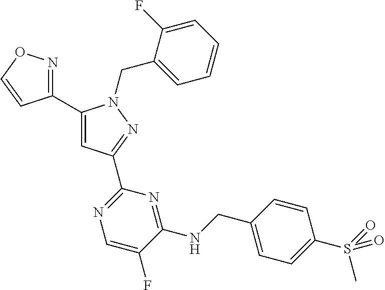 [SMILES: CS(=O)(=O)c1ccc(CNc2nc(-c3cc(-c4ccon4)n(Cc4ccccc4F)n3)ncc2F)cc1] | I-287 |
| 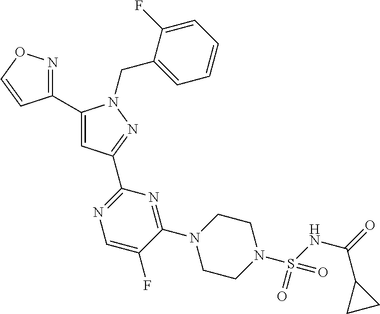 [SMILES: O=C(NS(=O)(=O)N1CCN(c2nc(-c3cc(-c4ccon4)n(Cc4ccccc4F)n3)ncc2F)CC1)C1CC1] | I-285 |
| 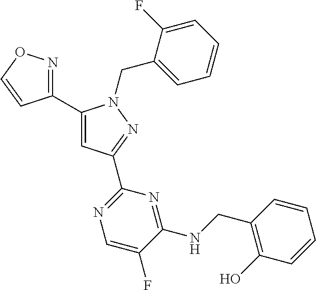 [SMILES: Oc1ccccc1CNc1nc(-c2cc(-c3ccon3)n(Cc3ccccc3F)n2)ncc1F] | I-288 |
| 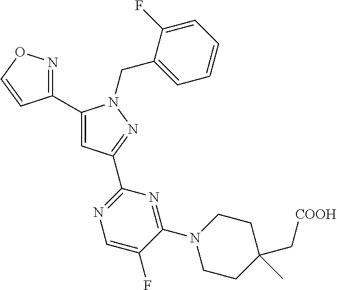 [SMILES: CC1(CC(=O)O)CCN(c2nc(-c3cc(-c4ccon4)n(Cc4ccccc4F)n3)ncc2F)CC1] | I-289 |
| 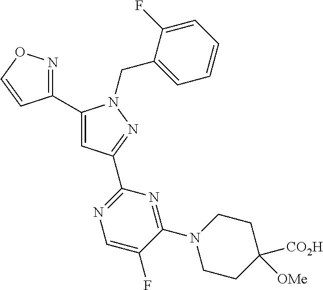 [SMILES: COC1(C(=O)O)CCN(c2nc(-c3cc(-c4ccon4)n(Cc4ccccc4F)n3)ncc2F)CC1] | I-293 |
| 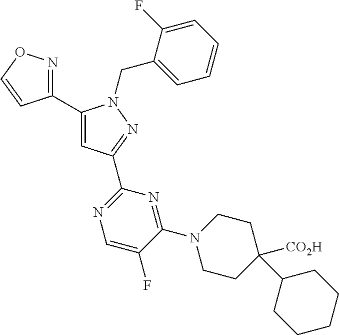 [SMILES: O=C(O)C1(C2CCCCC2)CCN(c2nc(-c3cc(-c4ccon4)n(Cc4ccccc4F)n3)ncc2F)CC1] | I-290 |
| 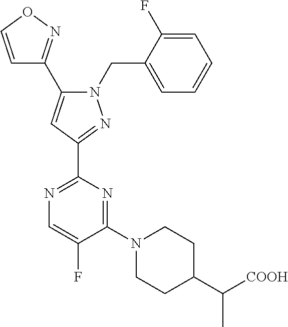 [SMILES: CC(C(=O)O)C1CCN(c2nc(-c3cc(-c4ccon4)n(Cc4ccccc4F)n3)ncc2F)CC1] | I-294 |
| 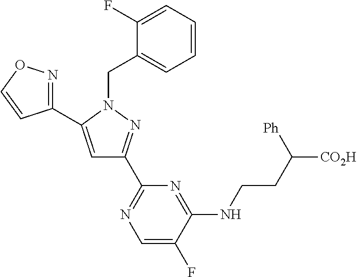 [SMILES: O=C(O)C(CCNc1nc(-c2cc(-c3ccon3)n(Cc3ccccc3F)n2)ncc1F)c1ccccc1] | I-292 |
| 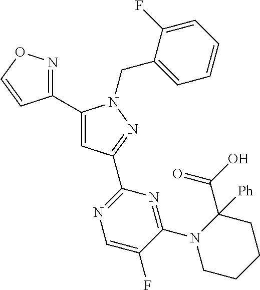 [SMILES: O=C(O)C1(c2ccccc2)CCCCN1c1nc(-c2cc(-c3ccon3)n(Cc3ccccc3F)n2)ncc1F] | I-291 |
| 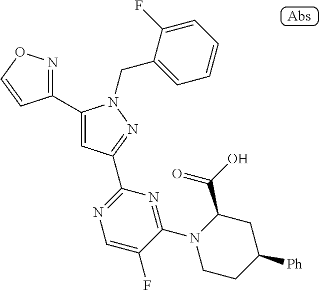 [SMILES: O=C(O)[C@H]1C[C@@H](c2ccccc2)CCN1c1nc(-c2cc(-c3ccon3)n(Cc3ccccc3F)n2)ncc1F] | I-295 |
| 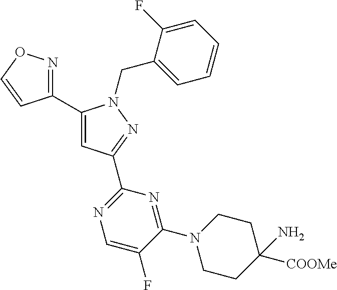 [SMILES: COC(=O)C1(N)CCN(c2nc(-c3cc(-c4ccon4)n(Cc4ccccc4F)n3)ncc2F)CC1] | I-298 |
| 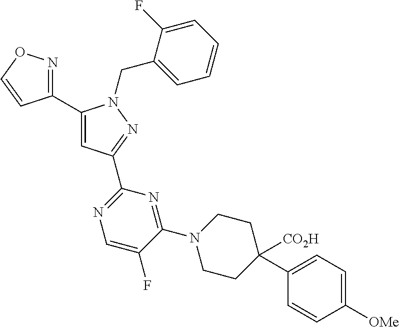 [SMILES: COc1ccc(C2(C(=O)O)CCN(c3nc(-c4cc(-c5ccon5)n(Cc5ccccc5F)n4)ncc3F)CC2)cc1] | I-296 |
| 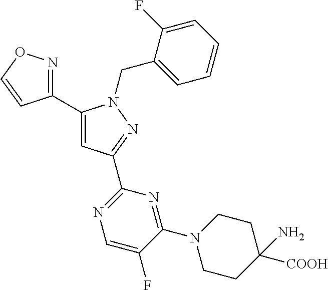 [SMILES: NC1(C(=O)O)CCN(c2nc(-c3cc(-c4ccon4)n(Cc4ccccc4F)n3)ncc2F)CC1] | I-299 |
| 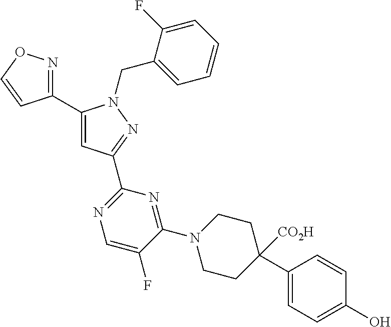 [SMILES: O=C(O)C1(c2ccc(O)cc2)CCN(c2nc(-c3cc(-c4ccon4)n(Cc4ccccc4F)n3)ncc2F)CC1] | I-297 |
| 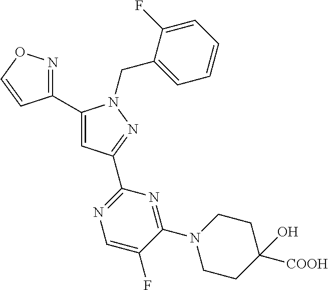 [SMILES: O=C(O)C1(O)CCN(c2nc(-c3cc(-c4ccon4)n(Cc4ccccc4F)n3)ncc2F)CC1] | I-300 |
| 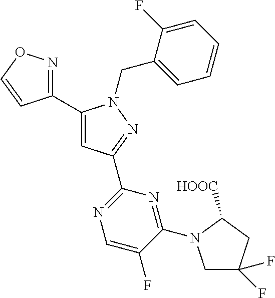 [SMILES: O=C(O)[C@@H]1CC(F)(F)CN1c1nc(-c2cc(-c3ccon3)n(Cc3ccccc3F)n2)ncc1F] | I-301 |
| 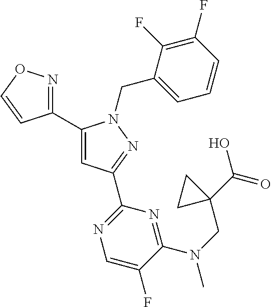 [SMILES: CN(CC1(C(=O)O)CC1)c1nc(-c2cc(-c3ccon3)n(Cc3cccc(F)c3F)n2)ncc1F] | I-304 |
| 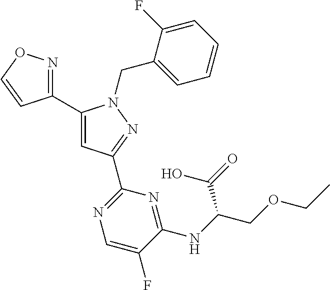 [SMILES: CCOC[C@H](Nc1nc(-c2cc(-c3ccon3)n(Cc3ccccc3F)n2)ncc1F)C(=O)O] | I-302 |
| 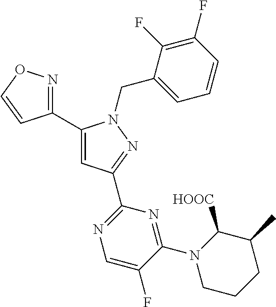 [SMILES: C[C@H]1CCCN(c2nc(-c3cc(-c4ccon4)n(Cc4cccc(F)c4F)n3)ncc2F)[C@H]1C(=O)O] | I-305 |
| 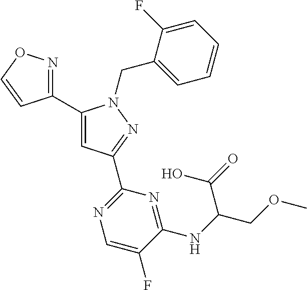 [SMILES: COCC(Nc1nc(-c2cc(-c3ccon3)n(Cc3ccccc3F)n2)ncc1F)C(=O)O] | I-303 |
| 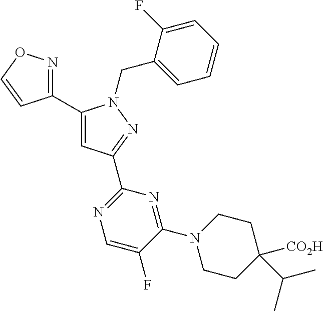 [SMILES: CC(C)C1(C(=O)O)CCN(c2nc(-c3cc(-c4ccon4)n(Cc4ccccc4F)n3)ncc2F)CC1] | I-306 |
| 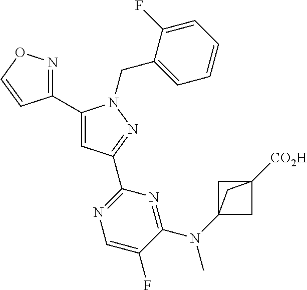 [SMILES: CN(c1nc(-c2cc(-c3ccon3)n(Cc3ccccc3F)n2)ncc1F)C12CC(C(=O)O)(C1)C2] | I-307 |
| 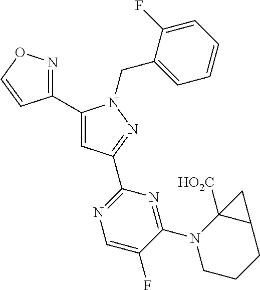 [SMILES: O=C(O)C12CC1CCCN2c1nc(-c2cc(-c3ccon3)n(Cc3ccccc3F)n2)ncc1F] | I-308 |
| 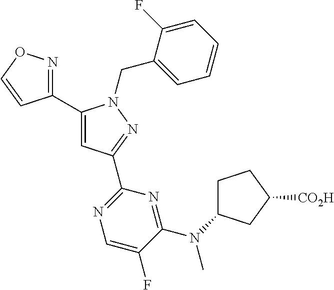 [SMILES: CN(c1nc(-c2cc(-c3ccon3)n(Cc3ccccc3F)n2)ncc1F)[C@@H]1CC[C@H](C(=O)O)C1] | I-309 |
| 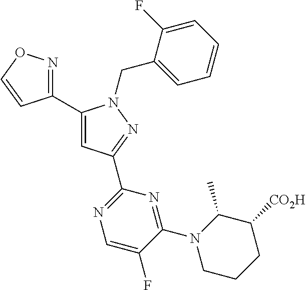 [SMILES: C[C@@H]1[C@H](C(=O)O)CCCN1c1nc(-c2cc(-c3ccon3)n(Cc3ccccc3F)n2)ncc1F] | I-310 |
| 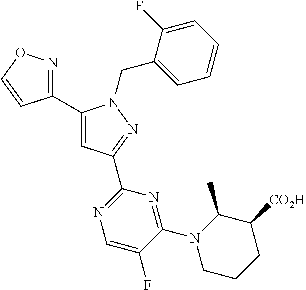 [SMILES: C[C@H]1[C@@H](C(=O)O)CCCN1c1nc(-c2cc(-c3ccon3)n(Cc3ccccc3F)n2)ncc1F] | I-311 |
| 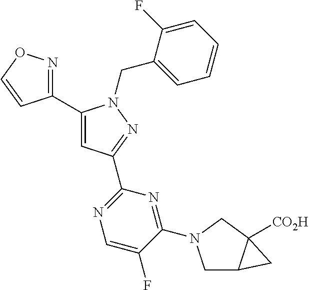 [SMILES: O=C(O)C12CC1CN(c1nc(-c3cc(-c4ccon4)n(Cc4ccccc4F)n3)ncc1F)C2] | I-312 |
| 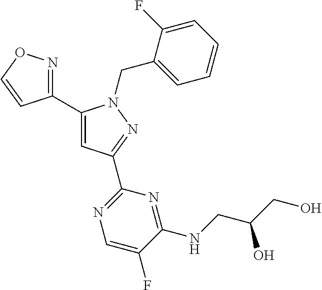 [SMILES: OC[C@@H](O)CNc1nc(-c2cc(-c3ccon3)n(Cc3ccccc3F)n2)ncc1F] | I-313 |
| 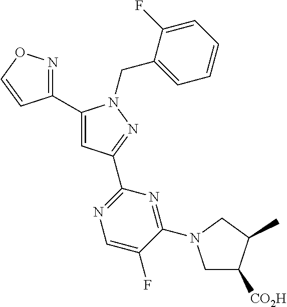 [SMILES: C[C@@H]1CN(c2nc(-c3cc(-c4ccon4)n(Cc4ccccc4F)n3)ncc2F)C[C@@H]1C(=O)O] | I-314 |
| 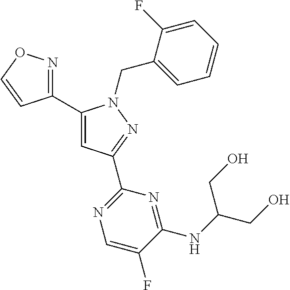 [SMILES: OCC(CO)Nc1nc(-c2cc(-c3ccon3)n(Cc3ccccc3F)n2)ncc1F] | I-315 |
| 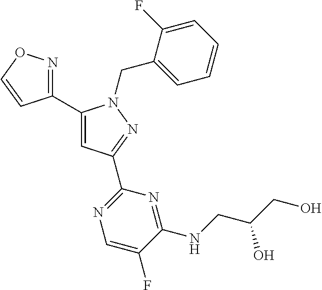 [SMILES: OC[C@H](O)CNc1nc(-c2cc(-c3ccon3)n(Cc3ccccc3F)n2)ncc1F] | I-316 |
| 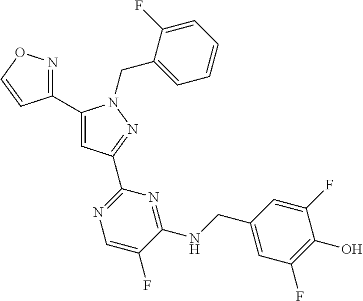 [SMILES: Oc1c(F)cc(CNc2nc(-c3cc(-c4ccon4)n(Cc4ccccc4F)n3)ncc2F)cc1F] | I-317 |
| 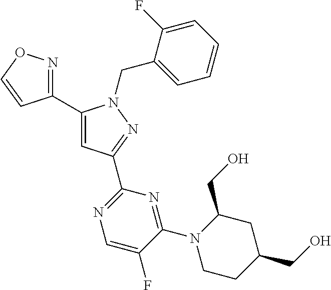 [SMILES: OC[C@H]1CCN(c2nc(-c3cc(-c4ccon4)n(Cc4ccccc4F)n3)ncc2F)[C@@H](CO)C1] | I-318 |
| 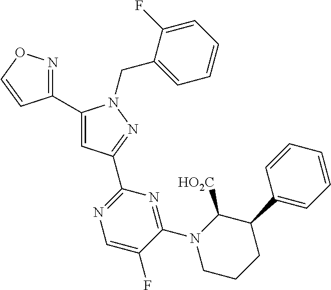 [SMILES: O=C(O)[C@H]1[C@@H](c2ccccc2)CCCN1c1nc(-c2cc(-c3ccon3)n(Cc3ccccc3F)n2)ncc1F] | I-319 |
| 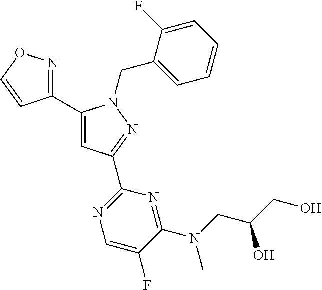 [SMILES: CN(C[C@H](O)CO)c1nc(-c2cc(-c3ccon3)n(Cc3ccccc3F)n2)ncc1F] | I-320 |
| 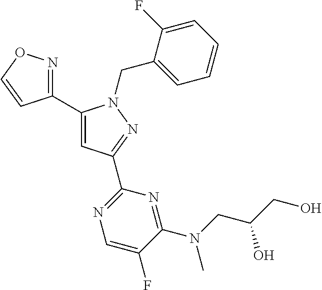 [SMILES: CN(C[C@@H](O)CO)c1nc(-c2cc(-c3ccon3)n(Cc3ccccc3F)n2)ncc1F] | I-321 |
| 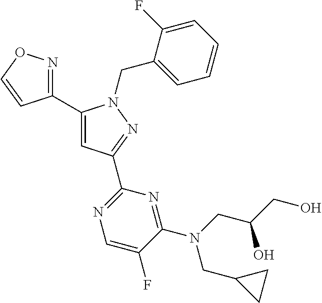 [SMILES: OC[C@@H](O)CN(CC1CC1)c1nc(-c2cc(-c3ccon3)n(Cc3ccccc3F)n2)ncc1F] | I-322 |
| 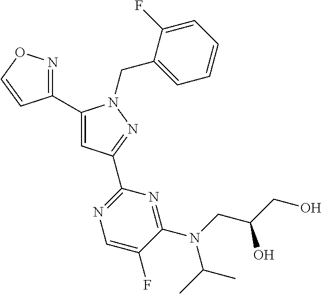 [SMILES: CC(C)N(C[C@H](O)CO)c1nc(-c2cc(-c3ccon3)n(Cc3ccccc3F)n2)ncc1F] | I-323 |
| 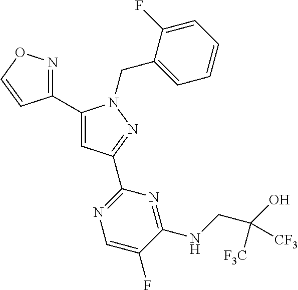 [SMILES: OC(CNc1nc(-c2cc(-c3ccon3)n(Cc3ccccc3F)n2)ncc1F)(C(F)(F)F)C(F)(F)F] | I-324 |
| 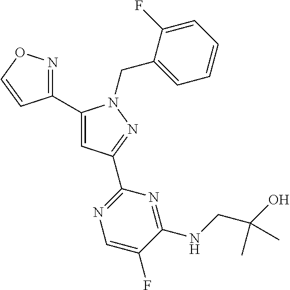 [SMILES: CC(C)(O)CNc1nc(-c2cc(-c3ccon3)n(Cc3ccccc3F)n2)ncc1F] | I-325 |
| 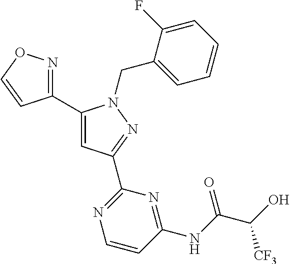 [SMILES: O=C(Nc1ccnc(-c2cc(-c3ccon3)n(Cc3ccccc3F)n2)n1)[C@H](O)C(F)(F)F] | I-326 |
| 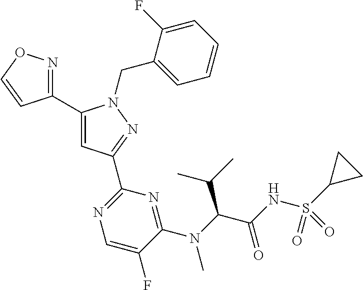 [SMILES: CC(C)[C@@H](C(=O)NS(=O)(=O)C1CC1)N(C)c1nc(-c2cc(-c3ccon3)n(Cc3ccccc3F)n2)ncc1F] | I-327 |
| 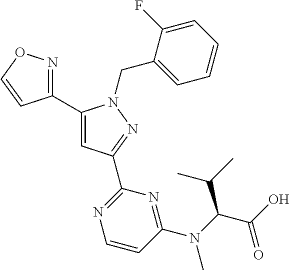 [SMILES: CC(C)[C@@H](C(=O)O)N(C)c1ccnc(-c2cc(-c3ccon3)n(Cc3ccccc3F)n2)n1] | I-328 |
| 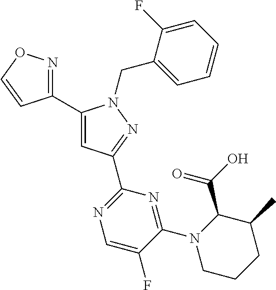 [SMILES: C[C@H]1CCCN(c2nc(-c3cc(-c4ccon4)n(Cc4ccccc4F)n3)ncc2F)[C@H]1C(=O)O] | I-329 |
| 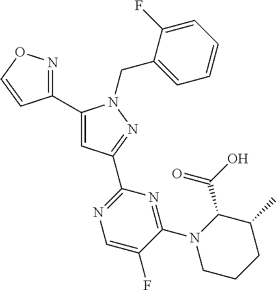 [SMILES: C[C@@H]1CCCN(c2nc(-c3cc(-c4ccon4)n(Cc4ccccc4F)n3)ncc2F)[C@@H]1C(=O)O] | I-330 |
| 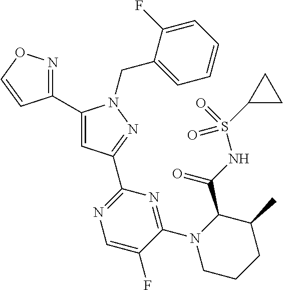 [SMILES: C[C@H]1CCCN(c2nc(-c3cc(-c4ccon4)n(Cc4ccccc4F)n3)ncc2F)[C@H]1C(=O)NS(=O)(=O)C1CC1] | I-331 |
| 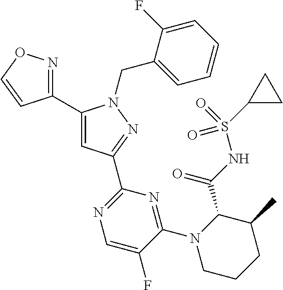 [SMILES: C[C@H]1CCCN(c2nc(-c3cc(-c4ccon4)n(Cc4ccccc4F)n3)ncc2F)[C@@H]1C(=O)NS(=O)(=O)C1CC1] | I-332 |
| 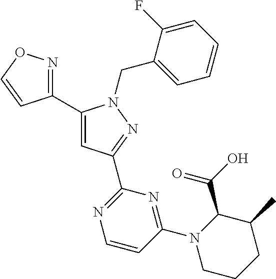 [SMILES: C[C@H]1CCCN(c2ccnc(-c3cc(-c4ccon4)n(Cc4ccccc4F)n3)n2)[C@H]1C(=O)O] | I-333 |
| 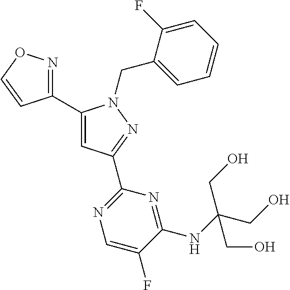 [SMILES: OCC(CO)(CO)Nc1nc(-c2cc(-c3ccon3)n(Cc3ccccc3F)n2)ncc1F] | I-334 |
| 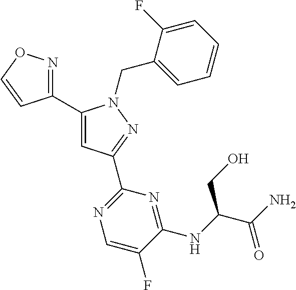 [SMILES: NC(=O)[C@H](CO)Nc1nc(-c2cc(-c3ccon3)n(Cc3ccccc3F)n2)ncc1F] | I-335 |
| 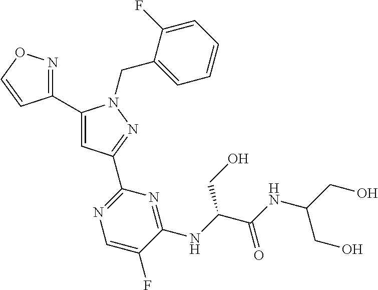 [SMILES: O=C(NC(CO)CO)[C@@H](CO)Nc1nc(-c2cc(-c3ccon3)n(Cc3ccccc3F)n2)ncc1F] | I-336 |
| 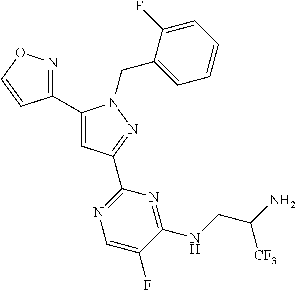 [SMILES: NC(CNc1nc(-c2cc(-c3ccon3)n(Cc3ccccc3F)n2)ncc1F)C(F)(F)F] | I-337 |
| 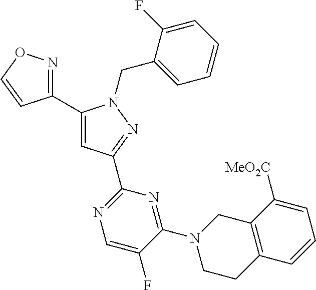 [SMILES: COC(=O)c1cccc2c1CN(c1nc(-c3cc(-c4ccon4)n(Cc4ccccc4F)n3)ncc1F)CC2] | I-338 |
| 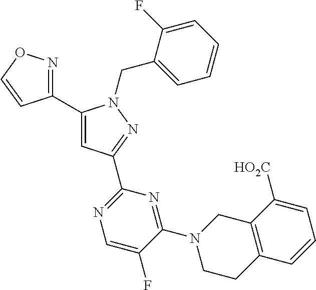 [SMILES: O=C(O)c1cccc2c1CN(c1nc(-c3cc(-c4ccon4)n(Cc4ccccc4F)n3)ncc1F)CC2] | I-339 |
| 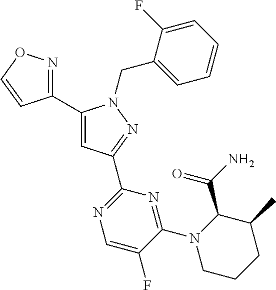 [SMILES: C[C@H]1CCCN(c2nc(-c3cc(-c4ccon4)n(Cc4ccccc4F)n3)ncc2F)[C@H]1C(N)=O] | I-340 |
| 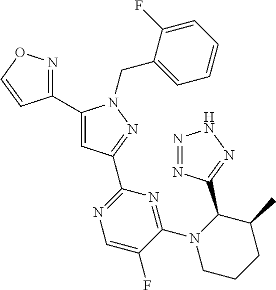 [SMILES: C[C@H]1CCCN(c2nc(-c3cc(-c4ccon4)n(Cc4ccccc4F)n3)ncc2F)[C@H]1c1nn[nH]n1] | I-341 |
| 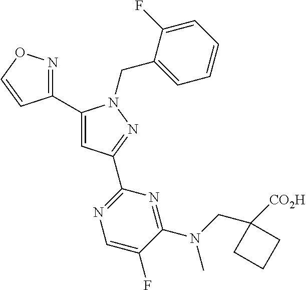 [SMILES: CN(CC1(C(=O)O)CCC1)c1nc(-c2cc(-c3ccon3)n(Cc3ccccc3F)n2)ncc1F] | I-342 |
| 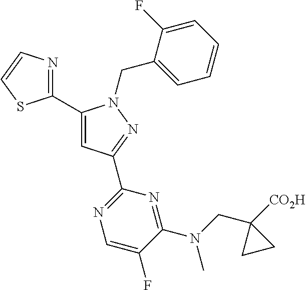 [SMILES: CN(CC1(C(=O)O)CC1)c1nc(-c2cc(-c3nccs3)n(Cc3ccccc3F)n2)ncc1F] | I-343 |
| 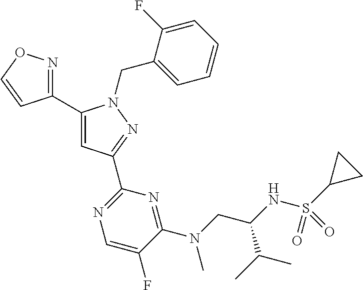 [SMILES: CC(C)[C@H](CN(C)c1nc(-c2cc(-c3ccon3)n(Cc3ccccc3F)n2)ncc1F)NS(=O)(=O)C1CC1] | I-344 |
| 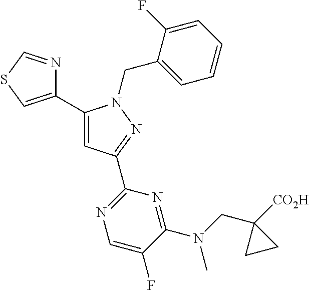 [SMILES: CN(CC1(C(=O)O)CC1)c1nc(-c2cc(-c3cscn3)n(Cc3ccccc3F)n2)ncc1F] | I-345 |
| 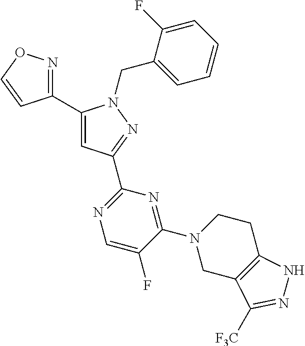 [SMILES: Fc1ccccc1Cn1nc(-c2ncc(F)c(N3CCc4[nH]nc(C(F)(F)F)c4C3)n2)cc1-c1ccon1] | I-346 |
| 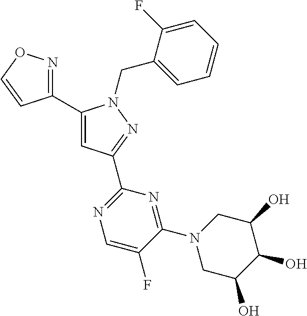 [SMILES: O[C@@H]1[C@H](O)CN(c2nc(-c3cc(-c4ccon4)n(Cc4ccccc4F)n3)ncc2F)C[C@@H]1O] | I-347 |
| 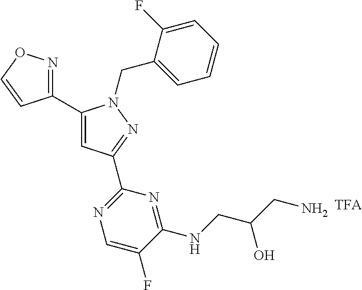 [SMILES: NCC(O)CNc1nc(-c2cc(-c3ccon3)n(Cc3ccccc3F)n2)ncc1F] | I-348 |
| 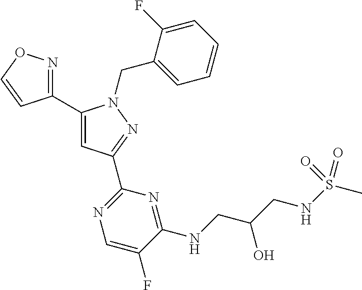 [SMILES: CS(=O)(=O)NCC(O)CNc1nc(-c2cc(-c3ccon3)n(Cc3ccccc3F)n2)ncc1F] | I-349 |
| 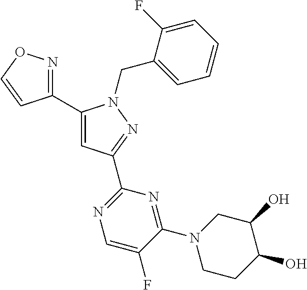 [SMILES: O[C@@H]1CN(c2nc(-c3cc(-c4ccon4)n(Cc4ccccc4F)n3)ncc2F)CC[C@@H]1O] | I-350 |
| 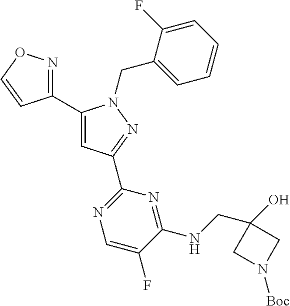 [SMILES: CC(C)(C)OC(=O)N1CC(O)(CNc2nc(-c3cc(-c4ccon4)n(Cc4ccccc4F)n3)ncc2F)C1] | I-351 |
| 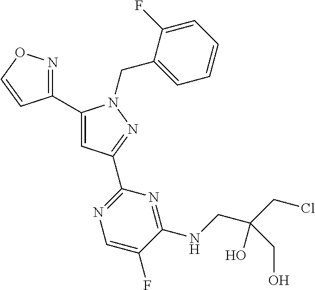 [SMILES: OCC(O)(CCl)CNc1nc(-c2cc(-c3ccon3)n(Cc3ccccc3F)n2)ncc1F] | I-352 |
| 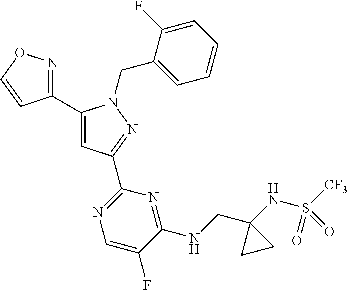 [SMILES: O=S(=O)(NC1(CNc2nc(-c3cc(-c4ccon4)n(Cc4ccccc4F)n3)ncc2F)CC1)C(F)(F)F] | I-354 |
| 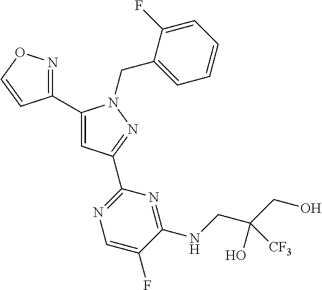 [SMILES: OCC(O)(CNc1nc(-c2cc(-c3ccon3)n(Cc3ccccc3F)n2)ncc1F)C(F)(F)F] | I-355 |
| 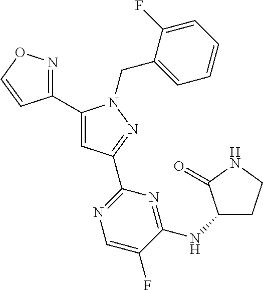 [SMILES: O=C1NCC[C@@H]1Nc1nc(-c2cc(-c3ccon3)n(Cc3ccccc3F)n2)ncc1F] | I-356 |
| 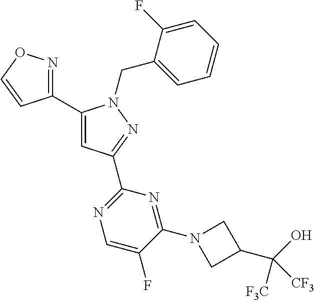 [SMILES: OC(C1CN(c2nc(-c3cc(-c4ccon4)n(Cc4ccccc4F)n3)ncc2F)C1)(C(F)(F)F)C(F)(F)F] | I-357 |
| 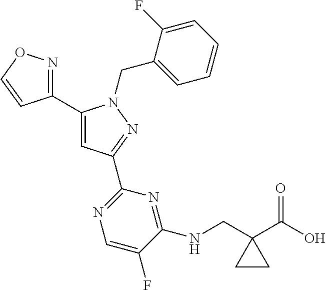 [SMILES: O=C(O)C1(CNc2nc(-c3cc(-c4ccon4)n(Cc4ccccc4F)n3)ncc2F)CC1] | I-358 |
| 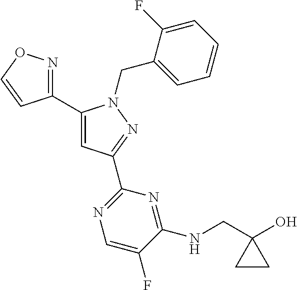 [SMILES: OC1(CNc2nc(-c3cc(-c4ccon4)n(Cc4ccccc4F)n3)ncc2F)CC1] | I-359 |
| 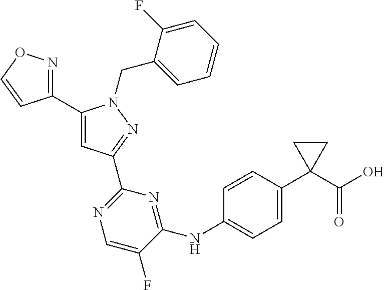 [SMILES: O=C(O)C1(c2ccc(Nc3nc(-c4cc(-c5ccon5)n(Cc5ccccc5F)n4)ncc3F)cc2)CC1] | I-360 |
| 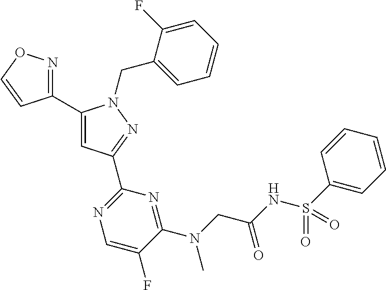 [SMILES: CN(CC(=O)NS(=O)(=O)c1ccccc1)c1nc(-c2cc(-c3ccon3)n(Cc3ccccc3F)n2)ncc1F] | I-361 |
| 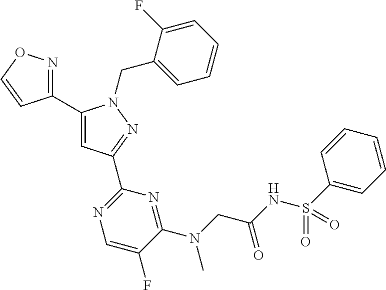 [SMILES: CN(CC(=O)NS(=O)(=O)c1ccccc1)c1nc(-c2cc(-c3ccon3)n(Cc3ccccc3F)n2)ncc1F] | I-362 |
| 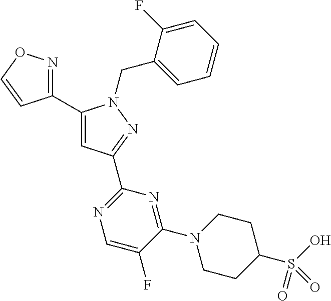 [SMILES: O=S(=O)(O)C1CCN(c2nc(-c3cc(-c4ccon4)n(Cc4ccccc4F)n3)ncc2F)CC1] | I-363 |
| 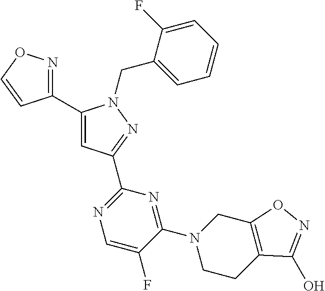 [SMILES: Oc1noc2c1CCN(c1nc(-c3cc(-c4ccon4)n(Cc4ccccc4F)n3)ncc1F)C2] | I-364 |
| 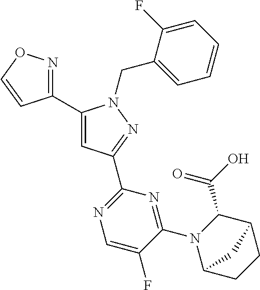 [SMILES: O=C(O)[C@@H]1[C@H]2CC[C@H](C2)N1c1nc(-c2cc(-c3ccon3)n(Cc3ccccc3F)n2)ncc1F] | I-365 |
| 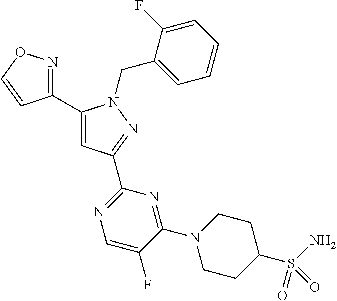 [SMILES: NS(=O)(=O)C1CCN(c2nc(-c3cc(-c4ccon4)n(Cc4ccccc4F)n3)ncc2F)CC1] | I-366 |
| 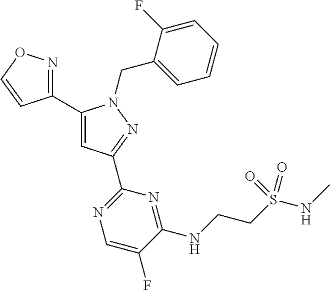 [SMILES: CNS(=O)(=O)CCNc1nc(-c2cc(-c3ccon3)n(Cc3ccccc3F)n2)ncc1F] | I-367 |
| 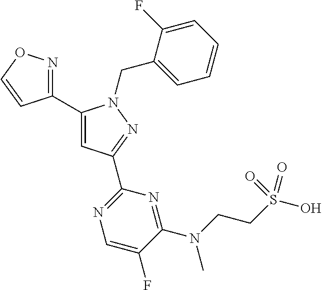 [SMILES: CN(CCS(=O)(=O)O)c1nc(-c2cc(-c3ccon3)n(Cc3ccccc3F)n2)ncc1F] | I-368 |
| 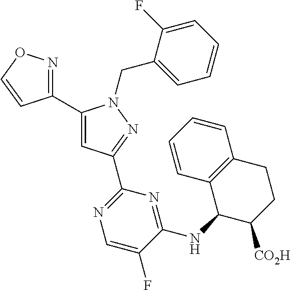 [SMILES: O=C(O)[C@@H]1CCc2ccccc2[C@@H]1Nc1nc(-c2cc(-c3ccon3)n(Cc3ccccc3F)n2)ncc1F] | I-369 |
| 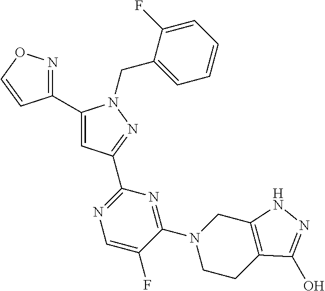 [SMILES: Oc1n[nH]c2c1CCN(c1nc(-c3cc(-c4ccon4)n(Cc4ccccc4F)n3)ncc1F)C2] | I-370 |
| 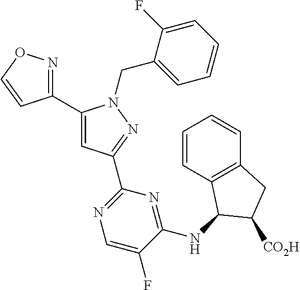 [SMILES: O=C(O)[C@@H]1Cc2ccccc2[C@@H]1Nc1nc(-c2cc(-c3ccon3)n(Cc3ccccc3F)n2)ncc1F] | I-371 |
| 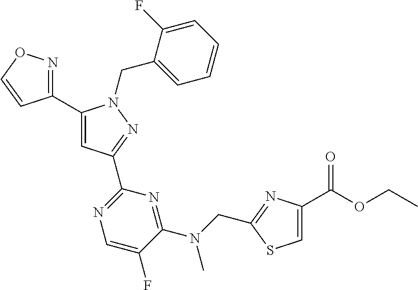 [SMILES: CCOC(=O)c1csc(CN(C)c2nc(-c3cc(-c4ccon4)n(Cc4ccccc4F)n3)ncc2F)n1] | I-372 |
| 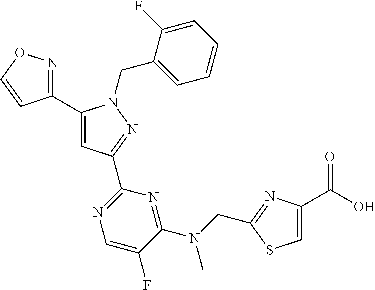 [SMILES: CN(Cc1nc(C(=O)O)cs1)c1nc(-c2cc(-c3ccon3)n(Cc3ccccc3F)n2)ncc1F] | I-373 |
| 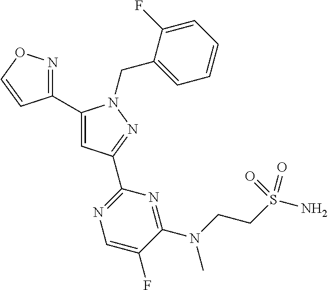 [SMILES: CN(CCS(N)(=O)=O)c1nc(-c2cc(-c3ccon3)n(Cc3ccccc3F)n2)ncc1F] | I-374 |
| 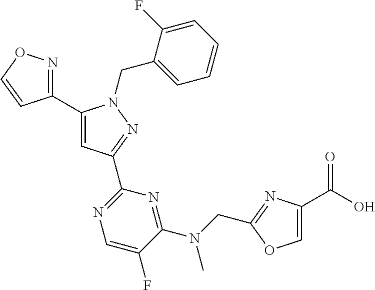 [SMILES: CN(Cc1nc(C(=O)O)co1)c1nc(-c2cc(-c3ccon3)n(Cc3ccccc3F)n2)ncc1F] | I-375 |
| 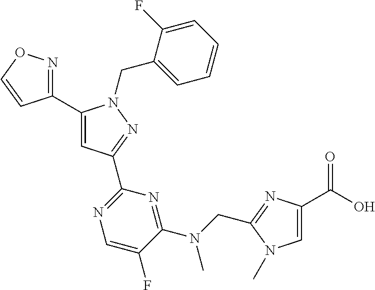 [SMILES: CN(Cc1nc(C(=O)O)cn1C)c1nc(-c2cc(-c3ccon3)n(Cc3ccccc3F)n2)ncc1F] | I-376 |
| 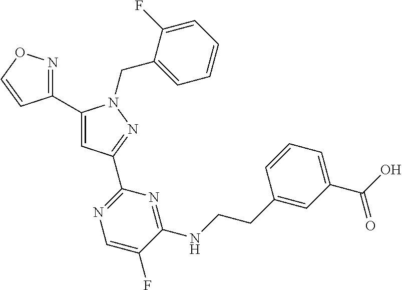 [SMILES: O=C(O)c1cccc(CCNc2nc(-c3cc(-c4ccon4)n(Cc4ccccc4F)n3)ncc2F)c1] | I-377 |
| 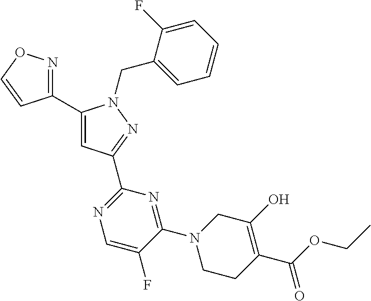 [SMILES: CCOC(=O)C1=C(O)CN(c2nc(-c3cc(-c4ccon4)n(Cc4ccccc4F)n3)ncc2F)CC1] | I-378 |
| 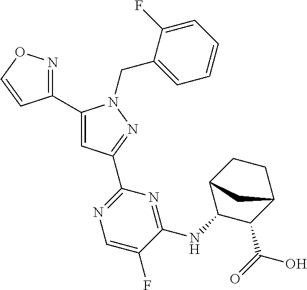 [SMILES: O=C(O)[C@H]1[C@H]2CC[C@H](C2)[C@H]1Nc1nc(-c2cc(-c3ccon3)n(Cc3ccccc3F)n2)ncc1F] | I-379 |
| 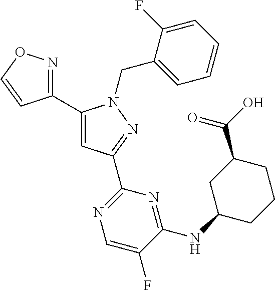 [SMILES: O=C(O)[C@H]1CCC[C@@H](Nc2nc(-c3cc(-c4ccon4)n(Cc4ccccc4F)n3)ncc2F)C1] | I-380 |
| 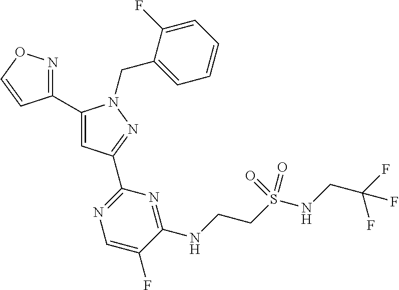 [SMILES: O=S(=O)(CCNc1nc(-c2cc(-c3ccon3)n(Cc3ccccc3F)n2)ncc1F)NCC(F)(F)F] | I-381 |
| 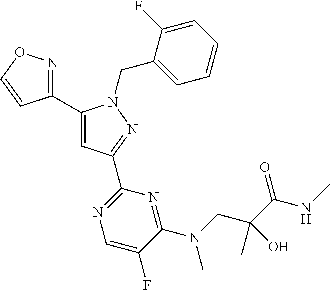 [SMILES: CNC(=O)C(C)(O)CN(C)c1nc(-c2cc(-c3ccon3)n(Cc3ccccc3F)n2)ncc1F] | I-382 |
| 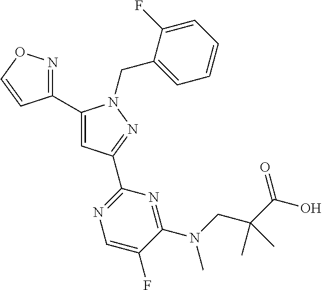 [SMILES: CN(CC(C)(C)C(=O)O)c1nc(-c2cc(-c3ccon3)n(Cc3ccccc3F)n2)ncc1F] | I-383 |
| 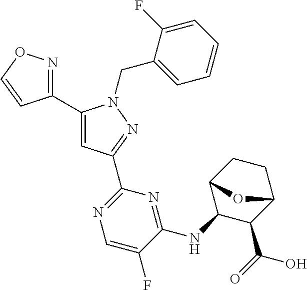 [SMILES: O=C(O)[C@H]1[C@@H](Nc2nc(-c3cc(-c4ccon4)n(Cc4ccccc4F)n3)ncc2F)[C@@H]2CC[C@H]1O2] | I-384 |
| 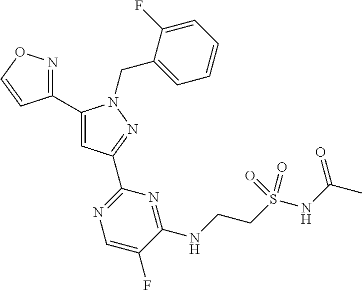 [SMILES: CC(=O)NS(=O)(=O)CCNc1nc(-c2cc(-c3ccon3)n(Cc3ccccc3F)n2)ncc1F] | I-385 |
| 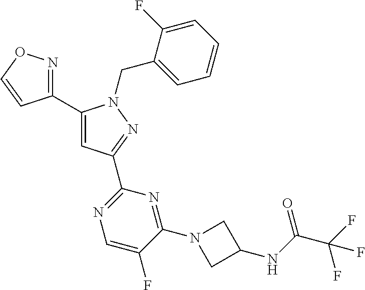 [SMILES: O=C(NC1CN(c2nc(-c3cc(-c4ccon4)n(Cc4ccccc4F)n3)ncc2F)C1)C(F)(F)F] | I-387 |
| 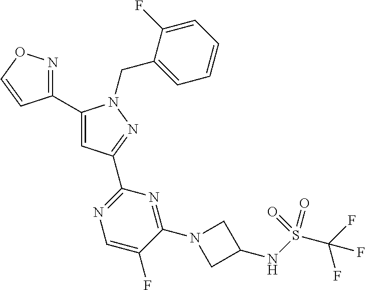 [SMILES: O=S(=O)(NC1CN(c2nc(-c3cc(-c4ccon4)n(Cc4ccccc4F)n3)ncc2F)C1)C(F)(F)F] | I-388 |
| 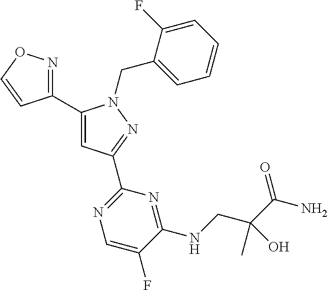 [SMILES: CC(O)(CNc1nc(-c2cc(-c3ccon3)n(Cc3ccccc3F)n2)ncc1F)C(N)=O] | I-389 |
| 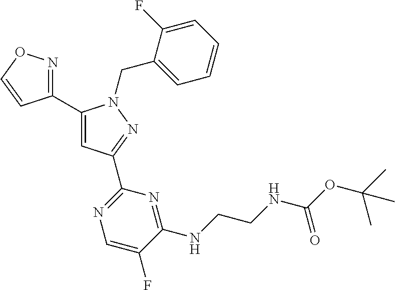 [SMILES: CC(C)(C)OC(=O)NCCNc1nc(-c2cc(-c3ccon3)n(Cc3ccccc3F)n2)ncc1F] | I-390 |
| 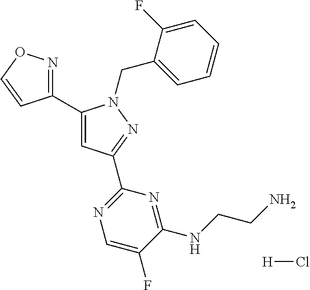 | I-391 |
| 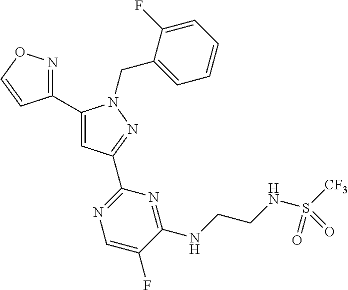 [SMILES: O=S(=O)(NCCNc1nc(-c2cc(-c3ccon3)n(Cc3ccccc3F)n2)ncc1F)C(F)(F)F] | I-392 |
| 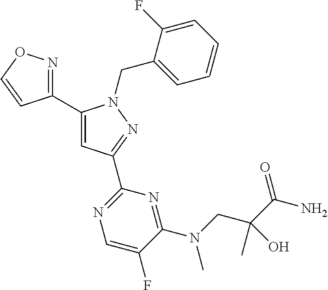 [SMILES: CN(CC(C)(O)C(N)=O)c1nc(-c2cc(-c3ccon3)n(Cc3ccccc3F)n2)ncc1F] | I-393 |
| 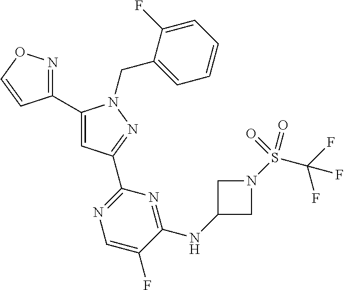 [SMILES: O=S(=O)(N1CC(Nc2nc(-c3cc(-c4ccon4)n(Cc4ccccc4F)n3)ncc2F)C1)C(F)(F)F] | I-394 |
| 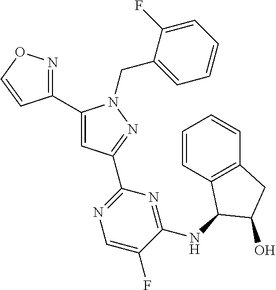 [SMILES: O[C@@H]1Cc2ccccc2[C@@H]1Nc1nc(-c2cc(-c3ccon3)n(Cc3ccccc3F)n2)ncc1F] | I-395 |
| 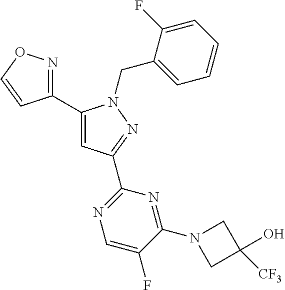 [SMILES: OC1(C(F)(F)F)CN(c2nc(-c3cc(-c4ccon4)n(Cc4ccccc4F)n3)ncc2F)C1] | I-396 |
| 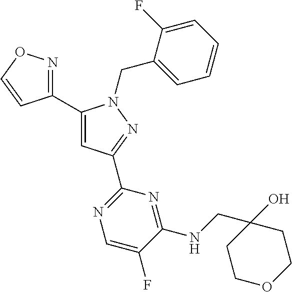 [SMILES: OC1(CNc2nc(-c3cc(-c4ccon4)n(Cc4ccccc4F)n3)ncc2F)CCOCC1] | I-397 |
| 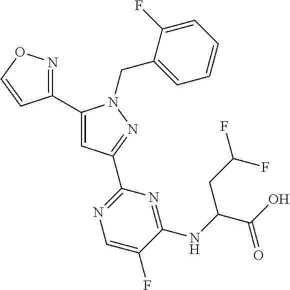 [SMILES: O=C(O)C(CC(F)F)Nc1nc(-c2cc(-c3ccon3)n(Cc3ccccc3F)n2)ncc1F] | I-398 |
| 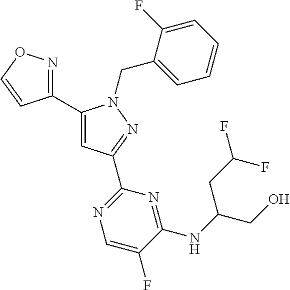 [SMILES: OCC(CC(F)F)Nc1nc(-c2cc(-c3ccon3)n(Cc3ccccc3F)n2)ncc1F] | I-399 |
| 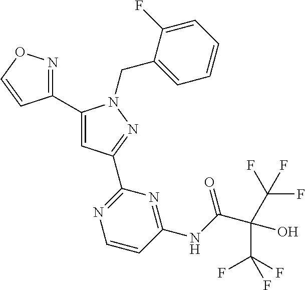 [SMILES: O=C(Nc1ccnc(-c2cc(-c3ccon3)n(Cc3ccccc3F)n2)n1)C(O)(C(F)(F)F)C(F)(F)F] | I-400 |
| 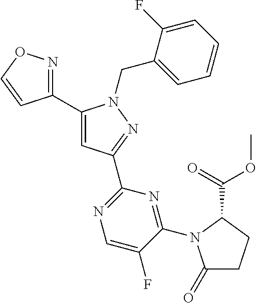 [SMILES: COC(=O)[C@@H]1CCC(=O)N1c1nc(-c2cc(-c3ccon3)n(Cc3ccccc3F)n2)ncc1F] | I-401 |
| 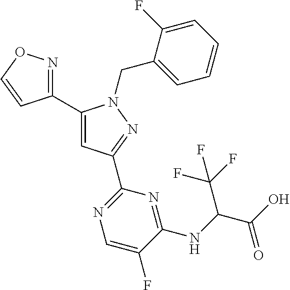 [SMILES: O=C(O)C(Nc1nc(-c2cc(-c3ccon3)n(Cc3ccccc3F)n2)ncc1F)C(F)(F)F] | I-403 |
| 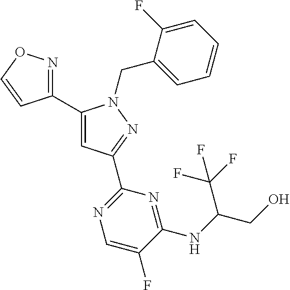 [SMILES: OCC(Nc1nc(-c2cc(-c3ccon3)n(Cc3ccccc3F)n2)ncc1F)C(F)(F)F] | I-404 |
| 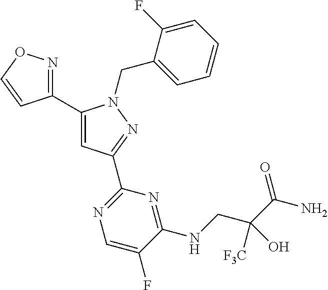 [SMILES: NC(=O)C(O)(CNc1nc(-c2cc(-c3ccon3)n(Cc3ccccc3F)n2)ncc1F)C(F)(F)F] | I-405 |
| 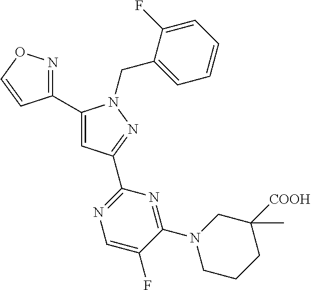 [SMILES: CC1(C(=O)O)CCCN(c2nc(-c3cc(-c4ccon4)n(Cc4ccccc4F)n3)ncc2F)C1] | I-406 |
| 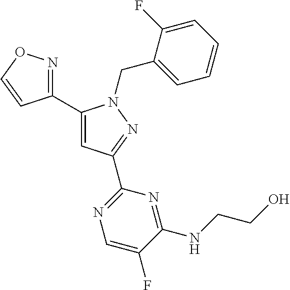 [SMILES: OCCNc1nc(-c2cc(-c3ccon3)n(Cc3ccccc3F)n2)ncc1F] | I-407 |
| 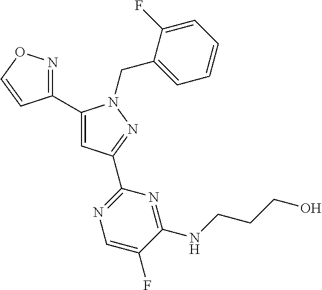 [SMILES: OCCCNc1nc(-c2cc(-c3ccon3)n(Cc3ccccc3F)n2)ncc1F] | I-408 |
| 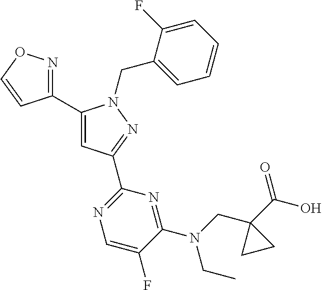 [SMILES: CCN(CC1(C(=O)O)CC1)c1nc(-c2cc(-c3ccon3)n(Cc3ccccc3F)n2)ncc1F] | I-409 |
| 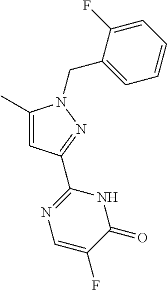 [SMILES: Cc1cc(-c2ncc(F)c(=O)[nH]2)nn1Cc1ccccc1F] | I-410 |
| 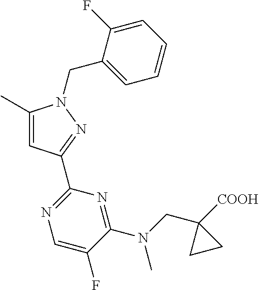 [SMILES: Cc1cc(-c2ncc(F)c(N(C)CC3(C(=O)O)CC3)n2)nn1Cc1ccccc1F] | I-411 |
| 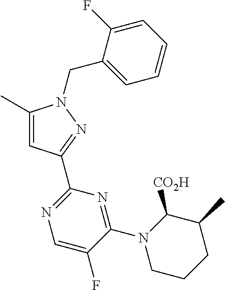 | I-412 |
| 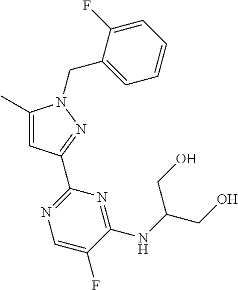 [SMILES: Cc1cc(-c2ncc(F)c(NC(CO)CO)n2)nn1Cc1ccccc1F] | I-413 |
| 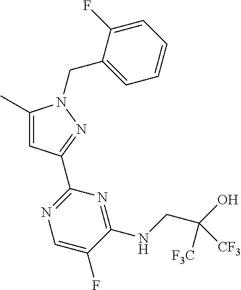 [SMILES: Cc1cc(-c2ncc(F)c(NCC(O)(C(F)(F)F)C(F)(F)F)n2)nn1Cc1ccccc1F] | I-414 |
| 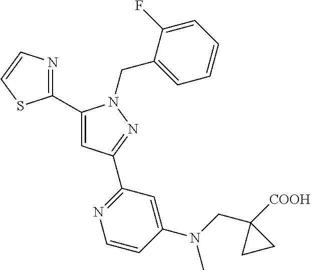 [SMILES: CN(CC1(C(=O)O)CC1)c1ccnc(-c2cc(-c3nccs3)n(Cc3ccccc3F)n2)c1] | I-415 |
| 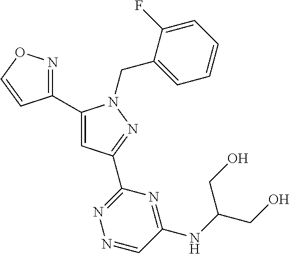 [SMILES: OCC(CO)Nc1cnnc(-c2cc(-c3ccon3)n(Cc3ccccc3F)n2)n1] | I-416 |
| 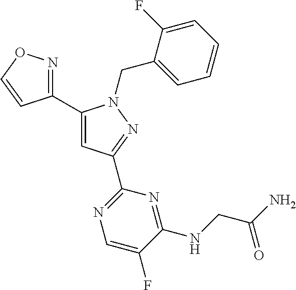 [SMILES: NC(=O)CNc1nc(-c2cc(-c3ccon3)n(Cc3ccccc3F)n2)ncc1F] | I-417 |
| 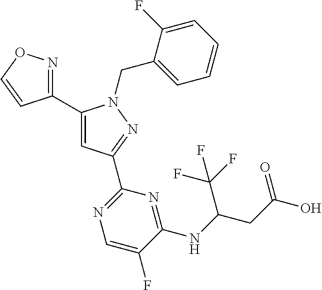 [SMILES: O=C(O)CC(Nc1nc(-c2cc(-c3ccon3)n(Cc3ccccc3F)n2)ncc1F)C(F)(F)F] | I-418 |
| 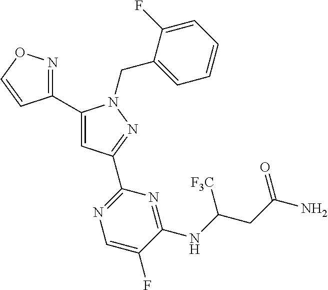 [SMILES: NC(=O)CC(Nc1nc(-c2cc(-c3ccon3)n(Cc3ccccc3F)n2)ncc1F)C(F)(F)F] | I-419 |
| 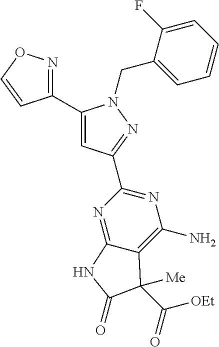 [SMILES: CCOC(=O)C1(C)C(=O)Nc2nc(-c3cc(-c4ccon4)n(Cc4ccccc4F)n3)nc(N)c21] | I-420 |
| 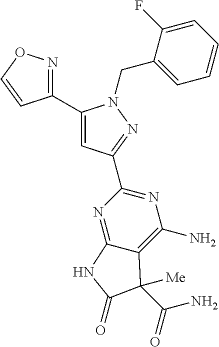 [SMILES: CC1(C(N)=O)C(=O)Nc2nc(-c3cc(-c4ccon4)n(Cc4ccccc4F)n3)nc(N)c21] | I-421 |
| 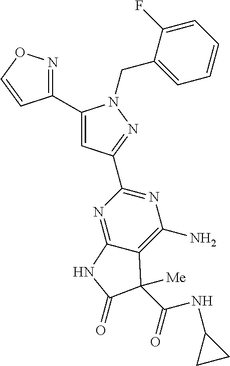 [SMILES: CC1(C(=O)NC2CC2)C(=O)Nc2nc(-c3cc(-c4ccon4)n(Cc4ccccc4F)n3)nc(N)c21] | I-422 |
| 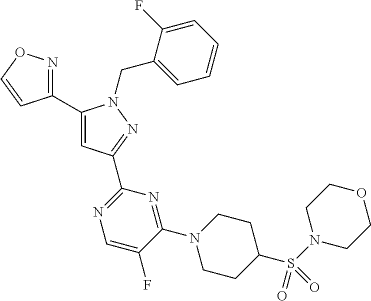 [SMILES: O=S(=O)(C1CCN(c2nc(-c3cc(-c4ccon4)n(Cc4ccccc4F)n3)ncc2F)CC1)N1CCOCC1] | I-423 |
| 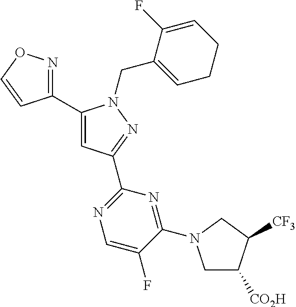 [SMILES: O=C(O)[C@@H]1CN(c2nc(-c3cc(-c4ccon4)n(CC4=CCCC=C4F)n3)ncc2F)C[C@H]1C(F)(F)F] | I-424 |
| 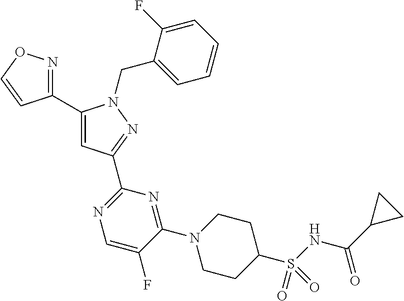 [SMILES: O=C(NS(=O)(=O)C1CCN(c2nc(-c3cc(-c4ccon4)n(Cc4ccccc4F)n3)ncc2F)CC1)C1CC1] | I-425 |
| 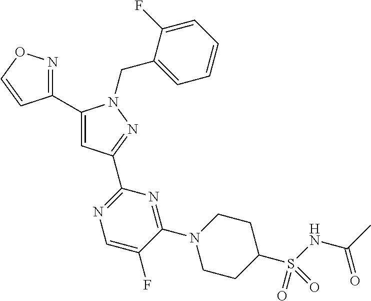 [SMILES: CC(=O)NS(=O)(=O)C1CCN(c2nc(-c3cc(-c4ccon4)n(Cc4ccccc4F)n3)ncc2F)CC1] | I-426 |
| 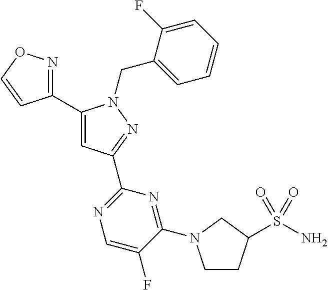 [SMILES: NS(=O)(=O)C1CCN(c2nc(-c3cc(-c4ccon4)n(Cc4ccccc4F)n3)ncc2F)C1] | I-427 |
| 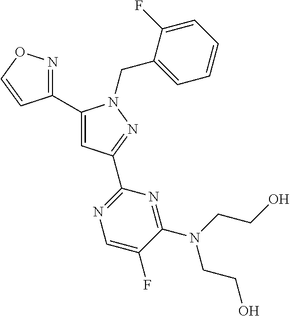 [SMILES: OCCN(CCO)c1nc(-c2cc(-c3ccon3)n(Cc3ccccc3F)n2)ncc1F] | I-428 |
| 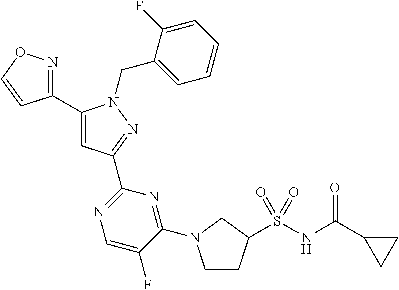 [SMILES: O=C(NS(=O)(=O)C1CCN(c2nc(-c3cc(-c4ccon4)n(Cc4ccccc4F)n3)ncc2F)C1)C1CC1] | I-429 |
| 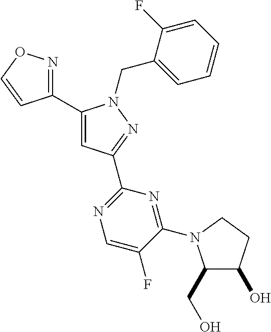 [SMILES: OC[C@@H]1[C@H](O)CCN1c1nc(-c2cc(-c3ccon3)n(Cc3ccccc3F)n2)ncc1F] | I-430 |
| 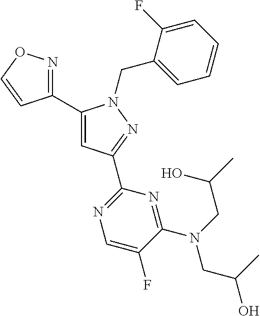 [SMILES: CC(O)CN(CC(C)O)c1nc(-c2cc(-c3ccon3)n(Cc3ccccc3F)n2)ncc1F] | I-431 |
| 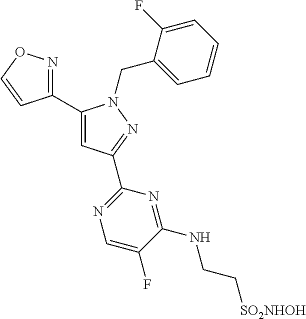 | I-432 |
| 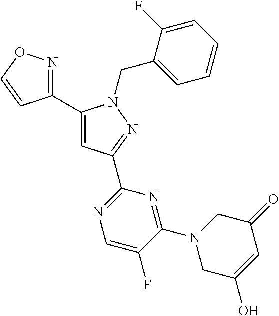 [SMILES: O=C1C=C(O)CN(c2nc(-c3cc(-c4ccon4)n(Cc4ccccc4F)n3)ncc2F)C1] | I-433 |
| 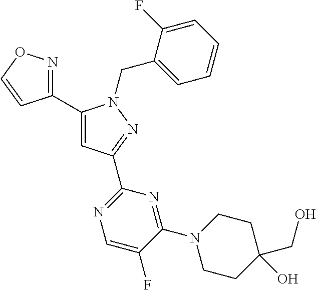 [SMILES: OCC1(O)CCN(c2nc(-c3cc(-c4ccon4)n(Cc4ccccc4F)n3)ncc2F)CC1] | I-434 |
| 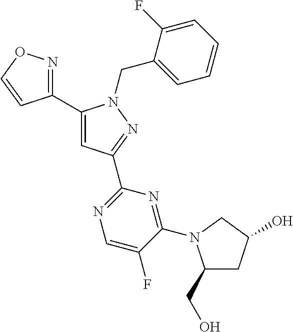 [SMILES: OC[C@@H]1C[C@@H](O)CN1c1nc(-c2cc(-c3ccon3)n(Cc3ccccc3F)n2)ncc1F] | I-435 |
| 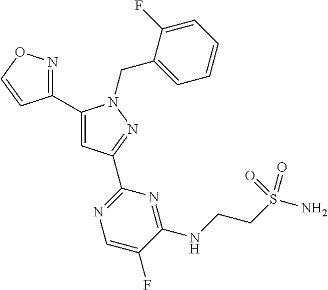 [SMILES: NS(=O)(=O)CCNc1nc(-c2cc(-c3ccon3)n(Cc3ccccc3F)n2)ncc1F] | I-436 |
| 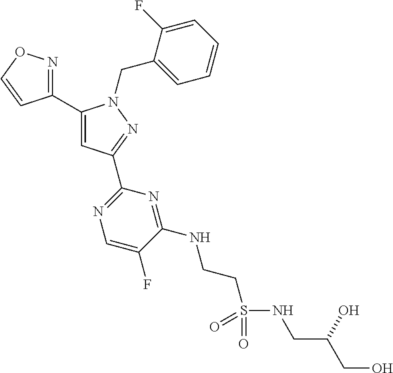 [SMILES: O=S(=O)(CCNc1nc(-c2cc(-c3ccon3)n(Cc3ccccc3F)n2)ncc1F)NC[C@H](O)CO] | I-437 |
| 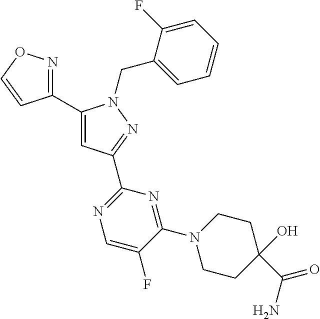 [SMILES: NC(=O)C1(O)CCN(c2nc(-c3cc(-c4ccon4)n(Cc4ccccc4F)n3)ncc2F)CC1] | I-438 |
| 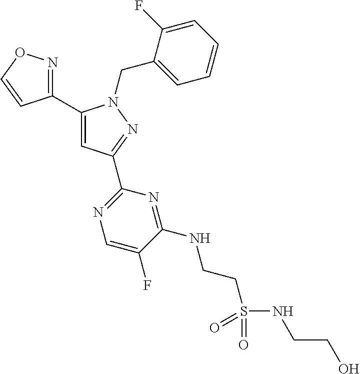 [SMILES: O=S(=O)(CCNc1nc(-c2cc(-c3ccon3)n(Cc3ccccc3F)n2)ncc1F)NCCO] | I-439 |
| 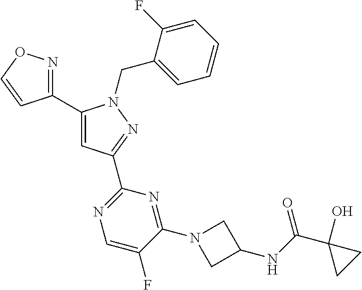 [SMILES: O=C(NC1CN(c2nc(-c3cc(-c4ccon4)n(Cc4ccccc4F)n3)ncc2F)C1)C1(O)CC1] | I-440 |
| 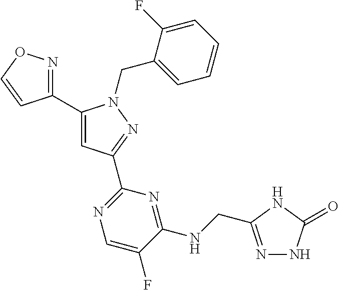 [SMILES: O=c1[nH]nc(CNc2nc(-c3cc(-c4ccon4)n(Cc4ccccc4F)n3)ncc2F)[nH]1] | I-441 |
| 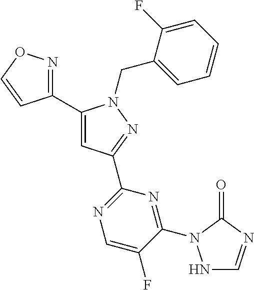 [SMILES: O=c1nc[nH]n1-c1nc(-c2cc(-c3ccon3)n(Cc3ccccc3F)n2)ncc1F] | I-442 |
| 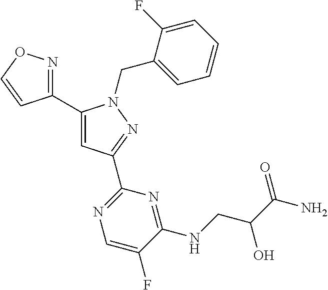 [SMILES: NC(=O)C(O)CNc1nc(-c2cc(-c3ccon3)n(Cc3ccccc3F)n2)ncc1F] | I-443 |
| 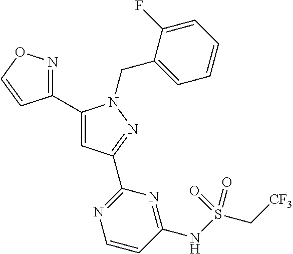 [SMILES: O=S(=O)(CC(F)(F)F)Nc1ccnc(-c2cc(-c3ccon3)n(Cc3ccccc3F)n2)n1] | I-444 |
| 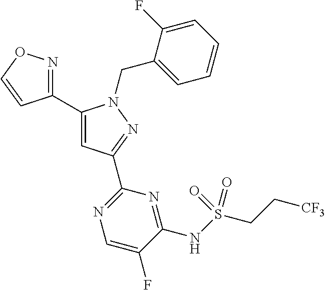 [SMILES: O=S(=O)(CCC(F)(F)F)Nc1nc(-c2cc(-c3ccon3)n(Cc3ccccc3F)n2)ncc1F] | I-445 |
| 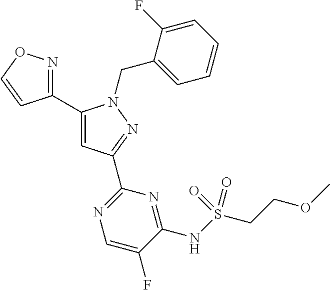 [SMILES: COCCS(=O)(=O)Nc1nc(-c2cc(-c3ccon3)n(Cc3ccccc3F)n2)ncc1F] | I-446 |
| 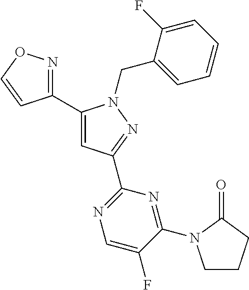 [SMILES: O=C1CCCN1c1nc(-c2cc(-c3ccon3)n(Cc3ccccc3F)n2)ncc1F] | I-447 |
| 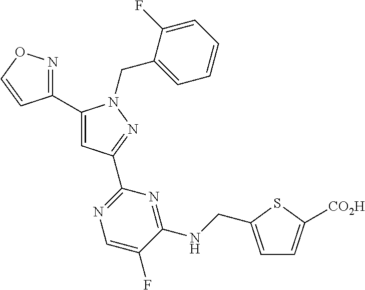 [SMILES: O=C(O)c1ccc(CNc2nc(-c3cc(-c4ccon4)n(Cc4ccccc4F)n3)ncc2F)s1] | I-448 |
| 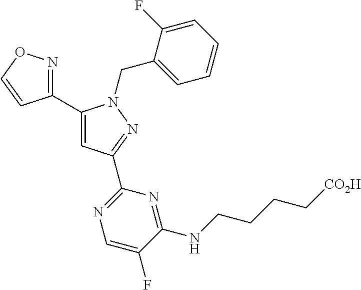 [SMILES: O=C(O)CCCCNc1nc(-c2cc(-c3ccon3)n(Cc3ccccc3F)n2)ncc1F] | I-449 |
| 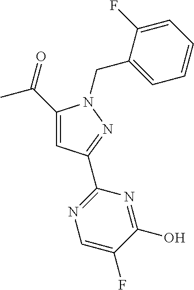 [SMILES: CC(=O)c1cc(-c2ncc(F)c(O)n2)nn1Cc1ccccc1F] | I-450 |
| 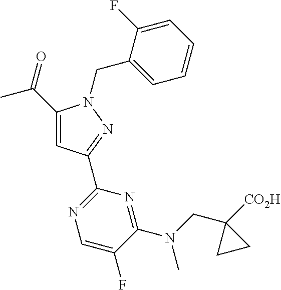 [SMILES: CC(=O)c1cc(-c2ncc(F)c(N(C)CC3(C(=O)O)CC3)n2)nn1Cc1ccccc1F] | I-451 |
| 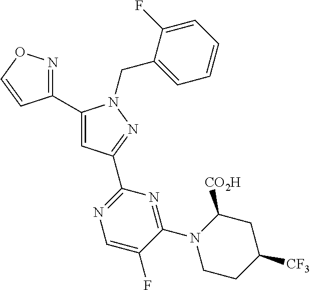 [SMILES: O=C(O)[C@H]1C[C@@H](C(F)(F)F)CCN1c1nc(-c2cc(-c3ccon3)n(Cc3ccccc3F)n2)ncc1F] | I-452 |
| 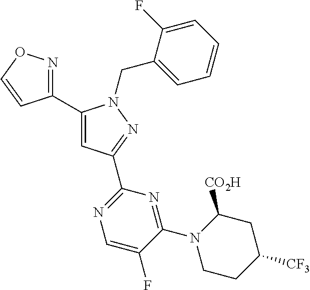 [SMILES: O=C(O)[C@H]1C[C@H](C(F)(F)F)CCN1c1nc(-c2cc(-c3ccon3)n(Cc3ccccc3F)n2)ncc1F] | I-453 |
| 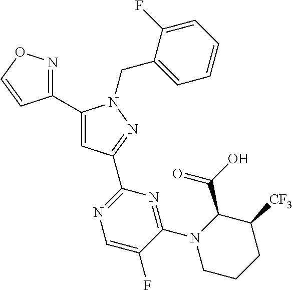 [SMILES: O=C(O)[C@H]1[C@@H](C(F)(F)F)CCCN1c1nc(-c2cc(-c3ccon3)n(Cc3ccccc3F)n2)ncc1F] | I-454 |
| 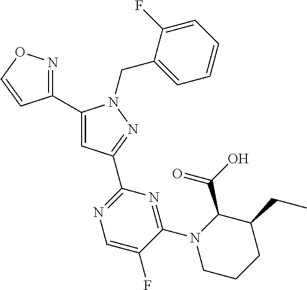 [SMILES: CC[C@H]1CCCN(c2nc(-c3cc(-c4ccon4)n(Cc4ccccc4F)n3)ncc2F)[C@H]1C(=O)O] | I-455 |

In other embodiments of the above methods, uses, compositions and kits, the sGC stimulator is a compound according to Formula IB, or a pharmaceutically acceptable salt thereof,

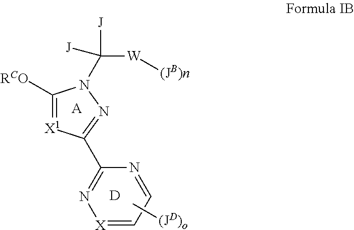

wherein X is selected from N or C;
X¹ is selected from N, CH, C(C₁₋₄ alkyl), C(C₁₋₄ fluoroalkyl), C(Cl), and CF;
W is either
i) absent, with J^{B} connected directly to the carbon atom bearing two J groups, each J is independently selected from hydrogen, methyl or fluorine, n is 1 and J^{B} is a C₁₋₆ alkyl chain optionally substituted by up to 6 instances of fluorine; or
ii) a ring B selected from phenyl or a 5 or 6-membered heteroaryl ring, containing 1 or 2 ring heteroatoms selected from N, O or S; wherein when W is ring B:
each J is hydrogen;
n is 0 or an integer selected from 1 to 3;
and each J^{B} is independently selected from halogen, —CN, a C₁₋₆ aliphatic, —OR^{B} or a C₃₋₈ cycloaliphatic group; wherein each said C₁₋₆ aliphatic and each said C₃₋₈ cycloaliphatic group is optionally and independently substituted with up to 3 instances of R³;
each R^{B} is independently selected from hydrogen, a C₁₋₆ aliphatic or a C₃₋₈ cycloaliphatic; wherein each said R^{B} that is a C₁₋₆ aliphatic and each said R^{B} that is a C₃₋₈ cycloaliphatic ring is optionally and independently substituted with up to 3 instances of R^{3a};
each R³ is independently selected from halogen, —CN, C₁₋₄ alkyl, C₁₋₄ haloalkyl, —O(C₁₋₄ alkyl) or —O(C₁₋₄ haloalkyl);
each R^{3a} is independently selected from halogen, —CN, C₁₋₄ alkyl, C₁₋₄ haloalkyl, —O(C₁₋₄ alkyl) or —O(C₁₋₄ haloalkyl);
   o is 0 or an integer selected from 1 to 3;
   each J^{D} is either absent or independently selected from hydrogen, halogen, —CN, —NO₂, —OR^{D}, —SR^{D}, —C(O)R^{D}, —C(O)OR^{D}, —OC(O)R^{D}, —C(O)N(R^{D})₂, —N(R^{D})₂, —N(R^{d})C(O)R^{D}, —N(R^{d})C(O)OR^{D}, —N(R^{d})C(O)N(R^{D})₂, —OC(O)N(R^{D})₂, —SO₂R^{D}, —SO₂N(R^{D})₂, —N(R^{d})SO₂R^{D}, a C₁₋₆ aliphatic, —(C₁₋₆ aliphatic)-R^{D}, a C₃₋₈ cycloaliphatic ring, a 6 to 10-membered aryl ring, a 4 to 8-membered heterocyclic ring or a 5 to 10-membered heteroaryl ring; wherein each said 4 to 8-membered heterocyclic ring and each said 5 to 10-membered heteroaryl ring contains between 1 and 3 heteroatoms independently selected from O, N or S; and wherein each of said C₁₋₆ aliphatic chains, each said C₃₋₈ cycloaliphatic ring, each said 6 to 10-membered aryl ring, each said 4 to 8-membered heterocyclic ring and each said 5 to 10-membered heteroaryl ring is optionally and independently substituted with up to 5 instances of R⁵;
   each R^{D} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 to 6-membered heteroaryl ring contains between 1 and 3 heteroatoms independently selected from O, N or S; and wherein each of said C₁₋₆ aliphatic chains, each said C₃₋₈ cycloaliphatic ring, each said 4 to 8-membered heterocyclic ring, each said phenyl and each said 5 to 6-membered heteroaryl ring is optionally and independently substituted with up to 5 instances of R^{5a}; wherein when any R^{D} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —C(O)—, —N(R^{d})— or —O—;
   each R^{d} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 or 6-membered heteroaryl ring contains between 1 and 3 heteroatoms independently selected from O, N or S; and wherein each of said C₁₋₆ aliphatic chains, each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5b};
   each R^{f} is independently selected from a C₁₋₃ alkyl, a C₃₋₈ cycloaliphatic ring, a 4 to 8-membered heterocyclic ring, phenyl or a 5 to 6-membered heteroaryl ring; wherein each said 4 to 8-membered heterocyclic ring and each said 5 to 6-membered heteroaryl ring contains between 1 and 4 heteroatoms independently selected from O, N or S; and wherein each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5c};
   when J^{D} is —C(O)N(R^{D})₂, —N(R^{D})₂, —N(R^{d})C(O)N(R^{D})₂, —OC(O)N(R^{D})₂ or —SO₂N(R^{D})₂, the two R^{D} groups together with the nitrogen atom attached to the two R^{D} groups may form a 4 to 8-membered heterocyclic ring or a 5-membered heteroaryl ring; wherein each said 4 to 8-membered heterocyclic ring and each said 5-membered heteroaryl ring optionally contains up to 3 additional heteroatoms independently selected from N, O or S, in addition to the nitrogen atom to which the two R^{D} groups are attached; and wherein each said 4 to 8-membered heterocyclic ring and each said 5-membered heteroaryl ring is optionally and independently substituted by up to 5 instances of R^{5d};
   when J^{D} is —N(R^{d})C(O)R^{D}, the R^{D} group together with the carbon atom attached to the R^{D} group, with the nitrogen atom attached to the R^{d} group, and with the R^{d} group may form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S, in addition to the nitrogen atom to which the R^{d} group is attached; and wherein each said 4 to 8-membered heterocyclic ring and each said 5-membered heteroaryl ring is optionally and independently substituted by up to 5 instances of R^{5d};
   when J^{D} is —N(R^{d})C(O)OR^{D}, the R^{D} group together with the oxygen atom attached to the R^{D} group, with the carbon atom of the —C(O)— portion of the —N(R^{d})C(O)OR^{D} group, with the nitrogen atom attached to the R^{d} group, and with said R^{d} group, may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R^{5d};
   when J^{D} is —N(R^{d})C(O)N(R^{D})₂, one of the R^{D} groups attached to the nitrogen atom, together with said nitrogen atom, and with the N atom attached to the R^{d} group and said R^{d} group may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R^{5d};
   when J^{D} is —N(R^{d})SO₂R^{D}, the R^{D} group together with the sulfur atom attached to the R^{D} group, with the nitrogen atom attached to the R^{d} group, and with said R^{d} group may combine to form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R^{5d};
   each R⁵ is independently selected from halogen, —CN, C₁₋₆ alkyl, —(C₁₋₆alkyl)-R⁶, —OR⁶, —SR⁶, —COR⁶, —OC(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)₂, —SO₂N(R⁶)(CO)—R⁶, —N(R⁶)SO₂R⁶, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl chains, said C₇₋₁₂ aralkyl, said C₃₋₈ cycloalkyl ring, said 4 to 7-membered heterocyclic ring, said 5 or 6-membered heteroaryl ring or said phenyl group is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
   alternatively, two instances of R⁵ attached to the same or different atoms of J^{D}, together with said atom or atoms of J^{D} to which they are attached, may form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —CONH₂, —OH or halogen; wherein R is hydrogen or a C₁₋₂ alkyl;
   each R^{5a} is independently selected from halogen, —CN, C₁₋₆ alkyl, —(C₁₋₆ alkyl)-R^{6a}, —OR^{6a}, —SR^{6a}, —COR^{6a}, —OC(O)R^{6a}, —C(O)OR^{6a}, —C(O)N(R^{6a})₂, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})(CO)—R^{6a}, —N(R^{6a})SO₂R^{6a}, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl chains, each said C₇₋₁₂ aralkyl, said C₃₋₈ cycloalkyl ring, said 4 to 7-membered heterocyclic ring, said 5 or 6-membered heteroaryl ring or phenyl group is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —COO(C₁₋₄ alkyl), —CONH₂, —O(C₁₋₄ alkyl), —O(C₁₋₄ haloalkyl) or oxo;
   each R^{5b} is independently selected from halogen, —CN, C₁₋₆ alkyl, —(C₁₋₆alkyl)-R^{6a}, —OR^{6a}, —SR^{6a}, —COR^{6a}, —OC(O)R^{6a}, —C(O)OR^{6a}, —C(O)N(R^{6a})₂, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})(CO)—R^{6a}, —N(R^{6a})SO₂R^{6a}, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl chains, each said C₇₋₁₂ aralkyl, said C₃₋₈ cycloalkyl ring, said 4 to 7-membered heterocyclic ring, said 5 or 6-membered heteroaryl ring or phenyl group is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —COO(C₁₋₄ alkyl), —CONH₂, —O(C₁₋₄ alkyl), —O(C₁₋₄ haloalkyl) or oxo;
   alternatively, two instances of R^{5a} or two instances of R^{5b} attached to the same or different atoms of R^{D} or R^{d}, respectively, together with said atom or atoms to which they are attached, may form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —CONH₂, —OH or halogen; wherein R is hydrogen or a C₁₋₂ alkyl;
   each R^{5c} is independently selected from halogen, —CN, C₁₋₆ alkyl, —(C₁₋₆alkyl)-R^{6b}, —OR^{6b}, —SR^{6b}, —COR^{6b}, —OC(O)R^{6b}, —C(O)OR^{6b}, —C(O)N(R^{6b})₂, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂OH, —SO₂NHOH, —SO₂N(R^{6b})(CO)—R^{6b}, —SO₂N(R^{6b})₂, —N(R^{6b})SO₂R^{6b}, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl chains, said C₇₋₁₂ aralkyl, said C₃₋₈ cycloalkyl ring, said 4 to 7-membered heterocyclic ring, said 5 or 6-membered heteroaryl ring or said phenyl groups is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
   alternatively, two instances of R^{5c} attached to the same or different atoms of R^{f}, together with said atom or atoms to which it is attached, may form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —CONH₂, —NR(CO)O(C₁₋₄ alkyl), —OH or halogen; wherein R is hydrogen or a C₁₋₂ alkyl;
   each R^{5d} is independently selected from halogen, —CN, C₁₋₆ alkyl, —(C₁₋₆alkyl)-R⁶, —OR⁶, —SR⁶, —COR⁶, —OC(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂, —SO₂OH, —SO₂NHOH, —SO₂N(R⁶)(CO)—R⁶, —SO₂R⁶, —SO₂N(R⁶)₂, —N(R⁶)SO₂R⁶, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl chains, said C₇₋₁₂ aralkyl, said C₃₋₈ cycloalkyl ring, said 4 to 7-membered heterocyclic ring, said 5 or 6-membered heteroaryl ring or said phenyl groups is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
   two instances of R⁵ or two instances of R^{5d}, attached to the same or different atoms of J^{D}, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —CONH₂, —NR(CO)O(C₁₋₄ alkyl), —OH or halogen; wherein R is hydrogen or a C₁₋₂ alkyl;
   each R⁶ is independently selected from hydrogen, a C₁₋₆ aliphatic, phenyl, benzyl, a C₃₋₈ cycloalkyl ring, a 4 to 7-membered heterocyclic ring or a 5 or 6-membered heteroaryl ring, wherein each of said C₁₋₆ aliphatic, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
   each R^{6a} is independently selected from hydrogen, a C₁₋₆ aliphatic, phenyl, benzyl, a C₃₋₈ cycloalkyl ring, a 4 to 7-membered heterocyclic ring or a 5 or 6-membered heteroaryl ring, wherein each of said C₁₋₆ aliphatic, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
   each R^{6b} is independently selected from hydrogen, a C₁₋₆ aliphatic, phenyl, benzyl, a C₃₋₈ cycloalkyl ring, a 4 to 7-membered heterocyclic ring or a 5 or 6-membered heteroaryl ring, wherein each of said C₁₋₆ aliphatic, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S; wherein
   two instances of R⁶ linked to the same nitrogen atom of R⁵ or R^{5d}, together with said nitrogen atom of R⁵ or R^{5d}, respectively, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S; two instances of R^{6a} linked to a nitrogen atom of R^{5a} or R^{5b}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
   two instances of R^{6b} linked to a nitrogen atom of R^{5c}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
   alternatively, two J^{D} groups attached to two vicinal ring D atoms, taken together with said two vicinal ring D atoms, may form a 5 to 7-membered heterocycle or a 5-membered heteroaryl ring that is fused to ring D; wherein said 5 to 7-membered heterocycle or said 5-membered ring heteroaryl contains from 1 to 3 heteroatoms independently selected from N, O or S; and wherein said 5 to 7-membered heterocycle or said 5-membered heteroaryl ring is optionally and independently substituted by up to 3 instances of oxo or —(Y)—R⁹;
   wherein Y is either absent or is a C₁₋₆ alkyl chain, optionally substituted by up to 6 instances of fluoro; and wherein when Y is said C₁₋₆ alkyl chain, up to 3 methylene units of this alkyl chain, can be replaced by a group selected from —O—, —C(O)— or —N((Y¹)—R⁹⁹)—;
   Y¹ is either absent or a C₁₋₆ alkyl chain, optionally substituted by up to 6 instances of fluoro;
   when Y¹ is absent, each R⁹⁹ is independently selected from hydrogen, C₁₋₆ alkyl optionally substituted with up to 9 fluorine atoms, —COR¹⁰, —C(O)OR¹⁰, —C(O)N(R¹⁰)₂, —C(O)N(R¹⁰)SO₂R¹⁰, —SO₂R¹⁰, —SO₂N(R¹⁰)₂, —SO₂N(R¹⁰)COOR¹⁰, —SO₂N(R¹⁰)C(O)R¹⁰, —SO₂OH, —SO₂NHOH, —SO₂N(R¹⁰)(CO)R¹⁰, a C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R^{11a};
   when Y¹ is present, each R⁹⁹ is independently selected from hydrogen, halogen, —CN, C₁₋₆ alkyl optionally substituted with up to 9 fluorine atoms, —COR¹⁰, —OR¹⁰, —OC(O)R¹⁰, —C(O)OR¹⁰, —C(O)N(R¹⁰)₂, —C(O)N(R¹⁰) SO₂R¹⁰, —SO₂R¹⁰, —SOR¹⁰, —SR¹⁰, —SO₂N(R¹⁰)₂, —SO₂N(R¹⁰)COOR¹⁰, —SO₂N(R¹⁰)C(O)R¹⁰, —SO₂OH, —SO₂NHOH, —SO₂N(R¹⁰)(CO)R¹⁰, a C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R^{11a};
   each R⁹ is independently selected from hydrogen, —CN, —OR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R₁₀)COOR¹⁰, —SO₂N(R¹⁰)C(O)R¹⁰, —N(R¹⁰)SO₂R¹⁰, —SO₂OH, —SO₂NHOH, —SO₂N(R¹⁰)(CO)—R¹⁰, a C₃₋₆ cycloalkyl ring, a 4-8-membered heterocyclic ring, a phenyl ring or a 5-6 membered heteroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R^{11a};
   each R¹⁰ is independently selected from hydrogen, a C₁₋₆ alkyl, —(C₁₋₆ alkyl)-R¹³, phenyl, benzyl, a C₃₋₆ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of R^{11b};
   each R¹³ is independently selected from a phenyl, a benzyl, a C₃₋₆ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each said phenyl, each of said benzyl, each said C₃₋₈ cycloalkyl group, each said 4 to 7-membered heterocyclic ring and each 5 or 6-membered heteroaryl ring is optionally and independently substituted with up to 3 instances of R^{11c};
   each R^{11a} is independently selected from halogen, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹;
   each R^{11b} is independently selected from halogen, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹; and
   each R^{11c} is independently selected from halogen, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²¹;
   each R¹² is selected from hydrogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo;
   each R¹²¹ is selected from C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo;
   R^{C} is selected from hydrogen, C₁₋₆ aliphatic, —(C₁₋₆ alkyl)-R^{N}, a 5 or 6-membered heteroaryl, phenyl, a 4 to 7 membered heterocyclic, a C₃₋₈ cycloaliphatic, —C(O)R⁷, —C(O)OR⁷, —C(O)N(R⁷)₂, and —C(O)N(R⁷)SO₂R⁷; wherein each of said 5 or 6-membered heteroaryl ring and 4 to 7-membered heterocyclic ring contains up to 4 ring heteroatoms independently selected from N, O and S; wherein each said C₁₋₆ aliphatic and each C₁₋₆ alkyl portion of said —(C₁₋₆ alkyl)-R^{N}, is optionally and independently substituted with up to 6 instances of halogen and up to 2 instances of —CN, —COOR⁸, —OR⁸, oxo, —N(R⁸)₂, —C(O)N(R⁸)₂, —N(R⁸)C(O)R⁸, —N(R⁸)C(O)OR⁸, —N(R⁸)C(O)N(R⁸)₂, —SO₂R⁸, —SO₂N(R⁸)₂, —NHOR⁸, —SO₂N(R⁸)COOR⁸, —SO₂N(R⁸)C(O)R⁸ and —N(R⁸)SO₂R⁸;
   wherein each R⁷ is independently selected from hydrogen, C₁₋₆ alkyl, C₁₋₆ fluoroalkyl, a C₃₋₈ cycloalkyl ring, phenyl, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
   each R⁸ is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
   each R^{N} is independently selected from a phenyl ring, a monocyclic 5 or 6-membered heteroaryl ring, a monocyclic C₃₋₆ cycloaliphatic ring, or a monocyclic 4 to 6-membered heterocycle; wherein said monocyclic 5 or 6-membered heteroaryl ring or said monocyclic 4 to 6-membered heterocycle contain between 1 and 4 heteroatoms selected from N, O or S; wherein said monocyclic 5 or 6-membered heteroaryl ring is not a 1,3,5-triazinyl ring; and wherein said phenyl, said monocyclic 5 to 6-membered heteroaryl ring, said monocyclic C₃₋₆ cycloaliphatic ring, or said monocyclic 4 to 6-membered heterocycle is optionally and independently substituted with up to 6 instances of fluoro and/or up to 3 instances of J^{M};
   each J^{M} is independently selected from —CN, a C₁₋₆ aliphatic, —OR^{M}, —SR^{M}, —N(R^{M})₂, a C₃₋₈ cycloaliphatic ring or a 4 to 8-membered heterocyclic ring; wherein said 4 to 8-membered heterocyclic ring contains 1 or 2 heteroatoms independently selected from N, O or S; wherein each said C₁₋₆ aliphatic, each said C₃₋₈ cycloaliphatic ring and each said 4 to 8-membered heterocyclic ring, is optionally and independently substituted with up to 3 instances of R^{7c}; and
   each R^{M} is independently selected from hydrogen, a C₁₋₆ aliphatic, a C₃₋₈ cycloaliphatic ring or a 4 to 8-membered heterocyclic ring; wherein each said 4 to 8-membered heterocylic ring contains between 1 and 3 heteroatoms independently selected from O, N or S;
   each R^{7c} is independently selected from halogen, —CN, —NO₂, C₁₋₄ alkyl, C₁₋₄ haloalkyl, C₃₋₈ cycloalkyl ring, —OR^{8b}, —SR^{8b}, —N(R^{8b})₂, —C(O)O(C₁₋₄ alkyl), —C(O)OH, —NR(CO)CO(C₁₋₄ alkyl) or an oxo group; wherein each said cycloalkyl group is optionally and independently substituted with up to 3 instances of halogen;
   each R^{8b} is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
   provided that the compound is not one represented by the general structure:

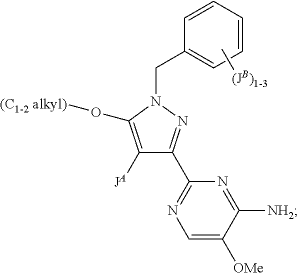

wherein J^{A} is either hydrogen or C₁₋₄ alkyl; and J^{B} is either halogen or C₁₋₄ (alkoxy).

In some embodiments of the above methods, uses, compositions and kits, the sGC stimulator is a compound of Formula IB, wherein W is absent. In some of these embodiments, the compound is a compound of Formula IIaB:

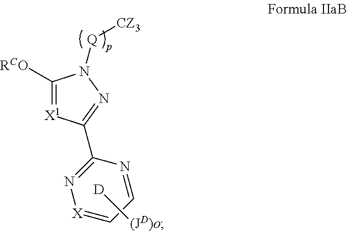

wherein Q represents a —CZ₂— group; each Z is independently selected from hydrogen or fluorine; and p is an integer selected from 1, 2, 3, 4 and 5. In some embodiments of Formula IIaB up to 5 instances of Z are fluorine and the remaining instances of Z are hydrogen.

In other embodiments of the above methods, uses, compositions and kits, the sGC stimulator is a compound of Formula IB, and has Formula IIIaB:

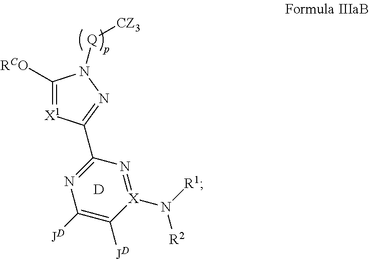

In some of these embodiments wherein the sGC stimulator is one of Formula IIIaB, X is N, and the moiety —N(R¹)(R²) is absent. In other of these embodiments, X is C, and the moiety —N(R¹)(R²) is present. In some of the embodiments in which the —N(R¹)(R²) moiety is present:

R¹ and R², together with the nitrogen atom to which they are attached, form a 4 to 8-membered heterocyclic ring or 5-membered heteroaryl ring; wherein said 4 to 8-membered heterocyclic ring or 5-membered heteroaryl ring optionally contains, in addition to the nitrogen atom to which both R¹ and R² are attached, up to 3 ring heteroatoms independently selected from N, O or S, and is optionally substituted by up to 5 instances of R^{5e};
each R^{5e} is independently selected from halogen, —CN, C₁₋₆ alkyl, —(C₁₋₄ alkyl)-R⁶, a C₃₋₈ cycloalkyl ring, C₁₋₄ (cyanoalkyl), —OR⁶, —SR⁶, —OCOR⁶, —COR⁶, —C(O)OR⁶, —C(O)N(R⁶)₂, —N(R⁶)C(O)R⁶, —N(R⁶)₂, —SO₂R⁶, —SO₂N(R⁶)₂, —N(R⁶)SO₂R⁶, —SO₂OH, —SO₂NHOH, —SO₂N(R⁶)(CO)—R⁶, benzyl, phenyl or an oxo group; wherein each said phenyl ring and each said benzyl group, is optionally and independently substituted with up to 3 instances of halogen, —OH, —NH₂, —NH(C₁₋₄ alkyl), —N(C₁₋₄ alkyl)₂, —CN, C₁₋₄ alkyl, C₁₋₄ haloalkyl, —CONH₂, —O(C₁₋₄ alkyl) or —O(C₁₋₄ haloalkyl); and wherein each said C₁₋₆ alkyl or C₁₋₄ alkyl chains and each said C₃₋₈ cycloalkyl ring is optionally and independently substituted with up to 3 instances of halogen; wherein
each R⁶ is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
alternatively, two of the instances of R^{5e} attached to the same or different atoms of said ring formed by R¹, R² and the nitrogen to which R¹ and R² are attached, together with said atom or atoms, optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —CONH₂, —C(O)OH, —NR(CO)O(C₁₋₄ alkyl), —OH or halogen; wherein R is hydrogen or a C₁₋₂ alkyl;
In some of these embodiments, alternatively, R¹ and R² are each independently selected from hydrogen, C₁₋₆ alkyl, a C₃₋₈ cycloalkyl ring, a 4 to 8-membered heterocyclic ring, a 5 or 6-membered heteroaryl, phenyl or a C₁₋₆ alkyl-R^{Y}; wherein each of said 4 to 8-membered heterocyclic ring and each of said 5 or 6-membered heteroaryl ring contains up to 3 ring heteroatoms independently selected from N, O and S; and wherein each of said C₁₋₆ alkyl, C₃₋₈ cycloalkyl ring, 4 to 8-membered heterocyclic ring group, 5 or 6-membered heteroaryl, phenyl and C₁₋₆ alkyl-R^{Y} is optionally and independently substituted with up to 5 instances of R^{5f};
R^{Y} is selected from a C₃₋₈ cycloalkyl ring, a 4 to 8-membered heterocyclic ring, phenyl, or a 5 to 6-membered heteroaryl ring; wherein each of said 4 to 8-membered heterocyclic ring or 5 to 6-membered heteroaromatic ring contains between 1 and 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaryl ring is optionally substituted with up to 5 instances of R^{5g};
each R^{5f} is independently selected from halogen, —CN, C₁₋₆ alkyl, —(C₁₋₄ alkyl)-R^{6a}, a C₇₋₁₂ aralkyl, C₃₋₈ cycloalkyl ring, C₁₋₄ (cyanoalkyl), —OR^{6a}, —SR^{6a}, —OCOR^{6a}, —COR^{6a}, —C(O)OR^{6a}, —C(O)N(R^{6a})₂, —N(R^{6a})C(O)R^{6a}, —N(R^{6a})₂, —SO₂R^{6a}, —SO₂N(R^{6a})₂, —N(R^{6a})SO₂R^{6a}, phenyl or an oxo group; wherein each said phenyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₇₋₁₂ aralkyl, each said C₁₋₄ alkyl chain and each said C₃₋₈ cycloalkyl group is optionally and independently substituted with up to three instances of halogen;
each R^{6a} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen;
each R^{5g} is independently selected from halogen, —CN, C₁₋₆ alkyl, —(C₁₋₄ alkyl)-R^{6b}, a benzyl, C₃₋₈ cycloalkyl ring, C₁₋₄ (cyanoalkyl), —OR^{6b}, —SR^{6b}, —OCOR^{6b}, —COR^{6b}, —C(O)OR^{6b}, —C(O)N(R^{6b})₂, —N(R^{6b})C(O)R^{6b}, —N(R^{6b})₂, —SO₂R^{6b}, —SO₂OH, —SO₂NHOH, —SO₂N(R^{6b})(CO)—R^{6b}, —SO₂N(R^{6b})₂, —N(R^{6b})SO₂R^{6b}, phenyl or an oxo group; wherein each said phenyl and each said benzyl group is optionally and independently substituted with up to 3 instances of halogen, —OH, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl); and wherein each said C₁₋₄ alkyl chain and each said C₃₋₈ cycloalkyl group is optionally and independently substituted with up to 3 instances of halogen;
each R^{6b} is independently selected from hydrogen, a C₁₋₆ alkyl, a C₂₋₄ alkenyl, phenyl, benzyl, or a C₃₋₈ cycloalkyl ring; wherein each said C₁₋₆ alkyl, each said C₂₋₄ alkenyl, each said phenyl, each said benzyl and each said C₃₋₈ cycloalkyl group is optionally and independently substituted with up to 3 instances of halogen.
In some embodiments, alternatively, two instances of R^{5g} attached to the same or different ring atoms of R^{Y}, together with said ring atom or atoms, form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or a 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR″(CO)CO(C₁₋₄ alkyl), —OH or halogen; and R″ is hydrogen or a C₁₋₂ alkyl.
In those embodiments when one of R¹ or R² is the C₃₋₈ cycloalkyl ring, 4 to 8-membered heterocyclic ring or 5 or 6-membered heteroaryl substituted with up to 5 instances of R^{5f}, two of the instances of R^{5f} attached to the same or different ring atoms of said R¹ or R², together with said atom or atoms, form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)CO(C₁₋₄ alkyl), —NR′(CO)CO(C₁₋₄ alkyl) or halogen; wherein R′ is hydrogen or a C₁₋₂ alkyl.

In some embodiments of the above methods, uses, compositions and kits, the sGC stimulator is a compound of Formulae IB, IIaB or IIIaB, wherein X is N. In other embodiments, X is C.

In some of the embodiments of the above methods, uses, compositions an kits, the sGC stimulator is a compound of Formula IB, and has Formula IVaB:

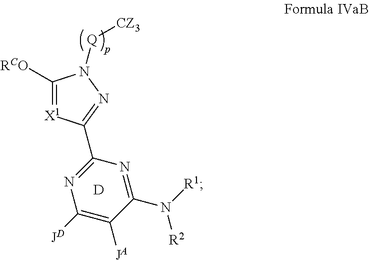

wherein J^{D} is absent or is selected from halogen, methyl, hydroxyl, methoxy, trifluoromethyl, trifluoromethoxy or —NR^{a}R^{b}; in some of these embodiments, R^{a} and R^{b} are each independently selected from hydrogen, C₁₋₆ alkyl or a 3-6 cycloalkyl ring; alternatively, R^{a} and R^{b}, together with the nitrogen atom to which they are both attached, may form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 5 instances of fluorine; J^{A} is selected from hydrogen or fluorine; and R¹ and R² are as defined supra.

In other embodiments of the above methods, uses, compositions and kits, the sGC stimulator is a compound of Formula I, having Formula IIbB, or is a pharmaceutically acceptable salt thereof:

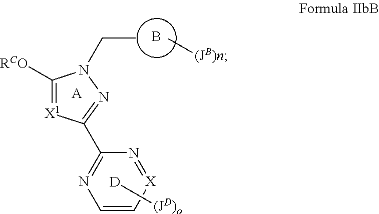

In some of these embodiments, ring B is a phenyl. In other embodiments, ring B is a 5 or 6-membered heteroaryl ring, containing 1 or 2 ring heteroatoms selected from N, O or S.

In some embodiments of the above methods, uses, compositions and kits, the sGC stimulator is a compound of Formula IIbB, wherein X is C. In some of these embodiments, X is optionally substituted by J^{D}. In other embodiments of the compounds of Formula IIbB, X is N.

In some embodiments of the compounds of Formula IIbB, each J^{D} is independently selected from halogen, a C₁₋₆ aliphatic, C₁₋₆ haloaliphatic, —N(R^{D})₂, —N(R^{d})COR^{D}, —N(R^{d})COOR^{D}, —OR^{D}, —N(R^{d})SO₂R^{D} oxo or an optionally substituted C₃₋₈ cycloaliphatic ring. In other embodiments, o is 2 and each J^{D} is independently selected from a halogen atom or —N(R^{D})₂, —N(R^{d})COR^{D}, —OH, —N(R^{d})COOR^{D}, or —N(R^{d})SO₂R^{D}. In still other embodiments, o is 2 and one instance of J^{D} is fluoro or chloro and the other instance of J^{D} is —OH. In yet other embodiments, o is 2 and one instance of J^{D} is —NH₂ and the other one is independently selected from —N(R^{D})₂, wherein at least one instance of R^{D} is not hydrogen; or is —NHCOR^{D}, —N(R^{d})COOR^{D} or —N(R^{d})SO₂R^{D}. In further embodiments, o is 2 and one instance of J^{D} is independently selected from —N(R^{D})₂ or —NHCOR^{D} and the other instance of J^{D} is selected from fluoro or chloro. In further embodiments, o is 1 and J^{D} is amino.

In some embodiments of the above methods, uses, compositions and kits, the sGC stimulator is a compound of Formula IB, having Formula IIIbB:

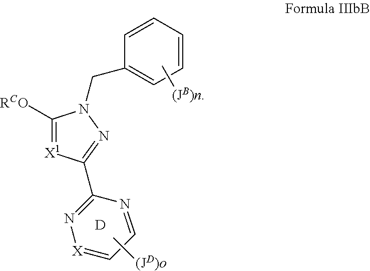

In other embodiments of the above methods, uses, compositions and kits, the sGC stimulator is a compound of Formula IB, the compound having Formula IVbB:

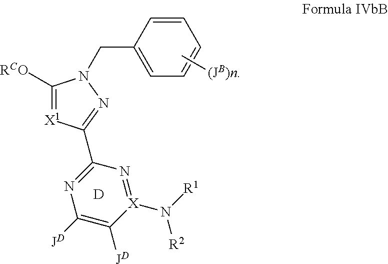

In some of these embodiments wherein the sGC stimulator is a compound of Formula IVbB, X is N; in these embodiments, —NR¹R² is absent. In other embodiments, X is C.

In some embodiments of the above methods, uses, compositions and kits, the sGC stimulator is a compound of Formula IB, the compound having Formula VbB:

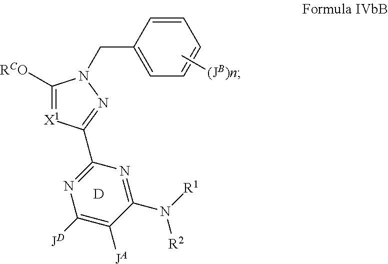

wherein, J^{D} is absent or is selected from halogen, methyl, hydroxyl, methoxy, trifluoromethyl, trifluoromethoxy or —NR^{a}R^{b}; in some of these embodiments, R^{a} and R^{b} are each independently selected from hydrogen, C₁₋₆ alkyl or a 3-6 cycloalkyl ring; alternatively, R^{a} and R^{b}, together with the nitrogen atom to which they are both attached, may form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 5 instances of fluorine; and J^{A} is selected from hydrogen or fluorine.

In some embodiments of the above methods, uses, compositions and kits, the sGC stimulator is a compound of Formula IIbB, wherein ring B is phenyl or a 6-membered heteroaryl ring. In some of these embodiments, n is an integer selected from 1, 2, or 3 and each J^{B} is independently selected from halogen, a C₁₋₆ aliphatic or —OR^{B}. In other embodiments, each J^{B} is independently selected from halogen. In still other embodiments, each J^{B} is independently selected from fluoro or chloro. In yet other embodiments, each J^{B} is fluoro. In further embodiments, each J^{B} is methyl or ethyl.

In some embodiments of the above methods, uses, compositions and kits, the sGC stimulator is a compound of any one of Formulae IIbB, IIIbB, IVbB or VbB, wherein n is 1. In some of these embodiments, J^{B} is selected from halogen. In other embodiments, J^{B} is fluoro or chloro. In still other embodiments, J^{B} is fluoro.

In some embodiments of the above methods, uses, compositions and kits, the sGC stimulator is a compound of any one of Formulae IIbB, IIIbB, IVbB and VBB, wherein at least one J^{B} is ortho to the attachment of the methylene linker between ring B and ring A. In some of these embodiments, each J^{B} is independently selected from halogen. In other embodiments, each J^{B} is independently selected from fluoro or chloro. In still other embodiments, each J^{B} is fluoro. In some of the embodiments wherein n is 1, the J^{B} ortho to the attachment of the methylene linker between ring B and ring A is fluoro.

In some embodiments of the above methods, uses, compositions and kits, the sGC stimulator is a compound of any one of Formulae IIbB, IIIbB, IVbB and VbB, wherein ring B is a 6-membered heteroaryl ring. In some of these embodiments, B is a pyridyl ring. In other of these embodiments, ring B is a pyrimidinyl ring.

In some embodiments of the above methods, uses, compositions and kits, the sGC stimulator is a compound of Formula IB, wherein o is an integer selected from 1, 2, or 3. In some of these embodiments, each J^{D} is independently selected from halogen, a C₁₋₆ aliphatic, C₁₋₆ haloaliphatic, —N(R^{D})₂, —N(R^{d})C(O)R^{D}, —N(R^{d})C(O)OR^{D}, —N(R^{d})C(O)N(R^{D})₂, —SO₂R^{D}, —SO₂N(R^{D})₂, —N(R^{d})SO₂R^{D}, —SR^{D}, —OR^{D} or an optionally substituted C₃₋₈ cycloaliphatic ring. In other embodiments, each J^{D} is independently selected from methyl, trifluoromethyl, chloro, fluoro, —N(R^{D})₂, N(R^{d})C(O)R^{D}, —N(R^{d})SO₂R^{D}, or —OR^{D}. In some of these embodiments, R^{d} is independently selected from hydrogen or C₁₋₄ alkyl. In yet other embodiments, o is 1 or 2 and at least one instance of J^{D} is independently selected from fluoro, chloro, hydroxyl and amino. In further embodiments, o is an integer selected from 1 or 2.

In some embodiments of the above methods, uses, compositions and kits, the sGC stimulator is a compound of Formula IB having one of Formulae VIbB, VIIbB, VaB or VIaB:

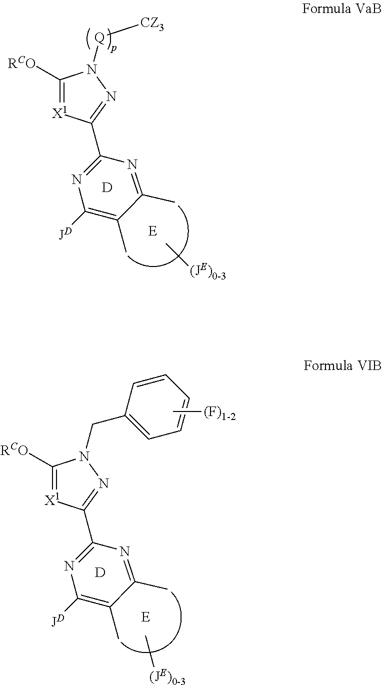

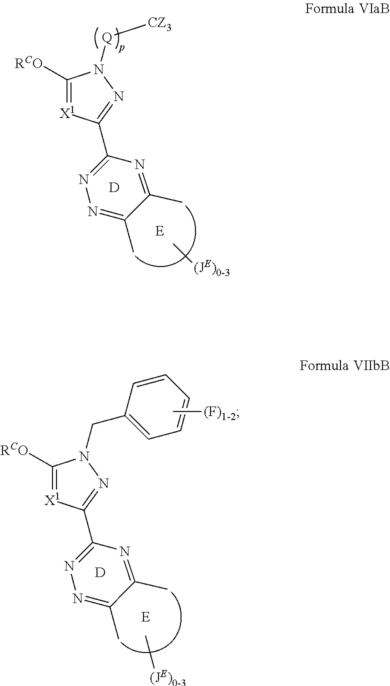

wherein ring E is a 5 or 6-membered heterocyclic ring, containing up to 3 heteroatoms selected from N, O and S; and wherein each J^{E} is independently selected from oxo or —(Y)—R⁹. In some of these embodiments, J^{D} is absent or is selected from halogen, —NH₂, or —OH. In other embodiments, ring E is a heterocyclic ring containing one nitrogen ring atom and at least one instance of J^{E} is oxo. In still other embodiments, one J^{E} is oxo and two other instances of J^{E} are independently selected from —(Y)—R⁹. In further embodiments, each —(Y)—R⁹ is independently selected from a C₁₋₆ alkyl; a 5 or 6-membered heteroaryl ring containing between 1 and 3 heteroatoms independently selected from N, O or S and optionally substituted by one or more instances of C₁₋₆ alkyl or halogen; and —(CO)NH—R¹⁰. In further embodiments, R¹⁰ is a C₃₋₆ cycloalkyl ring.

In some embodiments of the above methods, uses, compositions and kits, the sGC stimulator is a compound of Formula IB having one of Formulae VIIaB or VIIIbB:

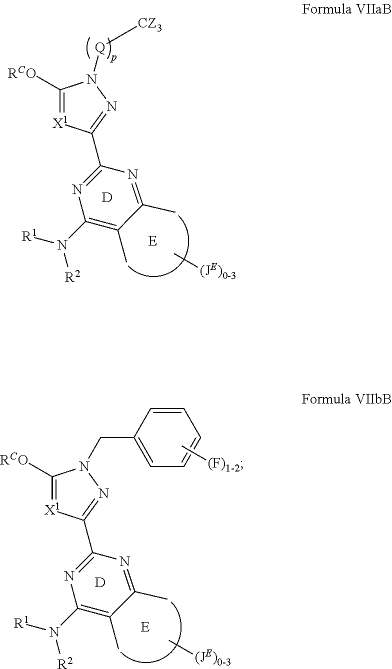

In some of these embodiments wherein the sGC stimulator is a compound of Formulae VIIaB or VIIIbB, one instance of J^{E} is oxo and two other instances of J^{E} are independently selected from C₁₋₆ alkyl; a 5 or 6-membered heteroaryl ring, containing between 1 and 3 heteroatoms independently selected from N, O, or S and optionally substituted by one or more instances of C₁₋₆ alkyl or halogen; and —(CO)NH—R¹⁰. In some of these embodiments, R¹⁰ is a C₃₋₆ cycloalkyl ring.

In some embodiments of the above methods, uses, compositions and kits, the sGC stimulator is a compound of Formula IB having one of Formulae VIIIaB or XIXbB:

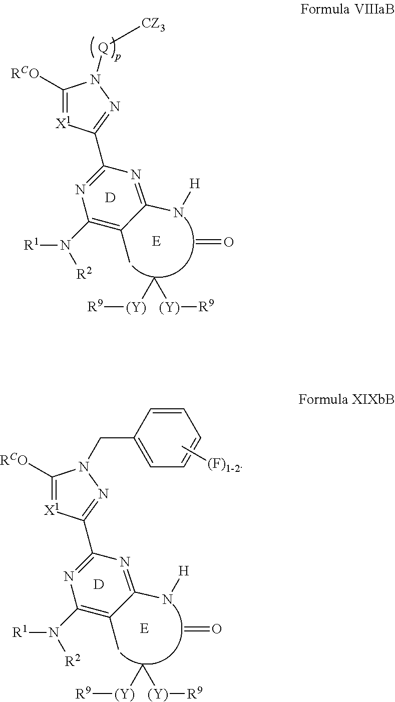

In these embodiments, both —(Y)—R⁹ substituents are attached to any ring carbon anywhere on the ring, provided that both —(Y)—R⁹ substituents are attached to the same ring carbon.

In other embodiments of the above methods, uses, compositions and kits, the sGC stimulator is a compound of Formula IB having one of Formulae XIXaB, XaB, XbB, XIbB:

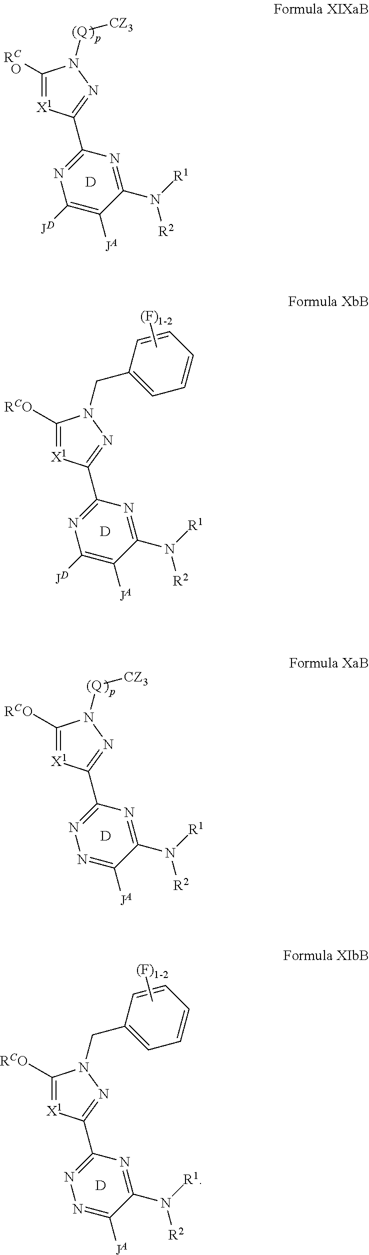

In these embodiments, each J^{D} is independently selected from —NH₂ or is absent; and wherein each J^{A} is alternatively: i) when R¹ and R² are not simultaneously hydrogen, each J^{A} is independently selected from hydrogen or halogen; or ii) when R¹ and R² are both simultaneously hydrogen, each J^{A} is independently selected from —C(O)R^{D}, —C(O)OR^{D}, —OC(O)R^{D}, —C(O)N(R^{D})₂, —N(R^{D})₂, —N(R^{d})C(O)R^{D}, —N(R^{d})C(O)OR^{D}, —N(R^{d})C(O)N(R^{D})₂, —OC(O)N(R^{D})₂, —SO₂R^{D}, —SO₂N(R^{D})₂ or —N(R^{d})SO₂R^{D}. In some of these embodiments, J^{A} is —NH₂, —OH, or hydrogen.

In some embodiments of the above methods, uses, compositions and kits, the sGC stimulator is a compound of Formula IB wherein R^{C} is C₁₋₆ aliphatic optionally substituted with up to 6 instances of fluoro. In some embodiments, R^{C} is C₁₋₆ alkyl optionally substituted with up to 6 instances of fluoro. In other embodiments, R^{C} is ethyl or methyl; the ethyl or methyl may be optionally substituted with up to 5 instances of fluoro. In still other embodiments, R^{C} is a C₃₋₆ cycloaliphatic, optionally substituted with up to 4 instances of fluoro.

In some embodiments of the above methods, uses, compositions and kits, the sGC stimulator is a compound of Formula IB selected from those depicted in Table XB below:

TABLE XB

| | |
|---|---|
| 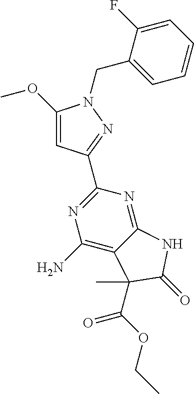 [SMILES: CCOC(=O)C1(C)C(=O)Nc2nc(-c3cc(OC)n(Cc4ccccc4F)n3)nc(N)c21] | I-1 |
| 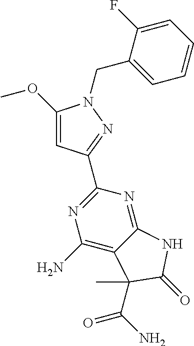 [SMILES: COc1cc(-c2nc(N)c3c(n2)NC(=O)C3(C)C(N)=O)nn1Cc1ccccc1F] | I-2 |
| 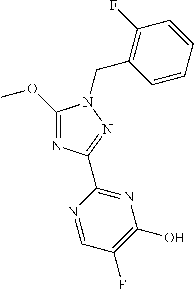 [SMILES: COc1nc(-c2ncc(F)c(O)n2)nn1Cc1ccccc1F] | I-3 |
| 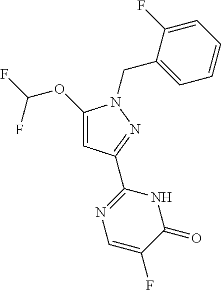 [SMILES: O=c1[nH]c(-c2cc(OC(F)F)n(Cc3ccccc3F)n2)ncc1F] | I-4 |
| 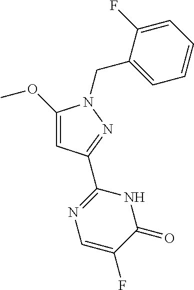 [SMILES: COc1cc(-c2ncc(F)c(=O)[nH]2)nn1Cc1ccccc1F] | I-5 |
| 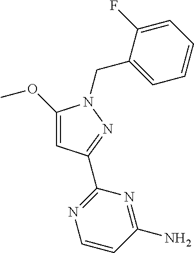 [SMILES: COc1cc(-c2nccc(N)n2)nn1Cc1ccccc1F] | I-6 |
| 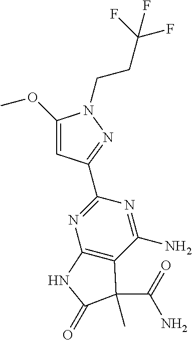 [SMILES: COc1cc(-c2nc(N)c3c(n2)NC(=O)C3(C)C(N)=O)nn1CCC(F)(F)F] | I-7 |
| 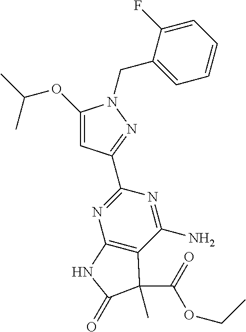 [SMILES: CCOC(=O)C1(C)C(=O)Nc2nc(-c3cc(OC(C)C)n(Cc4ccccc4F)n3)nc(N)c21] | I-8 |
| 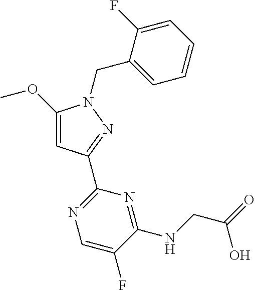 [SMILES: COc1cc(-c2ncc(F)c(NCC(=O)O)n2)nn1Cc1ccccc1F] | I-9 |
| 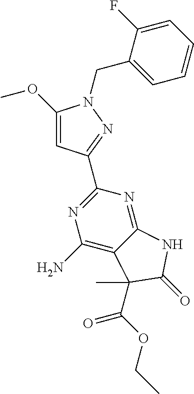 [SMILES: CCOC(=O)C1(C)C(=O)Nc2nc(-c3cc(OC)n(Cc4ccccc4F)n3)nc(N)c21] | I-1 |
| 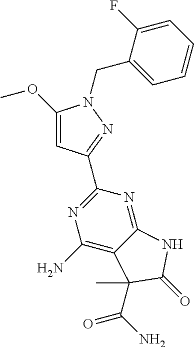 [SMILES: COc1cc(-c2nc(N)c3c(n2)NC(=O)C3(C)C(N)=O)nn1Cc1ccccc1F] | I-2 |
| 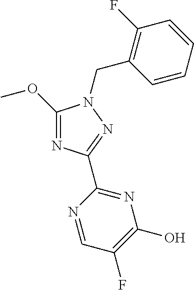 [SMILES: COc1nc(-c2ncc(F)c(O)n2)nn1Cc1ccccc1F] | I-3 |
| 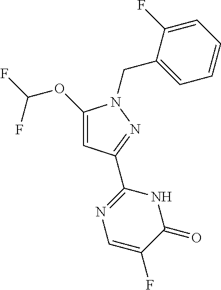 [SMILES: O=c1[nH]c(-c2cc(OC(F)F)n(Cc3ccccc3F)n2)ncc1F] | I-4 |
| 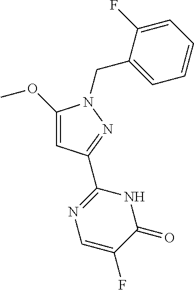 [SMILES: COc1cc(-c2ncc(F)c(=O)[nH]2)nn1Cc1ccccc1F] | I-5 |
| 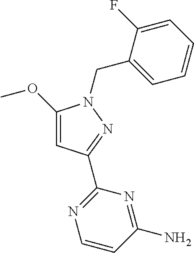 [SMILES: COc1cc(-c2nccc(N)n2)nn1Cc1ccccc1F] | I-6 |
| 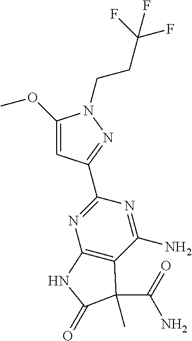 [SMILES: COc1cc(-c2nc(N)c3c(n2)NC(=O)C3(C)C(N)=O)nn1CCC(F)(F)F] | I-7 |
| 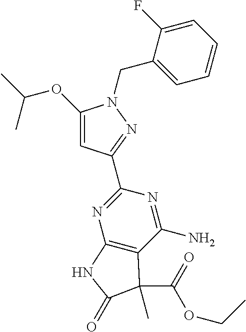 [SMILES: CCOC(=O)C1(C)C(=O)Nc2nc(-c3cc(OC(C)C)n(Cc4ccccc4F)n3)nc(N)c21] | I-8 |
| 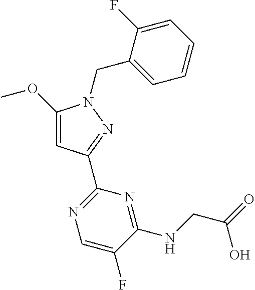 [SMILES: COc1cc(-c2ncc(F)c(NCC(=O)O)n2)nn1Cc1ccccc1F] | I-9 |
| 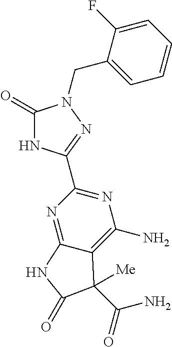 [SMILES: CC1(C(N)=O)C(=O)Nc2nc(-c3nn(Cc4ccccc4F)c(=O)[nH]3)nc(N)c21] | I-10 |
| 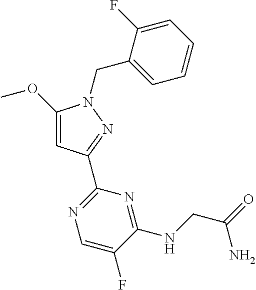 [SMILES: COc1cc(-c2ncc(F)c(NCC(N)=O)n2)nn1Cc1ccccc1F] | I-11 |
| 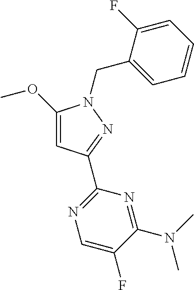 | I-12 |
| 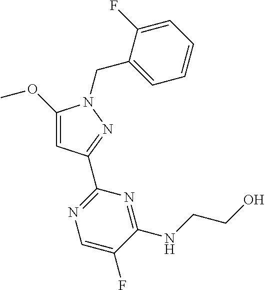 [SMILES: COc1cc(-c2ncc(F)c(NCCO)n2)nn1Cc1ccccc1F] | I-13 |
| 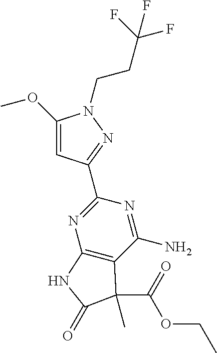 [SMILES: CCOC(=O)C1(C)C(=O)Nc2nc(-c3cc(OC)n(CCC(F)(F)F)n3)nc(N)c21] | I-14 |
| 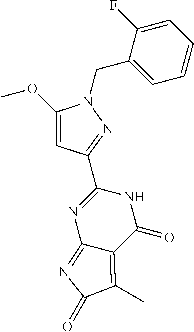 [SMILES: COc1cc(-c2nc3c(c(=O)[nH]2)=C(C)C(=O)N=3)nn1Cc1ccccc1F] | I-15 |
| 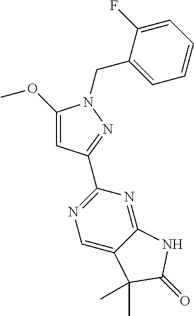 [SMILES: COc1cc(-c2ncc3c(n2)NC(=O)C3(C)C)nn1Cc1ccccc1F] | I-16 |
| 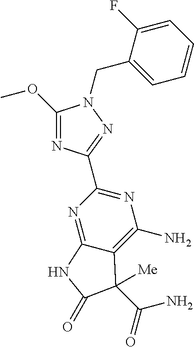 [SMILES: COc1nc(-c2nc(N)c3c(n2)NC(=O)C3(C)C(N)=O)nn1Cc1ccccc1F] | I-17 |
| 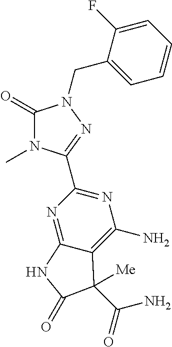 [SMILES: Cn1c(-c2nc(N)c3c(n2)NC(=O)C3(C)C(N)=O)nn(Cc2ccccc2F)c1=O] | I-18 |
| 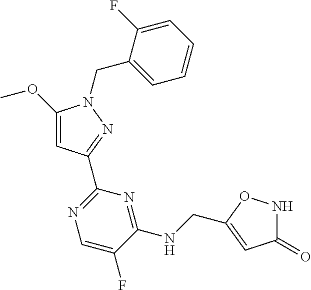 [SMILES: COc1cc(-c2ncc(F)c(NCc3cc(=O)[nH]o3)n2)nn1Cc1ccccc1F] | I-19 |
| 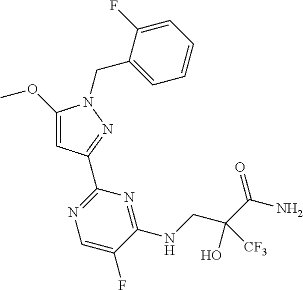 [SMILES: COc1cc(-c2ncc(F)c(NCC(O)(C(N)=O)C(F)(F)F)n2)nn1Cc1ccccc1F] | I-20 |
| 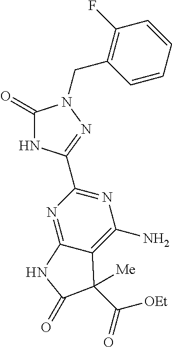 [SMILES: CCOC(=O)C1(C)C(=O)Nc2nc(-c3nn(Cc4ccccc4F)c(=O)[nH]3)nc(N)c21] | I-21 |
| 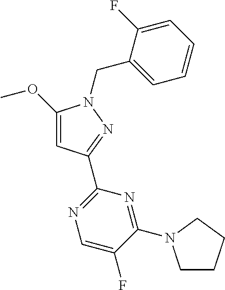 [SMILES: COc1cc(-c2ncc(F)c(N3CCCC3)n2)nn1Cc1ccccc1F] | I-22 |
| 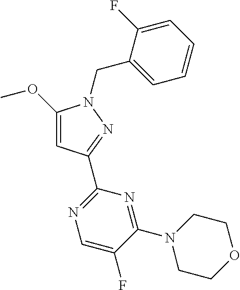 [SMILES: COc1cc(-c2ncc(F)c(N3CCOCC3)n2)nn1Cc1ccccc1F] | I-23 |
| 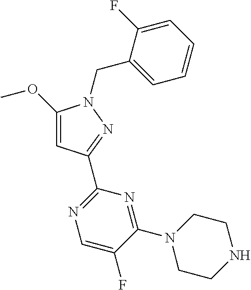 [SMILES: COc1cc(-c2ncc(F)c(N3CCNCC3)n2)nn1Cc1ccccc1F] | I-24 |
| 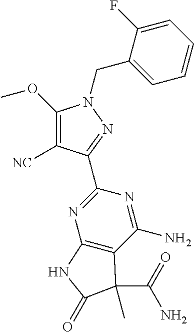 [SMILES: COc1c(C#N)c(-c2nc(N)c3c(n2)NC(=O)C3(C)C(N)=O)nn1Cc1ccccc1F] | I-25 |
| 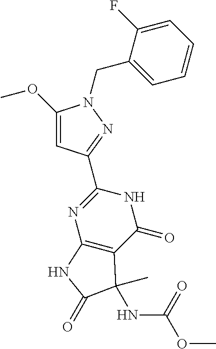 | I-26 |
| 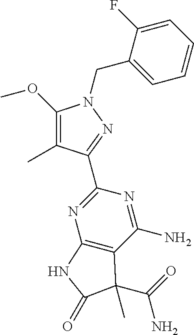 [SMILES: COc1c(C)c(-c2nc(N)c3c(n2)NC(=O)C3(C)C(N)=O)nn1Cc1ccccc1F] | I-27 |
| 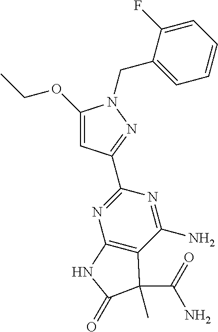 [SMILES: CCOc1cc(-c2nc(N)c3c(n2)NC(=O)C3(C)C(N)=O)nn1Cc1ccccc1F] | I-28 |
| 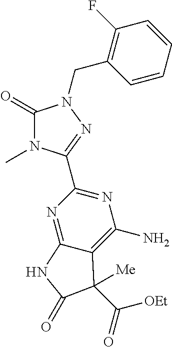 [SMILES: CCOC(=O)C1(C)C(=O)Nc2nc(-c3nn(Cc4ccccc4F)c(=O)n3C)nc(N)c21] | I-29 |
| 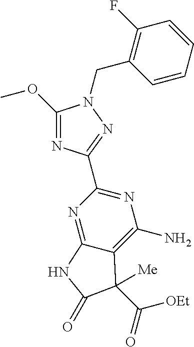 [SMILES: CCOC(=O)C1(C)C(=O)Nc2nc(-c3nc(OC)n(Cc4ccccc4F)n3)nc(N)c21] | I-30 |
| 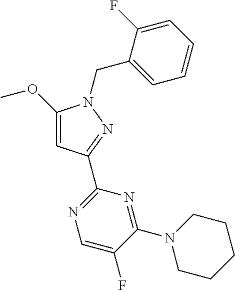 [SMILES: COc1cc(-c2ncc(F)c(N3CCCCC3)n2)nn1Cc1ccccc1F] | I-31 |
| 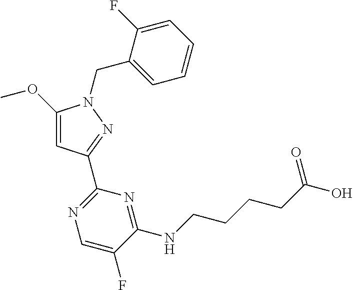 [SMILES: COc1cc(-c2ncc(F)c(NCCCCC(=O)O)n2)nn1Cc1ccccc1F] | I-32 |
| 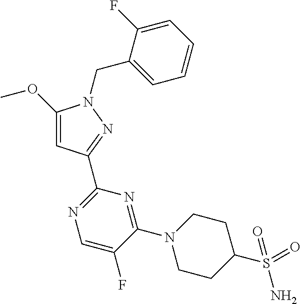 [SMILES: COc1cc(-c2ncc(F)c(N3CCC(S(N)(=O)=O)CC3)n2)nn1Cc1ccccc1F] | I-33 |
| 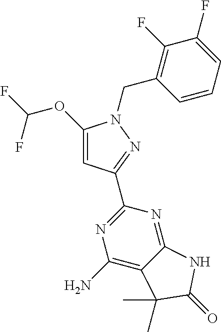 [SMILES: CC1(C)C(=O)Nc2nc(-c3cc(OC(F)F)n(Cc4cccc(F)c4F)n3)nc(N)c21] | I-34 |
| 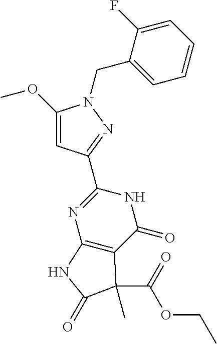 [SMILES: CCOC(=O)C1(C)C(=O)Nc2nc(-c3cc(OC)n(Cc4ccccc4F)n3)[nH]c(=O)c21] | I-35 |
| 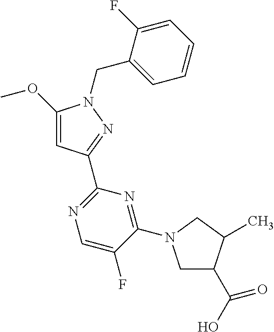 [SMILES: COc1cc(-c2ncc(F)c(N3CC(C)C(C(=O)O)C3)n2)nn1Cc1ccccc1F] | I-36 |
| 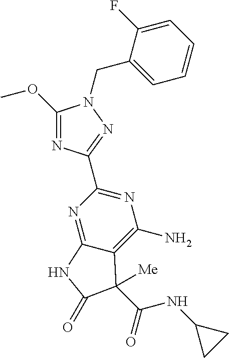 [SMILES: COc1nc(-c2nc(N)c3c(n2)NC(=O)C3(C)C(=O)NC2CC2)nn1Cc1ccccc1F] | I-37 |
| 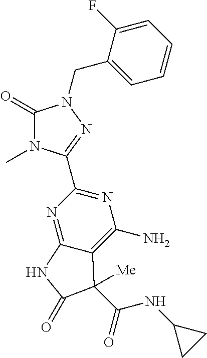 [SMILES: Cn1c(-c2nc(N)c3c(n2)NC(=O)C3(C)C(=O)NC2CC2)nn(Cc2ccccc2F)c1=O] | I-38 |
| 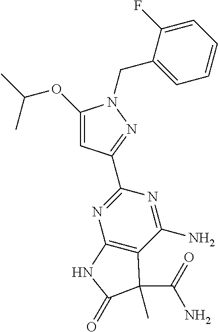 [SMILES: CC(C)Oc1cc(-c2nc(N)c3c(n2)NC(=O)C3(C)C(N)=O)nn1Cc1ccccc1F] | I-39 |
| 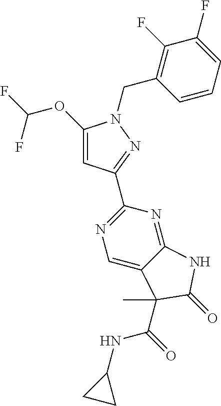 [SMILES: CC1(C(=O)NC2CC2)C(=O)Nc2nc(-c3cc(OC(F)F)n(Cc4cccc(F)c4F)n3)ncc21] | I-40 |
| 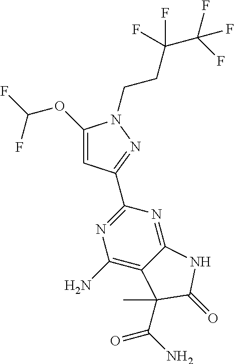 [SMILES: CC1(C(N)=O)C(=O)Nc2nc(-c3cc(OC(F)F)n(CCC(F)(F)C(F)(F)F)n3)nc(N)c21] | I-41 |
| 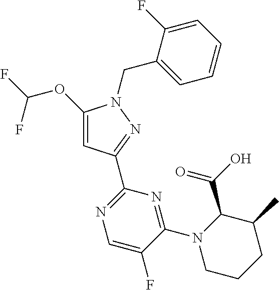 [SMILES: C[C@H]1CCCN(c2nc(-c3cc(OC(F)F)n(Cc4ccccc4F)n3)ncc2F)[C@H]1C(=O)O] | I-42 |
| 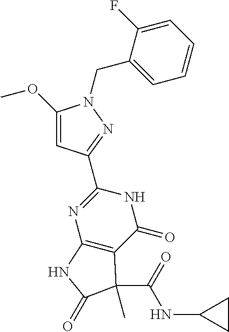 [SMILES: COc1cc(-c2nc3c(c(=O)[nH]2)C(C)(C(=O)NC2CC2)C(=O)N3)nn1Cc1ccccc1F] | I-43 |
| 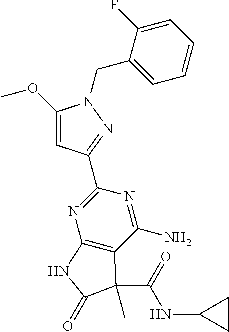 [SMILES: COc1cc(-c2nc(N)c3c(n2)NC(=O)C3(C)C(=O)NC2CC2)nn1Cc1ccccc1F] | I-44 |
| 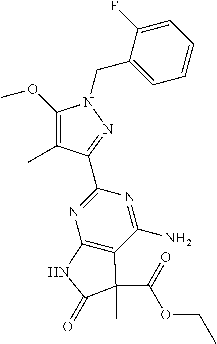 [SMILES: CCOC(=O)C1(C)C(=O)Nc2nc(-c3nn(Cc4ccccc4F)c(OC)c3C)nc(N)c21] | I-45 |
| 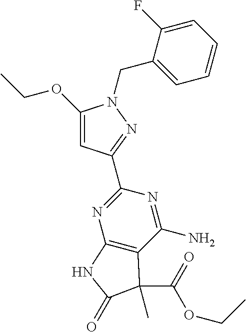 [SMILES: CCOC(=O)C1(C)C(=O)Nc2nc(-c3cc(OCC)n(Cc4ccccc4F)n3)nc(N)c21] | I-46 |
| 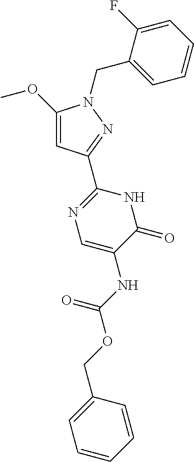 [SMILES: COc1cc(-c2ncc(NC(=O)OCc3ccccc3)c(=O)[nH]2)nn1Cc1ccccc1F] | I-47 |
| 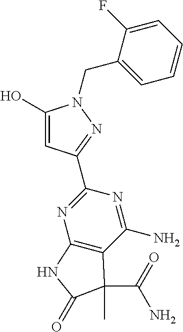 [SMILES: CC1(C(N)=O)C(=O)Nc2nc(-c3cc(O)n(Cc4ccccc4F)n3)nc(N)c21] | I-48 |
| 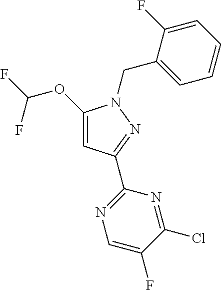 [SMILES: Fc1ccccc1Cn1nc(-c2ncc(F)c(Cl)n2)cc1OC(F)F] | I-49 |
| 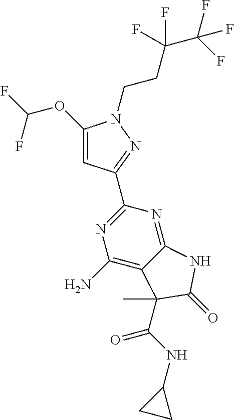 [SMILES: CC1(C(=O)NC2CC2)C(=O)Nc2nc(-c3cc(OC(F)F)n(CCC(F)(F)C(F)(F)F)n3)nc(N)c21] | I-50 |
| 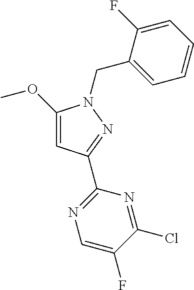 [SMILES: COc1cc(-c2ncc(F)c(Cl)n2)nn1Cc1ccccc1F] | I-51 |
| 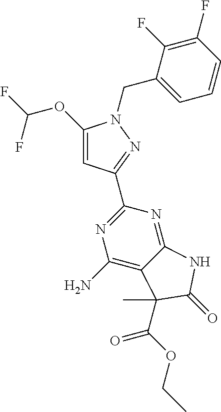 [SMILES: CCOC(=O)C1(C)C(=O)Nc2nc(-c3cc(OC(F)F)n(Cc4cccc(F)c4F)n3)nc(N)c21] | I-52 |
| 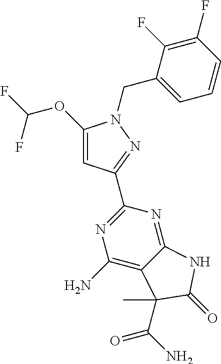 [SMILES: CC1(C(N)=O)C(=O)Nc2nc(-c3cc(OC(F)F)n(Cc4cccc(F)c4F)n3)nc(N)c21] | I-53 |
| 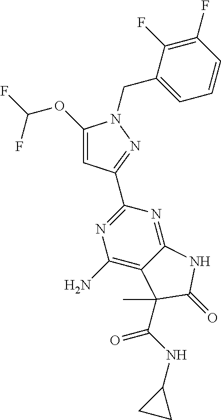 [SMILES: CC1(C(=O)NC2CC2)C(=O)Nc2nc(-c3cc(OC(F)F)n(Cc4cccc(F)c4F)n3)nc(N)c21] | I-54 |
| 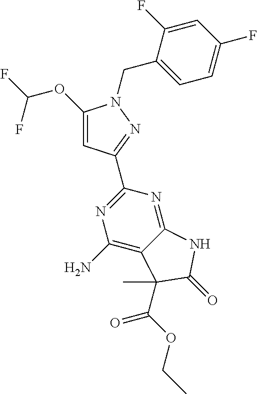 [SMILES: CCOC(=O)C1(C)C(=O)Nc2nc(-c3cc(OC(F)F)n(Cc4ccc(F)cc4F)n3)nc(N)c21] | I-55 |
| 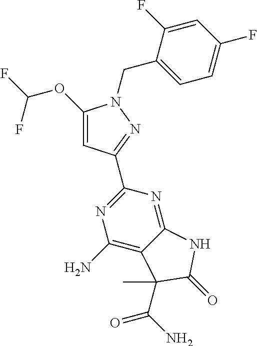 [SMILES: CC1(C(N)=O)C(=O)Nc2nc(-c3cc(OC(F)F)n(Cc4ccc(F)cc4F)n3)nc(N)c21] | I-56 |
| 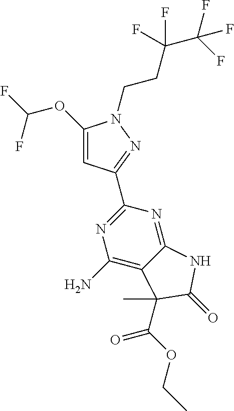 [SMILES: CCOC(=O)C1(C)C(=O)Nc2nc(-c3cc(OC(F)F)n(CCC(F)(F)C(F)(F)F)n3)nc(N)c21] | I-57 |
| 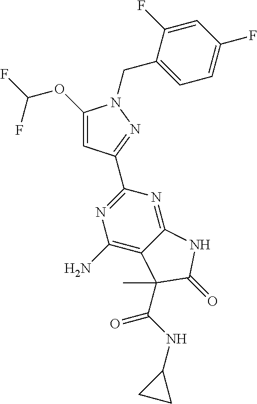 [SMILES: CC1(C(=O)NC2CC2)C(=O)Nc2nc(-c3cc(OC(F)F)n(Cc4ccc(F)cc4F)n3)nc(N)c21] | I-58 |
| 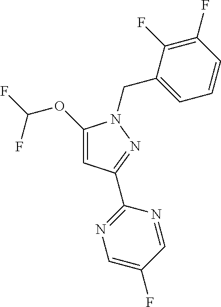 [SMILES: Fc1cnc(-c2cc(OC(F)F)n(Cc3cccc(F)c3F)n2)nc1] | I-59 |
| 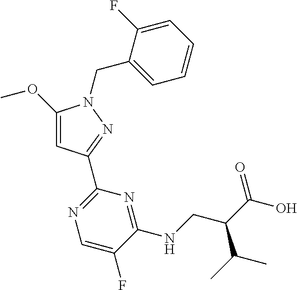 [SMILES: COc1cc(-c2ncc(F)c(NC[C@@H](C(=O)O)C(C)C)n2)nn1Cc1ccccc1F] | I-60 |
| 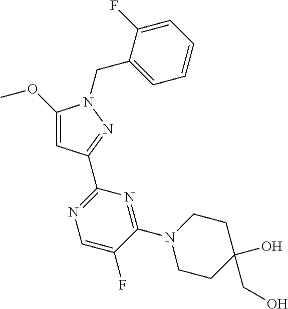 [SMILES: COc1cc(-c2ncc(F)c(N3CCC(O)(CO)CC3)n2)nn1Cc1ccccc1F] | I-61 |
| 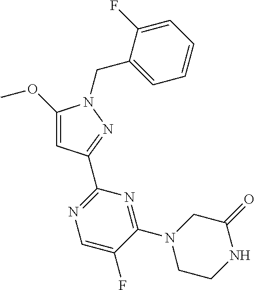 [SMILES: COc1cc(-c2ncc(F)c(N3CCNC(=O)C3)n2)nn1Cc1ccccc1F] | I-62 |
| 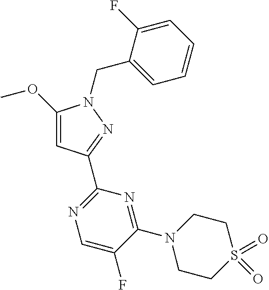 [SMILES: COc1cc(-c2ncc(F)c(N3CCS(=O)(=O)CC3)n2)nn1Cc1ccccc1F] | I-63 |
| 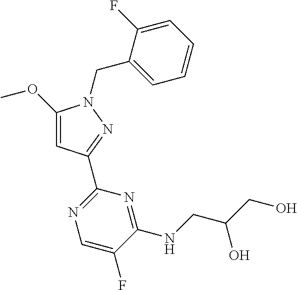 [SMILES: COc1cc(-c2ncc(F)c(NCC(O)CO)n2)nn1Cc1ccccc1F] | I-64 |
| 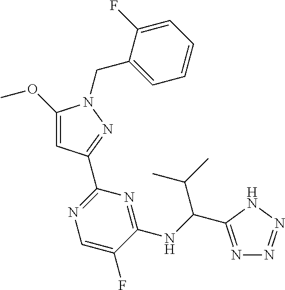 [SMILES: COc1cc(-c2ncc(F)c(NC(c3nnn[nH]3)C(C)C)n2)nn1Cc1ccccc1F] | I-65 |
| 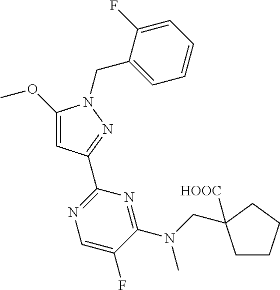 [SMILES: COc1cc(-c2ncc(F)c(N(C)CC3(C(=O)O)CCCC3)n2)nn1Cc1ccccc1F] | I-66 |
| 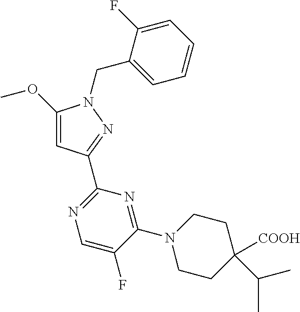 [SMILES: COc1cc(-c2ncc(F)c(N3CCC(C(=O)O)(C(C)C)CC3)n2)nn1Cc1ccccc1F] | I-67 |
| 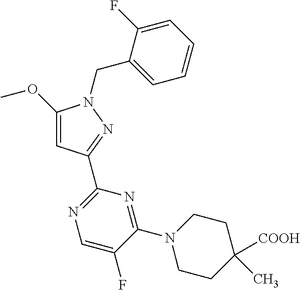 [SMILES: COc1cc(-c2ncc(F)c(N3CCC(C)(C(=O)O)CC3)n2)nn1Cc1ccccc1F] | I-68 |
| 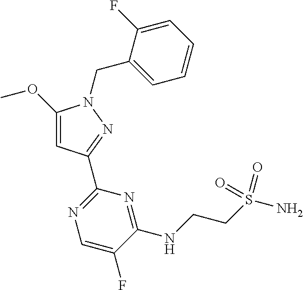 [SMILES: COc1cc(-c2ncc(F)c(NCCS(N)(=O)=O)n2)nn1Cc1ccccc1F] | I-69 |
| 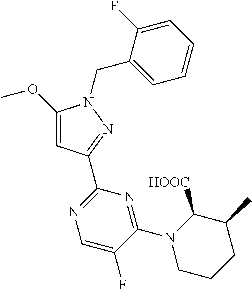 | I-70 |
| 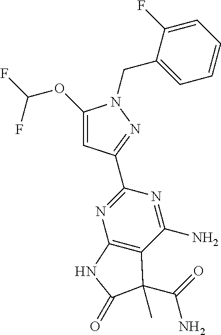 [SMILES: CC1(C(N)=O)C(=O)Nc2nc(-c3cc(OC(F)F)n(Cc4ccccc4F)n3)nc(N)c21] | I-71 |
| 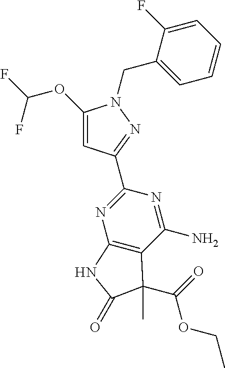 [SMILES: CCOC(=O)C1(C)C(=O)Nc2nc(-c3cc(OC(F)F)n(Cc4ccccc4F)n3)nc(N)c21] | I-72 |

In further embodiments of the above methods, uses, compositions and kits, the sGC stimulator is a compound selected from those depicted in Table XC below:

TABLE XC

| | |
|---|---|
| 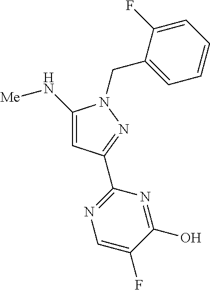 [SMILES: CNc1cc(-c2ncc(F)c(O)n2)nn1Cc1ccccc1F] | 1 |
| 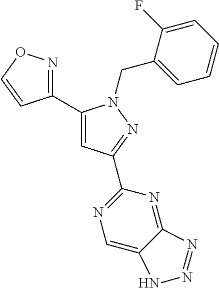 [SMILES: Fc1ccccc1Cn1nc(-c2ncc3[nH]nnc3n2)cc1-c1ccon1] | 2 |
| 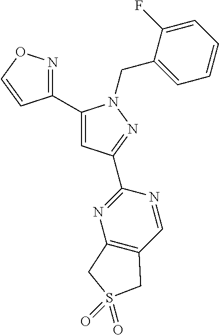 [SMILES: O=S1(=O)Cc2cnc(-c3cc(-c4ccon4)n(Cc4ccccc4F)n3)nc2C1] | 3 |
| 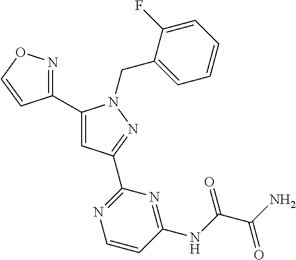 [SMILES: NC(=O)C(=O)Nc1ccnc(-c2cc(-c3ccon3)n(Cc3ccccc3F)n2)n1] | 4 |
| 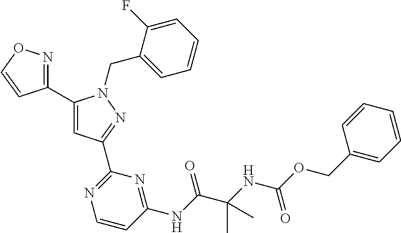 [SMILES: CC(C)(NC(=O)OCc1ccccc1)C(=O)Nc1ccnc(-c2cc(-c3ccon3)n(Cc3ccccc3F)n2)n1] | 5 |
| 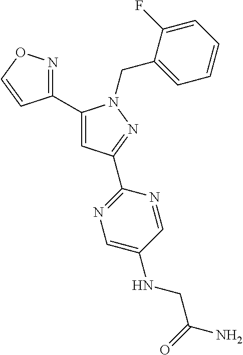 [SMILES: NC(=O)CNc1cnc(-c2cc(-c3ccon3)n(Cc3ccccc3F)n2)nc1] | 6 |
| 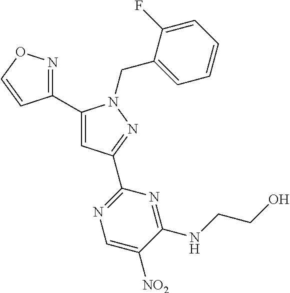 [SMILES: O=[N+]([O-])c1cnc(-c2cc(-c3ccon3)n(Cc3ccccc3F)n2)nc1NCCO] | 7 |
| 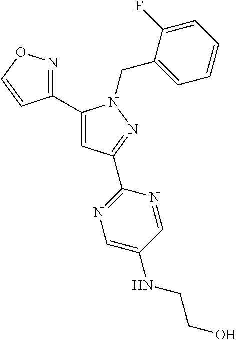 [SMILES: OCCNc1cnc(-c2cc(-c3ccon3)n(Cc3ccccc3F)n2)nc1] | 8 |
| 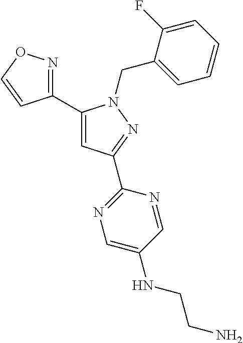 [SMILES: NCCNc1cnc(-c2cc(-c3ccon3)n(Cc3ccccc3F)n2)nc1] | 9 |
| 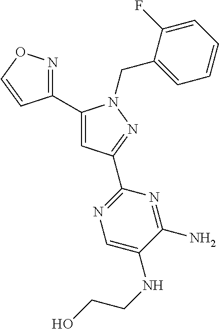 [SMILES: Nc1nc(-c2cc(-c3ccon3)n(Cc3ccccc3F)n2)ncc1NCCO] | 10 |
| 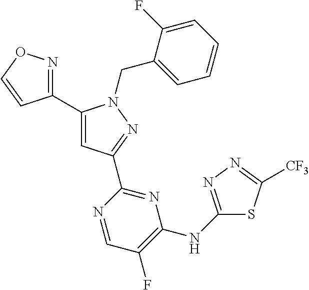 [SMILES: Fc1ccccc1Cn1nc(-c2ncc(F)c(Nc3nnc(C(F)(F)F)s3)n2)cc1-c1ccon1] | 11 |
| 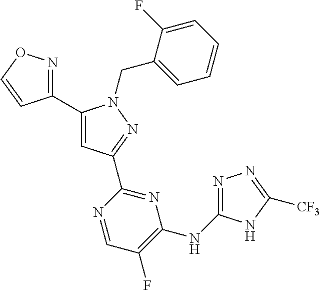 [SMILES: Fc1ccccc1Cn1nc(-c2ncc(F)c(Nc3nnc(C(F)(F)F)[nH]3)n2)cc1-c1ccon1] | 12 |
| 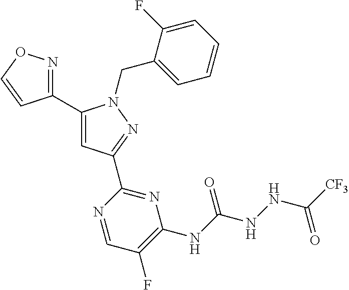 [SMILES: O=C(NNC(=O)C(F)(F)F)Nc1nc(-c2cc(-c3ccon3)n(Cc3ccccc3F)n2)ncc1F] | 13 |
| 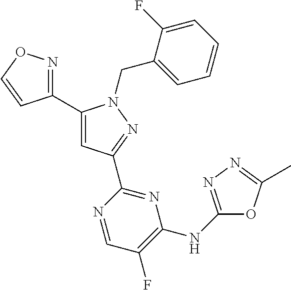 [SMILES: Cc1nnc(Nc2nc(-c3cc(-c4ccon4)n(Cc4ccccc4F)n3)ncc2F)o1] | 14 |
| 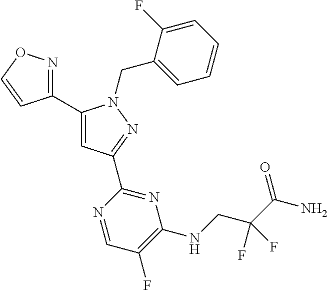 [SMILES: NC(=O)C(F)(F)CNc1nc(-c2cc(-c3ccon3)n(Cc3ccccc3F)n2)ncc1F] | 15 |
| 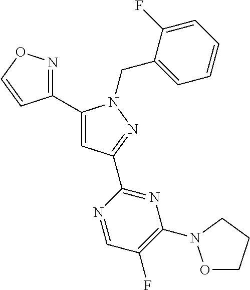 [SMILES: Fc1ccccc1Cn1nc(-c2ncc(F)c(N3CCCO3)n2)cc1-c1ccon1] | 16 |
| 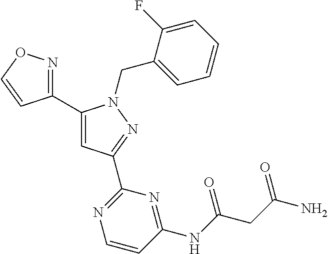 [SMILES: NC(=O)CC(=O)Nc1ccnc(-c2cc(-c3ccon3)n(Cc3ccccc3F)n2)n1] | 17 |
| 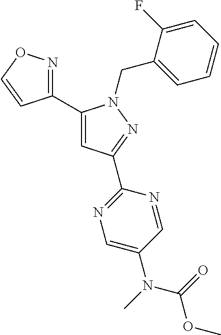 [SMILES: COC(=O)N(C)c1cnc(-c2cc(-c3ccon3)n(Cc3ccccc3F)n2)nc1] | 18 |
| 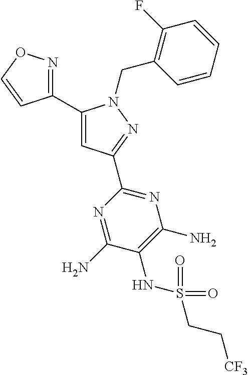 [SMILES: Nc1nc(-c2cc(-c3ccon3)n(Cc3ccccc3F)n2)nc(N)c1NS(=O)(=O)CCC(F)(F)F] | 19 |
| 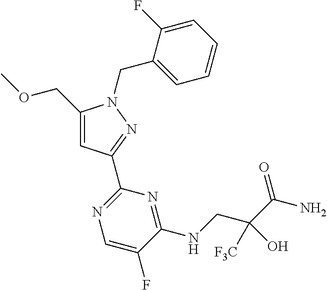 [SMILES: COCc1cc(-c2ncc(F)c(NCC(O)(C(N)=O)C(F)(F)F)n2)nn1Cc1ccccc1F] | 20 |
| 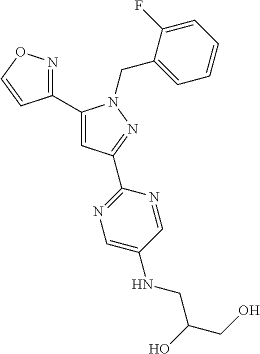 [SMILES: OCC(O)CNc1cnc(-c2cc(-c3ccon3)n(Cc3ccccc3F)n2)nc1] | 21 |
| 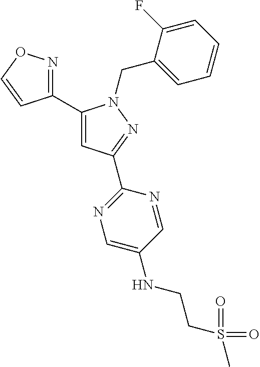 [SMILES: CS(=O)(=O)CCNc1cnc(-c2cc(-c3ccon3)n(Cc3ccccc3F)n2)nc1] | 22 |
| 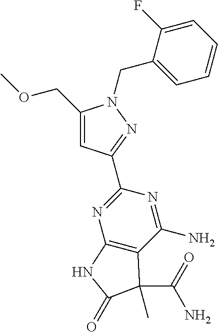 [SMILES: COCc1cc(-c2nc(N)c3c(n2)NC(=O)C3(C)C(N)=O)nn1Cc1ccccc1F] | 23 |
| 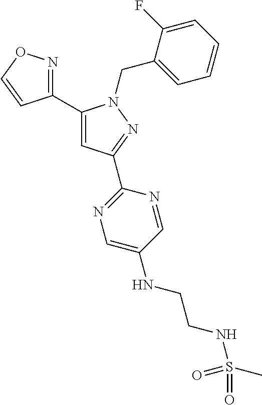 [SMILES: CS(=O)(=O)NCCNc1cnc(-c2cc(-c3ccon3)n(Cc3ccccc3F)n2)nc1] | 24 |
| 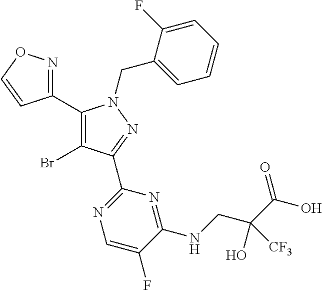 [SMILES: O=C(O)C(O)(CNc1nc(-c2nn(Cc3ccccc3F)c(-c3ccon3)c2Br)ncc1F)C(F)(F)F] | 25 |
| 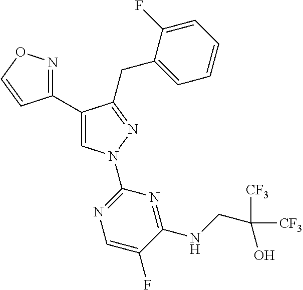 [SMILES: OC(CNc1nc(-n2cc(-c3ccon3)c(Cc3ccccc3F)n2)ncc1F)(C(F)(F)F)C(F)(F)F] | 26 |
| 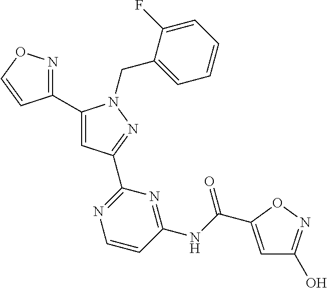 [SMILES: O=C(Nc1ccnc(-c2cc(-c3ccon3)n(Cc3ccccc3F)n2)n1)c1cc(O)no1] | 27 |
| 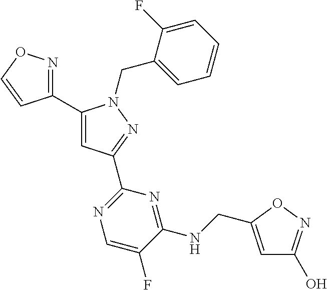 [SMILES: Oc1cc(CNc2nc(-c3cc(-c4ccon4)n(Cc4ccccc4F)n3)ncc2F)on1] | 28 |
| 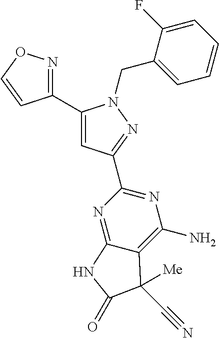 [SMILES: CC1(C#N)C(=O)Nc2nc(-c3cc(-c4ccon4)n(Cc4ccccc4F)n3)nc(N)c21] | 29 |
| 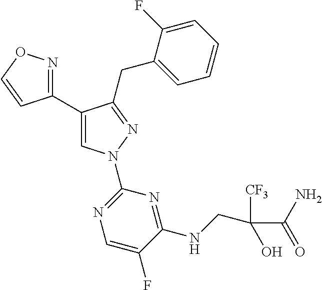 [SMILES: NC(=O)C(O)(CNc1nc(-n2cc(-c3ccon3)c(Cc3ccccc3F)n2)ncc1F)C(F)(F)F] | 30 |
| 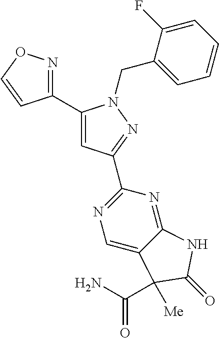 [SMILES: CC1(C(N)=O)C(=O)Nc2nc(-c3cc(-c4ccon4)n(Cc4ccccc4F)n3)ncc21] | 31 |
| 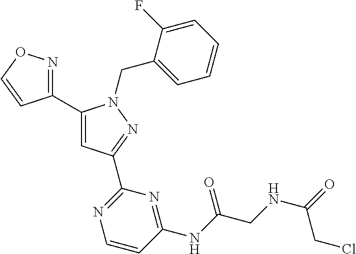 [SMILES: O=C(CCl)NCC(=O)Nc1ccnc(-c2cc(-c3ccon3)n(Cc3ccccc3F)n2)n1] | 32 |
| 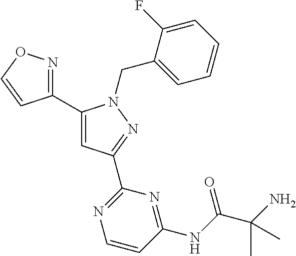 [SMILES: CC(C)(N)C(=O)Nc1ccnc(-c2cc(-c3ccon3)n(Cc3ccccc3F)n2)n1] | 33 |
| 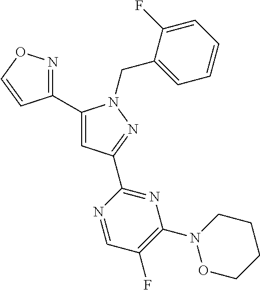 [SMILES: Fc1ccccc1Cn1nc(-c2ncc(F)c(N3CCCCO3)n2)cc1-c1ccon1] | 34 |
| 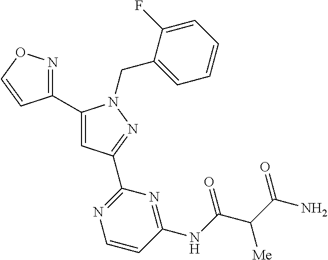 [SMILES: CC(C(N)=O)C(=O)Nc1ccnc(-c2cc(-c3ccon3)n(Cc3ccccc3F)n2)n1] | 35 |
| 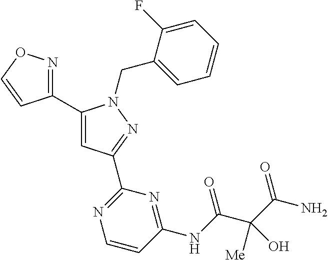 [SMILES: CC(O)(C(N)=O)C(=O)Nc1ccnc(-c2cc(-c3ccon3)n(Cc3ccccc3F)n2)n1] | 36 |
| 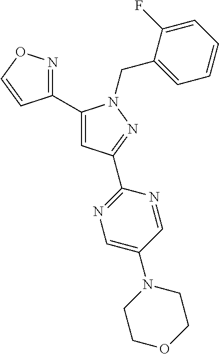 [SMILES: Fc1ccccc1Cn1nc(-c2ncc(N3CCOCC3)cn2)cc1-c1ccon1] | 37 |
| 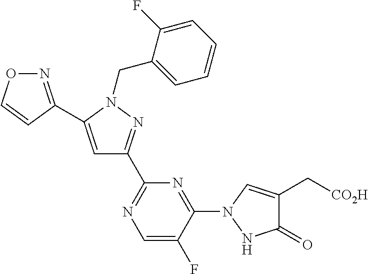 [SMILES: O=C(O)Cc1cn(-c2nc(-c3cc(-c4ccon4)n(Cc4ccccc4F)n3)ncc2F)[nH]c1=O] | 38 |
| 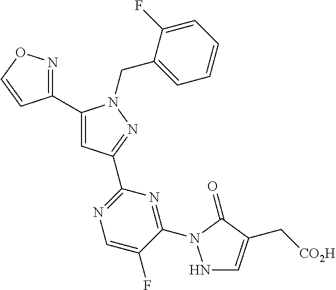 [SMILES: O=C(O)Cc1c[nH]n(-c2nc(-c3cc(-c4ccon4)n(Cc4ccccc4F)n3)ncc2F)c1=O] | 39 |
| 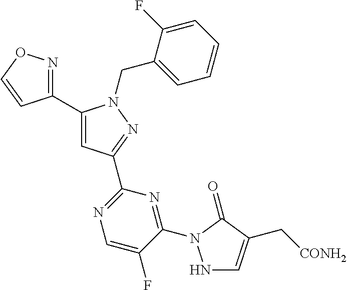 [SMILES: NC(=O)Cc1c[nH]n(-c2nc(-c3cc(-c4ccon4)n(Cc4ccccc4F)n3)ncc2F)c1=O] | 40 |
| 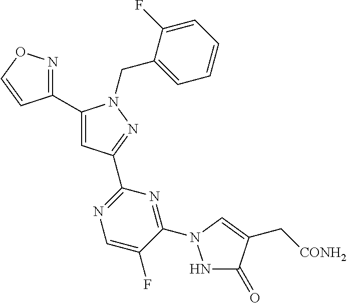 [SMILES: NC(=O)Cc1cn(-c2nc(-c3cc(-c4ccon4)n(Cc4ccccc4F)n3)ncc2F)[nH]c1=O] | 41 |
| 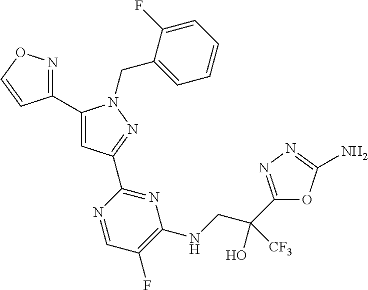 [SMILES: Nc1nnc(C(O)(CNc2nc(-c3cc(-c4ccon4)n(Cc4ccccc4F)n3)ncc2F)C(F)(F)F)o1] | 42 |
| 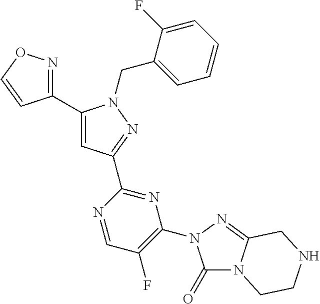 [SMILES: O=c1n(-c2nc(-c3cc(-c4ccon4)n(Cc4ccccc4F)n3)ncc2F)nc2n1CCNC2] | 43 |
| 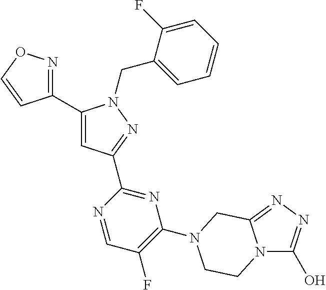 [SMILES: Oc1nnc2n1CCN(c1nc(-c3cc(-c4ccon4)n(Cc4ccccc4F)n3)ncc1F)C2] | 44 |
| 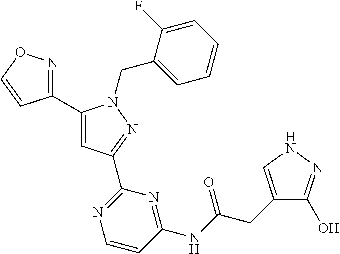 [SMILES: O=C(Cc1c[nH]nc1O)Nc1ccnc(-c2cc(-c3ccon3)n(Cc3ccccc3F)n2)n1] | 45 |
| 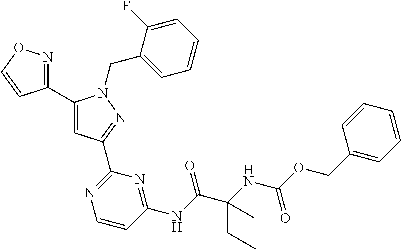 [SMILES: CCC(C)(NC(=O)OCc1ccccc1)C(=O)Nc1ccnc(-c2cc(-c3ccon3)n(Cc3ccccc3F)n2)n1] | 46 |
| 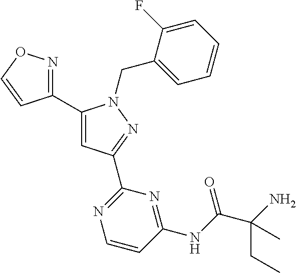 [SMILES: CCC(C)(N)C(=O)Nc1ccnc(-c2cc(-c3ccon3)n(Cc3ccccc3F)n2)n1] | 47 |
| 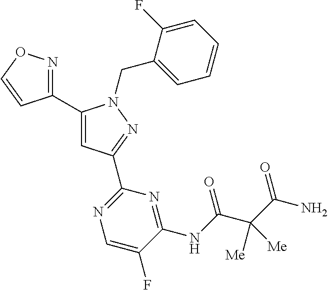 [SMILES: CC(C)(C(N)=O)C(=O)Nc1nc(-c2cc(-c3ccon3)n(Cc3ccccc3F)n2)ncc1F] | 48 |
| 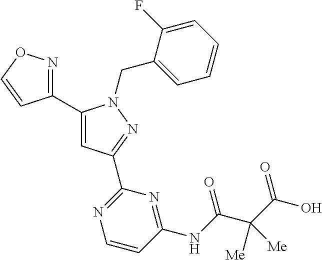 [SMILES: CC(C)(C(=O)O)C(=O)Nc1ccnc(-c2cc(-c3ccon3)n(Cc3ccccc3F)n2)n1] | 49 |
| 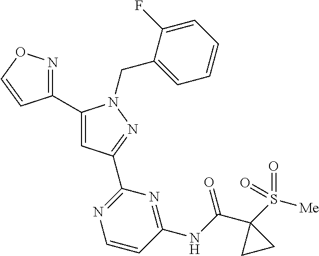 [SMILES: CS(=O)(=O)C1(C(=O)Nc2ccnc(-c3cc(-c4ccon4)n(Cc4ccccc4F)n3)n2)CC1] | 50 |
| 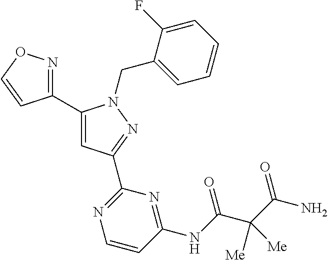 [SMILES: CC(C)(C(N)=O)C(=O)Nc1ccnc(-c2cc(-c3ccon3)n(Cc3ccccc3F)n2)n1] | 51 |
| 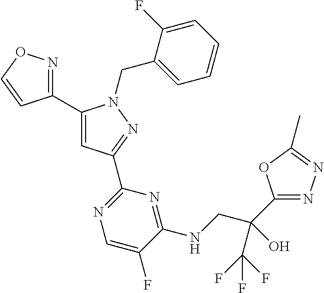 [SMILES: Cc1nnc(C(O)(CNc2nc(-c3cc(-c4ccon4)n(Cc4ccccc4F)n3)ncc2F)C(F)(F)F)o1] | 52 |
| 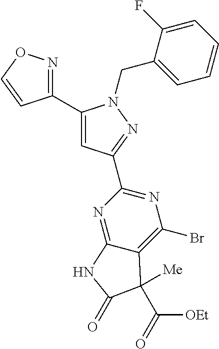 [SMILES: CCOC(=O)C1(C)C(=O)Nc2nc(-c3cc(-c4ccon4)n(Cc4ccccc4F)n3)nc(Br)c21] | 53 |
| 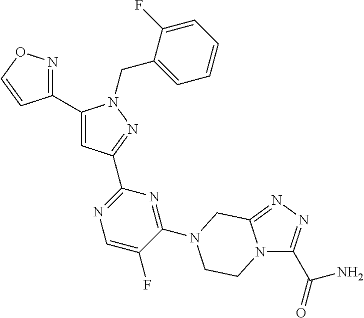 [SMILES: NC(=O)c1nnc2n1CCN(c1nc(-c3cc(-c4ccon4)n(Cc4ccccc4F)n3)ncc1F)C2] | 54 |
| 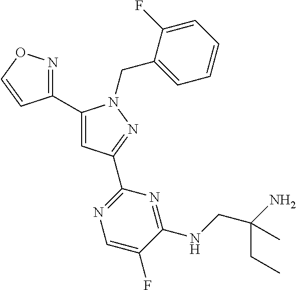 [SMILES: CCC(C)(N)CNc1nc(-c2cc(-c3ccon3)n(Cc3ccccc3F)n2)ncc1F] | 55 |
| 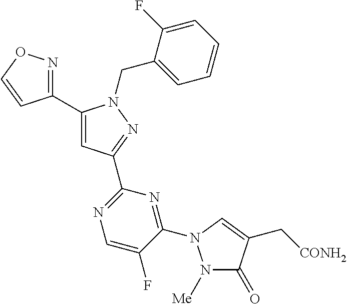 [SMILES: Cn1c(=O)c(CC(N)=O)cn1-c1nc(-c2cc(-c3ccon3)n(Cc3ccccc3F)n2)ncc1F] | 56 |
| 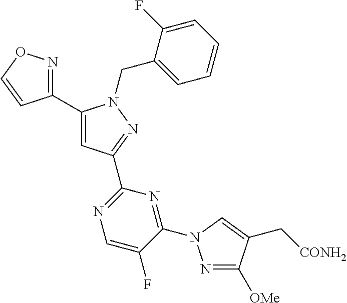 [SMILES: COc1nn(-c2nc(-c3cc(-c4ccon4)n(Cc4ccccc4F)n3)ncc2F)cc1CC(N)=O] | 57 |
| 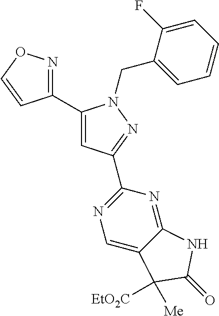 [SMILES: CCOC(=O)C1(C)C(=O)Nc2nc(-c3cc(-c4ccon4)n(Cc4ccccc4F)n3)ncc21] | 58 |
| 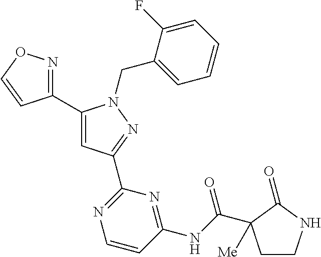 [SMILES: CC1(C(=O)Nc2ccnc(-c3cc(-c4ccon4)n(Cc4ccccc4F)n3)n2)CCNC1=O] | 59 |
| 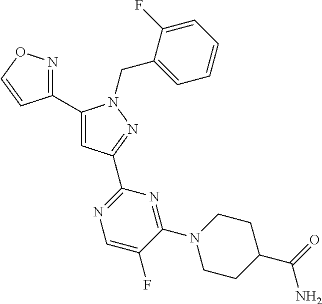 [SMILES: NC(=O)C1CCN(c2nc(-c3cc(-c4ccon4)n(Cc4ccccc4F)n3)ncc2F)CC1] | 60 |
| 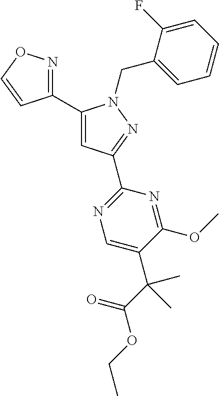 [SMILES: CCOC(=O)C(C)(C)c1cnc(-c2cc(-c3ccon3)n(Cc3ccccc3F)n2)nc1OC] | 61 |
| 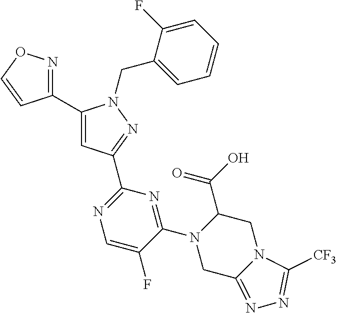 [SMILES: O=C(O)C1Cn2c(nnc2C(F)(F)F)CN1c1nc(-c2cc(-c3ccon3)n(Cc3ccccc3F)n2)ncc1F] | 62 |
| 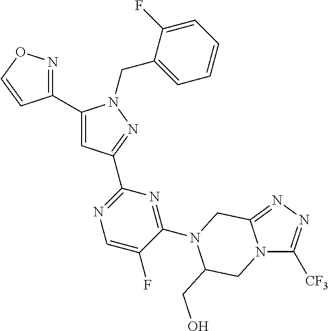 [SMILES: OCC1Cn2c(nnc2C(F)(F)F)CN1c1nc(-c2cc(-c3ccon3)n(Cc3ccccc3F)n2)ncc1F] | 63 |
| 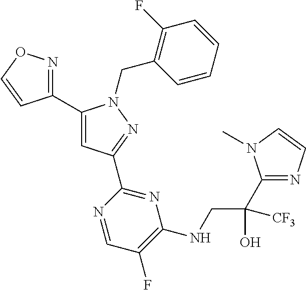 [SMILES: Cn1ccnc1C(O)(CNc1nc(-c2cc(-c3ccon3)n(Cc3ccccc3F)n2)ncc1F)C(F)(F)F] | 64 |
| 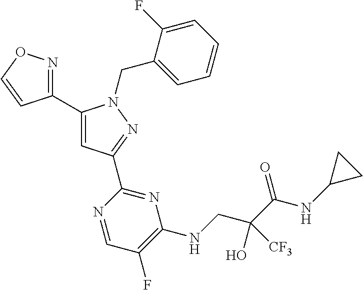 [SMILES: O=C(NC1CC1)C(O)(CNc1nc(-c2cc(-c3ccon3)n(Cc3ccccc3F)n2)ncc1F)C(F)(F)F] | 65 |
| 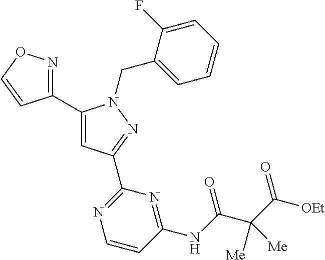 [SMILES: CCOC(=O)C(C)(C)C(=O)Nc1ccnc(-c2cc(-c3ccon3)n(Cc3ccccc3F)n2)n1] | 66 |
| 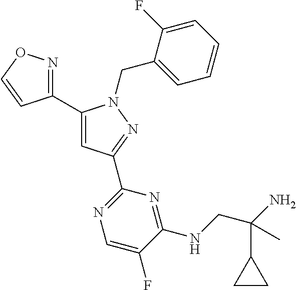 [SMILES: CC(N)(CNc1nc(-c2cc(-c3ccon3)n(Cc3ccccc3F)n2)ncc1F)C1CC1] | 67 |
| 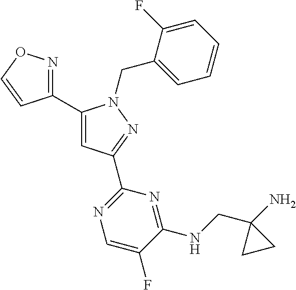 [SMILES: NC1(CNc2nc(-c3cc(-c4ccon4)n(Cc4ccccc4F)n3)ncc2F)CC1] | 68 |
| 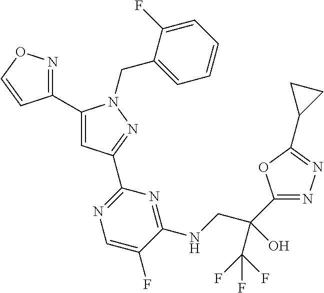 [SMILES: OC(CNc1nc(-c2cc(-c3ccon3)n(Cc3ccccc3F)n2)ncc1F)(c1nnc(C2CC2)o1)C(F)(F)F] | 69 |
| 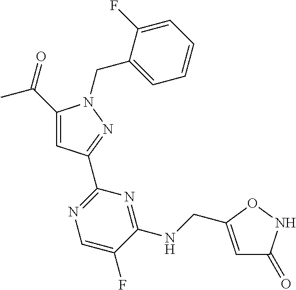 [SMILES: CC(=O)c1cc(-c2ncc(F)c(NCc3cc(=O)[nH]o3)n2)nn1Cc1ccccc1F] | 70 |
| 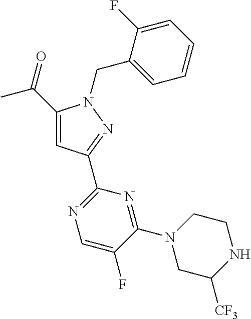 [SMILES: CC(=O)c1cc(-c2ncc(F)c(N3CCNC(C(F)(F)F)C3)n2)nn1Cc1ccccc1F] | 71 |
| 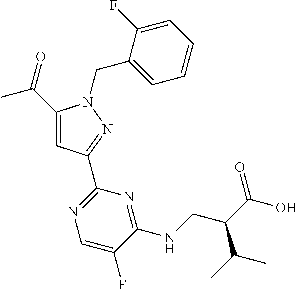 [SMILES: CC(=O)c1cc(-c2ncc(F)c(NC[C@@H](C(=O)O)C(C)C)n2)nn1Cc1ccccc1F] | 72 |
| 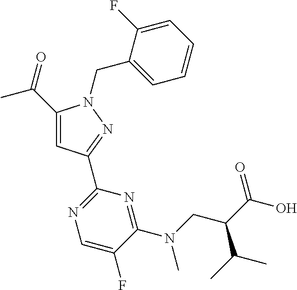 [SMILES: CC(=O)c1cc(-c2ncc(F)c(N(C)C[C@@H](C(=O)O)C(C)C)n2)nn1Cc1ccccc1F] | 73 |
| 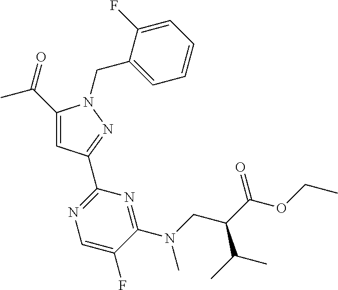 [SMILES: CCOC(=O)[C@H](CN(C)c1nc(-c2cc(C(C)=O)n(Cc3ccccc3F)n2)ncc1F)C(C)C] | 74 |
| 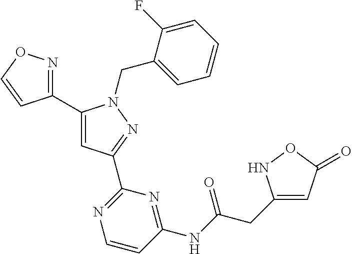 [SMILES: O=C(Cc1cc(=O)o[nH]1)Nc1ccnc(-c2cc(-c3ccon3)n(Cc3ccccc3F)n2)n1] | 75 |
| 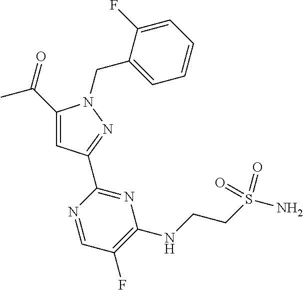 [SMILES: CC(=O)c1cc(-c2ncc(F)c(NCCS(N)(=O)=O)n2)nn1Cc1ccccc1F] | 76 |
| 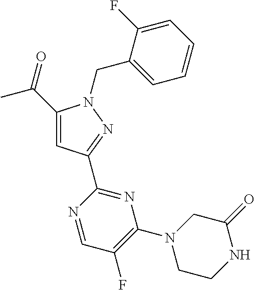 [SMILES: CC(=O)c1cc(-c2ncc(F)c(N3CCNC(=O)C3)n2)nn1Cc1ccccc1F] | 77 |
| 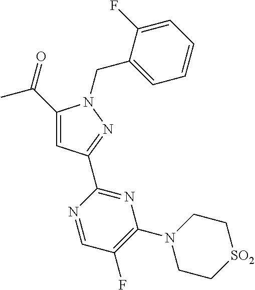 [SMILES: CC(=O)c1cc(-c2ncc(F)c(N3CCS(=O)(=O)CC3)n2)nn1Cc1ccccc1F] | 78 |
| 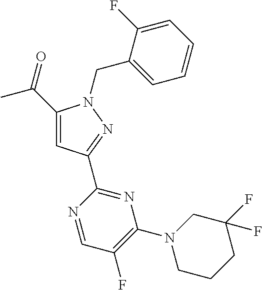 [SMILES: CC(=O)c1cc(-c2ncc(F)c(N3CCCC(F)(F)C3)n2)nn1Cc1ccccc1F] | 79 |
| 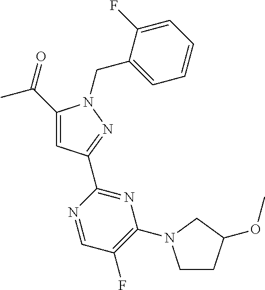 [SMILES: COC1CCN(c2nc(-c3cc(C(C)=O)n(Cc4ccccc4F)n3)ncc2F)C1] | 80 |
| 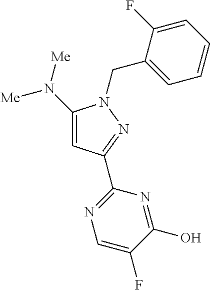 [SMILES: CN(C)c1cc(-c2ncc(F)c(O)n2)nn1Cc1ccccc1F] | 81 |
| 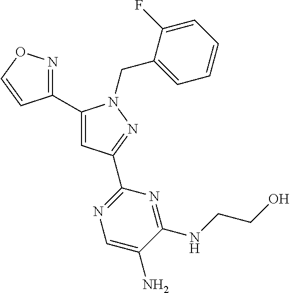 [SMILES: Nc1cnc(-c2cc(-c3ccon3)n(Cc3ccccc3F)n2)nc1NCCO] | 82 |
| 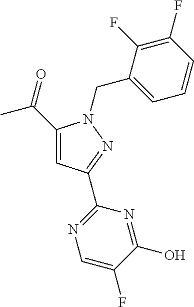 [SMILES: CC(=O)c1cc(-c2ncc(F)c(O)n2)nn1Cc1cccc(F)c1F] | 83 |
| 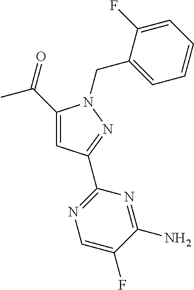 [SMILES: CC(=O)c1cc(-c2ncc(F)c(N)n2)nn1Cc1ccccc1F] | 84 |
| 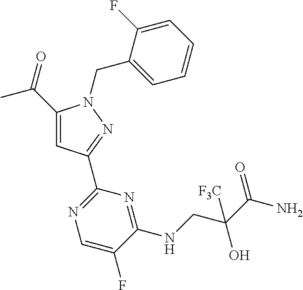 [SMILES: CC(=O)c1cc(-c2ncc(F)c(NCC(O)(C(N)=O)C(F)(F)F)n2)nn1Cc1ccccc1F] | 85 |
| 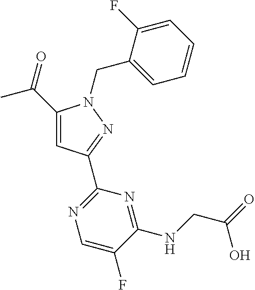 [SMILES: CC(=O)c1cc(-c2ncc(F)c(NCC(=O)O)n2)nn1Cc1ccccc1F] | 86 |
| 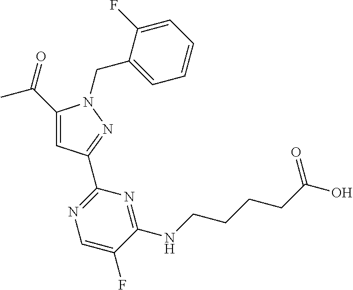 [SMILES: CC(=O)c1cc(-c2ncc(F)c(NCCCCC(=O)O)n2)nn1Cc1ccccc1F] | 87 |
| 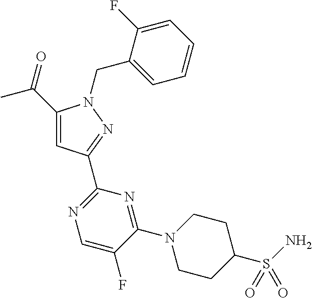 [SMILES: CC(=O)c1cc(-c2ncc(F)c(N3CCC(S(N)(=O)=O)CC3)n2)nn1Cc1ccccc1F] | 88 |
| 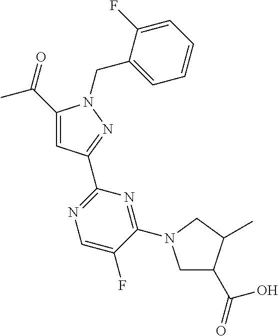 [SMILES: CC(=O)c1cc(-c2ncc(F)c(N3CC(C)C(C(=O)O)C3)n2)nn1Cc1ccccc1F] | 89 |
| 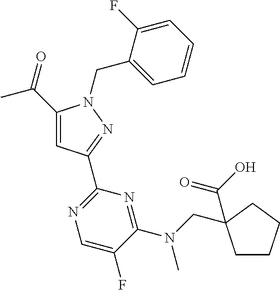 [SMILES: CC(=O)c1cc(-c2ncc(F)c(N(C)CC3(C(=O)O)CCCC3)n2)nn1Cc1ccccc1F] | 90 |
| 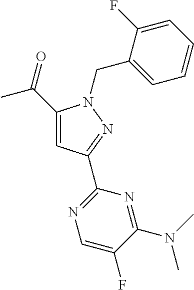 [SMILES: CC(=O)c1cc(-c2ncc(F)c(N(C)C)n2)nn1Cc1ccccc1F] | 91 |
| 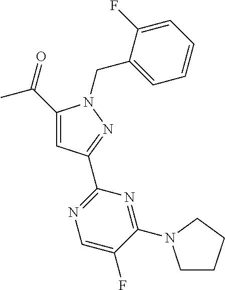 [SMILES: CC(=O)c1cc(-c2ncc(F)c(N3CCCC3)n2)nn1Cc1ccccc1F] | 92 |
| 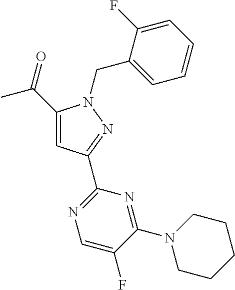 [SMILES: CC(=O)c1cc(-c2ncc(F)c(N3CCCCC3)n2)nn1Cc1ccccc1F] | 93 |
| 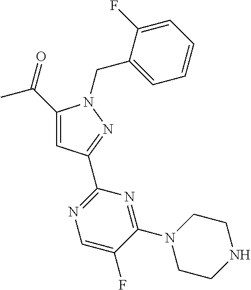 [SMILES: CC(=O)c1cc(-c2ncc(F)c(N3CCNCC3)n2)nn1Cc1ccccc1F] | 94 |
| 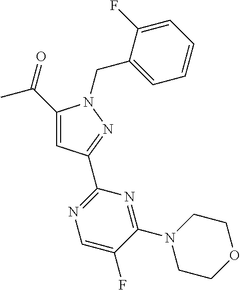 [SMILES: CC(=O)c1cc(-c2ncc(F)c(N3CCOCC3)n2)nn1Cc1ccccc1F] | 95 |
| 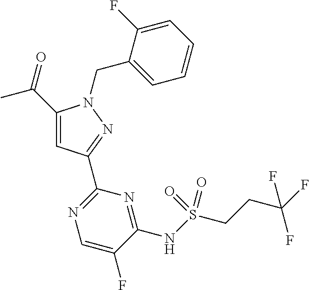 [SMILES: CC(=O)c1cc(-c2ncc(F)c(NS(=O)(=O)CCC(F)(F)F)n2)nn1Cc1ccccc1F] | 96 |
| 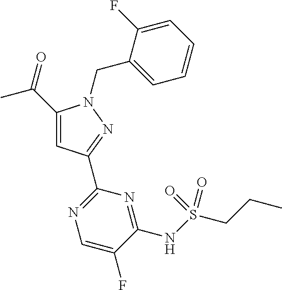 [SMILES: CCCS(=O)(=O)Nc1nc(-c2cc(C(C)=O)n(Cc3ccccc3F)n2)ncc1F] | 97 |
| 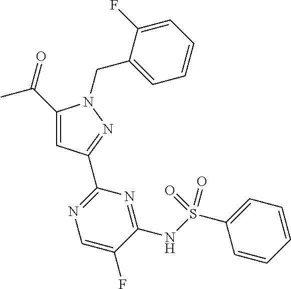 [SMILES: CC(=O)c1cc(-c2ncc(F)c(NS(=O)(=O)c3ccccc3)n2)nn1Cc1ccccc1F] | 98 |
| 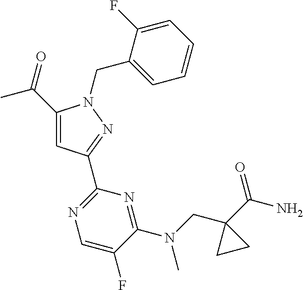 [SMILES: CC(=O)c1cc(-c2ncc(F)c(N(C)CC3(C(N)=O)CC3)n2)nn1Cc1ccccc1F] | 99 |
| 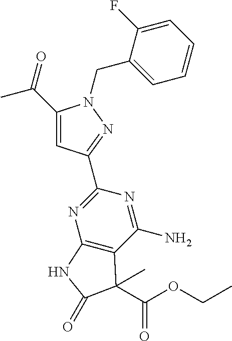 [SMILES: CCOC(=O)C1(C)C(=O)Nc2nc(-c3cc(C(C)=O)n(Cc4ccccc4F)n3)nc(N)c21] | 100 |
| 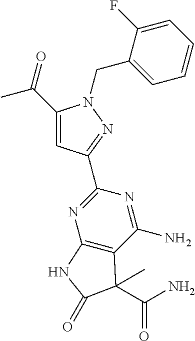 [SMILES: CC(=O)c1cc(-c2nc(N)c3c(n2)NC(=O)C3(C)C(N)=O)nn1Cc1ccccc1F] | 101 |
| 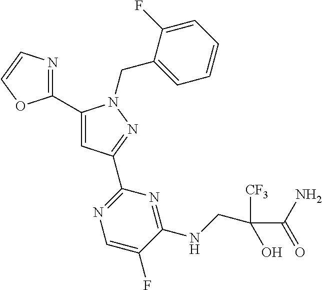 [SMILES: NC(=O)C(O)(CNc1nc(-c2cc(-c3ncco3)n(Cc3ccccc3F)n2)ncc1F)C(F)(F)F] | 102 |
| 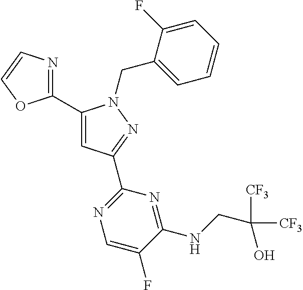 [SMILES: OC(CNc1nc(-c2cc(-c3ncco3)n(Cc3ccccc3F)n2)ncc1F)(C(F)(F)F)C(F)(F)F] | 103 |
| 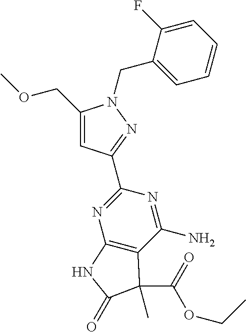 [SMILES: CCOC(=O)C1(C)C(=O)Nc2nc(-c3cc(COC)n(Cc4ccccc4F)n3)nc(N)c21] | 104 |
| 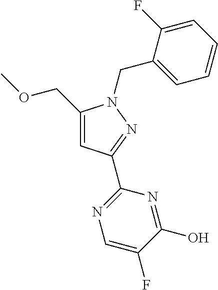 [SMILES: COCc1cc(-c2ncc(F)c(O)n2)nn1Cc1ccccc1F] | 105 |
| 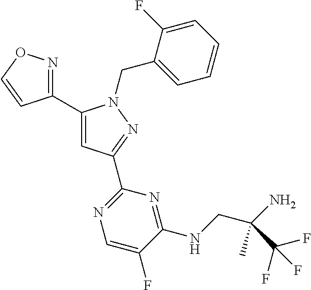 [SMILES: C[C@@](N)(CNc1nc(-c2cc(-c3ccon3)n(Cc3ccccc3F)n2)ncc1F)C(F)(F)F] | 106 |
| 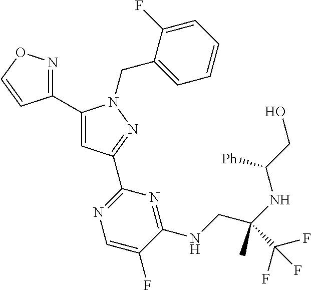 [SMILES: C[C@@](CNc1nc(-c2cc(-c3ccon3)n(Cc3ccccc3F)n2)ncc1F)(N[C@@H](CO)c1ccccc1)C(F)(F)F] | 107 |
| 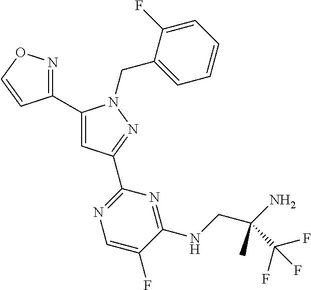 [SMILES: C[C@](N)(CNc1nc(-c2cc(-c3ccon3)n(Cc3ccccc3F)n2)ncc1F)C(F)(F)F] | 108 |
| 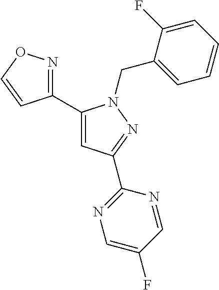 [SMILES: Fc1cnc(-c2cc(-c3ccon3)n(Cc3ccccc3F)n2)nc1] | 109 |

In yet other embodiments of the above methods, uses, pharmaceutical compositions and kits, the sGC stimulator is a compound depicted below:

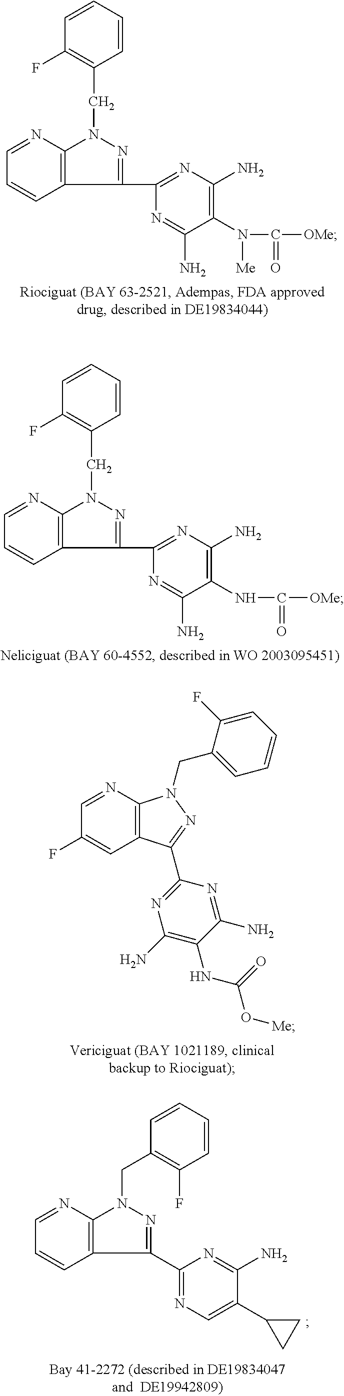 [SMILES: COC(=O)N(C)c1c(N)nc(-c2nn(Cc3ccccc3F)c3ncccc23)nc1N ; COC(=O)Nc1c(N)nc(-c2nn(Cc3ccccc3F)c3ncccc23)nc1N ; COC(=O)Nc1c(N)nc(-c2nn(Cc3ccccc3F)c3ncc(F)cc23)nc1N ; Nc1nc(-c2nn(Cc3ccccc3F)c3ncccc23)ncc1C1CC1]

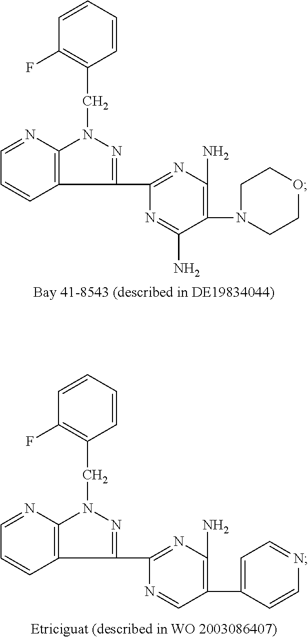 [SMILES: Nc1nc(-c2nn(Cc3ccccc3F)c3ncccc23)nc(N)c1N1CCOCC1 ; Nc1nc(-c2nn(Cc3ccccc3F)c3ncccc23)ncc1-c1ccncc1]

or one of the compounds depicted below and described in US20130072492 (WO 2011149921):

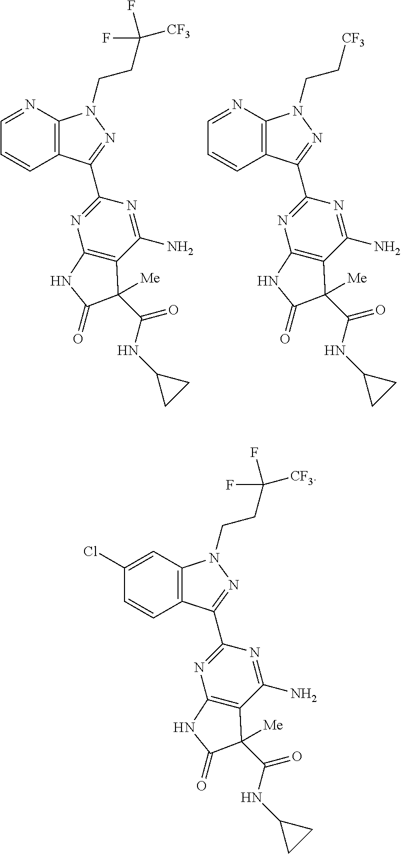 [SMILES: CC1(C(=O)NC2CC2)C(=O)Nc2nc(-c3nn(CCC(F)(F)C(F)(F)F)c4ncccc34)nc(N)c21 ; CC1(C(=O)NC2CC2)C(=O)Nc2nc(-c3nn(CCC(F)(F)F)c4ncccc34)nc(N)c21 ; CC1(C(=O)NC2CC2)C(=O)Nc2nc(-c3nn(CCC(F)(F)C(F)(F)F)c4cc(Cl)ccc34)nc(N)c21]

In some embodiments of the above methods, uses, compositions and kits, the sGC stimulator is a compounds selected from those depicted in Table XD below:

TABLE XD

| | |
|---|---|
| 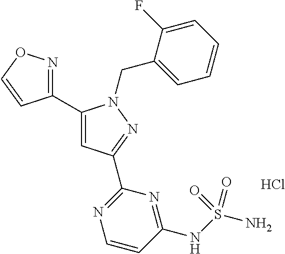 [SMILES: NS(=O)(=O)Nc1ccnc(-c2cc(-c3ccon3)n(Cc3ccccc3F)n2)n1 ; Cl] | I-456 |
| 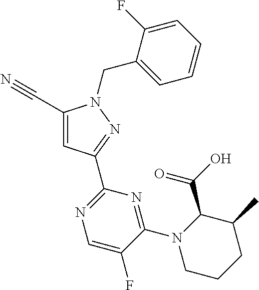 [SMILES: C[C@H]1CCCN(c2nc(-c3cc(C#N)n(Cc4ccccc4F)n3)ncc2F)[C@H]1C(=O)O] | I-457 |
| 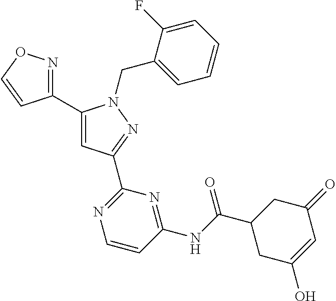 [SMILES: O=C1C=C(O)CC(C(=O)Nc2ccnc(-c3cc(-c4ccon4)n(Cc4ccccc4F)n3)n2)C1] | I-458 |
| 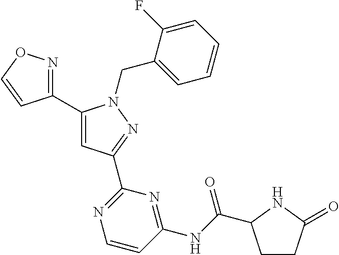 [SMILES: O=C1CCC(C(=O)Nc2ccnc(-c3cc(-c4ccon4)n(Cc4ccccc4F)n3)n2)N1] | I-459 |
| 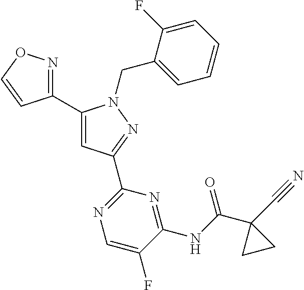 [SMILES: N#CC1(C(=O)Nc2nc(-c3cc(-c4ccon4)n(Cc4ccccc4F)n3)ncc2F)CC1] | I-460 |
| 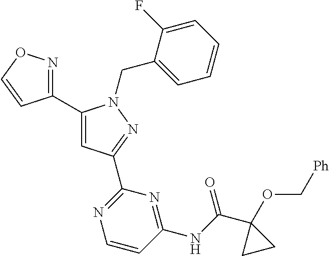 [SMILES: O=C(Nc1ccnc(-c2cc(-c3ccon3)n(Cc3ccccc3F)n2)n1)C1(OCc2ccccc2)CC1] | I-461 |
| 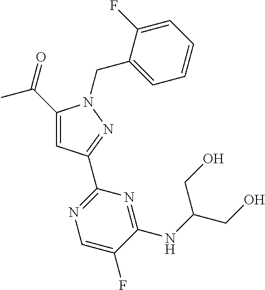 [SMILES: CC(=O)c1cc(-c2ncc(F)c(NC(CO)CO)n2)nn1Cc1ccccc1F] | I-462 |
| 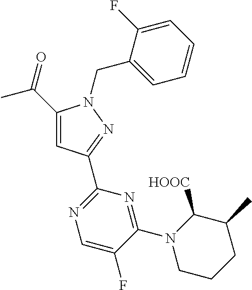 [SMILES: CC(=O)c1cc(-c2ncc(F)c(N3CCC[C@H](C)[C@@H]3C(=O)O)n2)nn1Cc1ccccc1F] | I-463 |
| 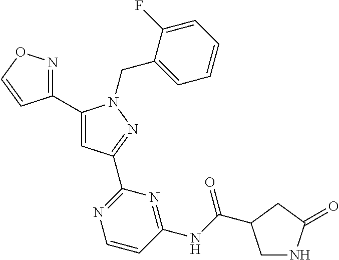 [SMILES: O=C1CC(C(=O)Nc2ccnc(-c3cc(-c4ccon4)n(Cc4ccccc4F)n3)n2)CN1] | I-464 |
| 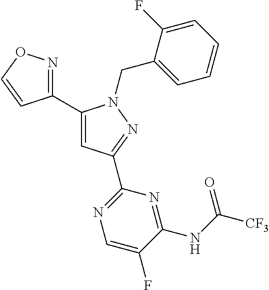 [SMILES: O=C(Nc1nc(-c2cc(-c3ccon3)n(Cc3ccccc3F)n2)ncc1F)C(F)(F)F] | I-465 |
| 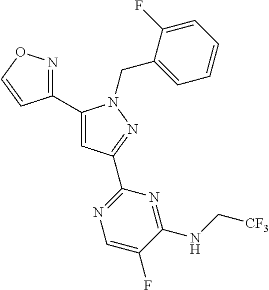 [SMILES: Fc1ccccc1Cn1nc(-c2ncc(F)c(NCC(F)(F)F)n2)cc1-c1ccon1] | I-466 |
| 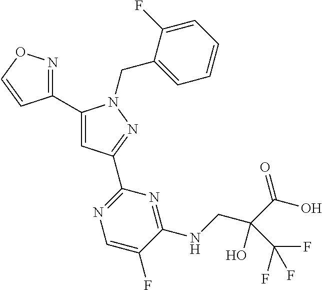 [SMILES: O=C(O)C(O)(CNc1nc(-c2cc(-c3ccon3)n(Cc3ccccc3F)n2)ncc1F)C(F)(F)F] | I-467 |
| 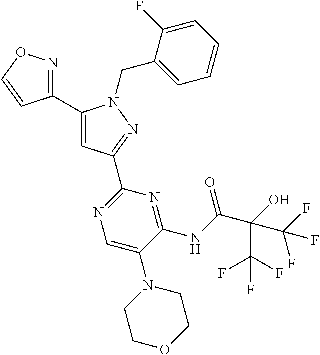 [SMILES: O=C(Nc1nc(-c2cc(-c3ccon3)n(Cc3ccccc3F)n2)ncc1N1CCOCC1)C(O)(C(F)(F)F)C(F)(F)F] | I-468 |
| 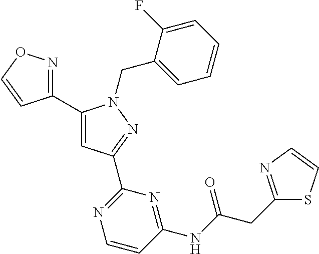 [SMILES: O=C(Cc1nccs1)Nc1ccnc(-c2cc(-c3ccon3)n(Cc3ccccc3F)n2)n1] | I-469 |
| 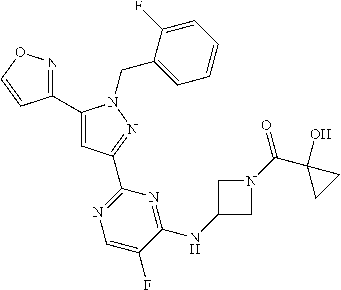 [SMILES: O=C(N1CC(Nc2nc(-c3cc(-c4ccon4)n(Cc4ccccc4F)n3)ncc2F)C1)C1(O)CC1] | I-470 |
| 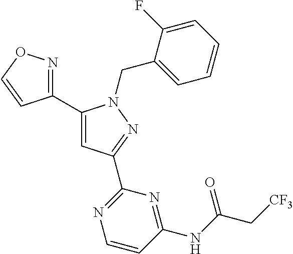 [SMILES: O=C(CC(F)(F)F)Nc1ccnc(-c2cc(-c3ccon3)n(Cc3ccccc3F)n2)n1] | I-471 |
| 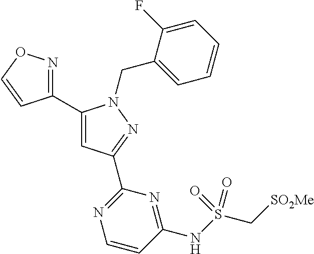 [SMILES: CS(=O)(=O)CS(=O)(=O)Nc1ccnc(-c2cc(-c3ccon3)n(Cc3ccccc3F)n2)n1] | I-472 |
| 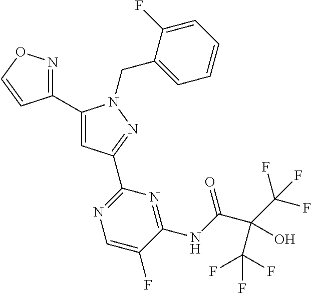 [SMILES: O=C(Nc1nc(-c2cc(-c3ccon3)n(Cc3ccccc3F)n2)ncc1F)C(O)(C(F)(F)F)C(F)(F)F] | I-473 |
| 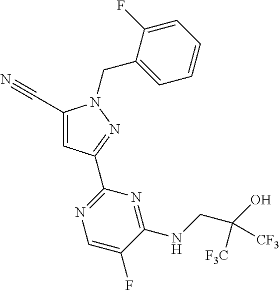 [SMILES: N#Cc1cc(-c2ncc(F)c(NCC(O)(C(F)(F)F)C(F)(F)F)n2)nn1Cc1ccccc1F] | I-474 |
| 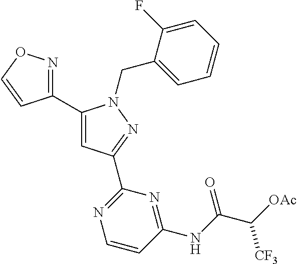 [SMILES: CC(=O)O[C@@H](C(=O)Nc1ccnc(-c2cc(-c3ccon3)n(Cc3ccccc3F)n2)n1)C(F)(F)F] | I-475 |
| 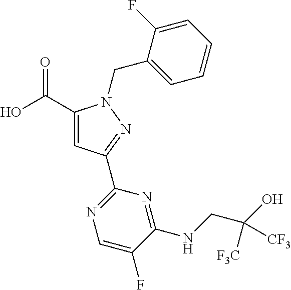 [SMILES: O=C(O)c1cc(-c2ncc(F)c(NCC(O)(C(F)(F)F)C(F)(F)F)n2)nn1Cc1ccccc1F] | I-476 |
| 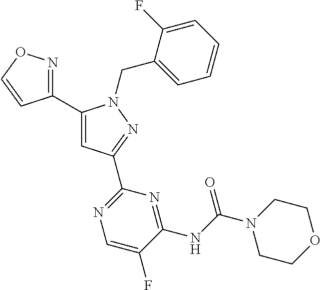 [SMILES: O=C(Nc1nc(-c2cc(-c3ccon3)n(Cc3ccccc3F)n2)ncc1F)N1CCOCC1] | I-477 |
| 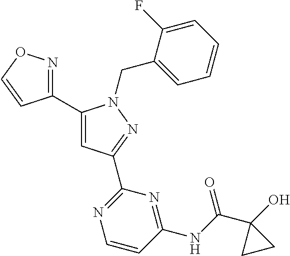 [SMILES: O=C(Nc1ccnc(-c2cc(-c3ccon3)n(Cc3ccccc3F)n2)n1)C1(O)CC1] | I-478 |
| 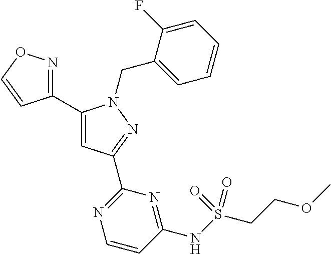 [SMILES: COCCS(=O)(=O)Nc1ccnc(-c2cc(-c3ccon3)n(Cc3ccccc3F)n2)n1] | I-479 |
| 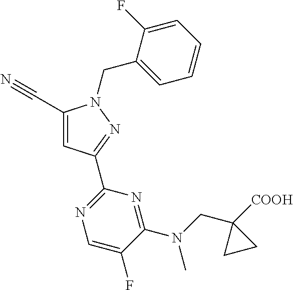 [SMILES: CN(CC1(C(=O)O)CC1)c1nc(-c2cc(C#N)n(Cc3ccccc3F)n2)ncc1F] | I-480 |
| 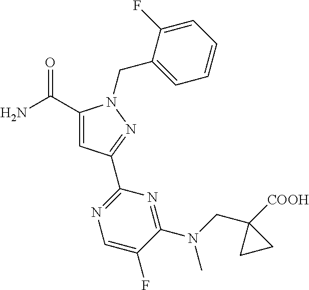 [SMILES: CN(CC1(C(=O)O)CC1)c1nc(-c2cc(C(N)=O)n(Cc3ccccc3F)n2)ncc1F] | I-481 |
| 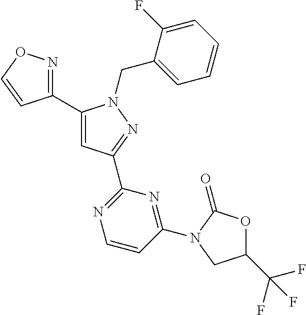 [SMILES: O=C1OC(C(F)(F)F)CN1c1ccnc(-c2cc(-c3ccon3)n(Cc3ccccc3F)n2)n1] | I-482 |
| 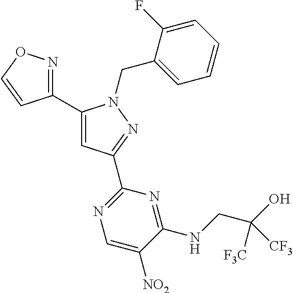 [SMILES: O=[N+]([O-])c1cnc(-c2cc(-c3ccon3)n(Cc3ccccc3F)n2)nc1NCC(O)(C(F)(F)F)C(F)(F)F] | I-483 |
| 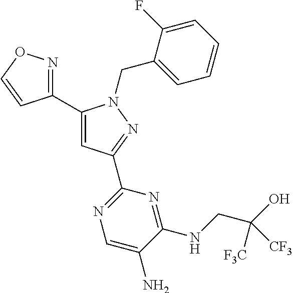 [SMILES: Nc1cnc(-c2cc(-c3ccon3)n(Cc3ccccc3F)n2)nc1NCC(O)(C(F)(F)F)C(F)(F)F] | I-484 |
| 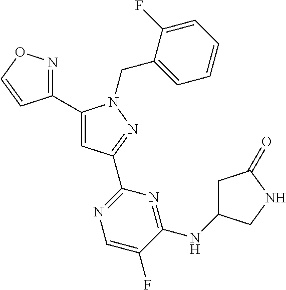 [SMILES: O=C1CC(Nc2nc(-c3cc(-c4ccon4)n(Cc4ccccc4F)n3)ncc2F)CN1] | I-485 |
| 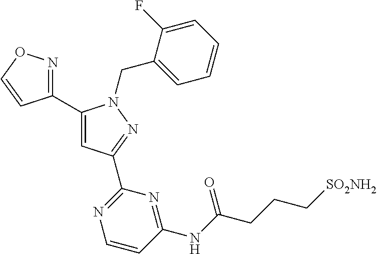 [SMILES: NS(=O)(=O)CCCC(=O)Nc1ccnc(-c2cc(-c3ccon3)n(Cc3ccccc3F)n2)n1] | I-486 |
| 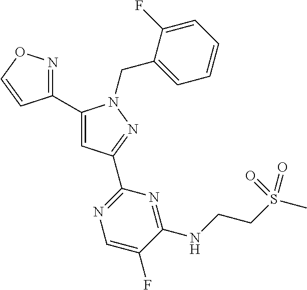 [SMILES: CS(=O)(=O)CCNc1nc(-c2cc(-c3ccon3)n(Cc3ccccc3F)n2)ncc1F] | I-487 |
| 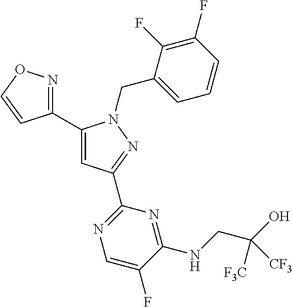 [SMILES: OC(CNc1nc(-c2cc(-c3ccon3)n(Cc3cccc(F)c3F)n2)ncc1F)(C(F)(F)F)C(F)(F)F] | I-488 |
| 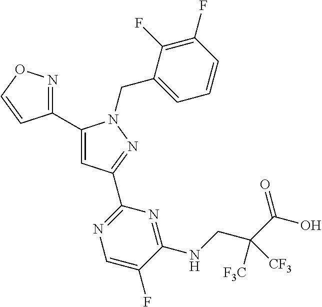 [SMILES: O=C(O)C(CNc1nc(-c2cc(-c3ccon3)n(Cc3cccc(F)c3F)n2)ncc1F)(C(F)(F)F)C(F)(F)F] | I-489 |
| 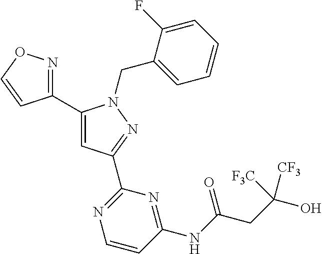 [SMILES: O=C(CC(O)(C(F)(F)F)C(F)(F)F)Nc1ccnc(-c2cc(-c3ccon3)n(Cc3ccccc3F)n2)n1] | I-490 |
| 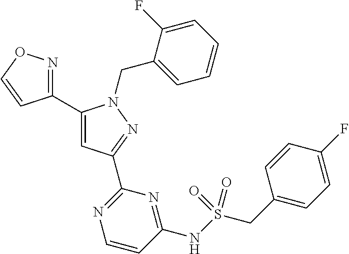 [SMILES: O=S(=O)(Cc1ccc(F)cc1)Nc1ccnc(-c2cc(-c3ccon3)n(Cc3ccccc3F)n2)n1] | I-491 |
| 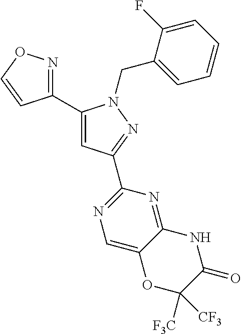 [SMILES: O=C1Nc2nc(-c3cc(-c4ccon4)n(Cc4ccccc4F)n3)ncc2OC1(C(F)(F)F)C(F)(F)F] | I-492 |
| 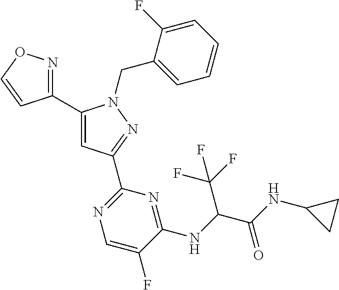 [SMILES: O=C(NC1CC1)C(Nc1nc(-c2cc(-c3ccon3)n(Cc3ccccc3F)n2)ncc1F)C(F)(F)F] | I-493 |
| 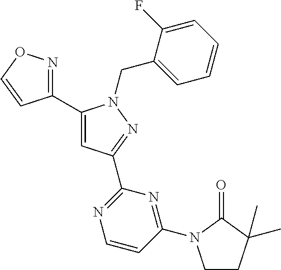 [SMILES: CC1(C)CCN(c2ccnc(-c3cc(-c4ccon4)n(Cc4ccccc4F)n3)n2)C1=O] | I-494 |
| 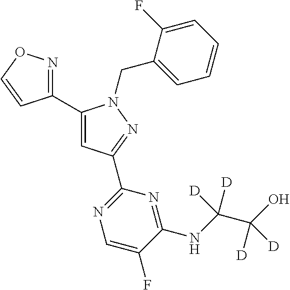 [SMILES: [2H]C([2H])(O)C([2H])([2H])Nc1nc(-c2cc(-c3ccon3)n(Cc3ccccc3F)n2)ncc1F] | I-495 |
| 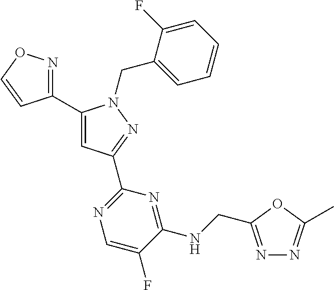 [SMILES: Cc1nnc(CNc2nc(-c3cc(-c4ccon4)n(Cc4ccccc4F)n3)ncc2F)o1] | I-496 |
| 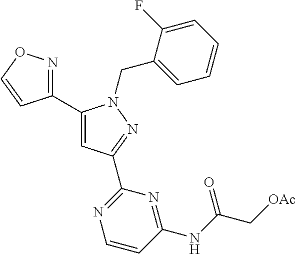 [SMILES: CC(=O)OCC(=O)Nc1ccnc(-c2cc(-c3ccon3)n(Cc3ccccc3F)n2)n1] | I-497 |
| 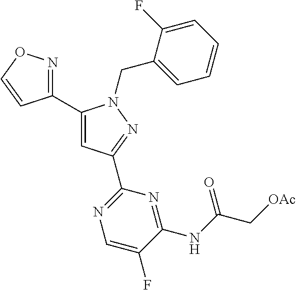 [SMILES: CC(=O)OCC(=O)Nc1nc(-c2cc(-c3ccon3)n(Cc3ccccc3F)n2)ncc1F] | I-498 |
| 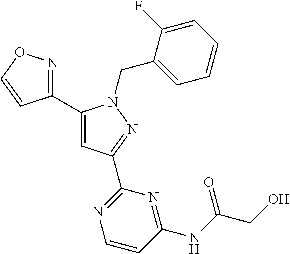 [SMILES: O=C(CO)Nc1ccnc(-c2cc(-c3ccon3)n(Cc3ccccc3F)n2)n1] | I-499 |
| 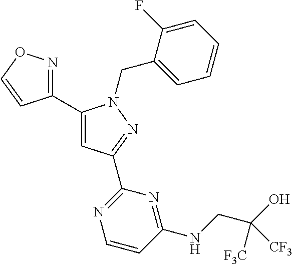 [SMILES: OC(CNc1ccnc(-c2cc(-c3ccon3)n(Cc3ccccc3F)n2)n1)(C(F)(F)F)C(F)(F)F] | I-500 |
| 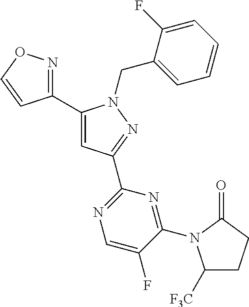 [SMILES: O=C1CCC(C(F)(F)F)N1c1nc(-c2cc(-c3ccon3)n(Cc3ccccc3F)n2)ncc1F] | I-501 |
| 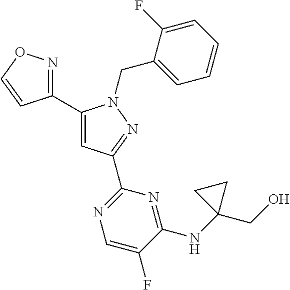 [SMILES: OCC1(Nc2nc(-c3cc(-c4ccon4)n(Cc4ccccc4F)n3)ncc2F)CC1] | I-502 |
| 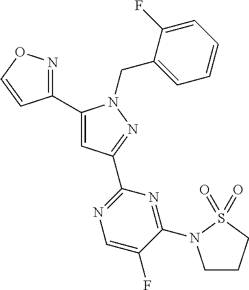 [SMILES: O=S1(=O)CCCN1c1nc(-c2cc(-c3ccon3)n(Cc3ccccc3F)n2)ncc1F] | I-503 |
| 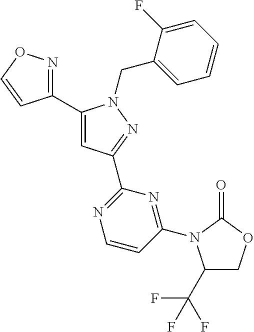 [SMILES: O=C1OCC(C(F)(F)F)N1c1ccnc(-c2cc(-c3ccon3)n(Cc3ccccc3F)n2)n1] | I-504 |
| 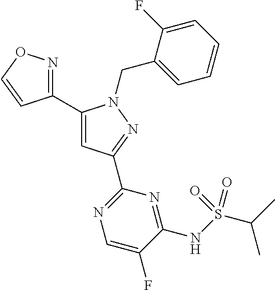 [SMILES: CC(C)S(=O)(=O)Nc1nc(-c2cc(-c3ccon3)n(Cc3ccccc3F)n2)ncc1F] | I-505 |
| 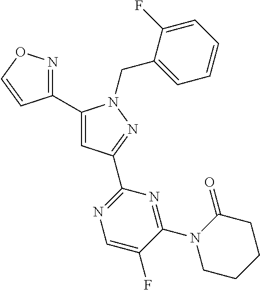 [SMILES: O=C1CCCCN1c1nc(-c2cc(-c3ccon3)n(Cc3ccccc3F)n2)ncc1F] | I-506 |
| 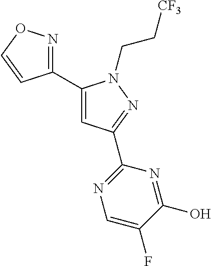 [SMILES: Oc1nc(-c2cc(-c3ccon3)n(CCC(F)(F)F)n2)ncc1F] | I-507 |
| 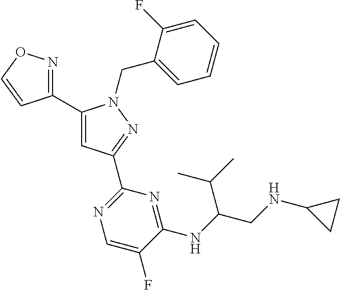 [SMILES: CC(C)C(CNC1CC1)Nc1nc(-c2cc(-c3ccon3)n(Cc3ccccc3F)n2)ncc1F] | I-508 |
| 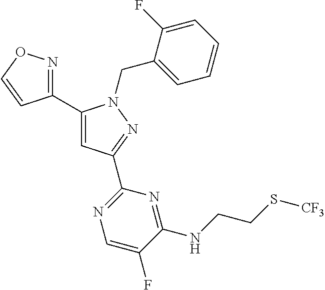 [SMILES: Fc1ccccc1Cn1nc(-c2ncc(F)c(NCCSC(F)(F)F)n2)cc1-c1ccon1] | I-509 |
| 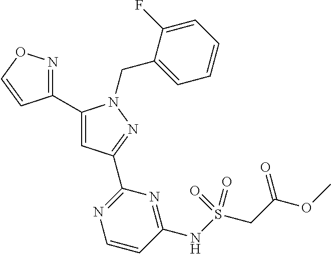 [SMILES: COC(=O)CS(=O)(=O)Nc1ccnc(-c2cc(-c3ccon3)n(Cc3ccccc3F)n2)n1] | I-510 |
| 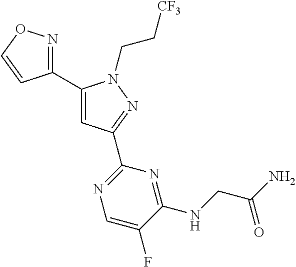 [SMILES: NC(=O)CNc1nc(-c2cc(-c3ccon3)n(CCC(F)(F)F)n2)ncc1F] | I-511 |
| 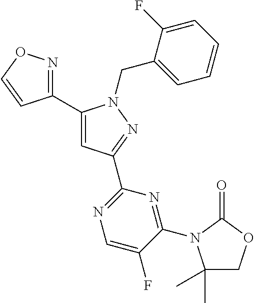 [SMILES: CC1(C)COC(=O)N1c1nc(-c2cc(-c3ccon3)n(Cc3ccccc3F)n2)ncc1F] | I-512 |
| 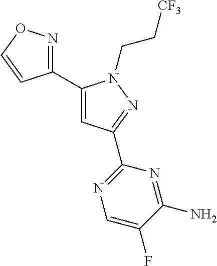 [SMILES: Nc1nc(-c2cc(-c3ccon3)n(CCC(F)(F)F)n2)ncc1F] | I-513 |
| 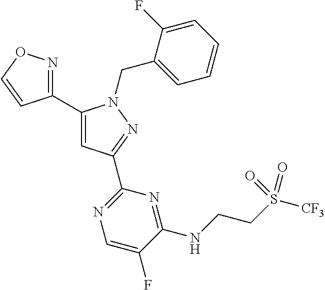 [SMILES: O=S(=O)(CCNc1nc(-c2cc(-c3ccon3)n(Cc3ccccc3F)n2)ncc1F)C(F)(F)F] | I-514 |
| 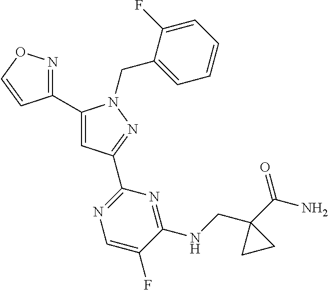 [SMILES: NC(=O)C1(CNc2nc(-c3cc(-c4ccon4)n(Cc4ccccc4F)n3)ncc2F)CC1] | I-515 |
| 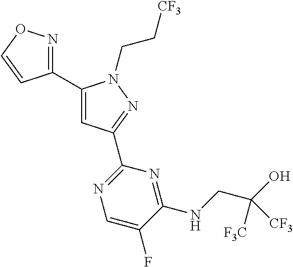 [SMILES: OC(CNc1nc(-c2cc(-c3ccon3)n(CCC(F)(F)F)n2)ncc1F)(C(F)(F)F)C(F)(F)F] | I-516 |
| 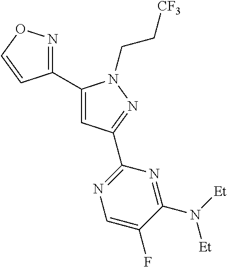 [SMILES: CCN(CC)c1nc(-c2cc(-c3ccon3)n(CCC(F)(F)F)n2)ncc1F] | I-517 |
| 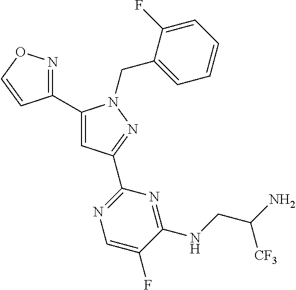 [SMILES: NC(CNc1nc(-c2cc(-c3ccon3)n(Cc3ccccc3F)n2)ncc1F)C(F)(F)F] | I-518 |
| 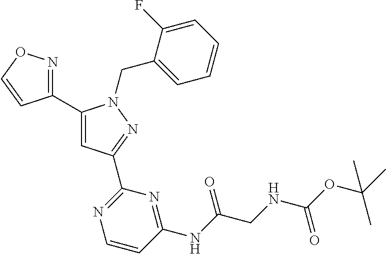 [SMILES: CC(C)(C)OC(=O)NCC(=O)Nc1ccnc(-c2cc(-c3ccon3)n(Cc3ccccc3F)n2)n1] | I-519 |
| 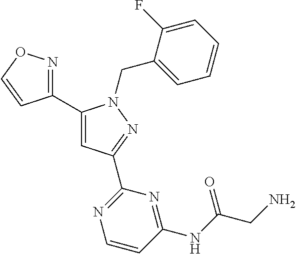 [SMILES: NCC(=O)Nc1ccnc(-c2cc(-c3ccon3)n(Cc3ccccc3F)n2)n1] | I-520 |
| 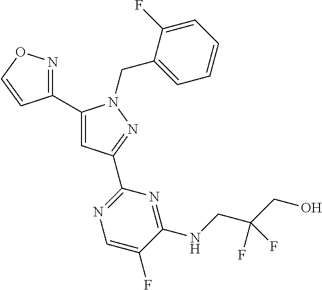 [SMILES: OCC(F)(F)CNc1nc(-c2cc(-c3ccon3)n(Cc3ccccc3F)n2)ncc1F] | I-521 |
| 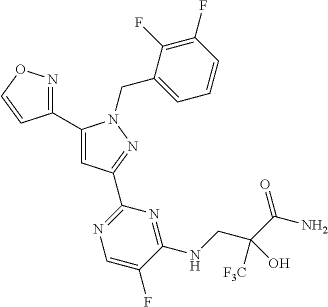 [SMILES: NC(=O)C(O)(CNc1nc(-c2cc(-c3ccon3)n(Cc3cccc(F)c3F)n2)ncc1F)C(F)(F)F] | I-522 |
| 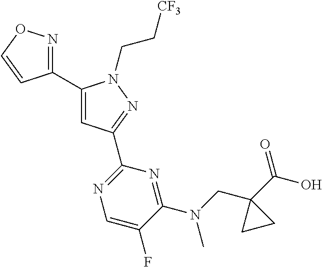 [SMILES: CN(CC1(C(=O)O)CC1)c1nc(-c2cc(-c3ccon3)n(CCC(F)(F)F)n2)ncc1F] | I-523 |
| 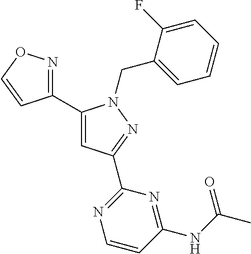 [SMILES: CC(=O)Nc1ccnc(-c2cc(-c3ccon3)n(Cc3ccccc3F)n2)n1] | I-524 |
| 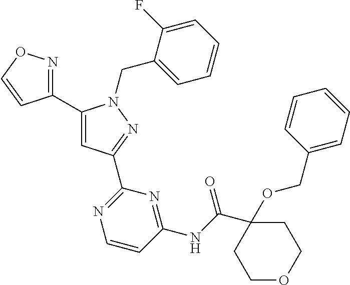 [SMILES: O=C(Nc1ccnc(-c2cc(-c3ccon3)n(Cc3ccccc3F)n2)n1)C1(OCc2ccccc2)CCOCC1] | I-525 |
| 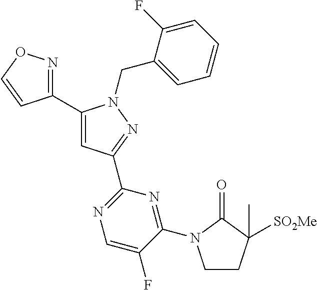 [SMILES: CC1(S(C)(=O)=O)CCN(c2nc(-c3cc(-c4ccon4)n(Cc4ccccc4F)n3)ncc2F)C1=O] | I-526 |
| 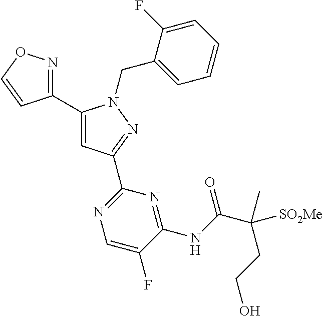 [SMILES: CC(CCO)(C(=O)Nc1nc(-c2cc(-c3ccon3)n(Cc3ccccc3F)n2)ncc1F)S(C)(=O)=O] | I-527 |
| 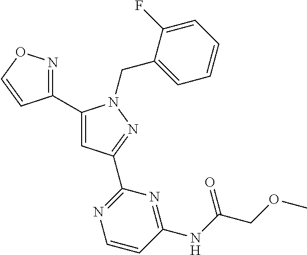 [SMILES: COCC(=O)Nc1ccnc(-c2cc(-c3ccon3)n(Cc3ccccc3F)n2)n1] | I-528 |
| 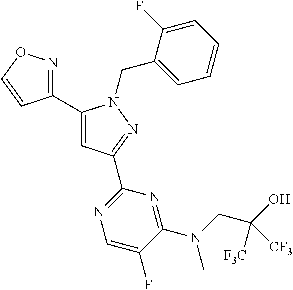 [SMILES: CN(CC(O)(C(F)(F)F)C(F)(F)F)c1nc(-c2cc(-c3ccon3)n(Cc3ccccc3F)n2)ncc1F] | I-529 |
| 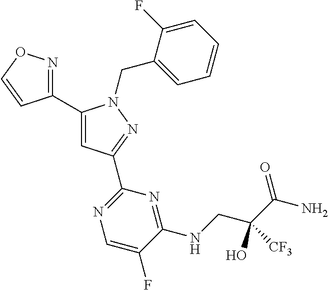 [SMILES: NC(=O)[C@@](O)(CNc1nc(-c2cc(-c3ccon3)n(Cc3ccccc3F)n2)ncc1F)C(F)(F)F] | I-530 |
| 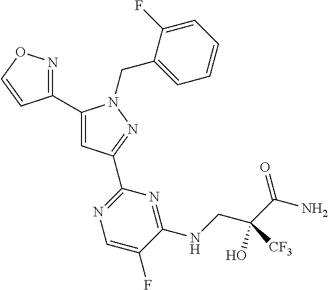 [SMILES: NC(=O)[C@](O)(CNc1nc(-c2cc(-c3ccon3)n(Cc3ccccc3F)n2)ncc1F)C(F)(F)F] | I-531 |
| 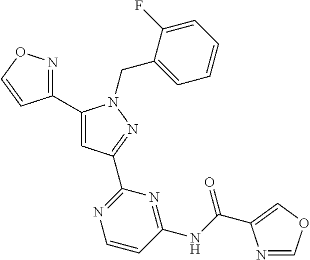 [SMILES: O=C(Nc1ccnc(-c2cc(-c3ccon3)n(Cc3ccccc3F)n2)n1)c1cocn1] | I-532 |
| 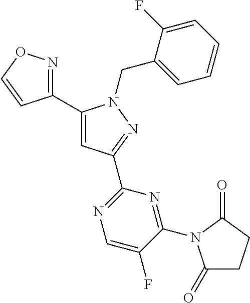 [SMILES: O=C1CCC(=O)N1c1nc(-c2cc(-c3ccon3)n(Cc3ccccc3F)n2)ncc1F] | I-533 |
| 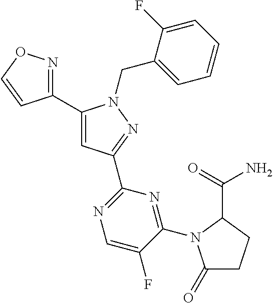 [SMILES: NC(=O)C1CCC(=O)N1c1nc(-c2cc(-c3ccon3)n(Cc3ccccc3F)n2)ncc1F] | I-534 |
| 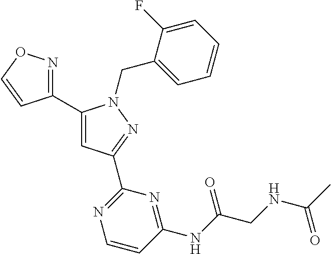 [SMILES: CC(=O)NCC(=O)Nc1ccnc(-c2cc(-c3ccon3)n(Cc3ccccc3F)n2)n1] | I-535 |
| 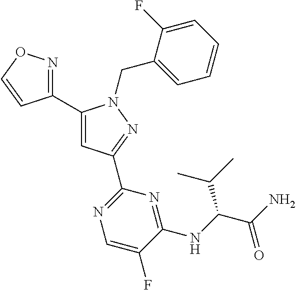 [SMILES: CC(C)[C@@H](Nc1nc(-c2cc(-c3ccon3)n(Cc3ccccc3F)n2)ncc1F)C(N)=O] | I-536 |
| 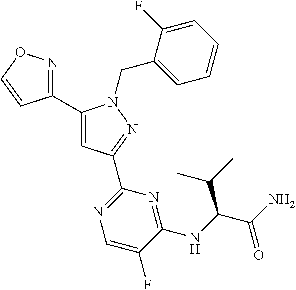 [SMILES: CC(C)[C@H](Nc1nc(-c2cc(-c3ccon3)n(Cc3ccccc3F)n2)ncc1F)C(N)=O] | I-537 |
| 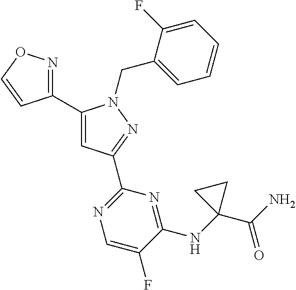 [SMILES: NC(=O)C1(Nc2nc(-c3cc(-c4ccon4)n(Cc4ccccc4F)n3)ncc2F)CC1] | I-538 |
| 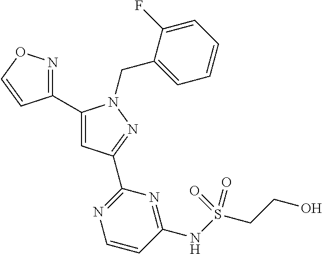 [SMILES: O=S(=O)(CCO)Nc1ccnc(-c2cc(-c3ccon3)n(Cc3ccccc3F)n2)n1] | I-539 |
| 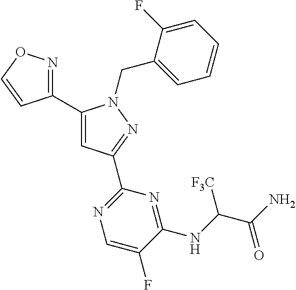 [SMILES: NC(=O)C(Nc1nc(-c2cc(-c3ccon3)n(Cc3ccccc3F)n2)ncc1F)C(F)(F)F] | I-540 |
| 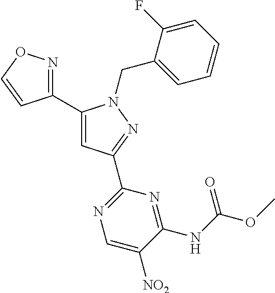 [SMILES: COC(=O)Nc1nc(-c2cc(-c3ccon3)n(Cc3ccccc3F)n2)ncc1[N+](=O)[O-]] | I-541 |
| 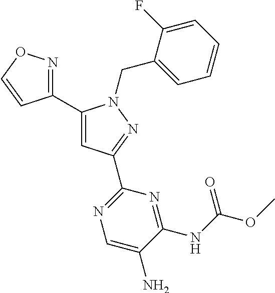 [SMILES: COC(=O)Nc1nc(-c2cc(-c3ccon3)n(Cc3ccccc3F)n2)ncc1N] | I-542 |
| 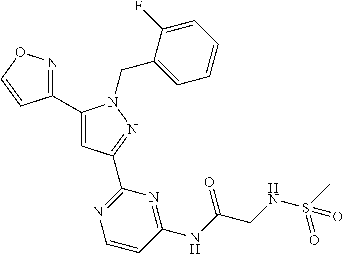 [SMILES: CS(=O)(=O)NCC(=O)Nc1ccnc(-c2cc(-c3ccon3)n(Cc3ccccc3F)n2)n1] | I-543 |
| 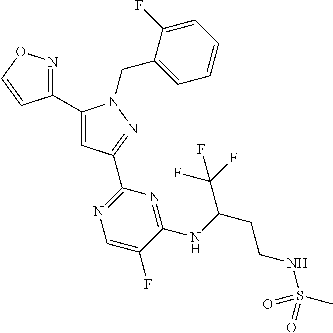 [SMILES: CS(=O)(=O)NCCC(Nc1nc(-c2cc(-c3ccon3)n(Cc3ccccc3F)n2)ncc1F)C(F)(F)F] | I-544 |
| 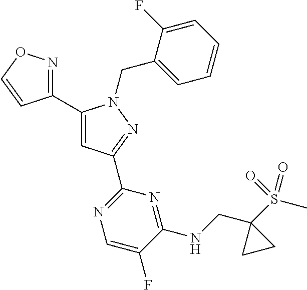 [SMILES: CS(=O)(=O)C1(CNc2nc(-c3cc(-c4ccon4)n(Cc4ccccc4F)n3)ncc2F)CC1] | I-545 |
| 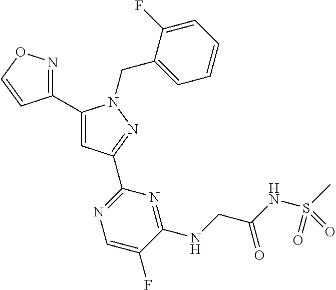 [SMILES: CS(=O)(=O)NC(=O)CNc1nc(-c2cc(-c3ccon3)n(Cc3ccccc3F)n2)ncc1F] | I-546 |
| 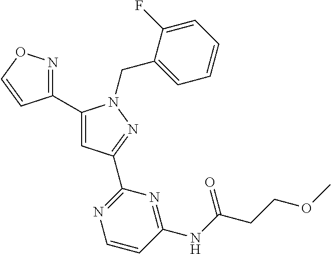 [SMILES: COCCC(=O)Nc1ccnc(-c2cc(-c3ccon3)n(Cc3ccccc3F)n2)n1] | I-547 |
| 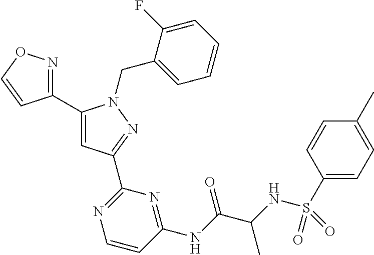 [SMILES: Cc1ccc(S(=O)(=O)NC(C)C(=O)Nc2ccnc(-c3cc(-c4ccon4)n(Cc4ccccc4F)n3)n2)cc1] | I-548 |
| 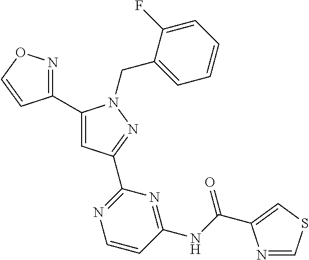 [SMILES: O=C(Nc1ccnc(-c2cc(-c3ccon3)n(Cc3ccccc3F)n2)n1)c1cscn1] | I-549 |
| 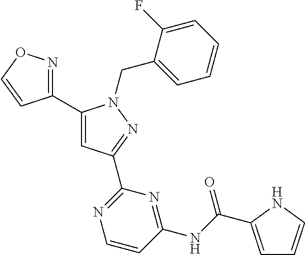 [SMILES: O=C(Nc1ccnc(-c2cc(-c3ccon3)n(Cc3ccccc3F)n2)n1)c1ccc[nH]1] | I-550 |
| 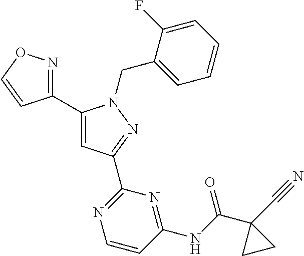 [SMILES: N#CC1(C(=O)Nc2ccnc(-c3cc(-c4ccon4)n(Cc4ccccc4F)n3)n2)CC1] | I-551 |
| 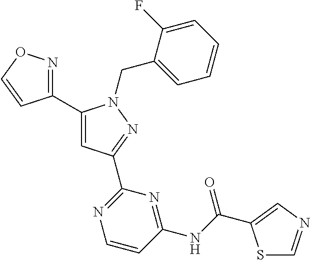 [SMILES: O=C(Nc1ccnc(-c2cc(-c3ccon3)n(Cc3ccccc3F)n2)n1)c1cncs1] | I-552 |
| 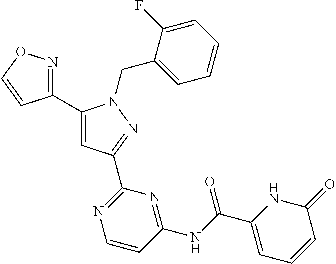 [SMILES: O=C(Nc1ccnc(-c2cc(-c3ccon3)n(Cc3ccccc3F)n2)n1)c1cccc(=O)[nH]1] | I-553 |
| 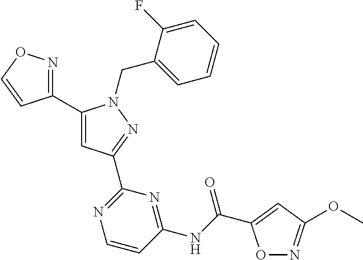 [SMILES: COc1cc(C(=O)Nc2ccnc(-c3cc(-c4ccon4)n(Cc4ccccc4F)n3)n2)on1] | I-554 |
| 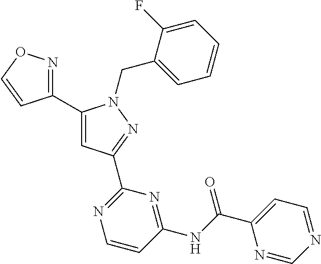 [SMILES: O=C(Nc1ccnc(-c2cc(-c3ccon3)n(Cc3ccccc3F)n2)n1)c1ccncn1] | I-555 |
| 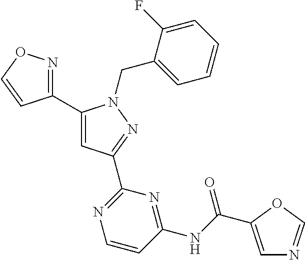 [SMILES: O=C(Nc1ccnc(-c2cc(-c3ccon3)n(Cc3ccccc3F)n2)n1)c1cnco1] | I-556 |
| 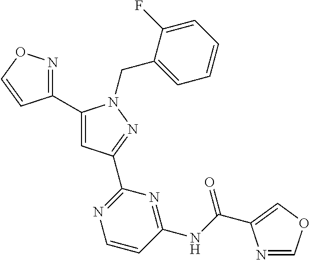 [SMILES: O=C(Nc1ccnc(-c2cc(-c3ccon3)n(Cc3ccccc3F)n2)n1)c1cocn1] | I-557 |
| 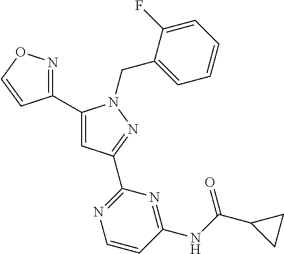 [SMILES: O=C(Nc1ccnc(-c2cc(-c3ccon3)n(Cc3ccccc3F)n2)n1)C1CC1] | I-558 |
| 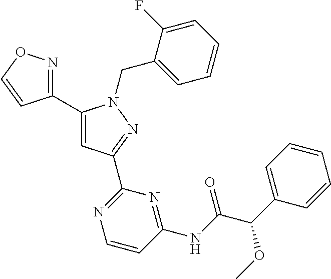 [SMILES: CO[C@H](C(=O)Nc1ccnc(-c2cc(-c3ccon3)n(Cc3ccccc3F)n2)n1)c1ccccc1] | I-559 |
| 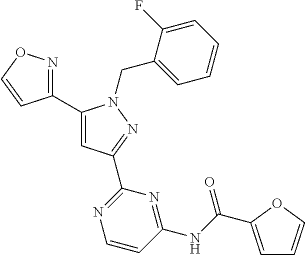 [SMILES: O=C(Nc1ccnc(-c2cc(-c3ccon3)n(Cc3ccccc3F)n2)n1)c1ccco1] | I-560 |
| 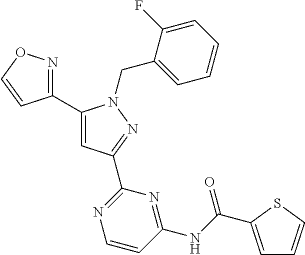 [SMILES: O=C(Nc1ccnc(-c2cc(-c3ccon3)n(Cc3ccccc3F)n2)n1)c1cccs1] | I-561 |
| 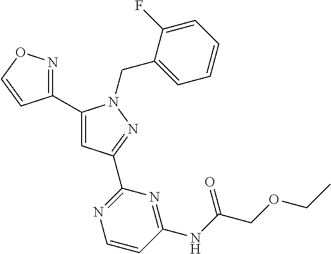 [SMILES: CCOCC(=O)Nc1ccnc(-c2cc(-c3ccon3)n(Cc3ccccc3F)n2)n1] | I-562 |
| 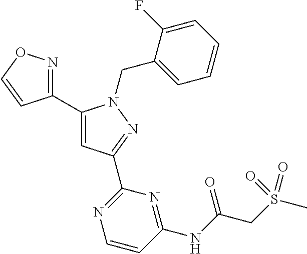 [SMILES: CS(=O)(=O)CC(=O)Nc1ccnc(-c2cc(-c3ccon3)n(Cc3ccccc3F)n2)n1] | I-563 |
| 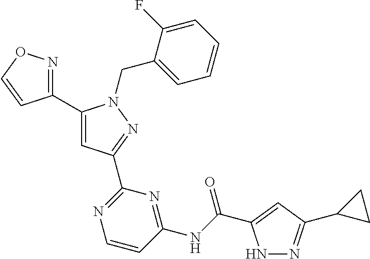 [SMILES: O=C(Nc1ccnc(-c2cc(-c3ccon3)n(Cc3ccccc3F)n2)n1)c1cc(C2CC2)n[nH]1] | I-564 |
| 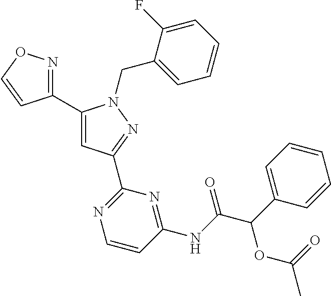 [SMILES: CC(=O)OC(C(=O)Nc1ccnc(-c2cc(-c3ccon3)n(Cc3ccccc3F)n2)n1)c1ccccc1] | I-565 |
| 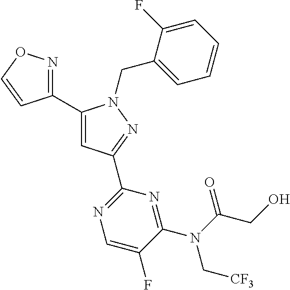 [SMILES: O=C(CO)N(CC(F)(F)F)c1nc(-c2cc(-c3ccon3)n(Cc3ccccc3F)n2)ncc1F] | I-566 |
| 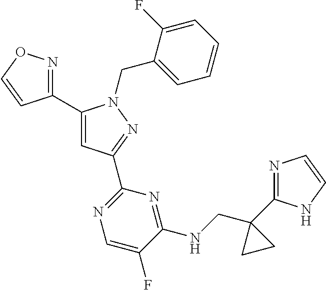 [SMILES: Fc1ccccc1Cn1nc(-c2ncc(F)c(NCC3(c4ncc[nH]4)CC3)n2)cc1-c1ccon1] | I-567 |
| 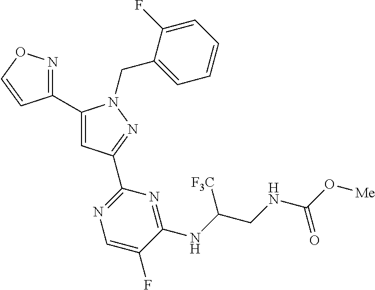 [SMILES: COC(=O)NCC(Nc1nc(-c2cc(-c3ccon3)n(Cc3ccccc3F)n2)ncc1F)C(F)(F)F] | I-568 |
| 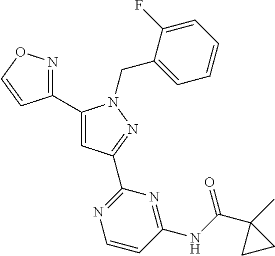 [SMILES: CC1(C(=O)Nc2ccnc(-c3cc(-c4ccon4)n(Cc4ccccc4F)n3)n2)CC1] | I-569 |
| 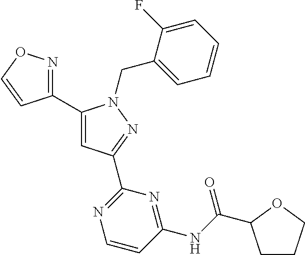 [SMILES: O=C(Nc1ccnc(-c2cc(-c3ccon3)n(Cc3ccccc3F)n2)n1)C1CCCO1] | I-570 |
| 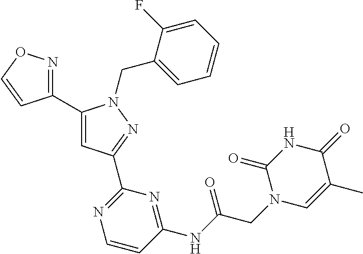 [SMILES: Cc1cn(CC(=O)Nc2ccnc(-c3cc(-c4ccon4)n(Cc4ccccc4F)n3)n2)c(=O)[nH]c1=O] | I-571 |
| 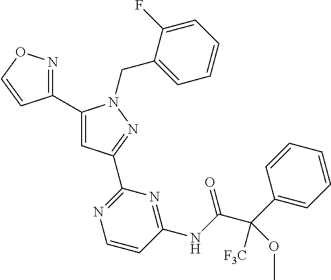 [SMILES: COC(C(=O)Nc1ccnc(-c2cc(-c3ccon3)n(Cc3ccccc3F)n2)n1)(c1ccccc1)C(F)(F)F] | I-572 |
| 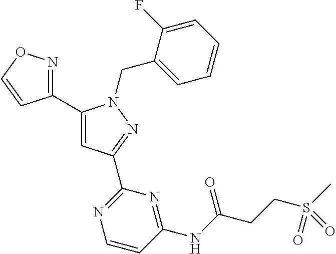 [SMILES: CS(=O)(=O)CCC(=O)Nc1ccnc(-c2cc(-c3ccon3)n(Cc3ccccc3F)n2)n1] | I-573 |
| 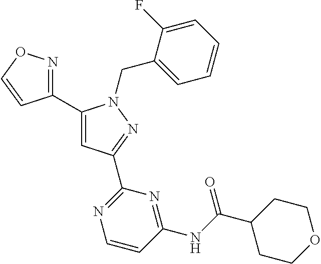 [SMILES: O=C(Nc1ccnc(-c2cc(-c3ccon3)n(Cc3ccccc3F)n2)n1)C1CCOCC1] | I-574 |
| 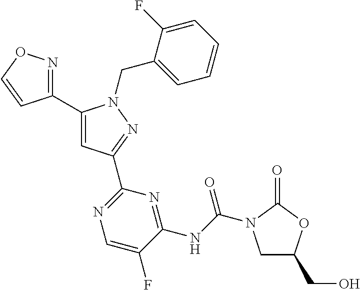 [SMILES: O=C(Nc1nc(-c2cc(-c3ccon3)n(Cc3ccccc3F)n2)ncc1F)N1C[C@H](CO)OC1=O] | I-575 |
| 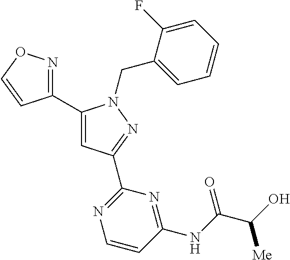 [SMILES: C[C@H](O)C(=O)Nc1ccnc(-c2cc(-c3ccon3)n(Cc3ccccc3F)n2)n1] | I-576 |
| 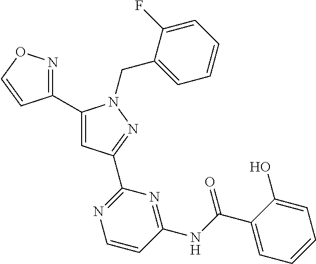 [SMILES: O=C(Nc1ccnc(-c2cc(-c3ccon3)n(Cc3ccccc3F)n2)n1)c1ccccc1O] | I-577 |
| 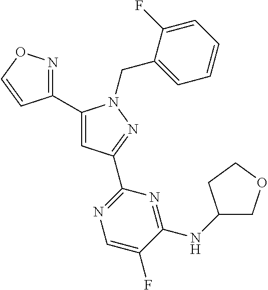 [SMILES: Fc1ccccc1Cn1nc(-c2ncc(F)c(NC3CCOC3)n2)cc1-c1ccon1] | I-578 |
| 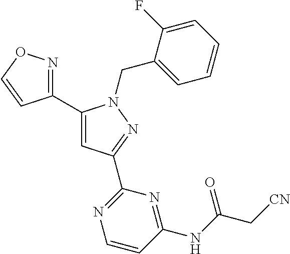 [SMILES: N#CCC(=O)Nc1ccnc(-c2cc(-c3ccon3)n(Cc3ccccc3F)n2)n1] | I-579 |
| 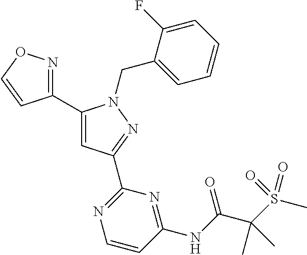 [SMILES: CC(C)(C(=O)Nc1ccnc(-c2cc(-c3ccon3)n(Cc3ccccc3F)n2)n1)S(C)(=O)=O] | I-580 |
| 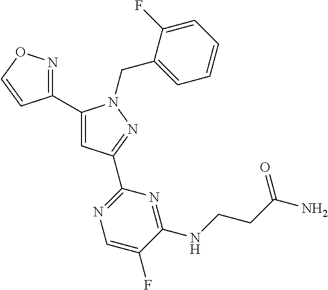 [SMILES: NC(=O)CCNc1nc(-c2cc(-c3ccon3)n(Cc3ccccc3F)n2)ncc1F] | I-581 |
| 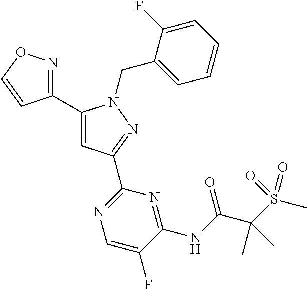 [SMILES: CC(C)(C(=O)Nc1nc(-c2cc(-c3ccon3)n(Cc3ccccc3F)n2)ncc1F)S(C)(=O)=O] | I-582 |
| 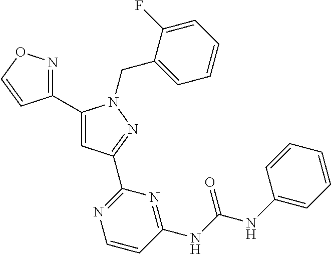 [SMILES: O=C(Nc1ccccc1)Nc1ccnc(-c2cc(-c3ccon3)n(Cc3ccccc3F)n2)n1] | I-583 |
| 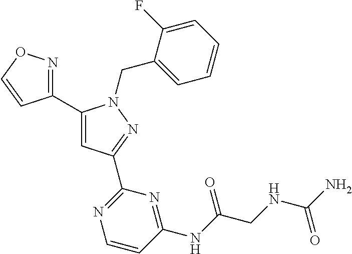 [SMILES: NC(=O)NCC(=O)Nc1ccnc(-c2cc(-c3ccon3)n(Cc3ccccc3F)n2)n1] | I-584 |
| 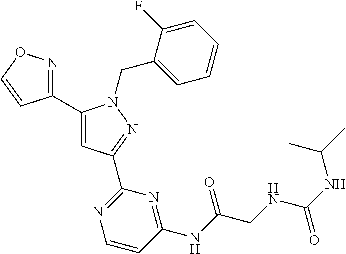 [SMILES: CC(C)NC(=O)NCC(=O)Nc1ccnc(-c2cc(-c3ccon3)n(Cc3ccccc3F)n2)n1] | I-585 |
| 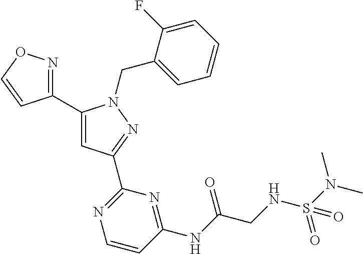 [SMILES: CN(C)S(=O)(=O)NCC(=O)Nc1ccnc(-c2cc(-c3ccon3)n(Cc3ccccc3F)n2)n1] | I-586 |
| 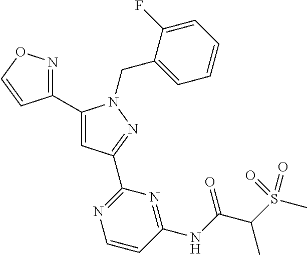 [SMILES: CC(C(=O)Nc1ccnc(-c2cc(-c3ccon3)n(Cc3ccccc3F)n2)n1)S(C)(=O)=O] | I-587 |
| 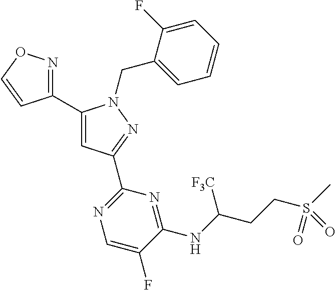 [SMILES: CS(=O)(=O)CCC(Nc1nc(-c2cc(-c3ccon3)n(Cc3ccccc3F)n2)ncc1F)C(F)(F)F] | I-588 |
| 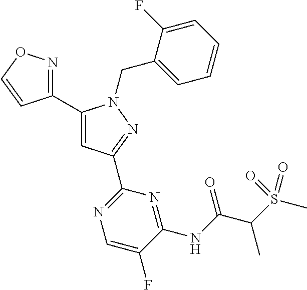 [SMILES: CC(C(=O)Nc1nc(-c2cc(-c3ccon3)n(Cc3ccccc3F)n2)ncc1F)S(C)(=O)=O] | I-589 |
| 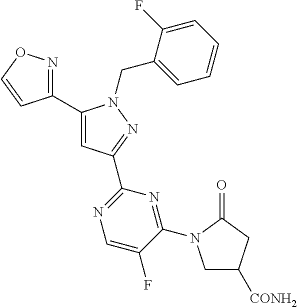 [SMILES: NC(=O)C1CC(=O)N(c2nc(-c3cc(-c4ccon4)n(Cc4ccccc4F)n3)ncc2F)C1] | I-590 |
| 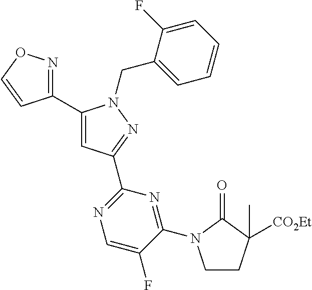 [SMILES: CCOC(=O)C1(C)CCN(c2nc(-c3cc(-c4ccon4)n(Cc4ccccc4F)n3)ncc2F)C1=O] | I-591 |
| 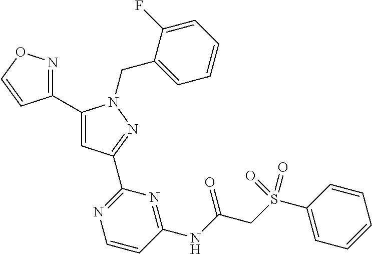 [SMILES: O=C(CS(=O)(=O)c1ccccc1)Nc1ccnc(-c2cc(-c3ccon3)n(Cc3ccccc3F)n2)n1] | I-592 |
| 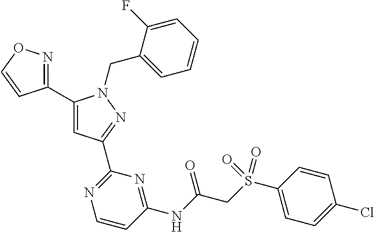 [SMILES: O=C(CS(=O)(=O)c1ccc(Cl)cc1)Nc1ccnc(-c2cc(-c3ccon3)n(Cc3ccccc3F)n2)n1] | I-593 |
| 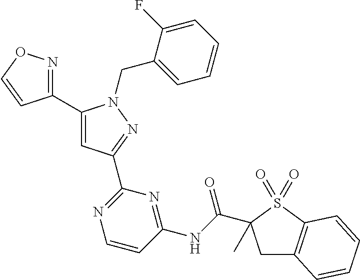 [SMILES: CC1(C(=O)Nc2ccnc(-c3cc(-c4ccon4)n(Cc4ccccc4F)n3)n2)Cc2ccccc2S1(=O)=O] | I-594 |
| 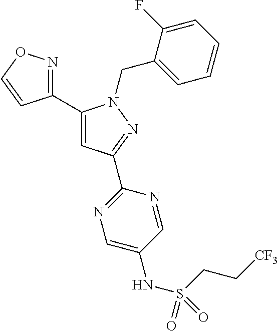 [SMILES: O=S(=O)(CCC(F)(F)F)Nc1cnc(-c2cc(-c3ccon3)n(Cc3ccccc3F)n2)nc1] | I-595 |
| 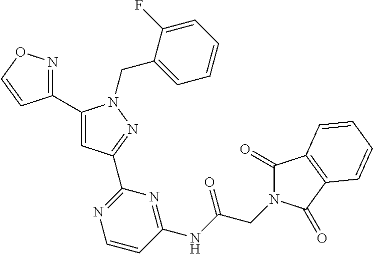 [SMILES: O=C(CN1C(=O)c2ccccc2C1=O)Nc1ccnc(-c2cc(-c3ccon3)n(Cc3ccccc3F)n2)n1] | I-596 |
| 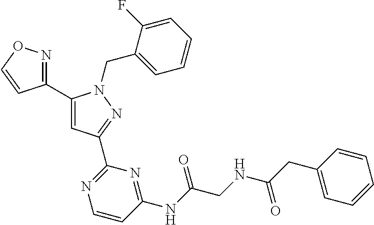 [SMILES: O=C(Cc1ccccc1)NCC(=O)Nc1ccnc(-c2cc(-c3ccon3)n(Cc3ccccc3F)n2)n1] | I-597 |
| 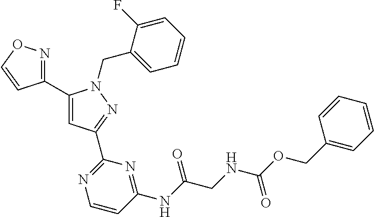 [SMILES: O=C(CNC(=O)OCc1ccccc1)Nc1ccnc(-c2cc(-c3ccon3)n(Cc3ccccc3F)n2)n1] | I-598 |
| 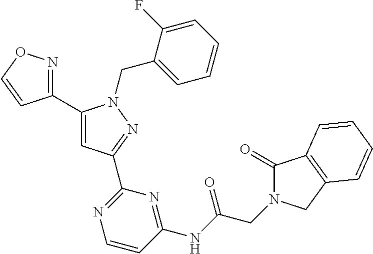 [SMILES: O=C(CN1Cc2ccccc2C1=O)Nc1ccnc(-c2cc(-c3ccon3)n(Cc3ccccc3F)n2)n1] | I-599 |
| 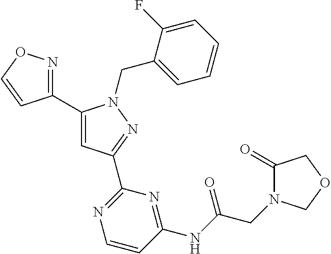 [SMILES: O=C(CN1COCC1=O)Nc1ccnc(-c2cc(-c3ccon3)n(Cc3ccccc3F)n2)n1] | I-600 |
| 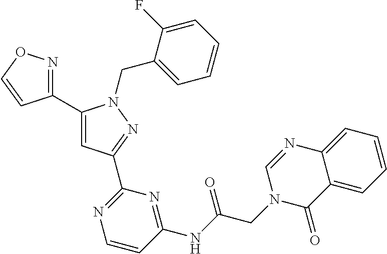 [SMILES: O=C(Cn1cnc2ccccc2c1=O)Nc1ccnc(-c2cc(-c3ccon3)n(Cc3ccccc3F)n2)n1] | I-601 |
| 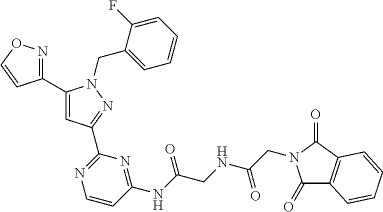 [SMILES: O=C(CN1C(=O)c2ccccc2C1=O)NCC(=O)Nc1ccnc(-c2cc(-c3ccon3)n(Cc3ccccc3F)n2)n1] | I-602 |
| 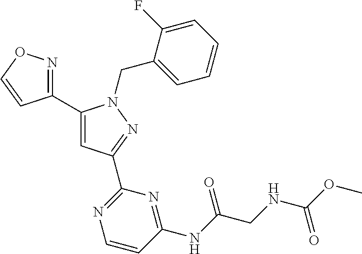 [SMILES: COC(=O)NCC(=O)Nc1ccnc(-c2cc(-c3ccon3)n(Cc3ccccc3F)n2)n1] | I-603 |
| 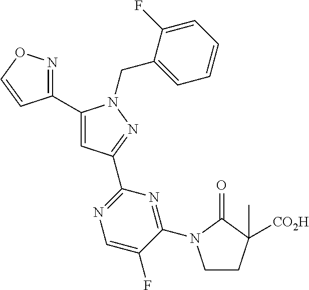 [SMILES: CC1(C(=O)O)CCN(c2nc(-c3cc(-c4ccon4)n(Cc4ccccc4F)n3)ncc2F)C1=O] | I-604 |
| 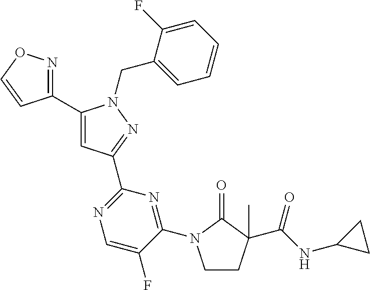 [SMILES: CC1(C(=O)NC2CC2)CCN(c2nc(-c3cc(-c4ccon4)n(Cc4ccccc4F)n3)ncc2F)C1=O] | I-605 |
| 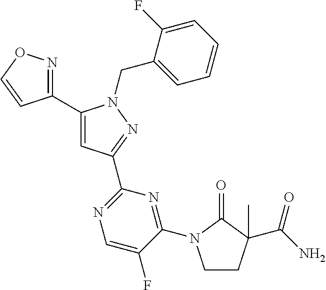 [SMILES: CC1(C(N)=O)CCN(c2nc(-c3cc(-c4ccon4)n(Cc4ccccc4F)n3)ncc2F)C1=O] | I-606 |
| 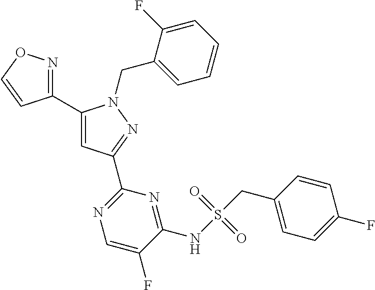 [SMILES: O=S(=O)(Cc1ccc(F)cc1)Nc1nc(-c2cc(-c3ccon3)n(Cc3ccccc3F)n2)ncc1F] | I-607 |
| 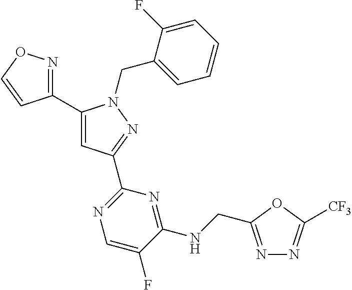 [SMILES: Fc1ccccc1Cn1nc(-c2ncc(F)c(NCc3nnc(C(F)(F)F)o3)n2)cc1-c1ccon1] | I-608 |
| 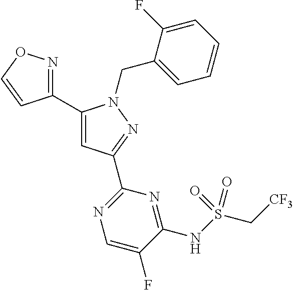 [SMILES: O=S(=O)(CC(F)(F)F)Nc1nc(-c2cc(-c3ccon3)n(Cc3ccccc3F)n2)ncc1F] | I-609 |
| 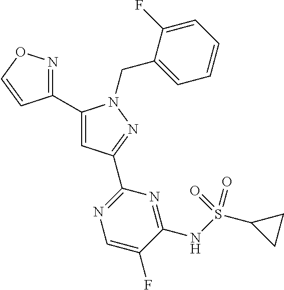 [SMILES: O=S(=O)(Nc1nc(-c2cc(-c3ccon3)n(Cc3ccccc3F)n2)ncc1F)C1CC1] | I-610 |
| 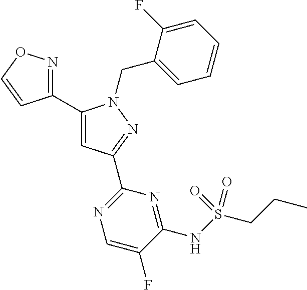 [SMILES: CCCS(=O)(=O)Nc1nc(-c2cc(-c3ccon3)n(Cc3ccccc3F)n2)ncc1F] | I-611 |
| 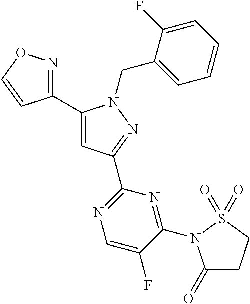 [SMILES: O=C1CCS(=O)(=O)N1c1nc(-c2cc(-c3ccon3)n(Cc3ccccc3F)n2)ncc1F] | I-612 |
| 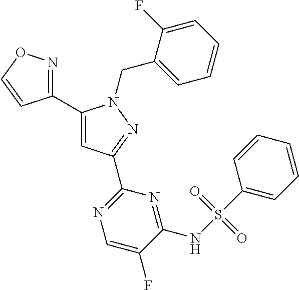 [SMILES: O=S(=O)(Nc1nc(-c2cc(-c3ccon3)n(Cc3ccccc3F)n2)ncc1F)c1ccccc1] | I-613 |
| 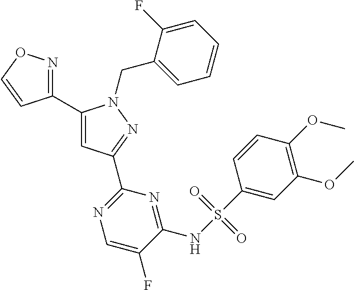 [SMILES: COc1ccc(S(=O)(=O)Nc2nc(-c3cc(-c4ccon4)n(Cc4ccccc4F)n3)ncc2F)cc1OC] | I-614 |
| 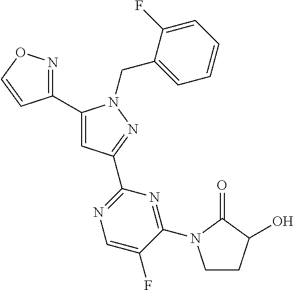 [SMILES: O=C1C(O)CCN1c1nc(-c2cc(-c3ccon3)n(Cc3ccccc3F)n2)ncc1F] | I-615 |
| 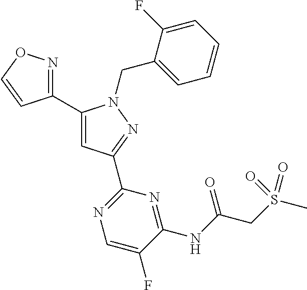 [SMILES: CS(=O)(=O)CC(=O)Nc1nc(-c2cc(-c3ccon3)n(Cc3ccccc3F)n2)ncc1F] | I-616 |
| 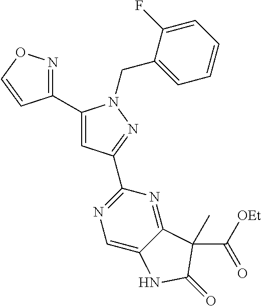 [SMILES: CCOC(=O)C1(C)C(=O)Nc2cnc(-c3cc(-c4ccon4)n(Cc4ccccc4F)n3)nc21] | I-617 |
| 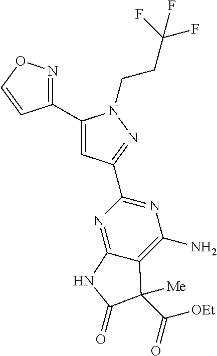 [SMILES: CCOC(=O)C1(C)C(=O)Nc2nc(-c3cc(-c4ccon4)n(CCC(F)(F)F)n3)nc(N)c21] | I-618 |
| 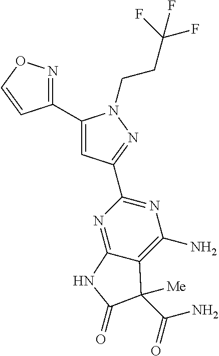 [SMILES: CC1(C(N)=O)C(=O)Nc2nc(-c3cc(-c4ccon4)n(CCC(F)(F)F)n3)nc(N)c21] | I-619 |
| 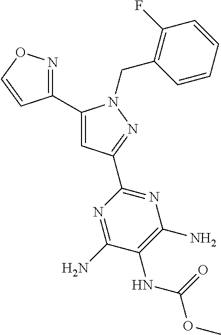 [SMILES: COC(=O)Nc1c(N)nc(-c2cc(-c3ccon3)n(Cc3ccccc3F)n2)nc1N] | I-620 |
| 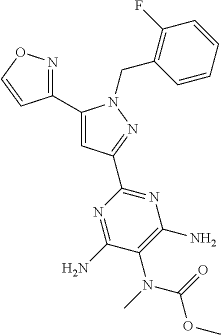 [SMILES: COC(=O)N(C)c1c(N)nc(-c2cc(-c3ccon3)n(Cc3ccccc3F)n2)nc1N] | I-621 |
| 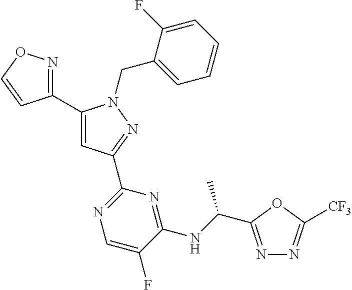 [SMILES: C[C@@H](Nc1nc(-c2cc(-c3ccon3)n(Cc3ccccc3F)n2)ncc1F)c1nnc(C(F)(F)F)o1] | I-622 |
| 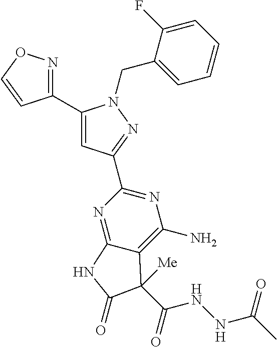 [SMILES: CC(=O)NNC(=O)C1(C)C(=O)Nc2nc(-c3cc(-c4ccon4)n(Cc4ccccc4F)n3)nc(N)c21] | I-623 |
| 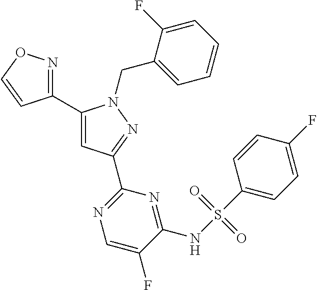 [SMILES: O=S(=O)(Nc1nc(-c2cc(-c3ccon3)n(Cc3ccccc3F)n2)ncc1F)c1ccc(F)cc1] | I-624 |
| 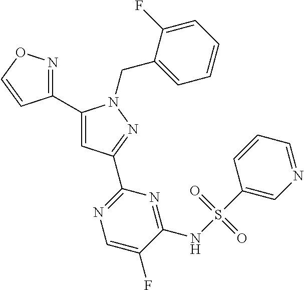 [SMILES: O=S(=O)(Nc1nc(-c2cc(-c3ccon3)n(Cc3ccccc3F)n2)ncc1F)c1cccnc1] | I-625 |
| 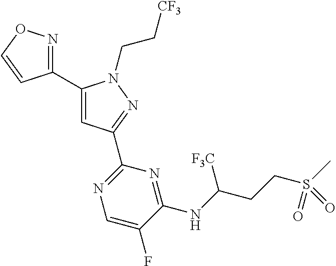 [SMILES: CS(=O)(=O)CCC(Nc1nc(-c2cc(-c3ccon3)n(CCC(F)(F)F)n2)ncc1F)C(F)(F)F] | I-626 |
| 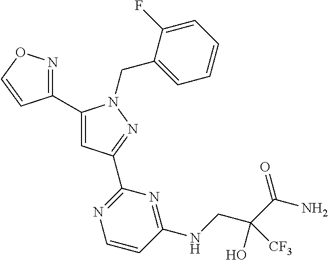 [SMILES: NC(=O)C(O)(CNc1ccnc(-c2cc(-c3ccon3)n(Cc3ccccc3F)n2)n1)C(F)(F)F] | I-627 |
| 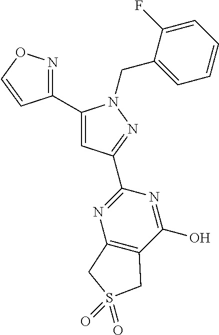 [SMILES: O=S1(=O)Cc2nc(-c3cc(-c4ccon4)n(Cc4ccccc4F)n3)nc(O)c2C1] | I-628 |
| 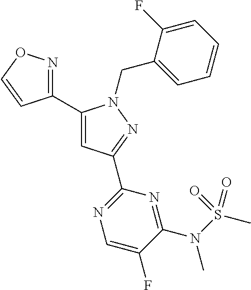 [SMILES: CN(c1nc(-c2cc(-c3ccon3)n(Cc3ccccc3F)n2)ncc1F)S(C)(=O)=O] | I-629 |
| 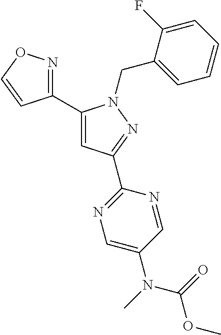 [SMILES: COC(=O)N(C)c1cnc(-c2cc(-c3ccon3)n(Cc3ccccc3F)n2)nc1] | I-630 |
| 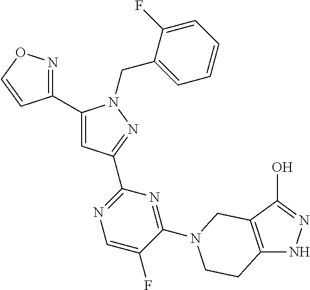 [SMILES: Oc1n[nH]c2c1CN(c1nc(-c3cc(-c4ccon4)n(Cc4ccccc4F)n3)ncc1F)CC2] | I-631 |
| 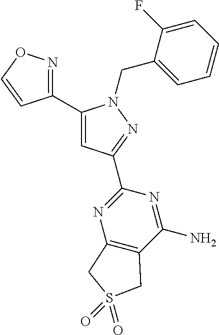 [SMILES: Nc1nc(-c2cc(-c3ccon3)n(Cc3ccccc3F)n2)nc2c1CS(=O)(=O)C2] | I-632 |
| 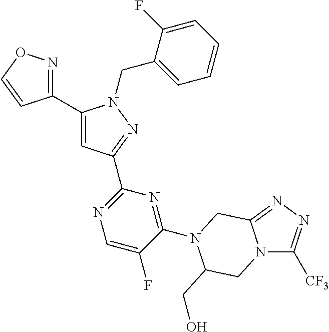 [SMILES: OCC1Cn2c(nnc2C(F)(F)F)CN1c1nc(-c2cc(-c3ccon3)n(Cc3ccccc3F)n2)ncc1F] | I-633 |
| 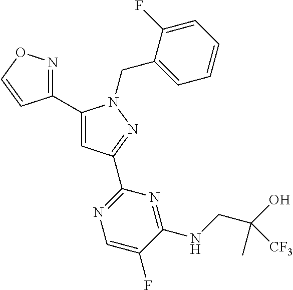 [SMILES: CC(O)(CNc1nc(-c2cc(-c3ccon3)n(Cc3ccccc3F)n2)ncc1F)C(F)(F)F] | I-634 |

### DEFINITIONS AND GENERAL TERMINOLOGY

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry”, Thomas Sorrell, University Science Books, Sausalito: 1999, and “March's Advanced Organic Chemistry”, 5^{th} Ed., Smith, M. B. and March, J., eds. John Wiley & Sons, New York: 2001, which are herein incorporated by reference in their entirety.

As described herein, compounds of Formula I may be optionally substituted with one or more substituents, such as illustrated generally below, or as exemplified by particular classes, subclasses and species of the invention. The phrase “optionally substituted” is used interchangeably with the phrase “substituted or unsubstituted.” In general, the term “substituted” refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position unless otherwise specified. As will be apparent to one of ordinary skill in the art, groups such as —H, halogen, —NO₂, —CN, —OH, —NH₂ or —OCF₃ would not be substitutable groups.

The phrase “up to”, as used herein, refers to zero or any integer number that is equal to or less than the number following the phrase. For example, “up to 3” means any one of 0, 1, 2, or 3. As described herein, a specified number range of atoms includes any integer therein. For example, a group having from 1-4 atoms could have 1, 2, 3 or 4 atoms. When any variable occurs more than one time at any position, its definition on each occurrence is independent from every other occurrence.

Selection of substituents and combinations envisioned by this disclosure are only those that result in the formation of stable or chemically feasible compounds. Such choices and combinations will be apparent to those of ordinary skill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. In some embodiments, a stable compound is one that is not substantially altered when kept at a temperature of 25° C. or less, in the absence of moisture or other chemically reactive conditions, for at least a week. A chemically feasible compound is a compound that can be prepared by a person skilled in the art based on the disclosures herein supplemented, if necessary, relevant knowledge of the art.

A compound, such as the compounds of Formula I or other compounds herein disclosed, may be present in its free form (e.g. an amorphous form, or a crystalline form or a polymorph). Under certain conditions, compounds may also form co-forms. As used herein, the term co-form is synonymous with the term multi-component crystalline form. When one of the components in the co-form has clearly transferred a proton to the other component, the resulting co-form is referred to as a “salt”. The formation of a salt is determined by how large the difference is in the pKas between the partners that form the mixture. For purposes of this disclosure, compounds include pharmaceutically acceptable salts, even if the term “pharmaceutically acceptable salts” is not explicitly noted.

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are also within the scope of the invention. As an example, a substituent drawn as below:

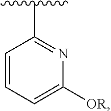

wherein R may be hydrogen, would include both compounds shown below:

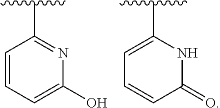

The present disclosure also embraces isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The term “aliphatic” or “aliphatic group”, as used herein, means a straight-chain (i.e., unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms. In other embodiments, aliphatic groups contain 1-4 aliphatic carbon atoms and in yet other embodiments, aliphatic groups contain 1-3 aliphatic carbon atoms. Suitable aliphatic groups include, but are not limited to, linear or branched, substituted or unsubstituted alkyl, alkenyl, or alkynyl groups. Specific examples of aliphatic groups include, but are not limited to: methyl, ethyl, propyl, butyl, isopropyl, isobutyl, vinyl, sec-butyl, tert-butyl, butenyl, propargyl, acetylene and the like. To be perfectly clear, the term “aliphatic chain” may be used interchangeably with the term “aliphatic” or “aliphatic group”.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “carbocyclic” refers to a ring system formed only by carbon and hydrogen atoms. Unless otherwise specified, throughout this disclosure, carbocycle is used as a synonym of “non-aromatic carbocycle” or “cycloaliphatic”. In some instances the term can be used in the phrase “aromatic carbocycle”, and in this case it refers to an “aryl group” as defined below.

The term “cycloaliphatic” (or “non-aromatic carbocycle”, “non-aromatic carbocyclyl”, “non-aromatic carbocyclic”) refers to a cyclic hydrocarbon that is completely saturated or that contains one or more units of unsaturation but which is not aromatic, and which has a single point of attachment to the rest of the molecule. Unless otherwise specified, a cycloaliphatic group may be monocyclic, bicyclic, tricyclic, fused, spiro or bridged. In one embodiment, the term “cycloaliphatic” refers to a monocyclic C₃-C₁₂ hydrocarbon or a bicyclic C₇-C₁₂ hydrocarbon. In some embodiments, any individual ring in a bicyclic or tricyclic ring system has 3-7 members. Suitable cycloaliphatic groups include, but are not limited to, cycloalkyl, cycloalkenyl, and cycloalkynyl. Examples of aliphatic groups include cyclopropyl, cyclobutyl, cyclopentyl, cyclopentenyl, cyclohexyl, cyclohexenyl, cycloheptyl, cycloheptenyl, norbornyl, cyclooctyl, cyclononyl, cyclodecyl, cycloundecyl, cyclododecyl, and the like.

The term “cycloaliphatic” also includes polycyclic ring systems in which the non-aromatic carbocyclic ring can be “fused” to one or more aromatic or non-aromatic carbocyclic or heterocyclic rings or combinations thereof, as long as the radical or point of attachment is on the non-aromatic carbocyclic ring.

“Cycloalkyl”, as used herein, refers to a ring system in which is completely saturated and which has a single point of attachment to the rest of the molecule. Unless otherwise specified, a cycloalkyl group may be monocyclic, bicyclic, tricyclic, fused, spiro or bridged. In one embodiment, the term “cycloalkyl” refers to a monocyclic C₃-C₁₂ saturated hydrocarbon or a bicyclic C₇-C₁₂ saturated hydrocarbon. In some embodiments, any individual ring in a bicyclic or tricyclic ring system has 3-7 members. Suitable cycloalkyl groups include, but are not limited to cyclopropyl, cyclobutyl, cyclopentyl, cyclohexyl, cycloheptyl, cycloheptenyl, norbornyl, cyclooctyl, cyclononyl, cyclodecyl, cycloundecyl, cyclododecyl, and the like.

“Heterocycle” (or “heterocyclyl” or “heterocyclic), as used herein, refers to a ring system in which one or more ring members are an independently selected heteroatom, which is completely saturated or that contains one or more units of unsaturation but which is not aromatic, and which has a single point of attachment to the rest of the molecule. Unless otherwise specified, through this disclosure, heterocycle is used as a synonym of “non-aromatic heterocycle”. In some instances the term can be used in the phrase “aromatic heterocycle”, and in this case it refers to a “heteroaryl group” as defined below. The term heterocycle also includes fused, spiro or bridged heterocyclic ring systems. Unless otherwise specified, a heterocycle may be monocyclic, bicyclic or tricyclic. In some embodiments, the heterocycle has 3-18 ring members in which one or more ring members is a heteroatom independently selected from oxygen, sulfur or nitrogen, and each ring in the system contains 3 to 7 ring members. In other embodiments, a heterocycle may be a monocycle having 3-7 ring members (2-6 carbon atoms and 1-4 heteroatoms) or a bicycle having 7-10 ring members (4-9 carbon atoms and 1-6 heteroatoms). Examples of bicyclic heterocyclic ring systems include, but are not limited to: adamantanyl, 2-oxa-bicyclo[2.2.2]octyl, 1-aza-bicyclo[2.2.2]octyl.

As used herein, the term “heterocycle” also includes polycyclic ring systems wherein the heterocyclic ring is fused with one or more aromatic or non-aromatic carbocyclic or heterocyclic rings, or with combinations thereof, as long as the radical or point of attachment is on the heterocyclic ring.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

As used herein, the term “aryl” (as in “aryl ring” or “aryl group”), used alone or as part of a larger moiety, as in “aralkyl”, “aralkoxy”, “aryloxyalkyl”, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. The term also includes polycyclic ring systems where the aryl ring is fused with one or more aromatic or non-aromatic carbocyclic or heterocyclic rings, or with combinations thereof, as long as the radical or point of attachment is in the aryl ring. Examples of aryl rings include, but are not limited to, phenyl, naphthyl, indanyl, indenyl, tetralin, fluorenyl, and anthracenyl.

The term “aralkyl” refers to a radical having an aryl ring substituted with an alkylene group, wherein the open end of the alkylene group allows the aralkyl radical to bond to another part of the compound of Formula I. The alkylene group is a bivalent, straight-chain or branched, saturated hydrocarbon group. As used herein, the term “C₇₋₁₂ aralkyl” means an aralkyl radical wherein the total number of carbon atoms in the aryl ring and the alkylene group combined is 7 to 12. Examples of “aralkyl” include, but not limited to, a phenyl ring substituted by a C₁₋₆ alkylene group, e.g., benzyl and phenylethyl, and a naphthyl group substituted by a C₁₋₂ alkylene group.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. Bicyclic 6, 5 heteroaromatic system, as used herein, for example, is a six membered heteroaromatic ring fused to a second five membered ring wherein the radical or point of attachment is on the six-membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo” (or “cyclic”, or “cyclic moiety”) encompasses mono-, bi- and tri-cyclic ring systems including cycloaliphatic, heterocyclic, aryl or heteroaryl, each of which has been previously defined.

“Fused” bicyclic ring systems comprise two rings which share two adjoining ring atoms.

“Bridged” bicyclic ring systems comprise two rings which share three or four adjacent ring atoms. As used herein, the term “bridge” refers to an atom or a chain of atoms connecting two different parts of a molecule. The two atoms that are connected through the bridge (usually but not always, two tertiary carbon atoms) are referred to as “bridgeheads”. In addition to the bridge, the two bridgeheads are connected by at least two individual atoms or chains of atoms. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. “Spiro” bicyclic ring systems share only one ring atom (usually a quaternary carbon atom) between the two rings.

The term “ring atom” refers to an atom such as C, N, O or S that is part of the ring of an aromatic ring, a cycloaliphatic ring, a heterocyclic or a heteroaryl ring. A “substitutable ring atom” is a ring carbon or nitrogen atom bonded to at least one hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to one or more moiety other than hydrogen and no hydrogens are available for substitution.

“Heteroatom” refers to one or more of oxygen, sulfur, nitrogen, phosphorus, or silicon, including any oxidized form of nitrogen, sulfur, phosphorus, or silicon, the quaternized form of any basic nitrogen, or a substitutable nitrogen of a heterocyclic or heteroaryl ring, for example N (as in 3,4-dihydro-2H-pyrrolyl), NH (as in pyrrolidinyl) or NR⁺ (as in N-substituted pyrrolidinyl).

In some embodiments, two independent occurrences of a variable may be taken together with the atom(s) to which each variable is bound to form a 5-8-membered, heterocyclyl, aryl, or heteroaryl ring or a 3-8-membered cycloaliphatic ring. Exemplary rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, cycloaliphatic or aryl ring, wherein the group is attached to the rest of the molecule by a single point of attachment; and b) two independent occurrences of a substituent that are bound to different atoms and are taken together with both of those atoms to form a heterocyclyl, heteroaryl, cycloaliphatic or aryl ring, wherein the ring that is formed has two points of attachment with the rest of the molecule. For example, where a phenyl group is substituted with two occurrences of —OR^{o} as in Formula D1:

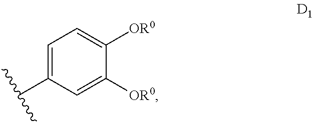

these two occurrences of —OR^{o} are taken together with the carbon atoms to which they are bound to form a fused 6-membered oxygen containing ring as in Formula D2:

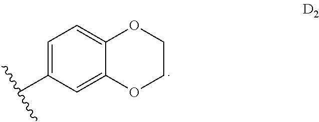 [SMILES: *c1ccc2c(c1)OCCO2 ; "."]

It will be appreciated that a variety of other rings can be formed when two independent occurrences of a substituent are taken together with the atom(s) to which each substituent is bound and that the examples detailed above are not intended to be limiting.

In some embodiments, an alkyl or aliphatic chain can be optionally interrupted with another atom or group. This means that a methylene unit of the alkyl or aliphatic chain can optionally be replaced with said other atom or group. Unless otherwise specified, the optional replacements form a chemically stable compound. Optional interruptions can occur both within the chain and/or at either end of the chain; i.e. both at the point of attachment(s) to the rest of the molecule and/or at the terminal end. Two optional replacements can also be adjacent to each other within a chain so long as it results in a chemically stable compound. Unless otherwise specified, if the replacement or interruption occurs at a terminal end of the chain, the replacement atom is bound to an H on the terminal end. For example, if —CH₂CH₂CH₃ were optionally interrupted with —O—, the resulting compound could be —OCH₂CH₃, —CH₂OCH₃, or —CH₂CH₂OH. In another example, if the divalent linker —CH₂CH₂CH₂— were optionally interrupted with —O—, the resulting compound could be —OCH₂CH₂—, —CH₂OCH₂—, or —CH₂CH₂O—. The optional replacements can also completely replace all of the carbon atoms in a chain. For example, a C₃ aliphatic can be optionally replaced by —N(R′)—, —C(O)—, and —N(R′)— to form —N(R′)C(O)N(R′)— (a urea).

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent at the end of the substituent bound to the rest of the chemical structure. Alkylcarboxy (e.g., alkyl-C(O)O— or alkyl-O(CO)—) and alkylcarboxyaryl (e.g., alkyl-C(O)O-aryl- or alkyl-O(CO)-aryl-) are examples of carboxy groups used internally.

As described herein, a bond drawn from a substituent to the center of one ring within a multiple-ring system (as shown below), represents substitution of the substituent at any substitutable position in any of the rings within the multiple ring system. For example, formula D3 represents possible substitution in any of the positions shown in formula D4:

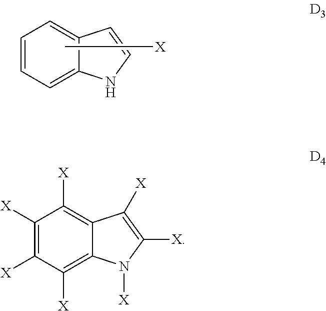

This also applies to multiple ring systems fused to optional ring systems (which would be represented by dotted lines). For example, in Formula D5, X is an optional substituent both for ring A and ring B.

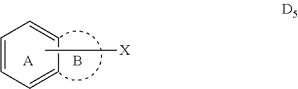

If, however, two rings in a multiple ring system each have different substituents drawn from the center of each ring, then, unless otherwise specified, each substituent only represents substitution on the ring to which it is attached. For example, in Formula D6, Y is an optional substituent for ring A only, and X is an optional substituent for ring B only.

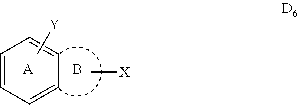

As used herein, the terms “alkoxy” or “alkylthio” refer to an alkyl group, as previously defined, attached to the molecule, or to another chain or ring, through an oxygen (“alkoxy” i.e., —O-alkyl) or a sulfur (“alkylthio” i.e., —S-alkyl) atom.

The terms Cₙ₋ₘ “alkoxyalkyl”, Cₙ₋ₘ “alkoxyalkenyl”, Cₙ₋ₘ “alkoxyaliphatic”, and Cₙ₋ₘ “alkoxyalkoxy” mean alkyl, alkenyl, aliphatic or alkoxy, as the case may be, substituted with one or more alkoxy groups, wherein the combined total number of carbons of the alkyl and alkoxy groups, alkenyl and alkoxy groups, aliphatic and alkoxy groups or alkoxy and alkoxy groups, combined, as the case may be, is between the values of n and m. For example, a C₄₋₆ alkoxyalkyl has a total of 4-6 carbons divided between the alkyl and alkoxy portion; e.g. it can be —CH₂OOCH₂CH₂CH₃, —CH₂CH₂OCH₂CH₃ or —CH₂CH₂CH₂OCH₃.

When the moieties described in the preceding paragraph are optionally substituted, they can be substituted in either or both of the portions on either side of the oxygen or sulfur. For example, an optionally substituted C₄ alkoxyalkyl could be, for instance, —CH₂CH₂OCH₂(Me)CH₃ or —CH₂(OH)OCH₂CH₂CH₃; a C₅ alkoxyalkenyl could be, for instance, —CH═CHO CH₂CH₂CH₃ or —CH═CHCH₂OOCH₂CH₃.

The terms aryloxy, arylthio, benzyloxy or benzylthio, refer to an aryl or benzyl group attached to the molecule, or to another chain or ring, through an oxygen (“aryloxy”, benzyloxy e.g., —O-Ph, —OCH₂Ph) or sulfur (“arylthio” e.g., —S-Ph, —S—CH₂Ph) atom. Further, the terms “aryloxyalkyl”, “benzyloxyalkyl” “aryloxyalkenyl” and “aryloxyaliphatic” mean alkyl, alkenyl or aliphatic, as the case may be, substituted with one or more aryloxy or benzyloxy groups, as the case may be. In this case, the number of atoms for each aryl, aryloxy, alkyl, alkenyl or aliphatic will be indicated separately. Thus, a 5-6-membered aryloxy(C₁₋₄alkyl) is a 5-6 membered aryl ring, attached via an oxygen atom to a C₁₋₄ alkyl chain which, in turn, is attached to the rest of the molecule via the terminal carbon of the C₁₋₄ alkyl chain.

As used herein, the terms “halogen” or “halo” mean F, Cl, Br, or I.

The terms “haloalkyl”, “haloalkenyl”, “haloaliphatic”, and “haloalkoxy” mean alkyl, alkenyl, aliphatic or alkoxy, as the case may be, substituted with one or more halogen atoms. For example a C₁₋₃ haloalkyl could be —CFHCH₂CHF₂ and a C₁₋₂ haloalkoxy could be —OC(Br)HCHF₂. This term includes perfluorinated alkyl groups, such as —CF₃ and —CF₂CF₃.

As used herein, the term “cyano” refers to —CN or —C≡N.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. For example a C₁₋₃ cyanoalkyl could be —C(CN)₂CH₂CH₃ and a C₁₋₂ cyanoalkenyl could be ═CHC(CN)H₂.

As used herein, an “amino” group refers to —NH₂.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups. For example a C₁₋₃ aminoalkyl could be —CH(NH₂)CH₂CH₂NH₂ and a C₁₋₂ aminoalkoxy could be —OCH₂CH₂NH₂.

The term “hydroxyl” or “hydroxy” refers to —OH.

The terms “hydroxyalkyl”, “hydroxyalkenyl”, “hydroxyaliphatic”, and “hydroxyalkoxy” mean alkyl, alkenyl, aliphatic or alkoxy, as the case may be, substituted with one or more —OH groups. For example a C₁₋₃ hydroxyalkyl could be —CH₂(CH₂OH)CH₃ and a C₄ hydroxyalkoxy could be —OCH₂C(CH₃)(OH)CH₃.

As used herein, a “carbonyl”, used alone or in connection with another group refers to —C(O)— or —C(O)H. For example, as used herein, an “alkoxycarbonyl,” refers to a group such as —C(O)O(alkyl).

As used herein, an “oxo” refers to ═O, wherein oxo is usually, but not always, attached to a carbon atom (e.g., it can also be attached to a sulfur atom). An aliphatic chain can be optionally interrupted by a carbonyl group or can optionally be substituted by an oxo group, and both expressions refer to the same: e.g. —CH₂—C(O)—CH₃.

As used herein, in the context of resin chemistry (e.g. using solid resins or soluble resins or beads), the term “linker” refers to a bifunctional chemical moiety attaching a compound to a solid support or soluble support.

In all other situations, a “linker”, as used herein, refers to a divalent group in which the two free valences are on different atoms (e.g. carbon or heteroatom) or are on the same atom but can be substituted by two different substituents. For example, a methylene group can be C₁ alkyl linker (—CH₂—) which can be substituted by two different groups, one for each of the free valences (e.g. as in Ph-CH₂-Ph, wherein methylene acts as a linker between two phenyl rings). Ethylene can be C₂ alkyl linker (—CH₂CH₂—) wherein the two free valences are on different atoms. The amide group, for example, can act as a linker when placed in an internal position of a chain (e.g. —CONH—). A linker can be the result of interrupting an aliphatic chain by certain functional groups or of replacing methylene units on said chain by said functional groups. E.g. a linker can be a C₁₋₆ aliphatic chain in which up to two methylene units are substituted by —C(O)— or —NH— (as in —CH₂—NH—CH₂—C(O)—CH₂— or —CH₂—NH—C(O)—CH₂—). An alternative way to define the same —CH₂—NH—CH₂—C(O)—CH₂— and —CH₂—NH—C(O)—CH₂— groups is as a C₃ alkyl chain optionally interrupted by up to two —C(O)— or —NH— moieties. Cyclic groups can also form linkers: e.g. a 1,6-cyclohexanediyl can be a linker between two R groups, as in

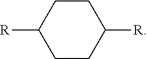

A linker can additionally be optionally substituted in any portion or position.

Divalent groups of the type R—CH═ or R₂C═, wherein both free valences are in the same atom and are attached to the same substituent, are also possible. In this case, they will be referred to by their IUPAC accepted names. For instance an alkylidene (such as, for example, a methylidene (═CH₂) or an ethylidene (═CH—CH₃)) would not be encompassed by the definition of a linker in this disclosure.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W. et al., “Protective Groups in Organic Synthesis”, Third Edition, John Wiley & Sons, New York: 1999, the entire contents of which is hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

As used herein, “amide coupling agent” or “amide coupling reagent” means a compound that reacts with the hydroxyl moiety of a carboxy moiety thereby rendering it susceptible to nucleophilic attack. Exemplary amide coupling agents include DIC (diisopropylcarbodiimide), EDCI (1-ethyl-3-(3-dimethylaminopropyl)carbodiimide), DCC (dicyclohexylcarbodiimide), BOP (benzotriazol-1-yloxy-tris(dimethylamino)-phosphonium hexafluorophosphate), pyBOP ((benzotriazol-1-yloxy)tripyrrolidinophosphonium hexafluorophosphate), etc.

The compounds of the invention are defined herein by their chemical structures and/or chemical names. Where a compound is referred to by both a chemical structure and a chemical name, and the chemical structure and chemical name conflict, the chemical structure is determinative of the compound's identity.

### Pharmaceutically Acceptable Salts

In some embodiments of the methods, uses, pharmaceutical compositions and kits, the sGC stimulator may be provided as (i) the compound itself (e.g., as the free base); (ii) a pharmaceutically acceptable salt of the compound; or (iii) part of a pharmaceutical composition. In some embodiments of the above methods, uses, pharmaceutical compositions and kits, the additional therapeutic agent may be provided as (i) the compound itself (e.g., as the free base); (ii) a pharmaceutically acceptable salt of the compound; (iii) or part of a pharmaceutical composition.

The phrase “pharmaceutically acceptable salt,” as used herein, refers to pharmaceutically acceptable organic or inorganic salts of a compound described herein. For use in medicine, the salts of the compounds described herein will be pharmaceutically acceptable salts. Other salts may, however, be useful in the preparation of the compounds described herein or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound described herein is acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N, N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound described herein is basic or contains a sufficiently basic bioisostere, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated herein by reference in its entirety. Compounds, compositions and kits of the invention are also useful for veterinary treatment of companion animals, exotic animals and farm animals, including, without limitation, dogs, cats, mice, rats, hamsters, gerbils, guinea pigs, rabbits, horses, pigs and cattle.

### Methods of Administration and Co-Administration

In some embodiments of the above methods and uses, the sGC stimulator is administered before a symptom of a neuromuscular disorder, (e.g., Muscular Dystrophy, DMD or BMD) fully develops in said patient. In other embodiments of the above methods and uses, the sGC stimulator is administered after one or more symptoms of a neuromuscular disorder (e.g., Muscular Dystrophy, DMD or BMD) develops in said patient.

As used herein, the terms “in combination” or “co-administration” can be used interchangeably to refer to the use of more than one therapy (e.g., a sGC stimulator and one or more additional therapeutic agents). The use of the terms does not restrict the order in which therapies (e.g., the sGC stimulator and the additional therapeutic agents) are administered to a subject.

In some embodiments, the sGC stimulator is administered prior to, at the same time or after the initiation of treatment with another therapeutic agent.

In some embodiments of the above methods and uses, the additional therapeutic agent and the sGC stimulator are administered simultaneously. In other embodiments of the above methods and uses, the additional therapeutic agent and the sGC stimulator are administered sequentially or separately.

In some embodiments, the above pharmaceutical compositions or kits comprise (a) an sGC stimulator as discussed above or a pharmaceutically acceptable salt thereof, and (b) a pharmaceutically acceptable carrier, vehicle or adjuvant. In some embodiments, the pharmaceutical composition or kit comprises (a) one or more additional therapeutic agents as discussed above, or a pharmaceutically acceptable salt thereof, and (b) a pharmaceutically acceptable carrier, vehicle or adjuvant. In some embodiments, the pharmaceutical composition comprises (i) an sGC stimulator as discussed above, or a pharmaceutically acceptable salt thereof, (ii) one or more additional therapeutic agents as discussed above, or a pharmaceutically acceptable salt thereof, and (iii) a pharmaceutically acceptable carrier, vehicle or adjuvant.

The sGC stimulators and pharmaceutical compositions described herein can be used in combination therapy with one or more additional therapeutic agents. For combination treatment with more than one active agent, the additional active agents may be in the same dosage form or in separate dosage forms. Wherein the additional active agents are present in separate dosage forms, the active agents may be administered separately or in conjunction with the sGC stimulator. In addition, the administration of one agent may be prior to, concurrent to, or subsequent to the administration of the other agent.

When co-administered with other agents, e.g., when co-administered with another sGC stimulator, steroid, etc, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.001 to about 100 mg/kg body weight/day, from about 0.001 to about 50 mg/kg body weight/day, from about 0.001 to about 30 mg/kg body weight/day, from about 0.001 to about 10 mg/kg body weight/day.

When “combination therapy” is employed, an effective amount can be achieved using a first amount of an sGC stimulator or a pharmaceutically acceptable salt thereof and a second amount of an additional suitable therapeutic agent (e.g. another sGC stimulator, a steroid, a NO modulator, a cGMP modulator, a therapeutic that increases the function or localization of dystrophin, etc.).

In one embodiment of this invention, the sGC stimulator and the additional therapeutic agent, are each administered in an effective amount (i.e., each in an amount which would be therapeutically effective if administered alone). In another embodiment, the sGC stimulator and the additional therapeutic agent, are each administered in an amount which alone does not provide a therapeutic effect (“a sub-therapeutic dose”). In yet another embodiment, the sGC stimulator can be administered in an effective amount, while the additional therapeutic agent is administered in a sub-therapeutic dose. In still another embodiment, the sGC stimulator can be administered in a sub-therapeutic dose, while the additional therapeutic agent, for example, a suitable anti-inflammatory agent is administered in an effective amount.

“Co-administration” encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, co-administration also encompasses use of each compound in a sequential manner in either order. When co-administration involves the separate administration of the first amount of a sGC stimulator and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a sGC stimulator and the second therapeutic agent can be administered in any order within about 24 hours of each other, within about 16 hours of each other, within about 8 hours of each other, within about 4 hours of each other, within about 1 hour of each other or within about 30 minutes of each other, within about 5 minutes of each other, etc.

More, specifically, a first therapy (e.g., a prophylactic or therapeutically used sGC stimulator) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks prior to), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks subsequent to) the administration of a second therapy (e.g., an additional therapeutic agent or prophylactic agent described herein) to a subject.

In some embodiments of the above methods, uses, compositions and kits, the additional therapeutic agent or agents may be selected from one or more of the following:

(1a) endothelium-derived releasing factor (EDRF); nitric oxide;

(1b) corticosteroids such as beclomethasone, methylprednisolone, betamethasone, prednisone, deflazacort, CAT-1004 (from Catabasis), prednisolone, triamcinolone, dexamethasone, fluticasone, flunisolide and hydrocortisone, and corticosteroid analogs such as budesonide;

(2) nitric oxide (NO) donors such as a nitrosothiol, a nitrite, a sydnonimine, a NONOate, a N-nitrosamine, a N-hydroxyl nitrosamine, a nitrosimine, nitrotyrosine, a diazetine dioxide, an oxatriazole 5-imine, an oxime, a hydroxylamine, a N-hydroxyguanidine, a hydroxyurea or a furoxan. Some examples of these types of compounds include: glyceryl trinitrate (also known as GTN, nitroglycerin, nitroglycerine, and trinitroglycerin), the nitrate ester of glycerol; sodium nitroprusside (SNP), wherein a molecule of nitric oxide is coordinated to iron metal forming a square bipyramidal complex; 3-morpholinosydnonimine (SIN-1), a zwitterionic compound formed by combination of a morpholine and a sydnonimine; S-nitroso-N-acetylpenicillamine (SNAP), an N-acetylated amino acid derivative with a nitrosothiol functional group; diethylenetriamine/NO (DETA/NO), a compound of nitric oxide covalently linked to diethylenetriamine; and NCX 4016, a m-nitroxymethyl phenyl ester of acetyl salicylic acid; other NO donors including the classic nitrovasodilators, such as organic nitrate and nitrite esters, nitroglycerin, amyl nitrite, isosorbide dinitrate, isosorbide 5-mononitrate, and nicorandil; isosorbide (Dilatrate®-SR, Imdur®, Ismo®, Isordil®, Isordil®, Titradose®, Monoket®), FK 409 (NOR-3, a non-thiol NO donor); FR 144420 (NOR-4); 3-morpholinosydnonimine; Linsidomine chlorohydrate (“SIN-1”); AZD3582 (CINOD lead compound), NCX 4016, NCX 701, NCX 1022, HCT 1026, NCX 1015, NCX 950, NCX 1000, NCX 1020, AZD 4717, NCX 1510/NCX 1512, NCX 2216, and NCX 4040 (all available from NicOx S.A.), S-nitrosoglutathione (GSNO), S-nitrosoglutathione mono-ethyl-ester (GSNO-ester), 6-(2-hydroxy-1-methyl-nitrosohydrazino)-N-methyl-1-hexanamine (NOC-9) or diethylamine NONOate; nitric oxide donors disclosed in U.S. Pat. Nos. 5,155,137, 5,366,997, 5,405,919, 5,650,442, 5,700,830, 5,632,981, 6,290,981, 5,691,423 5,721,365, 5,714,511, 6,511,911, and 5,814,666, Chrysselis et al. (2002) J Med Chem. 45:5406-9 (such as NO donors 14 and 17), and Nitric Oxide Donors for Pharmaceutical and Biological Research, Eds: Peng George Wang, Tingwei Bill Cai, Naoyuki Taniguchi, Wiley, 2005;

(3) other substances that enhance cGMP concentrations such as protoporphyrin IX, arachidonic acid and phenyl hydrazine derivatives;
(4) Nitric Oxide Synthase substrates such as n-hydroxyguanidine based analogs, such as N[G]-hydroxy-L-arginine (NOHA), 1-(3, 4-dimethoxy-2-chlorobenzylideneamino)-3-hydroxyguanidine, and PR5 (1-(3, 4-dimethoxy-2-chlorobenzylideneamino)-3-hydroxyguanidine); L-arginine; L-arginine derivatives (such as homo-Arg, homo-NOHA, N-tert-butyloxy- and N-(3-methyl-2-butenyl)oxy-L-arginine, canavanine, epsilon guanidine-carpoic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear —F, —Cl, -methyl, —OH substituents, respectively); guanidine derivatives such as 3-(trifluoromethyl) propylguanidine; and others reviewed in Cali et al. (2005, Current Topics in Medicinal Chemistry 5:721-736) and disclosed in the references cited therein;

(5) compounds which enhance eNOS or nNOS transcription: for example those described in WO 02/064146, WO 02/064545, WO 02/064546 and WO 02/064565, and corresponding patent publications US2003/0008915, US2003/0022935, US2003/0022939 and US2003/0055093; other eNOS transcriptional enhancers including those described in US20050101599 (e.g. 2,2-difluorobenzo[1,3]dioxol-5-carboxylic acid indan-2-ylamide, and 4-fluoro-N-(indan-2-yl)-benzamide), and Sanofi-Aventis compounds AVE3085 and AVE9488 (CA Registry NO. 916514-70-0; Schafer et al., Journal of Thrombosis and Homeostasis 2005; Volume 3, Supplement 1: abstract number P 1487);

(6) NO independent heme-independent sGC activators, including, but not limited to: BAY 58-2667 (cinaciguat, described in patent publication DE19943635)

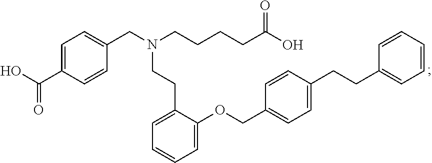 [SMILES: O=C(O)CCCCN(CCc1ccccc1OCc1ccc(CCc2ccccc2)cc1)Cc1ccc(C(=O)O)cc1 ; ";"]

HMR-1766 (ataciguat sodium, described in patent publication WO2000002851)

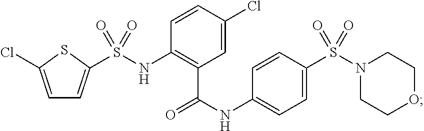 [SMILES: O=C(Nc1ccc(S(=O)(=O)N2CCOCC2)cc1)c1cc(Cl)ccc1NS(=O)(=O)c1ccc(Cl)s1]

S 3448 (2-(4-chloro-phenylsulfonylamino)-4,5-dimethoxy-N-(4-(thiomorpholine-4-sulfonyl)-phenyl)-benzamide (see patent publications DE19830430 and WO2000002851)

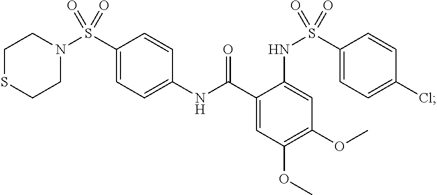 [SMILES: COc1cc(NS(=O)(=O)c2ccc(Cl)cc2)c(C(=O)Nc2ccc(S(=O)(=O)N3CCSCC3)cc2)cc1OC]

### HMR-1069 (Sanofi-Aventis).

(7) heme-dependent sGC stimulators including, but not limited to:

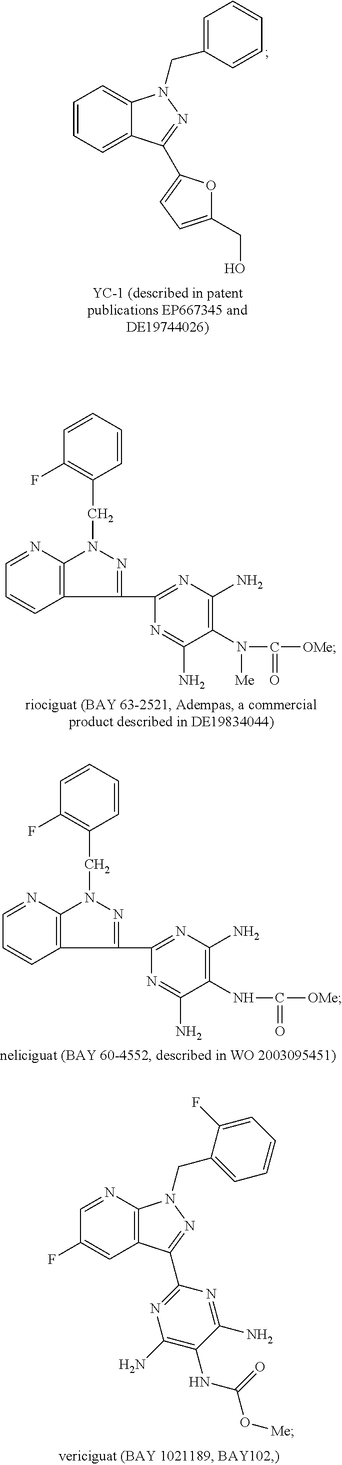 [SMILES: OCc1ccc(-c2nn(Cc3ccccc3)c3ccccc23)o1 ; COC(=O)N(C)c1c(N)nc(-c2nn(Cc3ccccc3F)c3ncccc23)nc1N ; COC(=O)Nc1c(N)nc(-c2nn(Cc3ccccc3F)c3ncccc23)nc1N ; COC(=O)Nc1c(N)nc(-c2nn(Cc3ccccc3F)c3ncc(F)cc23)nc1N]

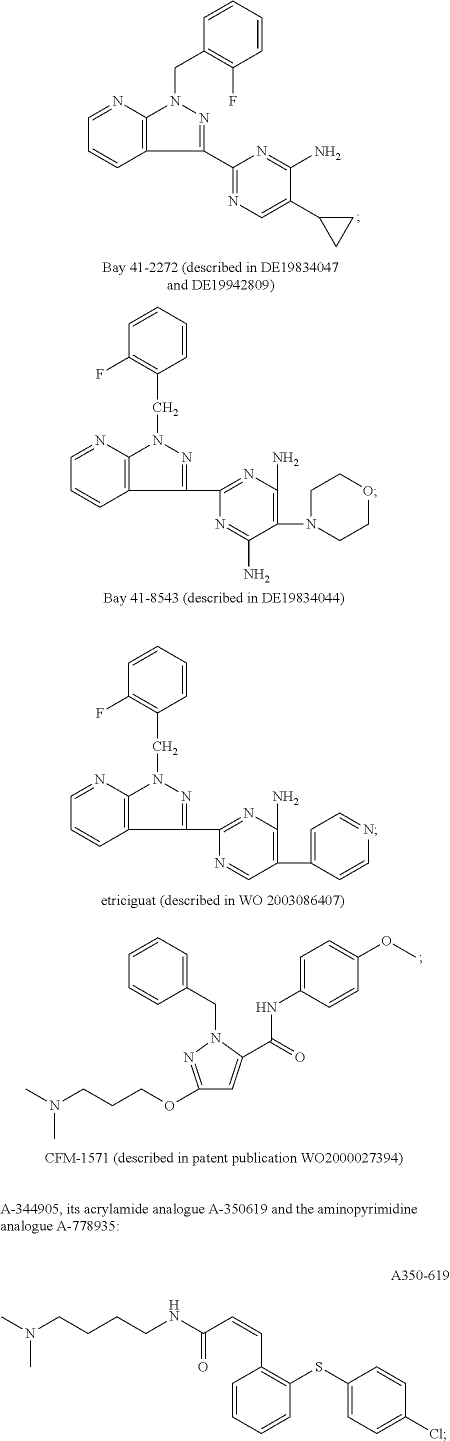

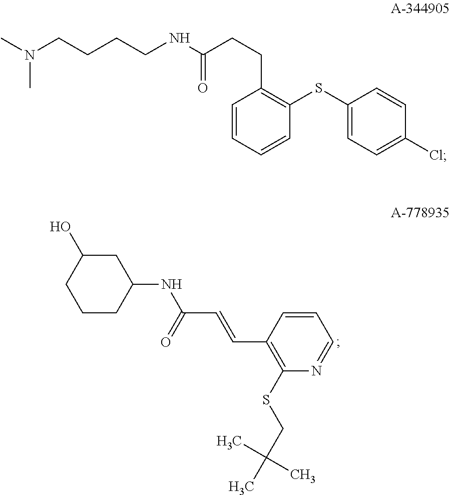 [SMILES: CN(C)CCCCNC(=O)CCc1ccccc1Sc1ccc(Cl)cc1 ; CC(C)(C)CSc1ncccc1/C=C/C(=O)NC1CCCC(O)C1]

or compounds disclosed in one of publications:

US20140088080 (WO2012165399), WO2014084312, U.S. Pat. No. 6,414,009, U.S. Pat. No. 6,462,068, U.S. Pat. No. 6,387,940, U.S. Pat. No. 6,410,740 (WO 98 16507), U.S. Pat. No. 6,451,805 (WO 98 23619), U.S. Pat. No. 6,180,656 (WO 98 16223), US20040235863 (WO2003004503), US 20060052397, U.S. Pat. No. 7,173,037 (WO2003095451), US 20060167016, U.S. Pat. No. 7,091,198 (WO2004009589), US 20060014951, U.S. Pat. No. 7,410,973 (WO2004009590), US 20100004235 (WO2007124854, e.g., Examples 1, 2, 3, 6, 7, 18 or 19), US20100029653 (WO 2008031513, e.g., Examples 1, 2, 3, 4 or 7), US20100113507 (WO2007128454, e.g, Example 1, 4 or 7), US 20110038857, U.S. Pat. No. 8,114,400 (WO2008061657), US20110218202 (WO 2010065275, e.g., Examples 1, 3, 59, 60 or 111), US20110245273 (WO 2010078900, e.g., Examples 1 or 5), US2012029002 (WO 2010079120), US20120022084, US 20130237551, U.S. Pat. No. 8,420,656 (WO 2011147809, WO 2011147810), US20130210824 (WO2013104598), US20130172372 (WO2012004259, e.g., Examples 2, 3 or 4), US20130267548 (WO2012059549, e.g., Examples 1, 2, 7, 8 or 13), WO 2012143510 (e.g., Examples 1, 2, 3, 4, 5, 6, 7, 8, 9 or 10), WO2012004258 (e.g., Examples 1, 18, 19 or 27), WO2012152629 (e.g., Examples 11 or 12), WO2012152630 (e.g., Examples 1, 5, 8, 11, 15 or 19), WO2012010577 (e.g., Examples 3-1, 4, 5 or 6), WO2012028647 (e.g., Examples 1, 2 or 3), WO2013104597 (e.g., Examples 16, 18, 22 or 23), WO2013131923 (e.g., Examples 1, 2, 7, 8 or 9), WO2013104703, WO2013004785 (e.g., Examples 1, 3 or 6), WO2013030288, US20090209556, U.S. Pat. No. 8,455,638, US20110118282 (WO2009032249), US20100292192, US20110201621, U.S. Pat. No. 7,947,664, U.S. Pat. No. 8,053,455 (WO2009094242), US20100216764, U.S. Pat. No. 8,507,512 (WO2010099054), US20110218202 (WO2010065275), US20130012511 (WO2011119518), US20130072492 (WO2011149921, e.g., Example #160, Example #164 and Example #181), US20130210798 (WO2012058132), U.S. Pat. No. 8,796,305 (WO2014068095), US20140128372 and US20140179672 (WO2014068099), U.S. Pat. No. 8,778,964 (US20140128386, US20140128424, WO2014068104), WO2014131741 and US20140249168 (WO2014131760);

(8) compounds that inhibit the degradation of cGMP, such as:

PDE5 inhibitors, such as, for example, sildenafil (Viagra®) and other related agents such as avanafil, lodenafil, mirodenafil, sildenafil citrate (Revatio®), tadalafil (Cialis® or Adcirca®), vardenafil (Levitra®) and udenafil; alprostadil; and dipyridamole;

(9) calcium channel blockers such as:

dihydropyridine calcium channel blockers: amlodipine (Norvasc®), aranidipine (Sapresta®), azelnidipine (Calblock®), barnidipine (HypoCa®), benidipine (Coniel®), cilnidipine (Atelec®, Cinalong®, Siscard®), clevidipine (Cleviprex®), diltiazem, efonidipine (Landel®), felodipine (Plendil®), lacidipine (Motens®, Lacipil®), lercanidipine (Zanidip®), manidipine (Calslot®, Madipine®), nicardipine (Cardene®, Carden SR®), nifedipine (Procardia®, Adalat®), nilvadipine (Nivadil®), nimodipine (Nimotop®), nisoldipine (Baymycard®, Sular®, Syscor®), nitrendipine (Cardif®, Nitrepin®, Baylotensin®), pranidipine (Acalas®), isradipine (Lomir®);

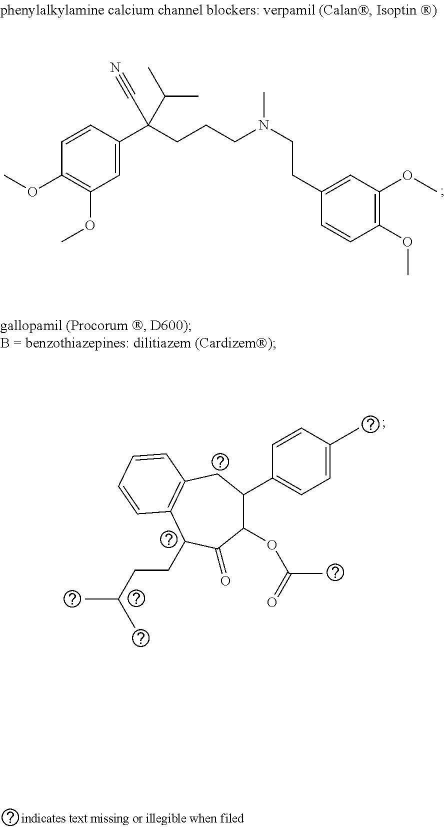

and nonselective calcium channel inhibitors such as: mibefradil, bepridil, fluspirilene and fendiline;

(10) endothelin receptor antagonists (ERAs): for instance the dual (ET_{A} and ET_{B}) endothelin receptor antagonist bosentan (marketed as Tracleer®); sitaxentan, marketed under the name Thelin®; ambrisentan marketed as Letairis® in U.S; and dual/nonselective endothelin antagonist Actelion-1, that entered clinical trials in 2008;

(11) prostacyclin derivatives or analogues: for instance prostacyclin (prostaglandin I₂), epoprostenol (synthetic prostacyclin, marketed as Flolan®); treprostinil (Remodulin®), iloprost (Ilomedin®), iloprost (marketed as Ventavis®); oral and inhaled forms of Remodulin® that are under development; beraprost, an oral prostanoid available in Japan and South Korea;

(12) antihyperlipidemics such as: bile acid sequestrants (e.g., cholestyramine, colestipol, colestilan and colesevelam); statins such as atorvastatin, simvastatin, lovastatin, fluvastatin, pitavastatin, rosuvastatin and pravastatin; cholesterol absorption inhibitors such as Ezetimibe; other lipid lowering agents such as icosapent ethyl ester, omega-3-acid ethyl esters, Reducol™; fibric acid derivatives such clofibrate, bezafibrate, clinofibrate, gemfibrozil (Lopid®, Jezid®), ronifibrate, binifibrate, fenofibrate, ciprofibrate, choline fenofibrate; nicotinic acid derivatives such as acipimox and niacin; also combinations of statins, niacin, intestinal cholesterol absorption-inhibiting supplements (ezetimibe and others) and fibrates; antiplatelet therapies such as clopidogrel bisulfate;

(13) anticoagulants, such as the following types:
coumarines (Vitamin K antagonists): Warfarin® (Coumadin®) mostly used in the US and UK; acenocoumarol and phenprocoumon, mainly used in other countries; phenindione;
heparin and derivative substances such as: heparin; low molecular weight heparin, fondaparinux and idraparinux;
direct thrombin inhibitors such as: argatroban, lepirudin, bivalirudin and dabigatran; ximelagatran (Exanta®), not approved in the US;
direct Factor Xa inhibitors such as apixaban, rivaroxaban (Xarelto®);
tissue plasminogen activators, used to dissolve clots and unblock arteries, such as alteplase;

(14) antiplatelet drugs: for instance thienopyridines such as lopidogrel and ticlopidine; dipyridamole; aspirin;

(15) ACE inhibitors, for example the following types:
sulfhydryl-containing agents such as Captopril (trade name Capoten®), the first ACE inhibitor and zofenopril;
dicarboxylate-containing agents such as enalapril (Vasotec/Renitec®); ramipril (Altace/Tritace/Ramace/Ramiwin®); quinapril (Accupril®), perindopril (Coversyl/Aceon®); lisinopril (Lisodur/Lopril/Novatec/Prinivil/Zestril®) and benazepril (Lotensin®);
phosphonate-containing agents such as: fosinopril;
naturally occurring ACE inhibitors such as: casokinins and lactokinins, which are breakdown products of casein and whey that occur naturally after ingestion of milk products, especially cultured milk; the lactotripeptides Val-Pro-Pro and Ile-Pro-Pro produced by the probiotic Lactobacillus helveticus or derived from casein also have ACE-inhibiting and antihypertensive functions;
other ACE inhibitors such as alacepril, delapril, cilazapril, imidapril, trandolapril, temocapril, moexipril, spirapril;

(16) supplemental oxygen therapy;

(17) beta blockers, such as the following types:
non-selective agents: alprenolol, bucindolol, carteolol, carvedilol (has additional α-blocking activity), labetalol (has additional α-blocking activity), nadolol, penbutolol (has intrinsic sympathomimetic activity), pindolol (has intrinsic sympathomimetic activity), oxprenonol, acebutolol, sotalol, mepindolol, celiprolol, arotinolol, tertatolol, amosulalol, nipradilol, propranolol and timolol;
β₁-selective agents: acebutolol (has intrinsic sympathomimetic activity), atenolol, betaxolol, bisoprolol, celiprolol, dobutamine hydrochloride, irsogladine maleate, carvedilol, talinolol, esmolol, metoprolol and nebivolol;
β₂-selective agents: Butaxamine® (weak α-adrenergic agonist activity);

(18) antiarrhythmic agents such as the following types:
Type I (sodium channel blockers): quinidine, lidocaine, phenytoin, propafenone
Type III (potassium channel blockers): amiodarone, dofetilide, sotalol
Type V: adenosine, digoxin;

(19) diuretics such as: thiazide diuretics, e.g., chlorothiazide, chlorthalidone, and hydrochlorothiazide, bendroflumethiazide, cyclopenthiazide, methyclothiazide, polythiazide, quinethazone, xipamide, metolazone, ondapamide, cicletanine; loop diuretics, such as furosemide and toresamide; potassium-sparing diuretics such as amiloride, spironolactone, canrenoate potassium, eplerenone and triamterene; combinations of these agents; other diuretics such as acetazolamid and carperitide;

(20) direct acting vasodilators such as hydralazine hydrochloride, diazoxide, sodium nitroprusside, cadralazine; other vasodilators such as isosorbide dinitrate and isosorbide 5-mononitrate;

(21) exogenous vasodilators such as:
Adenocard®, an adenosine agonist, primarily used as an anti-arrhythmic;
alpha blockers (which block the vasoconstricting effect of adrenaline):
   alpha-1-adrenoceptor antagonists such as prazosin, indoramin, urapidil, bunazosin, terazosin, doxazosin
atrial natriuretic peptide (ANP);
ethanol;
histamine-inducers, which complement proteins C3a, C4a and C5a and work by triggering histamine release from mast cells and basophil granulocytes;
tetrahydrocannabinol (THC), major active chemical in marijuana which has minor vasodilatory effects;
papaverine, an alkaloid found in the opium poppy papaver somniferum;

(22) bronchodilators: of which there are two major types: β₂ agonists and anticholinergics, exemplified below:
β₂ agonists: salbutamol or albuterol (common brand name: Ventolin®) and terbutaline are short acting β₂ agonists for rapid relief of COPD symptoms. Long acting β₂ agonists (LABAs) such as salmeterol and formoterol;
anticholinergics: ipratropium is the most widely prescribed short acting anticholinergic drug, tiotropium, the most commonly prescribed long-acting anticholinergic drug in COPD;
Theophylline®, a bronchodilator and phosphodiesterase inhibitor;

(23) dietary supplements such as, for example: omega-3 oils; folic acid, niacin, zinc, copper, Korean red ginseng root, ginkgo, pine bark, tribulus terrestris, arginine, avena sativa, horny goat weed, maca root, muira puama, saw palmetto, and Swedish flower pollen; vitamin C, vitamin E, vitamin K2; testosterone supplements, testosterone transdermal patch; zoraxel, naltrexone, bremelanotide (formerly PT-141), melanotan II, hMaxi-K; prelox: a proprietary mix/combination of naturally occurring ingredients, L-arginine aspartate and pycnogenol;

(24) PGD2 receptor antagonists including, but not limited to, compounds described as having PGD2 antagonizing activity in United States Published Applications US20020022218, US20010051624, and US20030055077, PCT Published Applications WO9700853, WO9825919, WO03066046, WO003066047, WO03101961, WO03101981, WO04007451, WO0178697, WO04032848, WO003097042, WO03097598, WO003022814, WO003022813, and WO04058164, European Patent Applications EP945450 and EP944614, and those listed in: Torisu et al. 2004 Bioorg Med Chem Lett 14:4557, Torisu et al. 2004 Bioorg Med Chem Lett 2004 14:4891, and Torisu et al. 2004 Bioorg & Med Chem 2004 12:4685;

(25) immunosuppressants such as cyclosporine (cyclosporine A, Sandimmune® Neoral®), tacrolimus (FK-506, Prograf®), rapamycin (sirolimus, Rapamune®) and other FK-506 type immunosuppressants, and mycophenolate, e.g., mycophenolate mofetil (CellCept®);

(26) non-steroidal anti-asthmatics such as β2-agonists (e.g., terbutaline, metaproterenol, fenoterol, isoetharine, albuterol, salmeterol, bitolterol and pirbuterol) and β2-agonist-corticosteroid combinations (e.g., salmeterol-fluticasone (Advair®), formoterol-budesonid (Symbicort®)), theophylline, cromolyn, cromolyn sodium, nedocromil, atropine, ipratropium, ipratropium bromide, leukotriene biosynthesis inhibitors (zileuton, BAY1005);

(27) non-steroidal anti-inflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac), fenamic acid derivatives (e.g., flufenamic acid, meclofenamic acid, mefenamic acid, niflumic acid and tolfenamic acid), biphenylcarboxylic acid derivatives (e.g., diflunisal and flufenisal), oxicams (e.g., isoxicam, piroxicam, sudoxicam and tenoxican), salicylates (e.g., acetyl salicylic acid and sulfasalazine) and the pyrazolones (e.g., apazone, bezpiperylon, feprazone, mofebutazone, oxyphenbutazone and phenylbutazone);

(28) cyclooxygenase-2 (COX-2) inhibitors such as celecoxib (Celebrex®), rofecoxib (Vioxx®), valdecoxib, etoricoxib, parecoxib and lumiracoxib;

(opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine;

(29) anti-diabetic agents such as insulin and insulin mimetics, sulfonylureas (e.g., glyburide, glybenclamide, glipizide, gliclazide, gliquidone, glimepiride, meglinatide, tolbutamide, chlorpropamide, acetohexamide, tolazamide), biguanides, e.g., metformin (Glucophage®), α-glucosidase inhibitors (such as acarbose, epalrestat, voglibose, miglitol), thiazolidinone compounds, e.g., rosiglitazone (Avandia®), troglitazone (Rezulin®), ciglitazone, pioglitazone (Actos®) and englitazone; insulin sensitizers such as pioglitazone and rosiglitazone; insulin secretagogues such as repaglinide, nateglinide and mitiglinide; incretin mimetics such as exanatide and liraglutide; amylin analogues such as pramlintide; glucose lowering agents such as chromium picolinate (optionally combined with biotin); dipeptidyl peptidase IV inhibitors such as sitagliptin, vildagliptin, saxagliptin, alogliptin and linagliptin; vaccines currently being developed for the treatment of diabetes; AVE-0277, Alum-GAD, BHT-3021, IBC-VS01; cytokine targeted therapies in development for the treatment of diabetes such as anakinra, canakinumab, diacerein, gevokizumab, LY-2189102, MABP-1, GIT-027; other drugs in development for the treatment of diabetes;

(30) HDL cholesterol-increasing agents such as anacetrapib, MK-524A, CER-001, DRL-17822, dalcetrapib, JTT-302, RVX-000222, TA-8995;

(31) antiobesity drugs such as methamphetamine hydrochloride, amfepramone hydrochloride (Tenuate®), phentermine (Ionamin®), benzfetamine hydrochloride (Didrex®), phendimetrazine tartrate (Bontril®, Prelu-2 ®, Plegine®), mazindol (Sanorex®), orlistat (Xenical®), sibutramine hydrochloride monohydrate (Meridia®, Reductil®), rimonabant (Acomplia®), amfepramone, chromium picolinate, RM-493, TZP-301; combination such as phentermine/topiramate, bupropion/naltrexone, sibutramine/metformin, bupropion SR/zonisamide SR, salmeterol, xinafoate/fluticasone propionate; lorcaserin hydrochloride, phentermine/topiramate, pupropion/naltrexone, cetilistat, exenatide, KI-0803, liraglutide, metformin hydrochloride, sibutramine/metformin, 876167, ALS-L-1023, bupropion SR/zonisamide SR, CORT-108297, canagliflozin, chromium picolinate, GSK-1521498, LY-377604, metreleptin, obinepitide, P-57AS3, PSN-821, salmeterol xinafoate/fluticasone propionate, sodium tungstate, somatropin (recombinant), TM-30339, TTP-435, tesamorelin, tesofensine, velneperit, zonisamide, BMS-830216, ALB-127158, AP-1030, ATHX-105, AZD-2820, AZD-8329, beloranib hemioxalate, CP-404, HPP-404, ISIS-FGFR4Rx, insulinotropin, KD-3010PF, 05212389, PP-1420, PSN-842, peptide YY3-36, resveratrol, S-234462; S-234462, sobetirome, TM-38837, Tetrahydrocannabivarin, ZYO-1, beta-lapachone;

(32) angiotensin receptor blockers such as losartan, valsartan, candesartan cilexetil, eprosaran, irbesartan, telmisartan, olmesartran medoxomil, azilsartan medoxomil;

(33) renin inhibitors such as aliskiren hemifumirate;

(34) centrally acting alpha-2-adrenoceptor agonists such as methyldopa, clonidine, guanfacine;

(35) adrenergic neuron blockers such as guanethidine, guanadrel;

(36) imidazoline I-1 receptor agonists such as rimenidine dihydrogen phosphate and moxonidine hydrochloride hydrate;

(37) aldosterone antagonists such as spironolactone and eplerenone;

(38) potassium channel activators such as pinacidil;

(39) dDopamine D1 agonists such as fenoldopam mesilate; other dopamine agonists such as ibopamine, dopexamine and docarpamine;

(40) 5-HT2 antagonists such as ketanserin;

(41) drugs that are currently being developed for the treatment of arterial hypertension;

(42) vasopressin antagonists such as tolvaptan;

(43) calcium channel sensitizers such as levosimendan or activators such as nicorandil;

(44) PDE-3 inhibitors such as amrinone, milrinone, enoximone, vesnarinone, pimobendan, olprinone;

(45) adenylate cyclase activators such as colforsin dapropate hydrochloride;

(46) positive inotropic agents such as digoxin and metildigoxin; metabolic cardiotonic agents such as ubidecarenone; brain naturetic peptides such as nesiritide;

(47) drugs that are currently in development for the treatment of heart failure;

(48) drugs currently in development for the treatment of pulmonary hypertension;

(49) drugs in current development for the treatment of female sexual dysfunction;

(50) drugs used for the treatment of erectile dysfunction such as alprostadil, aviptadil, phentolamine mesilate, weige, alprostadil;

(51) drugs currently in development for the treatment of male sexual dysfunction;

(52) drugs in development for the treatment of sleep apnea;

(53) drugs currently in development for the treatment of metabolic syndrome;

(54) drugs used for the treatment of Alzheimer's disease: e.g., cholinesterase inhibitors prescribed for mild to moderate Alzheimer's disease, including Razadyne® (galantamine), Exelon® (rivastigmine), and Aricept® (donepezil), Cognex® (tacrine); Namenda® (memantine), an N-methyl D-aspartate (NMDA) antagonist, and Aricept®, prescribed to treat moderate to severe Alzheimer's disease; vitamin E (an anti-oxidant);

(55) antidepressants: tricyclic antidepressants such as amitriptyline (Elavil®), desipramine (Norpramin®), imipramine (Tofranil®), amoxapine (Asendin®), nortriptyline; the selective serotonin reuptake inhibitors (SSRI's) such as paroxetine (Paxil®), fluoxetine (Prozac®), sertraline (Zoloft®), and citralopram (Celexa®); and others such as doxepin (Sinequan®) and trazodone (Desyrel®); SNRIs (e.g., venlafaxine and reboxetine); dopaminergic antidepressants (e.g., bupropion and amineptine);

(56) neuroprotective agents: e.g., memantine, L-dopa, bromocriptine, pergolide, talipexol, pramipexol, cabergoline, neuroprotective agents currently under investigation including anti-apoptotic drugs (CEP 1347 and CTCT346), lazaroids, bioenergetics, antiglutamatergic agents and dopamine receptors. Other clinically evaluated neuroprotective agents are, e.g., the monoamine oxidase B inhibitors selegiline and rasagiline, dopamine agonists, and the complex I mitochondrial fortifier coenzyme Q10;

(57) antipsychotic medications: e.g., ziprasidone (Geodon™), risperidone (Risperdal™), and olanzapine (Zyprexa™);

(58) ADHD medications: e.g., Adderall®, Dexedrine®, Dextrostat®, Spansule®, Adderall XR®, Vyvanse®, Focalin®, Methylin®, Ritalin®, Metadate ER®, Methylin ER®, Ritalin SR®, Metadate CD®, Ritalin LA®, Concerta®, Quillivant XR®, Focalin XR®, Daytrana® patch, Strattera®, Intuniv®, Wellbutrin®, Wellbutrin SR®, Wellbutrin XL®, Tofranil®, Pamelor®, Aventyl®, Norpramin®, Clonidine®, Catapres®, Kapvay®, Tenex®;

(59) therapeutics that increase the function or localization of dystrophin, including therapeutics that affect translation, stop codons and/or exon skipping, or that increase utrophin expression or therapeutics that are genetic modifiers; on-limiting examples include ataluran, biglycan, CAT-1000, catena, Cialis® (tadalafil), CoQ10/lisinopril, DT-200, drisapersen, eplerenone, eteplirsen, follistatin in AAV vector, GSK 24029681/Drisapersen, HT-100, IGF-1/Increlex, laminin 111, NBD Peptide, rycalARM201, SMT C1100, tamoxifen, VBP-15 or PTC compound.

Pharmaceutical Compositions and their Routes of Administration

The compounds herein disclosed, and their pharmaceutically acceptable salts, thereof may be formulated as pharmaceutical compositions or “formulations”.

A typical formulation is prepared by mixing a compound described herein, or a pharmaceutically acceptable salt thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound described herein is being formulated. Solvents are generally selected based on solvents recognized by persons skilled in the art as safe (e.g., one described in the GRAS (Generally Recognized as Safe) database) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g., enteric or slow release) preservatives, antioxidants, opaquing agents, glidants, processing aids, colorants, sweeteners, perfuming agents, flavoring agents and other known additives to provide an elegant presentation of the drug (i.e., a compound described herein or pharmaceutical composition thereof) or aid in the manufacturing of the pharmaceutical product (i.e., medicament).

The formulations may be prepared using conventional dissolution and mixing procedures. For example, the bulk drug substance (i.e., one or more of the compounds described herein, a pharmaceutically acceptable salt thereof, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8.

A compound described herein or a pharmaceutically acceptable salt thereof is typically formulated into pharmaceutical dosage forms to provide an easily controllable dosage of the drug and to enable patient compliance with the prescribed regimen. Pharmaceutical formulations of compounds described herein, or a pharmaceutically acceptable salt thereof, may be prepared for various routes and types of administration. Various dosage forms may exist for the same compound. The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total composition (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

The pharmaceutical compositions described herein will be formulated, dosed, and administered in a fashion, i.e., amounts, concentrations, schedules, course, vehicles, and route of administration, consistent with good medical practice. Factors for consideration in this context include the particular disorder being treated, the particular human or other mammal being treated, the clinical condition of the individual patient, the cause of the disorder, the site of delivery of the agent, the method of administration, the scheduling of administration, and other factors known to medical practitioners, such as the age, weight, and response of the individual patient.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease or disorder or one or more of its symptoms.

The term “prophylactically effective amount” refers to an amount effective in preventing or substantially lessening the chances of acquiring a disorder or in reducing the severity of the disorder or one or more of its symptoms before it is acquired or before the symptoms develop further.

In some embodiments, a prophylactically effective amount of an sGC stimulator is one that prevents or delays the occurrence, progression or reoccurrence of muscle wasting, muscle necrosis, muscle weakness or muscle ischemia. In further embodiments, a prophylactically effective amount of an sGC stimulator is one that prevents or delays the occurrence or reoccurrence of muscle wasting, muscle necrosis, muscle weakness or muscle ischemia in a subject suffering from a Muscular Dystrophy. In further embodiments, a prophylactically effective amount of an sGC stimulator is one that prevents or delays the progression of muscle wasting, muscle necrosis, muscle weakness or muscle ischemia in a subject suffering from a Muscular Dystrophy. In other embodiments, a prophylactically effective amount of an sGC stimulator is one that prevents or delays the occurrence or reoccurrence of muscle wasting, muscle necrosis, muscle weakness or muscle ischemia in a subject suffering with one of Duchenne or Becker Muscular Dystrophy. In other embodiments, a prophylactically effective amount of an sGC stimulator is one that prevents or delays the progression of muscle wasting, muscle necrosis, muscle weakness or muscle ischemia in a subject suffering with one of Duchenne or Becker Muscular Dystrophy. In other embodiments, a prophylactically effective amount of an sGC stimulator is one that prevents or delays the progression of muscle wasting, muscle necrosis, muscle weakness or muscle ischemia in a subject suffering with one of the other known types of Muscular Dystrophy.

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”.

“Sustained-release preparations” are the most common applications of controlled release. Suitable examples of sustained-release preparations include semipermeable matrices of solid hydrophobic polymers containing the compound, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Gastroretentive formulations” are preparations designed to have increased retention in the stomach cavity. In some cases, they are used where a drug is preferentially or primarily absorbed via the stomach, is designed to treat the stomach directly, or where drug dissolution or absorption is aided drug absorption is aided by prolonged exposure to gastric acids. Examples of gastroretentive formulations include but are not limited to, high-density formulations, where the density of the formulation is higher than gastric fluid; floating formulations, which can float on top of gastric fluids due to increased buoyancy or lower density of the formulation; temporarily expandable formulations that are temporarily larger than the gastric opening; muco- and bio-adhesive formulations; swellable gel formulations; and in situ gel forming formulations. (See, e.g., Bhardwaj, L. et al. African J. of Basic & Appl. Sci. 4(6): 300-312 (2011)).

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent disaggregation to fine particles. This provides a larger surface area exposed to the dissolution medium, resulting in a faster dissolution rate.

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The terms “administer”, “administering” or “administration” in reference to a compound, composition or formulation of the invention means introducing the compound into the system of the animal in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g.: orally (e.g. using capsules, powders, solutions, suspensions, tablets, sublingual tablets and the like), by inhalation (e.g. with an aerosol, gas, inhaler, nebulizer or the like), to the ear (e.g. using ear drops), topically (e.g. using creams, gels, liniments, lotions, ointments, pastes, transdermal patches, etc), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. In particular embodiments, the compositions are administered orally, intraperitoneally or intravenously.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution-retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

Formulations of a compound described herein that are suitable for oral administration may be prepared as discrete units such as tablets, pills, troches, lozenges, aqueous or oil suspensions, dispersible powders or granules, emulsions, hard or soft capsules, e.g., gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with a water-soluble carrier such as polyethyleneglycol or an oil medium, for example, peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g., for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations.

Oily suspensions may be formulated by suspending a compound described herein in a vegetable oil, for example arachis oil, olive oil, sesame oil or coconut oil, or in mineral oil such as liquid paraffin. The oily suspensions may contain a thickening agent, for example, beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds described herein contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable drug-depot forms are made by forming microencapsuled matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Drug-depot injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl ester wax, cetearyl alcohol, 2 octyldodecanol, benzyl alcohol and water.

For ophthalmic use, the pharmaceutical compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, between 0.075% and 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds described herein may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds described herein include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration may have a mean particle size in the range of, for example, 0.1 to 500 microns (including particles with a mean particle size in the range between 0.1 and 500 microns in increments such as 0.5, 1, 30, 35 microns, etc.), which may be administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient. In another aspect, a compound described herein or a pharmaceutically acceptable salt, co-crystal, solvate or pro-drug thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Kits

The pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound of Formula I described herein and a pharmaceutically acceptable carrier, vehicle or diluent; and b) another therapeutic agent and a pharmaceutically acceptable carrier, vehicle or diluent in one or more containers or separate packaging. The kits may optionally comprise instructions describing a method of using the pharmaceutical compositions in one or more of the methods described herein (e.g. preventing or treating one or more of the diseases and disorders described herein). The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or other compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLES

As used herein, all abbreviations, symbols and conventions are consistent with those used in the contemporary scientific literature. See, e.g. Janet S. Dodd, ed., The ACS Style Guide: A Manual for Authors and Editors, 2^{nd} Ed., Washington, D.C.: American Chemical Society, 1997, herein incorporated by reference in its entirety.

### Example 1

### Laser Doppler Blood Flow Evaluation after Muscle Stimulation in Mdx Mice with and without Treatment with an sGC Stimulator

### sGC Stimulator

The sGC stimulator used in this experiment was Compound A depicted below:

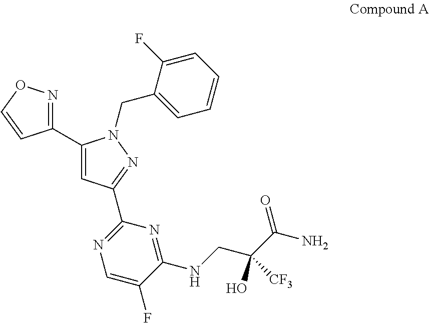 [SMILES: NC(=O)[C@@](O)(CNc1nc(-c2cc(-c3ccon3)n(Cc3ccccc3F)n2)ncc1F)C(F)(F)F]

The preparation and characterization of this compound was described in patent application publication WO2014144100, published 18 Sep. 2014.

### Animals

C57/BL6 mice were used as the healthy control animals.

Mdx animals are mice that have a spontaneous mutation in the dystrophin gene and are therefore a useful animal model for the study of DMD in humans.

### Preparation

Animals were placed under isoflurane anesthesia and the fur on the ventral surface of their hind legs was shaved with an electric trimmer. The remaining fur on both legs was removed via Nair application. This procedure was performed the day before laser Doppler blood flow evaluation to minimize the risk of skin irritation from confounding the Doppler blood flow readings.

### Doppler Blood Flow Evaluation

On the day of the Doppler blood flow evaluation, animals received treatment according to the doses summarized in Table XX via oral gavage (p.o.) 1 to 2 hours prior to muscle stimulation and Doppler blood flow assessment. Animals were anesthetized and maintained unconscious with isoflurane anesthesia and their legs were gently restrained using tape. A surface probe (12 mm circumference) was placed directly on the skin surface on the exposed areas of both legs for establishment of baseline blood perfusion readings. Following this assessment, electrical impulses were administered to animals on the right leg only as indicated in Table XX below. Stimulation probes were placed directly on the exposed skin surface of the animal's right leg and animals received one set of five stimulations (5×10 second stimulation time) with 5 seconds of rest between stimulations. Following the muscle stimulation, the Doppler surface probe was placed on the exposed areas of both legs to measure blood perfusion after stimulation treatment. Table XXX summarizes the electrical stimulation parameters.

TABLE XX
Study Design

| | Number | | |
|---|---|---|---|
| Group | of | | |
| Number | Animals | Treatment* | Tetanic muscle stimulation |
| 1 | 5 MDX | Vehicle | Laser Doppler Blood Flow |
| | 5 C57/ | | of stimulated and non- |
| | BL10 | | stimulated legs, pre- and post- |
| | | | stimulation |
| 2 | 5 MDX | Compound A | Laser Doppler Blood Flow |
| | | 3 mpk | of stimulated and non- |
| | | | stimulated legs, pre- and post- |
| | | | stimulation |
| 3 | 5 MDX | Compound B | Laser Doppler Blood Flow |
| | | 1 mpk | of stimulated and non- |
| | | | stimulated legs, pre- and post- |
| | | | stimulation |
| 4 | 5 MDX | Compound C | Laser Doppler Blood Flow |
| | | 0.3 mpk | of stimulated and non- |
| | | | stimulated legs, pre- and post- |
| | | | stimulation |
| 5 | 5 MDX | tadalafil | Laser Doppler Blood Flow |
| | | 1 mg/kg | of stimulated and non- |
| | | | stimulated legs, pre- and post- |
| | | | stimulation |

*P.O. Administration 1-2 hours prior to muscle stimulation/Doppler blood flow assessment.

TABLE XXX
electrical stimulation parameters

| | Stimulation Parameters | |
|---|---|---|
| Pulse Duration | 100 | μs |
| Frequency | 25 | hz |
| Stimulation | 10 | sec |
| Time | | |
| Time between | 5 | sec |
| Stimulation | | |
| Intensity | 3 | mA |

### Results

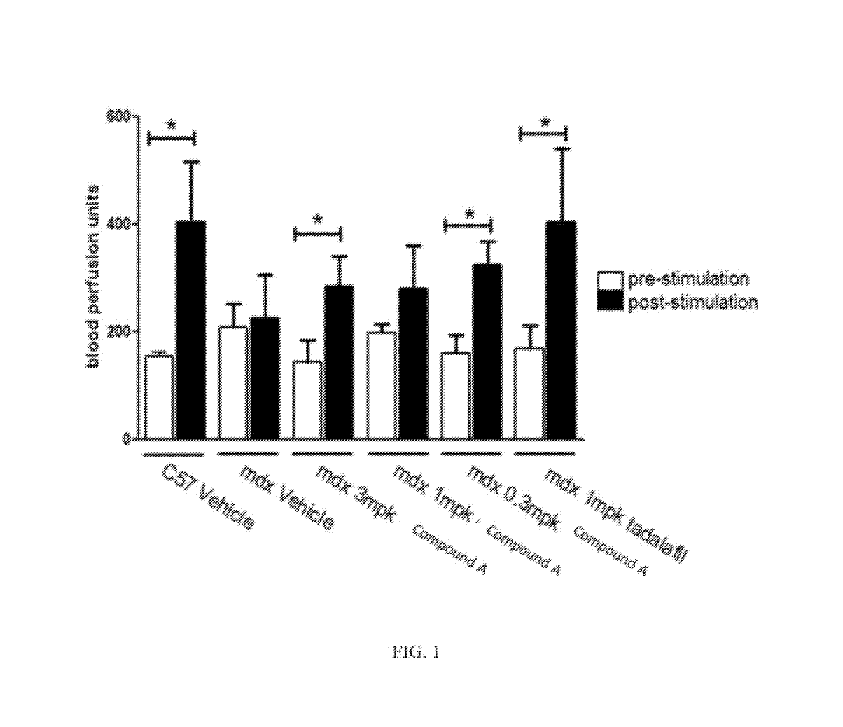

Results of this experiment are summarized in FIG. 1. Blood flow was measured using an OxyFlo 2000 laser Doppler perfusion monitoring system. Data are reported as arbitrary blood perfusion units which were not normalized. For each measurement, the Doppler readings were taken from 5 locations along the leg, and those 5 measurements were averaged to generate the value for that mouse (either pre- or post-stimulation). The data were analyzed using GraphPad Prism.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

1. A method of treating a neuromuscular disorder associated with loss or alteration of function of nitric oxide synthase (NOS) in a patient in need thereof, comprising administering to said patient a therapeutically effective amount of an sGC stimulator or a pharmaceutically acceptable salt thereof, wherein said neuromuscular disorder is associated with one or more mutations in genes associated with the dystrophin glycoprotein complex (DGC).

2. (canceled)

3. The method of claim 1, wherein said neuromuscular disorder is associated with one or more mutations in the dystrophin gene.

4. The method of claim 1, wherein the disorder is Muscular Dystrophy.

5. The method of claim 4, wherein the disorder is Duchenne Muscular Dystrophy.

6. The method of claim 4, wherein the disorder is Becker Muscular Dystrophy.

7. The method of claim 1, wherein said sGC stimulator or pharmaceutically acceptable salt thereof is administered as a monotherapy.

8. The method of claim 1, wherein said sGC stimulator or pharmaceutically acceptable salt thereof is administered in combination with a therapeutically or prophylactically effective amount of one or more additional therapeutic agents.

9. The method of claim 8, wherein the additional therapeutic agent is selected from an anti-inflammatory drug; nitric oxide; a NO-donor; a Nitric Oxide synthase substrate; a sGC stimulator; a sGC activator; a PDE5 inhibitor; a compound that is a genetic modifier; a compound that increases the function or localization of dystrophin; a compound that affects translation, stop codons and/or exon skipping; and a compound that increases utrophin expression.

10-19. (canceled)

20. The method of claim 1, wherein the patient in need thereof is an adult.

21. The method of claim 1, wherein the patient in need thereof is a child.

22-29. (canceled)

30. The method of claim 1, wherein treatment with a sGC stimulator or a pharmaceutically acceptable salt thereof results in an observable or measurable decrease in the progression of muscle wasting.

31. The method of claim 30, wherein the decrease in the progression of muscle wasting is measured by using a 6-minute walking distance test, a stair climbing test, a stair climbing test wherein the time required for going from seating to a standing position is determined, or by measuring improvements in tissue blood flow after exercise or muscle stimulation.

32-34. (canceled)

35. The method of claim 1, wherein treatment with a sGC stimulator or a pharmaceutically acceptable salt thereof results in an observable or measurable a) increase in the degree of muscle function, b) increase in muscle strength, c) decrease in fatigue, d) reduction in the risk of muscular injury, e) reduction in the level of muscle or bone deformity, and/or f) reduction in the rate of appearance of new muscle or bone deformity.

36. The method of claim 1, wherein treatment with a sGC stimulator or a pharmaceutically acceptable salt thereof results in an observable or measurable decrease in the degree of muscle necrosis, reduction in the level of muscle fibrosis, and/or reduction in the rate of appearance of new muscle fibrosis.

37-51. (canceled)

52. The method of claim 8, wherein the sGC stimulator is administered prior to, at the same time as, or after the initiation of treatment with another therapeutic agent.

53. (canceled)

54. A pharmaceutical composition comprising a sGC stimulator, or a pharmaceutically acceptable salt thereof, and optionally one or more additional therapeutic agents, for use in the treatment of a neuromuscular disorder associated with loss or alteration of function of nitric oxide synthase (NOS) in a patient in need thereof, wherein said neuromuscular disorder is associated with one or more mutations in genes associated with the dystrophin glycoprotein complex (DGC).

55. (canceled)

56. The method of claim 1, wherein the sGC stimulator is a compound according to Formula I′, or a pharmaceutically acceptable salt thereof, 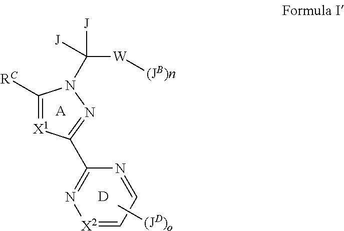
wherein X¹ is selected from N, CH, C(C₁₋₄ alkyl), C(C₁₋₄ haloalkyl), CCl and CF;
X² is independently selected from N or C;
W is either
i) absent, with J^{B} connected directly to the carbon atom bearing two J groups, each J is independently selected from hydrogen or methyl, n is 1 and J^{B} is a C₁₋₇ alkyl chain optionally substituted by up to 9 instances of fluorine; wherein, optionally, one —CH₂— unit of said C₁₋₇ alkyl chain can be replaced by —O— or —S—.
ii) a ring B that is a phenyl or a 5 or 6-membered heteroaryl ring, containing 1 or 2 ring heteroatoms selected from N, O or S; wherein with ring B being the phenyl or 5 or 6-membered heteroaryl ring; each J is hydrogen; n is an integer selected from 0 to 3; and each J^{B} is independently selected from halogen, —CN, a C₁₋₆ aliphatic, —OR^{B} or a C₃₋₈ cycloaliphatic group; wherein each said C₁₋₆ aliphatic and each said C₃₋₈ cycloaliphatic group is optionally and independently substituted with up to 3 instances of R³; each R^{B} is independently selected from hydrogen, a C₁₋₆ aliphatic or a C₃₋₈ cycloaliphatic; wherein each said R^{B} that is a C₁₋₆ aliphatic and each said R^{B} that is a C₃₋₈ cycloaliphatic ring is optionally and independently substituted with up to 3 instances of R^{3a};
each R³ is independently selected from halogen, —CN, C₁₋₄ alkyl, C₁₋₄ haloalkyl, —O(C₁₋₄ alkyl) or —O(C₁₋₄ haloalkyl);
each R^{3a} is independently selected from halogen, —CN, C₁₋₄ alkyl, C₁₋₄ haloalkyl, —O(C₁₋₄ alkyl) or —O(C₁₋₄ haloalkyl);
o is an integer selected from 1, 2 and 3;
each J^{D} is independently selected from J^{A}, halogen, —CN, —NO₂, —OR^{D}, —SR^{D}, —C(O)R^{D}, —C(O)OR^{D}, —OC(O)R^{D}, —C(O)N(R^{D})₂, —N(R^{D})₂, —N(R^{d})C(O)R^{D}, —N(R^{d})C(O)OR^{D}, —N(R^{d})C(O)N(R^{D})₂, —OC(O)N(R^{D})₂, —SO₂R^{D}, —SO₂N(R^{D})₂, —N(R^{d})SO₂R^{D}, a C₁₋₆ aliphatic, —(C₁₋₆ aliphatic)-R^{D}, a C₃₋₈ cycloaliphatic ring, a 6 to 10-membered aryl ring, a 4 to 8-membered heterocyclic ring or a 5 to 10-membered heteroaryl ring; wherein each said 4 to 8-membered heterocylic ring and each said 5 to 10-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{D} moiety, each said C₃₋₈ cycloaliphatic ring, each said 6 to 10-membered aryl ring, each said 4 to 8-membered heterocyclic ring and each said 5 to 10-membered heteroaryl ring is optionally and independently substituted with up to 5 instances of R^{5d};
J^{A} is selected from hydrogen, halogen, methyl, hydroxyl, methoxy, trifluoromethyl, trifluoromethoxy or —NR^{a}R^{b}; wherein R^{a} and R^{b} are each independently selected from hydrogen, C₁₋₆ alkyl or a 3-6 cycloalkyl ring; or wherein R^{a} and R^{b}, together with the nitrogen atom to which they are both attached, form a 4-8 membered heterocyclic ring, or a 5-membered heteroaryl ring optionally containing up to two additional heteroatoms selected from N, O and S; wherein each of said 4-8 membered heterocyclic ring and 5-membered heteroaryl ring is optionally and independently substituted by up to 6 instances of fluorine;
each R^{D} is independently selected from hydrogen, a C₁₋₆ aliphatic, —(C₁₋₆ aliphatic)-R^{f}, a C₃₋₈ cycloaliphatic ring, a 4 to 10-membered heterocyclic ring, phenyl or a 5 to 6-membered heteroaryl ring; wherein each said 4 to 10-membered heterocylic ring and each said 5 to 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 10-membered heterocyclic ring, each said phenyl and each said 5 to 6-membered heteroaryl ring is optionally and independently substituted with up to 5 instances of R^{5a}; wherein when any R^{D} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{d})—, —CO— or —O—; provided that when X¹ is one of CH, C(C₁₋₄ alkyl), C(C₁₋₄ haloalkyl), CCl or CF; X² is C; and at least one J^{D} is —N(R^{D})₂ and is attached to one of the pyrimidine ring D carbons ortho to the two nitrogen atoms of said ring D, one instance of R^{D} is not a pyridine or a pyrimidine;
each R^{d} is independently selected from hydrogen, a C₁₋₆ aliphatic, —(C₁₋₆ aliphatic)-R^{f}, a C₃₋₈ cycloaliphatic ring, a 4 to 8-membered heterocyclic ring, phenyl or a 5 to 6-membered heteroaryl ring; wherein each said 4 to 8-membered heterocylic ring and each said 5 or 6-membered heteroaryl ring contains between 1 and 3 heteroatoms independently selected from O, N or S; and wherein each said C₁₋₆ aliphatic, each said C₁₋₆ aliphatic portion of the —(C₁₋₆ aliphatic)-R^{f} moiety, each said C₃₋₈ cycloaliphatic ring, each said 4 to 8-membered heterocyclic ring, each said phenyl and each said 5 to 6-membered heteroaryl ring is optionally and independently substituted by up to 5 instances of R^{5b}; wherein when any R^{D} is one of a C₁₋₆ aliphatic or a —(C₁₋₆ aliphatic)-R^{f} group, one or two —CH₂— units that form said C₁₋₆ aliphatic chains may, optionally, be replaced by a group independently selected from —N(R^{d})—, —CO— or —O—;
each R^{f} is independently selected from a C₁₋₃ alkyl, a C₃₋₈ cycloaliphatic ring, a 4 to 10-membered heterocyclic ring, phenyl or a 5 to 6-membered heteroaryl ring; wherein each said 4 to 10-membered heterocylic ring and each said 5 to 6-membered heteroaryl ring contains between 1 and 4 heteroatoms independently selected from O, N or S; and wherein each said C₃₋₈ cycloaliphatic ring, each said 4 to 10-membered heterocyclic ring, each said phenyl and each said 5 to 6-membered heteroaryl ring is optionally and independently substituted by up to 5 instances of R^{5c};
when J^{D} is —C(O)N(R^{D})₂, —N(R^{D})₂, —N(R^{d})C(O)N(R^{D})₂, —OC(O)N(R^{D})₂ or —SO₂N(R^{D})₂, the two R^{D} groups together with the nitrogen atom attached to the two R^{D} groups may form a 4 to 8-membered heterocyclic ring or a 5-membered heteroaryl ring; wherein each said 4 to 8-membered heterocyclic ring and each said 5-membered heteroaryl ring optionally contains up to 3 additional heteroatoms independently selected from N, O or S, in addition to the nitrogen atom to which the two R^{D} groups are attached; and wherein each said 4 to 8-membered heterocyclic ring and each said 5-membered heteroaryl ring is optionally and independently substituted by up to 5 instances of R⁵;
when J^{D} is —N(R^{d})C(O)R^{D}, the R^{D} group together with the carbon atom attached to the R^{D} group, with the nitrogen atom attached to the R^{d} group, and with the R^{d} group may form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S, in addition to the nitrogen atom to which the R^{d} group is attached; and wherein each said 4 to 8-membered heterocyclic ring and each said 5-membered heteroaryl ring is optionally and independently substituted by up to 5 instances of R⁵;
when J^{D} is —N(R^{d})C(O)OR^{D}, the R^{D} group together with the oxygen atom attached to the R^{D} group, with the carbon atom of the —C(O)— portion of the —N(R^{d})C(O)OR^{D} group, with the nitrogen atom attached to the R^{d} group, and with said R^{d} group, may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})C(O)N(R^{D})₂, one of the R^{D} groups attached to the nitrogen atom, together with said nitrogen atom, and with the N atom attached to the R^{d} group and said R^{d} group may form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
when J^{D} is —N(R^{d})SO₂R^{D}, the R^{D} group together with the sulfur atom attached to the R^{D} group, with the nitrogen atom attached to the R^{d} group, and with said R^{d} group may combine to form a 4 to 8-membered heterocyclic ring; wherein said 4 to 8-membered heterocyclic ring optionally contains up to 2 additional heteroatoms independently selected from N, O or S, and is optionally and independently substituted by up to 5 instances of R⁵;
each R⁵ is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of the —(C₁₋₆ alkyl)-R⁶ moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R⁵, attached to the same or different atoms of J^{D}, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —CONH₂, —OH or halogen; wherein R is hydrogen or a C₁₋₂ alkyl;
each R^{5a} and each R^{5b} is independently selected from halogen, —CN, C₁₋₆ alkyl, —(C₁₋₆ alkyl)R^{6a}, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂OH, —SO₂NHOH, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₁₋₆ alkyl portion of the —(C₁₋₆ alkyl)R^{6a} moiety, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5a} or two instances of R^{5b} attached to the same or different atoms of R^{D} or R^{d}, respectively, together with said atom or atoms to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —C(O)NH₂, —NR(CO)O(C₁₋₄ alkyl), —OH or halogen; wherein R is hydrogen or a C₁₋₂ alkyl;
each R^{5c} is independently selected from halogen, —CN, C₁₋₆ alkyl, —(C₁₋₆ alkyl)-R^{6b}, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂OH, —SO₂NHOH, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR^{6b}, a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R^{6b} moiety, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5c} attached to the same or different atoms of R^{f}, together with said atom or atoms to which it is attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —CONH₂, —NR(CO)O(C₁₋₄ alkyl), —OH or halogen; wherein R is hydrogen or a C₁₋₂ alkyl;
each R^{5d} is independently selected from halogen, —CN, C₁₋₆ alkyl, —(C₁₋₆ alkyl)-R⁶, —OR⁶, —SR⁶, —COR⁶, —OC(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂, —SO₂R⁶, —SO₂OH, —SO₂NHOH, —SO₂N(R⁶)COR⁶, —SO₂N(R⁶)₂, —N(R⁶)SO₂R⁶, a C₇₋₁₂ aralkyl, a C₃₋₈ cycloalkyl ring, a 4 to 7-membered heterocyclic ring, a 5 or 6-membered heteroaryl ring, phenyl or an oxo group; wherein each 5 or 6-membered heteroaryl ring or 4 to 7-membered heterocyclic ring contains up to four ring heteroatoms independently selected from N, O and S, wherein each of said C₁₋₆ alkyl, C₁₋₆ alkyl portion of the —(C₁₋₆ alkyl)-R⁶moiety, C₇₋₁₂ aralkyl, C₃₋₈ cycloalkyl ring, 4 to 7-membered heterocyclic ring, 5 or 6-membered heteroaryl ring or phenyl group is optionally and independently substituted with up to 3 instances of halogen, C₁₋₄ alkyl, C₁₋₄ (haloalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ haloalkyl) or oxo;
two instances of R^{5d} attached to the same or different atoms of J^{D}, together with said atom or atoms of J^{D} to which they are attached, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship with respect to each other; wherein said 4 to 6-membered heterocycle or said 5 or 6-membered heteroaryl ring contains up to four ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)O(C₁₋₄ alkyl), —C(O)NH₂, —OH or halogen; wherein R is hydrogen or a C₁₋₂ alkyl;
each R⁶ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆ alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —COO(C₁₋₄ alkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S; wherein
two instances of R⁶ linked to the same nitrogen atom of R⁵ or R^{5d}, together with said nitrogen atom of R⁵ or R^{5d}, respectively, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6a} linked to a nitrogen atom of R^{5a} or R^{5b}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two instances of R^{6b} linked to a nitrogen atom of R^{5c}, together with said nitrogen, may form a 5 to 8-membered heterocyclic ring or a 5-membered heteroaryl ring; wherein each said 5 to 8-membered heterocyclic ring and each said 5-membered heteroaryl ring optionally contains up to 2 additional heteroatoms independently selected from N, O or S;
two J^{D} groups attached to two vicinal ring D atoms, taken together with said two vicinal ring D atoms, may form a 5 to 7-membered heterocycle or a 5-membered heteroaryl ring that is fused to ring D; wherein said 5 to 7-membered heterocycle or said 5-membered ring heteroaryl contains from 1 to 3 heteroatoms independently selected from N, O or S; and wherein said 5 to 7-membered heterocycle or said 5-membered heteroaryl ring is optionally and independently substituted by up to 3 instances of oxo or —(Y)—R⁹;
wherein Y is either absent or is a linkage in the form of a C₁₋₆ alkyl chain, optionally substituted by up to 6 instances of fluoro; and wherein when Y is said C₁₋₆ alkyl chain, up to 3 methylene units of this alkyl chain, can be replaced by a group selected from —O—, —C(O)— or —N((Y)—R⁹)—;
each R⁹ is independently selected from hydrogen, halogen, —CN, —OR¹⁰, —COR¹⁰, —OC(O)R¹⁰, —C(O)OR¹⁰, —C(O)N(R¹⁰)₂, —C(O)N(R¹⁰)SO₂R₁₀, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, C₃₋₆ cycloalkyl ring, a 4-8-membered heterocyclic ring, a phenyl ring or a 5-6 membered heteroaroaryl ring; wherein each said 4 to 8-membered heterocyclic ring or 5 to 6-membered heteroaryl ring contains up to 4 ring heteroatoms independently selected from N, O or S; and wherein each of said C₃₋₆ cycloalkyl rings, each of said 4 to 8-membered heterocyclic rings, each of said phenyl and each of said 5 to 6-membered heteroaryl rings is optionally and independently substituted with up to 3 instances of R¹¹;
each R¹⁰ is independently selected from hydrogen, a C₁₋₆ alkyl, —(C₁₋₆ alkyl)-R¹³, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, C₁₋₆ alkyl portion of said —(C₁₋₆ alkyl)-R¹³ moiety, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of R^{11a};
each R¹³ is independently selected from a phenyl, a benzyl, a C₃₋₆ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each said phenyl, each of said benzyl, each said C₃₋₈ cycloalkyl group, each said 4 to 7-membered heterocyclic ring and each 5 or 6-membered heteroaryl ring is optionally and independently substituted with up to 3 instances of R^{11b};
each R¹¹ is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²;
each R^{11a} is independently selected from halogen, oxo, C₁₋₆ alkyl, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²; and
each R^{11b} is independently selected from halogen, C₁₋₆ alkyl, oxo, —CN, —OR¹², —COR¹², —C(O)OR¹², —C(O)N(R¹²)₂, —N(R¹²)C(O)R¹², —N(R¹²)C(O)OR¹², —N(R¹²)C(O)N(R¹²)₂, —N(R¹²)₂, —SO₂R¹², —SO₂N(R¹²)₂ or —N(R¹²)SO₂R¹²; wherein each of said C₁₋₆ alkyl is optionally and independently substituted by up to 6 instances of fluoro and/or 3 instances of R¹²;
each R¹² is selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —CONH₂, —COO(C₁₋₄ alkyl), —O(C₁₋₄ alkyl), —O(C₁₋₄ fluoroalkyl) or oxo;
R^{C} is either
i) a ring C; or
ii) is selected from halogen, —CN, C₁₋₆ alkyl, —(C₁₋₆ alkyl)-R^{N}, —COR⁷, —C(O)OR⁷, —C(O)N(R⁷)₂, —N(R⁷)C(O)R⁷, —N(R⁷)C(O)OR⁷, —N(R⁷)C(O)N(R⁷)₂, —N(R⁷)₂, —SO₂R⁷, —SO₂N(R⁷)₂, —C(O)N(R⁷)SO₂R⁷, —SO₂N(R⁷)COOR⁷, —SO₂N(R⁷)C(O)R⁷ or —N(R⁷)SO₂R; wherein each said C₁₋₆ alkyl, each C₁₋₆ alkyl portion of said —(C₁₋₆ alkyl)-R^{N}, is optionally and independently substituted with up to 6 instances of fluoro and up to 2 instances of —CN, —OR⁸, oxo, —N(R⁸)₂, —N(R⁸)C(O)R⁸, —N(R⁸)C(O)R⁸, —C(O)N(R⁸)₂, —N(R⁸)C(O)N(R⁸)₂, —SO₂R⁸, —SO₂N(R⁸)₂, —NHOR⁸, —SO₂N(R⁸)COOR⁸, —SO₂N(R⁸)C(O)R⁸, —N(R⁷)SO₂R⁸;
wherein each R⁷ is independently selected from hydrogen, C₁₋₆ alkyl, C₁₋₆ fluoroalkyl, a C₃₋₈ cycloalkyl ring, phenyl, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R⁸ is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{N} is independently selected from a phenyl ring, a monocyclic 5 or 6-membered heteroaryl ring, a monocyclic C₃₋₆ cycloaliphatic ring, or a monocyclic 4 to 6-membered heterocycle; wherein said monocyclic 5 or 6-membered heteroaryl ring or said monocyclic 4 to 6-membered heterocycle contain between 1 and 4 heteroatoms selected from N, O or S; wherein said monocyclic 5 or 6-membered heteroaryl ring is not a 1,3,5-triazinyl ring; and wherein said phenyl, said monocyclic 5 to 6-membered heteroaryl ring, said monocyclic C₃₋₆ cycloaliphatic ring, or said monocyclic 4 to 6-membered heterocycle is optionally and independently substituted with up to 6 instances of fluoro and/or up to 3 instances of J^{M};
each J^{M} is independently selected from —CN, a C₁₋₆ aliphatic, —OR^{M}, —SR^{M}, —N(R^{M})₂, a C₃₋₈ cycloaliphatic ring or a 4 to 8-membered heterocyclic ring; wherein said 4 to 8-membered heterocyclic ring contains 1 or 2 heteroatoms independently selected from N, O or S; wherein each said C₁₋₆ aliphatic, each said C₃₋₈ cycloaliphatic ring and each said 4 to 8-membered heterocyclic ring, is optionally and independently substituted with up to 3 instances of R^{7c};
each R^{M} is independently selected from hydrogen, a C₁₋₆ aliphatic, a C₃₋₈ cycloaliphatic ring or a 4 to 8-membered heterocyclic ring; wherein each said 4 to 8-membered heterocylic ring contains between 1 and 3 heteroatoms independently selected from O, N or S; and wherein
ring C is a phenyl ring, a monocyclic 5 or 6-membered heteroaryl ring, a bicyclic 8 to 10-membered heteroaryl ring, a monocyclic 3 to 10-membered cycloaliphatic ring, or a monocyclic 4 to 10-membered heterocycle; wherein said monocyclic 5 or 6-membered heteroaryl ring, said bicyclic 8 to 10-membered heteroaryl ring, or said monocyclic 4 to 10-membered heterocycle contain between 1 and 4 heteroatoms selected from N, O or S; wherein said monocyclic 5 or 6-membered heteroaryl ring is not a 1,3,5-triazinyl ring; and wherein said phenyl, monocyclic 5 to 6-membered heteroaryl ring, bicyclic 8 to 10-membered heteroaryl ring, monocyclic 3 to 10-membered cycloaliphatic ring, or monocyclic 4 to 10-membered heterocycle is optionally and independently substituted with up to p instances of J^{C}′; wherein p is 0 or an integer selected from 1 to 3;
each J^{C} is independently selected from halogen, —CN, —NO₂, a C₁₋₆ aliphatic, —OR^{H}, —SR^{H}, —N(R^{H})₂, a C₃₋₈ cycloaliphatic ring or a 4 to 8-membered heterocyclic ring; wherein said 4 to 8-membered heterocyclic ring contains 1 or 2 heteroatoms independently selected from N, O or S; wherein each said C₁₋₆ aliphatic, each said C₃₋₈ cycloaliphatic ring and each said 4 to 8-membered heterocyclic ring, is optionally and independently substituted with up to 3 instances of R^{7d}; or
alternatively, two J^{C} groups attached to two vicinal ring C atoms, taken together with said two vicinal ring C atoms, form a 5 to 7-membered heterocycle that is a new ring fused to ring C; wherein said 5 to 7-membered heterocycle contains from 1 to 2 heteroatoms independently selected from N, O or S;
each R^{H} is independently selected from hydrogen, a C₁₋₆ aliphatic, a C₃₋₈ cycloaliphatic ring or a 4 to 8-membered heterocyclic ring; wherein each said 4 to 8-membered heterocylic ring contains between 1 and 3 heteroatoms independently selected from O, N or S; alternatively, two instances of R^{H} linked to the same nitrogen atom of —N(R^{H})₂, together with said nitrogen atom of —N(R^{H})₂, form a 4 to 8-membered heterocyclic ring or a 5-membered heteroaryl ring; wherein each said 4 to 8-membered heterocyclic ring and each said 5-membered heteroaryl ring optionally contains up to 2 additional heteroatoms independently selected from N, O or S;
each R^{7c} is independently selected from hydrogen, halogen, —CN, —NO₂, C₁₋₄ alkyl, C₁₋₄ haloalkyl, C₃₋₈ cycloalkyl ring, —OR^{8b}, —SR^{8b}, —N(R^{8b})₂, —C(O)O(C₁₋₄ alkyl), —C(O)OH, —NR(CO)CO(C₁₋₄ alkyl) or an oxo group; wherein each said cycloalkyl group is optionally and independently substituted with up to 3 instances of halogen; wherein each R^{8b} is independently selected from hydrogen, a C₁₋₄ alkyl, C₁₋₄ haloalkyl, a C₃₋₈ cycloalkyl ring or a C₃₋₈ (halocycloalkyl) ring; and
each R^{7d} is independently selected from hydrogen, halogen, —CN, —NO₂, C₁₋₄ alkyl, C₁₋₄ haloalkyl, C₃₋₈ cycloalkyl ring, —OR^{8c}, —SR^{8c}, —N(R^{8c})₂, or an oxo group; wherein each said cycloalkyl group is optionally and independently substituted with up to 3 instances of halogen; wherein each R^{o} is independently selected from hydrogen, a C₁₋₄ alkyl, C₁₋₄ haloalkyl, a C₃₋₈ cycloalkyl ring or a C₃₋₈ (halocycloalkyl) ring;
each R^{8b} is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{8c} is independently selected from hydrogen, C₁₋₆ alkyl, C₁₋₆ fluoroalkyl, a C₃₋₈ cycloalkyl ring, a 4 to 7-membered heterocyclic ring or a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; and
provided that the compound is not a compound depicted below: 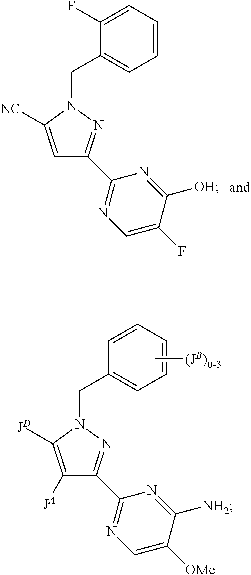
wherein J^{D} is either an ethylene or —N(Me)₂; J^{A} is either hydrogen or methyl and J^{B} is either fluoro or C₁₋₂ alkoxy.

57. The method of claim 1, wherein the sGC stimulator is a compound of Formula I, or a pharmaceutically acceptable salt thereof: 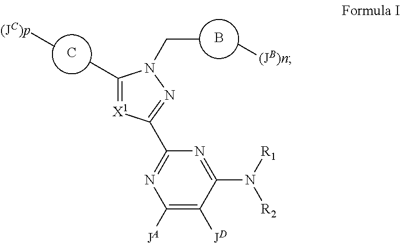
wherein:
X¹ is selected from N, CH, C(C₁₋₄ alkyl), C(C₁₋₄ haloalkyl), CCl and CF;
ring B is a phenyl or a 6-membered heteroaryl ring containing 1 or 2 ring nitrogen atoms, or ring B is a thiophene;
n is 0 or an integer selected from 1 to 3;
each J^{B} is independently selected from halogen, —CN, a C₁₋₆ aliphatic, —OR^{B} or a C₃₋₈ cycloaliphatic ring; wherein each of said C₁₋₆ aliphatic and each of said C₃₋₈ cycloaliphatic group is optionally substituted with up to 3 instances of halogen;
each R^{B} is independently selected from hydrogen, a C₁₋₆ aliphatic or a C₃₋₈ cycloaliphatic ring; wherein each of said C₁₋₆ aliphatic and each said C₃₋₈ cycloaliphatic ring is optionally substituted with up to 3 instances of halogen;
J^{A} is selected from hydrogen, halogen, methyl, methoxy, trifluoromethyl, trifluoromethoxy or —NR^{a}R^{b}, wherein R^{a} and R^{b} are each independently selected from hydrogen, C₁₋₆ alkyl or a 3-6 cycloalkyl ring;
J^{D} is absent or selected from halogen, —CN, —CF₃, methoxy, trifluoromethoxy, nitro, amino or methyl;
R¹ and R², together with the nitrogen atom to which they are attached, form a 4 to 8-membered heterocyclic ring or 5 or 6-membered heteroaryl ring; wherein said 4 to 8-membered heterocyclic ring or 5 or 6-membered heteroaryl ring optionally contains in addition to the nitrogen atom up to 3 ring heteroatoms independently selected from N, O or S, and is optionally substituted by up to 5 instances of R⁵; or
alternatively, R¹ and R² are each independently selected from hydrogen, C₁₋₆ alkyl, a C₃₋₈ cycloalkyl ring, a 4 to 8-membered heterocyclic ring, a 5 or 6-membered heteroaryl or a C₁₋₆ alkyl-R^{Y}; wherein each of said 4 to 8-membered heterocyclic ring and each of said 5 or 6-membered heteroaryl ring contains up to 3 ring heteroatoms independently selected from N, O and S; and wherein each of said C₁₋₆ alkyl, C₃₋₈ cycloalkyl ring, 4 to 8-membered heterocyclic ring group, 5 or 6-membered heteroaryl and the C₁₋₆ alkyl portion of said C₁₋₆ alkyl-R^{Y} is optionally and independently substituted with up to 5 instances of R^{5a}; provided that R¹ and R² are never simultaneously hydrogen;
alternatively, J^{D} and one of R¹ or R² can form a 5-6 membered heterocyclic ring containing up to two heteroatoms selected from O, N and S and optionally substituted with up to 3 instances of oxo or —(Y)—R⁹;
wherein Y is either absent or is a linkage in the form of a C₁₋₆ alkyl chain, optionally substituted by up to 6 instances of fluoro;
each R⁹ is independently selected from hydrogen, fluoro, —CN, —OR¹⁰, —SR¹⁰, —COR¹⁰, —OC(O)R¹⁰, —C(O)OR¹⁰, —C(O)N(R¹⁰)₂, —C(O)N(R¹⁰)SO₂R¹⁰, —N(R¹⁰)C(O)R¹⁰, —N(R¹⁰)C(O)OR¹⁰, —N(R¹⁰)C(O)N(R¹⁰)₂, —N(R¹⁰)₂, —SO₂R¹⁰, —SO₂N(R¹⁰)₂, —SO₂N(R¹⁰)COOR¹⁰, —SO₂N(R¹⁰)C(O)R¹⁰, —N(R¹⁰)SO₂R¹⁰, —(C═O)NHOR¹⁰, a C₃₋₆ cycloalkyl ring, a 4-8-membered heterocyclic ring or a 5-6 membered heteroaroaryl ring; wherein each said 4 to 8-membered heterocyclic ring or 5 to 6-membered heteroaromatic ring contains up to 4 ring heteroatoms independently selected from N, O or S; and wherein each of said C₃₋₆ cycloalkyl rings, each of said 4 to 8-membered heterocyclic rings and each of said 5 to 6-membered heteroaromatic rings is optionally substituted with up to 3 instances of R¹¹;
each R¹¹ is independently selected from halogen, C₁₋₆ alkyl, —CN, —OR¹², —SR¹², —COR¹², —OC(O)R¹², —C(O)OR¹², —C(O)N(R¹²)₂, —C(O)N(R¹²)SO₂R¹², —N(R¹²)C(O)R¹², —N(R¹²)C(O)OR¹², —N(R¹²)C(O)N(R¹²)₂, —N(R¹²)₂, —SO₂R¹², —SO₂N(R¹²)₂, —SO₂N(R¹²)COOR¹², —SO₂N(R¹²)C(O)R¹², —N(R¹²)SO₂R¹² and —N═OR¹²; wherein each of said C₁₋₆ alkyl is optionally and independently substituted by up to 3 instances of fluoro, —OH, —O(C₁₋₄ alkyl), phenyl and —O(C₁₋₄ fluoroalkyl)
wherein each R¹⁰ is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo; and
wherein each R¹² is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, C₁₋₄ (fluoroalkyl), —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ fluoroalkyl) or oxo;
R^{Y} is selected from a C₃₋₈ cycloalkyl ring, a 4 to 8-membered heterocyclic ring, phenyl, or a 5 to 6-membered heteroaromatic ring; wherein each of said 4 to 8-membered heterocyclic ring or 5 to 6-membered heteroaromatic ring contains up to 4 ring heteroatoms independently selected from N, O or S; and wherein each of said C₃₋₈ cycloalkyl ring, each of said 4 to 8-membered heterocyclic ring, each of said phenyl, and each of said 5 to 6-membered heteroaromatic ring is optionally substituted with up to 5 instances of R^{5c};
each R^{5c} is independently selected from halogen, —CN, C₁₋₆ alkyl, —OR^{6b}, —SR^{6b}, —COR^{6b}, —OC(O)R^{6b}, —C(O)OR^{6b}, —C(O)N(R^{6b})₂, —C(O)N(R^{6b})SO₂R^{6b}, —N(R^{6b})C(O)R^{6b}, —N(R^{6b})C(O)OR^{6b}, —N(R^{6b})C(O)N(R^{6b})₂, —N(R^{6b})₂, —SO₂R^{6b}, —SO₂N(R^{6b})₂, —SO₂N(R^{6b})COOR^{6b}, —SO₂N(R^{6b})C(O)R^{6b}, —N(R^{6b})SO₂R^{6b}, —(C═O)NHOR^{6b}, a C₃₋₈ cycloalkyl ring, a 4 to 7-membered heterocyclic ring, a 5 or 6-membered heteroaryl ring, phenyl, benzyl, an oxo group, or a bicyclic group; wherein each of said 5 or 6-membered heteroaryl ring and each of said 4 to 7-membered heterocyclic ring contains up to 4 ring heteroatoms independently selected from N, O and S; and wherein each of said C₁₋₆ alkyl, each of said C₃₋₈ cycloalkyl ring, each of said 4 to 7-membered heterocyclic ring, each of said 5 or 6-membered heteroaryl ring, each of said benzyl and each of said phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains a first ring and a second ring in a fused or bridged relationship, said first ring is a 4 to 7-membered heterocyclic ring, a 5 or 6-membered heteroaryl ring, phenyl or benzyl, and said second ring is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6b} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each 5 or 6-membered heteroaryl ring or 4 to 7-membered heterocyclic ring contains up to 4 ring heteroatoms independently selected from N, O and S; and wherein each of said C₁₋₆ alkyl, each said phenyl, each said benzyl, each said C₃₋₈ cycloalkyl group, each said 4 to 7-membered heterocyclic ring and each 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
two instances of R^{5c} attached to the same or different ring atoms of R^{Y}, together with said ring atom or atoms, may form a C₃₋₈ cycloalkyl ring, a 4 to 6-membered heterocyclic ring; a phenyl or a 5 or 6-membered heteroaryl ring, resulting in a bicyclic system wherein the two rings are in a spiro, fused or bridged relationship, wherein said 4 to 6-membered heterocycle or said 5 or 6-membered heteroaryl ring contains up to three heteroatoms independently selected from N, O or S; and wherein said C₃₋₈ cycloalkyl ring, 4 to 6-membered heterocyclic ring, phenyl or a 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR″(CO)CO(C₁₋₄ alkyl), —OH or halogen; wherein R″ is hydrogen or a C₁₋₂ alkyl;
each R^{5a} is independently selected from halogen, —CN, C₁₋₆ alkyl, —OR^{6a}, —SR^{6a}, —COR^{6a}, —OC(O)R^{6a}, —C(O)OR^{6a}, —C(O)N(R^{6a})₂, —C(O)N(R^{6a})SO₂R^{6a}, —N(R^{6a})C(O)R^{6a}, —N(R^{6a})C(O)OR^{6a}, —N(R^{6a})C(O)N(R^{6a})₂, —N(R^{6a})₂, —SO₂R^{6a}, —SO₂N(R^{6a})₂, —SO₂N(R^{6a})COOR^{6a}, —SO₂N(R^{6a})C(O)R^{6a}, —N(R^{6a})SO₂R^{6a}, —(C═O)NHOR^{6a}, a C₃₋₈ cycloalkyl ring, a 4 to 7-membered heterocyclic ring, a 5 or 6-membered heteroaryl ring, phenyl, benzyl, an oxo group or a bicyclic group; wherein each 5 or 6-membered heteroaryl ring or 4 to 7-membered heterocyclic ring contains up to 4 ring heteroatoms independently selected from N, O and S, wherein each of said C₁₋₆ alkyl, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, C₁₋₄ haloalkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R^{6a} is independently selected from hydrogen, a C₁₋₆ alkyl, phenyl, benzyl, a C₃₋₈ cycloalkyl ring, a 4 to 7-membered heterocyclic ring or a 5 or 6-membered heteroaryl ring, wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —C(O)NH₂, —C(O)N(C₁₋₆ alkyl)₂, —C(O)NH(C₁₋₆ alkyl), —C(O)N(C₁₋₆ haloalkyl)₂, —C(O)NH(C₁₋₆ haloalkyl), C(O)N(C₁₋₆ alkyl)(C₁₋₆ haloalkyl), —COO(C₁₋₆alkyl), —COO(C₁₋₆ haloalkyl), —O(C₁₋₄ alkyl), —O(C₁₋₄ haloalkyl) or oxo, wherein each of said 5 or 6-membered heteroaryl ring or 4 to 7-membered heterocyclic ring contains up to 4 ring heteroatoms independently selected from N, O and S; or
when one of R¹ or R² is the C₃₋₈ cycloalkyl ring, 4 to 8-membered heterocyclic ring or 5 or 6-membered heteroaryl substituted with up to 5 instances of R^{5a}, two of the instances of R^{5a} attached to the same or different ring atoms of said R¹ or R², together with said atom or atoms, may optionally form a C₃₋₈ cycloalkyl ring, a 4 to 6-membered heterocyclic ring, a phenyl or a 5 or 6-membered heterocyclic ring, resulting in a bicyclic system wherein the two rings are in a spiro, fused or bridged relationship, wherein said 4 to 6-membered heterocycle or said 5 or 6-membered heterocyclic ring contains up to two ring heteroatoms independently selected from N, O or S; and wherein said C₃₋₈ cycloalkyl ring, 4 to 6-membered heterocyclic ring, phenyl or 5 or 6-membered heterocyclic ring is optionally substituted by up to 2 instances of C₁₋₄ alkyl, C₁₋₄ haloalkyl, oxo, —(CO)CO(C₁₋₄ alkyl), —NR′(CO)CO(C₁₋₄ alkyl) or halogen; wherein R′ is hydrogen or a C₁₋₂ alkyl;
each R⁵ is independently selected from halogen, —CN, C₁₋₆ alkyl, —OR⁶, —SR⁶, —COR⁶, —OC(O)R⁶, —C(O)OR⁶, —C(O)N(R⁶)₂, —C(O)N(R⁶)SO₂R⁶, —N(R⁶)C(O)R⁶, —N(R⁶)C(O)OR⁶, —N(R⁶)C(O)N(R⁶)₂, —N(R⁶)₂, —SO₂R⁶, —SO₂N(R⁶)₂, —SO₂N(R⁶)COOR⁶, —SO₂N(R⁶)C(O)R⁶, —N(R⁶)SO₂R⁶, —(C═O)NHOR⁶, a C₃₋₈ cycloalkyl ring, a 4 to 7-membered heterocyclic ring, a 5 or 6-membered heteroaryl ring, phenyl, benzyl, an oxo group or a bicyclic group; wherein each of said 5 or 6-membered heteroaryl ring or 4 to 7-membered heterocyclic ring contains up to 4 ring heteroatoms independently selected from N, O and S; and wherein each of said C₁₋₆ alkyl, C₃₋₈ cycloalkyl ring, 4 to 7-membered heterocyclic ring, 5 or 6-membered heteroaryl ring, benzyl or phenyl group is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; wherein said bicyclic group contains ring one and ring two in a fused or bridged relationship, said ring one is a 4 to 7-membered heterocyclic ring, a 5 or 6-membered heteroaryl ring, phenyl or benzyl, and said ring two is a phenyl ring or a 5 or 6-membered heteroaryl ring containing up to 3 ring heteroatoms selected from N, O or S; and wherein said bicyclic group is optionally and independently substituted by up to six instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo;
each R⁶ is independently selected from hydrogen, a C₁₋₆ alkyl, phenyl, benzyl, a C₃₋₈ cycloalkyl ring or a 4 to 7-membered heterocyclic ring, a 5 or 6-membered heteroaryl ring; wherein each of said 5 or 6-membered heteroaryl ring or 4 to 7-membered heterocyclic ring contains up to 4 ring heteroatoms independently selected from N, O and S; and wherein each of said C₁₋₆ alkyl, each of said phenyl, each of said benzyl, each of said C₃₋₈ cycloalkyl group, each of said 4 to 7-membered heterocyclic ring and each of said 5 or 6-membered heteroaryl ring is optionally and independently substituted with up to 3 instances of halogen, C₁₋₄ alkyl, —OH, —NH₂, —NH(C₁₋₄ alkyl), —N(C₁₋₄ alkyl)₂, —CN, —COOH, —COO(C₁₋₄ alkyl), —O(C₁₋₄ alkyl), —O(C₁₋₄ haloalkyl) or oxo; or
when R¹ and R² attached to the nitrogen atom form the 4 to 8-membered heterocyclic ring or 5 or 6-membered heteroaryl ring substituted with up to 5 instances of R⁵, two of the instances of R⁵ attached to the same or different atoms of said ring, together with said atom or atoms, may optionally form a C₃₋₈ cycloalkyl ring, a 4 to 6-membered heterocyclic ring; a phenyl or a 5 or 6-membered heteroaryl ring, resulting in a bicyclic system wherein the two rings of the bicyclic system are in a spiro, fused or bridged relationship, wherein said 4 to 6-membered heterocycle or said 5 or 6-membered heteroaryl ring contains up to three ring heteroatoms independently selected from N, O or S; and wherein said C₃₋₈ cycloalkyl ring, 4 to 6-membered heterocyclic ring, phenyl or 5 or 6-membered heteroaryl ring is optionally and independently substituted by up to 3 instances of C₁₋₄ alkyl, C₁₋₄ haloalkyl, C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen; wherein R is hydrogen or a C₁₋₂ alkyl;
p is an integer selected from 0, 1 or 2;
ring C is a monocyclic 5-membered heteroaryl ring containing up to 4 ring heteroatoms selected from N, O or S; wherein said monocyclic 5-membered heteroaryl ring is not a 1,3,5-triazinyl ring;
each J^{C} is independently selected from halogen or a C₁₋₄ aliphatic optionally and independently substituted by up to 3 instances of C₁₋₄ alkoxy, C₁₋₄ haloalkoxy, oxo, —C(O)O(C₁₋₄ alkyl), —C(O)OH, —NR(CO)CO(C₁₋₄ alkyl), —OH or halogen.

58. The method of claim 1, wherein the sGC stimulator is selected from those depicted in one of Tables XA, XB or XC.

59. The method of claim 1, wherein the sGC stimulator is selected from riociguat, neliciguat, vericiguat, BAY-41-2272, BAY 41-8543, and etriciguat.
